(19) United States
(12) Patent Application Publication
(10) Pub. No.: US 2024/0167057 A1
(43) Pub. Date: May 23, 2024

(54) MODIFIED ALPHAVIRUS VECTORS

(71) Applicant: Gritstone bio, Inc., Emeryville, CA (US)

(72) Inventor: Sue-Jean Hong, Emeryville, CA (US)

(21) Appl. No.: 18/354,193

(22) Filed: Jul. 18, 2023

Related U.S. Application Data
(63) Continuation of application No. PCT/US2022/013004, filed on Jan. 19, 2022.
(60) Provisional application No. 63/139,297, filed on Jan. 19, 2021.

(51) Int. Cl. C12N 15/86; A61P 37/04

(52) U.S. Cl. CPC C12N 15/86; A61P 37/04; C12N 2770/36134; C12N 2770/36145

(57) ABSTRACT

Disclosed herein are vaccine compositions that include alphavirus derived vectors having multiple expression cassettes driven by multiple subgenomic promoters.

### CROSS REFERENCE TO RELATED APPLICATIONS

This application is a continuation of International Application No. PCT/US2022/013004, filed Jan. 19, 2022, which claims the benefit of U.S. Provisional Application No. 63/139,297, filed Jan. 19, 2021, which is hereby incorporated in its entirety by reference for all purposes.

### SEQUENCE LISTING

The instant application contains a Sequence Listing which has been submitted electronically in .xml format and is hereby incorporated by reference in its entirety. Said .xml copy, created on Oct. 18, 2023, is named GSO-101WOC1 and is 46,329,744 bytes in size.

### BACKGROUND

Alphaviruses are a group of small positive-sense single-stranded RNA viruses that are responsible for many diseases in humans and other animals. See, e.g., “The Alphaviruses: Gene Expression, Replication and Evolution,” Microbiological Reviews, September 1994, p. 491-562; Jose et al., “A structural and functional perspective of alphavirus replication and assembly,” Future Micriobol., 2009, v.4:837-856. Because of their high replication efficiency and specificity, alphaviruses have proven to be useful in the engineering of self-replicating RNA vectors for the expression of heterologous proteins in mammalian cells. See, e.g., Frolov et al., “Alphavirus-based expression vectors: strategies and applications”, PNAS, 1996, v. 93, pp. 11371-11377; Young Kim, et al., “Enhancement of protein expression by alphavirus replicons by designing self-replicating subgenomic RNAs”, PNAS, 2014, v.11:29, pp. 10708-10713. While some progress has been made in this field, improvements are needed in areas including: the efficiency of delivery of RNA vectors to targeted cells, the efficiency of expression of heterologous proteins from RNA vectors, the ease of “personalization” of the proteins to be expressed from RNA vectors, improvements in the immune response when certain heterologous proteins (e.g., neoantigens) are expressed from RNA vectors, and/or improvements in the efficiency of manufacture of such RNA vectors.

### SUMMARY

Provided for herein is a composition for delivery of an antigen expression system comprising a self-replicating alphavirus-based expression system, wherein the composition for delivery of the self-replicating alphavirus-based expression system comprises: the self-replicating alphavirus-based expression system, wherein the self-replicating alphavirus-based expression system comprises one or more vectors, wherein the one or more vectors comprise: (a) an RNA alphavirus backbone, wherein the RNA alphavirus backbone comprises: (i) at least one promoter nucleotide sequence, and (ii) at least one polyadenylation (poly(A)) sequence; and (b) at least two cassettes, wherein each of the cassettes independently comprise: (i) at least one antigen-encoding nucleic acid sequence comprising: a. an epitope-encoding nucleic acid sequence, b. optionally a 5′ linker sequence, and c. optionally a 3′ linker sequence; and (ii) optionally, at least one second poly(A) sequence, wherein the second poly(A) sequence is a native poly(A) sequence or an exogenous poly(A) sequence to the alphavirus, wherein a first of the at least two cassettes, oriented from 5′ to 3′, is operably linked to a promoter nucleotide sequence comprising a first subgenomic alphavirus-derived promoter (SGP1) comprising a core conserved promoter sequence comprising the polynucleotide sequence ctacggcTAAcctgaa(+1)tgga (SEQ ID NO: 27974), and wherein at least a second of the at least two cassettes is operably linked to a promoter nucleotide sequence comprising a second subgenomic alphavirus-derived promoter (SGP2) comprising the core conserved promoter sequence, and wherein the SGP1 and/or the SGP2 subgenomic promoter comprises an extended 3′ promoter region derived from an alphavirus encoded 3′ of the core conserved promoter sequence.

Also provided for herein is a composition for delivery of a payload comprising a self-replicating alphavirus-based expression system, wherein the composition for delivery of the self-replicating alphavirus-based expression system comprises: (A) the self-replicating alphavirus-based expression system, wherein the self-replicating alphavirus-based expression system comprises one or more vectors, wherein the one or more vectors comprise: (a) an RNA alphavirus backbone, wherein the RNA alphavirus backbone comprises: (i) at least one promoter nucleotide sequence, and (ii) at least one polyadenylation (poly(A)) sequence; and (b) at least two cassettes, wherein each of the cassettes independently comprise: (i) at least one payload-encoding nucleic acid sequence; and (ii) optionally, at least one second poly(A) sequence, wherein the second poly(A) sequence is a native poly(A) sequence or an exogenous poly(A) sequence to the alphavirus, wherein a first of the at least two cassettes, oriented from 5′ to 3′, is operably linked to a promoter nucleotide sequence comprising a first subgenomic alphavirus-derived promoter (SGP1) comprising a core conserved promoter sequence comprising the polynucleotide sequence ctacggcTAAcctgaa(+1)tgga (SEQ ID NO: 27974), and wherein at least a second of the at least two cassettes is operably linked to a promoter nucleotide sequence comprising a second subgenomic alphavirus-derived promoter (SGP2) comprising the core conserved promoter sequence, and wherein the SGP1 and/or the SGP2 subgenomic promoter comprises an extended 3′ promoter region derived from an alphavirus encoded 3′ of the core conserved promoter sequence.

In some aspects, the extended 3′ promoter region of SGP1 is different than the extended 3′ promoter region of SGP2.

In some aspects, either the SGP1 or the SGP2 subgenomic promoter, but not both, comprises an extended 3′ promoter region derived from an alphavirus encoded 3′ of the core conserved promoter sequence.

In some aspects, the extended 3′ promoter region comprises the polynucleotide sequence CTACGACAT (SEQ ID NO: 27997). In some aspects, the extended 3′ promoter region comprises the polynucleotide sequence CTACGACATAGTCTAGTCCGCCAAG (SEQ ID NO: 27975). In some aspects, the extended 3′ promoter region consists of the polynucleotide sequence CTACGACAT (SEQ ID NO: 27997). In some aspects, the extended 3′ promoter region consists of the polynucleotide sequence CTACGACATAGTCTAGTCCGCCAAG (SEQ ID NO: 27975). In some aspects, the extended 3′ promoter region comprises the polynucleotide sequence ATAGTCTAGTCCGCCAAG (SEQ ID NO: 27976).

In some aspects, (a) each of the subgenomic promoters comprise an extended 3′ promoter region comprising the polynucleotide sequence CTACGACAT (SEQ ID NO: 27997); and (b) only one of the SGP1 or the SGP2 subgenomic promoters, but not both, comprise an extended 3′ promoter region further comprising the polynucleotide sequence ATAGTCTAGTCCGCCAAG (SEQ ID NO: 27976), wherein the polynucleotide sequence ATAGTCTAGTCCGCCAAG (SEQ ID NO: 27976) is encoded 3′ of the polynucleotide sequence CTACGACAT (SEQ ID NO: 27997).

In some aspects, the SGP1 subgenomic promoter, the SGP2 subgenomic promoter, or both comprise an extended 5′ promoter region derived from an alphavirus encoded 5′ of the core conserved promoter sequence. In some aspects, the extended 5′ promoter region comprises a polynucleotide sequence derived from an alphavirus nonstructural protein 4 (nsp4) and is encoded 5′ of the core conserved promoter sequence. In some aspects, the extended 5′ promoter region is encoded immediately 5′ of the core conserved promoter sequence. In some aspects, the extended 5′ promoter region comprises the polynucleotide sequence ctct encoded immediately 5′ of the core conserved promoter sequence.

In some aspects, the extended 5′ promoter region comprises the polynucleotide sequence acttccatcatagttatggccatgactactctagctagcagtgttaaatcattcagctacctgagaggggcccctataactct (SEQ ID NO: 27977) encoded immediately 5′ of the core conserved promoter sequence. In some aspects, the extended 5′ promoter region comprises the polynucleotide sequence acctgagaggggcccctataactct (SEQ ID NO: 27978) encoded immediately 5′ of the core conserved promoter sequence. In some aspects, the extended 5′ promoter region comprises the polynucleotide sequence gggcccctataactct (SEQ ID NO: 27979) encoded immediately 5′ of the core conserved promoter sequence. In some aspects, the extended 5′ promoter region consists of the polynucleotide sequence gggcccctataactct (SEQ ID NO: 27979) encoded immediately 5′ of the core conserved promoter sequence.

In some aspects, the extended 5′ promoter region of the SGP2 subgenomic promoter comprises the polynucleotide sequence acttccatcatagttatggccatgactactctagctagcagtgttaaatcattcagctacctgagaggggcccctataactct (SEQ ID NO: 27977) encoded immediately 5′ of the core conserved promoter sequence. In some aspects, the extended 5′ promoter region of the SGP2 subgenomic promoter comprises the polynucleotide sequence acctgagaggggcccctataactct (SEQ ID NO: 27978) encoded immediately 5′ of the core conserved promoter sequence. In some aspects, the extended 5′ promoter region of the SGP2 subgenomic promoter comprises the polynucleotide sequence gggcccctataactct (SEQ ID NO: 27979) encoded immediately 5′ of the core conserved promoter sequence. In some aspects, the extended 5′ promoter region of the SGP2 subgenomic promoter consists of the polynucleotide sequence gggcccctataactct (SEQ ID NO: 27979) encoded immediately 5′ of the core conserved promoter sequence.

In some aspects, the at least one promoter nucleotide sequence of the RNA alphavirus backbone comprises the SGP1 subgenomic promoter.

In some aspects, the extended 3′ promoter region and/or the extended 5′ promoter region is derived from the same alphavirus as the alphavirus used to derive the core conserved promoter sequence.

In some aspects, the extended 3′ promoter region and/or the extended 5′ promoter region is capable of reducing or eliminating recombination between the SGP1 and the SGP2 subgenomic promoter. In some aspects, the extended 3′ promoter region and/or the extended 5′ promoter region comprises one or more transcriptional enhancer elements. In some aspects, the SGP2 subgenomic promoter is capable of promoting expression of a cassette at least 2-fold greater relative to the same cassette operably linked to the SGP1 subgenomic promoter. In some aspects, the extended 3′ promoter region and/or the extended 5′ promoter region of SGP2 is capable of promoting expression of a cassette at least 2-fold greater relative to the same cassette operably linked to the SGP1 subgenomic promoter. In some aspects, the SGP2 subgenomic promoter is capable of stimulating a stronger immune response to epitopes encoded by the cassette operably linked to the SGP2 subgenomic promoter following administration to a subject relative to the same cassette operably linked to the SGP1 subgenomic promoter. In some aspects, the extended 3′ promoter region and/or the extended 5′ promoter region of SGP2 is capable of stimulating a stronger immune response to epitopes encoded by the cassette operably linked to the SGP2 subgenomic promoter following administration to a subject relative to the same cassette operably linked to the SGP1 subgenomic promoter.

In some aspects, the SGP2 subgenomic promoter is encoded immediately 5′ of the second cassette, optionally immediately 5′ of a Kozak sequence of the second cassette.

In some aspects, either the SGP1 or the SGP2 subgenomic promoter comprises the polynucleotide sequence GGGCCCCTATAACTCTCTACGGCTAACCTGAATGGACTACGACATAGTCTAGTCCGC CAAG (SEQ ID NO: 27980). In some aspects, the SGP2 subgenomic promoter comprises the polynucleotide sequence GGGCCCCTATAACTCTCTACGGCTAACCTGAATGGACTACGACATAGTCTAGTCCGC CAAG (SEQ ID NO: 27980). In some aspects, the SGP1 subgenomic promoter comprises the polynucleotide sequence GGGCCCCTATAACTCTCTACGGCTAACCTGAATGGACTACGAC (SEQ ID NO: 27981).

In some aspects, (a) either the SGP1 or the SGP2 subgenomic promoter comprises the polynucleotide sequence GGGCCCCTATAACTCTCTACGGCTAACCTGAATGGACTACGACATAGTCTAGTCCGC CAAG (SEQ ID NO: 27980); and (b) the other subgenomic promoter comprises the polynucleotide sequence GGGCCCCTATAACTCTCTACGGCTAACCTGAATGGACTACGAC (SEQ ID NO: 27981) but does not comprise the polynucleotide sequence ATAGTCTAGTCCGCCAAG (SEQ ID NO: 27976). In some aspects, the SGP2 subgenomic promoter comprises the polynucleotide sequence GGGCCCCTATAACTCTCTACGGCTAACCTGAATGGACTACGAC (SEQ ID NO: 27981) and the SGP1 subgenomic promoter comprises the polynucleotide sequence GGGCCCCTATAACTCTCTACGGCTAACCTGAATGGACTACGAC (SEQ ID NO: 27981), wherein the SGP1 subgenomic promoter does not comprise the polynucleotide sequence ATAGTCTAGTCCGCCAAG (SEQ ID NO: 27976).

In some aspects, an ordered sequence of one or more of the nucleic acid sequences encoding the immunogenic polypeptide is described in the formula, from 5′ to 3′, comprising: P1-(L5b-Nc-L3d)X-P2-(L5b-Nc-L3d)X-Pa-(L5b-Nc-L3d)X-(G5e-Uf)Y-G3g wherein P1 comprises the SGP1 subgenomic promoter, P2 comprises the SGP2 subgenomic promoter where for Pa a=0 or 1 for additional cassettes, N comprises the epitope-encoding nucleic acid sequence, where c=1, L5 comprises the 5′ linker sequence, where b=0 or 1, L3 comprises the 3′ linker sequence, where d=0 or 1, G5 comprises one of the at least one nucleic acid sequences encoding a GPGPG amino acid linker (SEQ ID NO: 56), where e=0 or 1, G3 comprises one of the at least one nucleic acid sequences encoding a GPGPG amino acid linker (SEQ ID NO: 56), where g=0 or 1, U comprises one of the at least one MHC class II epitope-encoding nucleic acid sequence, where f=1, X=1 to 400, where for each X the corresponding Nc is a corresponding epitope-encoding nucleic acid sequence, and Y=0, 1, or 2, where for each Y the corresponding Uf is a universal MHC class II epitope-encoding nucleic acid sequence, optionally wherein the at least one universal sequence comprises at least one of Tetanus toxoid and PADRE.

In some aspects, for each X the corresponding Nc is a distinct epitope-encoding nucleic acid sequence. In some aspects, for each Y the corresponding Uf is a distinct MHC class II epitope-encoding nucleic acid sequence. In some aspects, each N encodes a MHC class I epitope 7-15 amino acids in length, a MHC class II epitope, an epitope capable of stimulating a B cell response, or combinations thereof, L5 is a native 5′ linker sequence that encodes a native N-terminal amino acid sequence of the epitope, and wherein the 5′ linker sequence encodes a peptide that is at least 2 amino acids in length, L3 is a native 3′ linker sequence that encodes a native C-terminal amino acid sequence of the epitope, and wherein the 3′ linker sequence encodes a peptide that is at least 2 amino acids in length.

In some aspects, the epitope-encoding nucleic acid sequence comprises: at least one alteration that makes the encoded epitope sequence distinct from the corresponding peptide sequence encoded by a wild-type nucleic acid sequence; a nucleic acid sequence encoding an infectious disease organism peptide selected from the group consisting of: a pathogen-derived peptide, a virus-derived peptide, a bacteria-derived peptide, a fungus-derived peptide, and a parasite-derived peptide, and optionally wherein the epitope-encoding nucleic acid sequence encodes a MHC class I or MHC class II epitope; or combinations thereof.

In some aspects, the composition further comprises a nanoparticulate delivery vehicle In some aspects, the nanoparticulate delivery vehicle is a lipid nanoparticle (LNP). In some aspects, the LNP comprises ionizable amino lipids. In some aspects, the ionizable amino lipids comprise MC3-like (dilinoleylmethyl-4-dimethylaminobutyrate) molecules. In some aspects, the nanoparticulate delivery vehicle encapsulates the antigen expression system.

In some aspects, the backbone comprises at least one nucleotide sequence of an Aura virus, a Fort Morgan virus, a Venezuelan equine encephalitis virus, a Ross River virus, a Semliki Forest virus, a Sindbis virus, or a Mayaro virus. In some aspects, the backbone comprises at least one nucleotide sequence of a Venezuelan equine encephalitis virus. In some aspects, the backbone comprises at least sequences for nonstructural protein-mediated amplification, a 26S promoter sequence, a poly(A) sequence, a nonstructural protein 1 (nsP1) gene, a nsP2 gene, a nsP3 gene, and a nsP4 gene encoded by the nucleotide sequence of the Aura virus, the Fort Morgan virus, the Venezuelan equine encephalitis virus, the Ross River virus, the Semliki Forest virus, the Sindbis virus, or the Mayaro virus. In some aspects, the backbone comprises at least sequences for nonstructural protein-mediated amplification, a 26S promoter sequence, and a poly(A) sequence encoded by the nucleotide sequence of the Aura virus, the Fort Morgan virus, the Venezuelan equine encephalitis virus, the Ross River virus, the Semliki Forest virus, the Sindbis virus, or the Mayaro virus. In some aspects, sequences for nonstructural protein-mediated amplification are selected from the group consisting of: an alphavirus 5′ UTR, a 51-nt CSE, a 24-nt CSE, a 26S subgenomic promoter sequence, a 19-nt CSE, an alphavirus 3′ UTR, or combinations thereof. In some aspects, the backbone does not encode structural virion proteins capsid, E2 and E1. In some aspects, the cassettes are inserted in place of structural virion proteins within the nucleotide sequence of the Aura virus, the Fort Morgan virus, the Venezuelan equine encephalitis virus, the Ross River virus, the Semliki Forest virus, the Sindbis virus, or the Mayaro virus. In some aspects, the Venezuelan equine encephalitis virus comprises the sequence of SEQ ID NO:3 or SEQ ID NO:5. In some aspects, the Venezuelan equine encephalitis virus comprises the sequence of SEQ ID NO:3 or SEQ ID NO:5 further comprising a deletion between base pair 7544 and 11176. In some aspects, the backbone comprises the sequence set forth in SEQ ID NO:6 or SEQ ID NO:7. In some aspects, the cassettes are inserted at position 7544 to replace the deletion between base pairs 7544 and 11176 as set forth in the sequence of SEQ ID NO:3 or SEQ ID NO:5.

In some aspects, one or more of the cassettes are at least 100, 200, 300, 400, 500, 600, 700, 800, or 900 nucleotides in length. In some aspects, one or more of the cassettes are at least 1000, 2000, 3000, 4000, 5000, 6000, 7000, 8000, 9000, or 10000 nucleotides in length. In some aspects, the one or more vectors are capable of driving expression of a cassette that is at least 3500 nucleotides in length. In some aspects, the one or more vectors are capable of driving expression of a cassette that is at least 6000 nucleotides in length.

In some aspects, at least one of the at least one antigen-encoding nucleic acid sequences comprises an epitope-encoding nucleic acid sequence that encodes an epitope that is presented by MHC class I. In some aspects, at least one of the at least one antigen-encoding nucleic acid sequences comprises an epitope-encoding nucleic acid sequence that encodes an epitope that is presented by MHC class II. In some aspects, at least one of the at least one antigen-encoding nucleic acid sequences comprises an epitope-encoding nucleic acid sequence that encodes a polypeptide sequence or portion thereof capable of stimulating a B cell response, optionally wherein the polypeptide sequence or portion thereof capable of stimulating a B cell response comprises a full-length protein, a protein domain, a protein subunit, or an antigenic fragment predicted or known to be capable of being bound by an antibody.

In some aspects, the at least one antigen-encoding nucleic acid sequence comprises two or more antigen-encoding nucleic acid sequences. In some aspects, each antigen-encoding nucleic acid sequence is linked directly to one another. In some aspects, each antigen-encoding nucleic acid sequence is linked to a distinct antigen-encoding nucleic acid sequence with a nucleic acid sequence encoding a linker.

In some aspects, at least one of the at least one antigen-encoding nucleic acid sequences comprises an epitope-encoding nucleic acid sequence that encodes two or more distinct epitopes predicted or validated to be capable of presentation by at least one HLA allele. In some aspects, the at least one antigen-encoding nucleic acid sequence comprises at least 2-10, 2, 3, 4, 5, 6, 7, 8, 9, or 10 nucleic acid sequences. In some aspects, the at least one antigen-encoding nucleic acid sequence comprises at least 11-20, 15-20, 11-100, 11-200, 11-300, 11-400, 11, 12, 13, 14, 15, 16, 17, 18, 19, 20 or up to 400 nucleic acid sequences. In some aspects, the at least one antigen-encoding nucleic acid sequence comprises at least 2-400 nucleic acid sequences and wherein at least two of the antigen-encoding nucleic acid sequences comprises an epitope-encoding nucleic acid sequence that encodes polypeptide sequences or portions thereof that are (1) presented by MHC class I, (2) presented by MHC class II, and/or (3) capable of stimulating a B cell response. In some aspects, at least two of the antigen-encoding nucleic acid sequences comprises an epitope-encoding nucleic acid sequence that encodes polypeptide sequences or portions thereof that are (1) presented by MHC class I, (2) presented by MHC class II, and/or (3) capable of stimulating a B cell response class.

In some aspects, when administered to the subject and translated, at least one of the epitopes encoded by the at least one epitopes-encoding nucleic acid sequence are presented on antigen presenting cells resulting in an immune response targeting at least one of the antigens on a cell surface. In some aspects, when administered to the subject and translated, at least one of the antigens encoded by the at least one antigen-encoding nucleic acid sequence results in an antibody response targeting at least one of the antigens. In some aspects, the at least one antigen-encoding nucleic acid sequences when administered to the subject and translated, at least one of the MHC class I or class II antigens are presented on antigen presenting cells resulting in an immune response targeting at least one of the antigens on a cell surface, and optionally wherein the expression of each of the at least one antigen-encoding nucleic acid sequences is driven by the at least one promoter nucleotide sequence.

In some aspects, each MHC class I epitope-encoding nucleic acid sequence encodes a polypeptide sequence between 8 and 35 amino acids in length, optionally 9-17, 9-25, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids in length. In some aspects, the at least one MHC class II epitope-encoding nucleic acid sequence is present. In some aspects, the at least one MHC class II epitope-encoding nucleic acid sequence is present and comprises at least one MHC class II nucleic acid sequence. In some aspects, the at least one MHC class II epitope-encoding nucleic acid sequence is 12-20, 12, 13, 14, 15, 16, 17, 18, 19, 20, or 20-40 amino acids in length. In some aspects, the at least one MHC class II epitope-encoding nucleic acid sequence is present and comprises at least one universal MHC class II epitope-encoding nucleic acid sequence, optionally wherein the at least one universal sequence comprises at least one of Tetanus toxoid and PADRE, and/or at least one MHC class II epitope-encoding nucleic acid sequence.

In some aspects, the at least one promoter nucleotide sequence or the second promoter nucleotide sequence is inducible. In some aspects, the at least one promoter nucleotide sequence or the second promoter nucleotide sequence is non-inducible. In some aspects, the at least one poly(A) sequence comprises a poly(A) sequence native to the backbone. In some aspects, the at least one poly(A) sequence comprises a poly(A) sequence exogenous to the backbone. In some aspects, the at least one poly(A) sequence is operably linked to at least one of the at least one antigen-encoding nucleic acid sequences. In some aspects, the at least one poly(A) sequence is at least 20, at least 30, at least 40, at least 50, at least 60, at least 70, at least 80, or at least 90 consecutive A nucleotides (SEQ ID NO: 27943). In some aspects, the at least one poly(A) sequence is at least 80 consecutive A nucleotides (SEQ ID NO: 27940). In some aspects, the at least one second poly(A) sequence is present. In some aspects, the at least one second poly(A) sequence comprises an SV40 poly(A) signal sequence or a Bovine Growth Hormone (BGH) poly(A) signal sequence, or a combination of two more SV40 poly(A) signal sequences or BGH poly(A) signal sequence. In some aspects, the at least one second poly(A) sequence comprises two or more second poly(A) sequences, optionally wherein the two or more second poly(A) sequences comprises two or more SV40 poly(A) signal sequences two or more BGH poly(A) signal sequences, or a combination of SV40 poly(A) signal sequences and BGH poly(A) signal sequences.

In some aspects, the antigen cassette further comprises at least one of: an intron sequence, an exogenous intron sequence, a Constitutive Transport Element (CTE), a RNA Transport Element (RTE), a woodchuck hepatitis virus posttranscriptional regulatory element (WPRE) sequence, an internal ribosome entry sequence (IRES) sequence, a nucleotide sequence encoding a 2A self cleaving peptide sequence, a nucleotide sequence encoding a Furin cleavage site, or a sequence in the 5′ or 3′ non-coding region known to enhance the nuclear export, stability, or translation efficiency of mRNA that is operably linked to at least one of the at least one antigen-encoding nucleic acid sequences. In some aspects, the antigen cassette further comprises a reporter gene, including but not limited to, green fluorescent protein (GFP), a GFP variant, secreted alkaline phosphatase, luciferase, a luciferase variant, or a detectable peptide or epitope. In some aspects, the detectable peptide or epitope is selected from the group consisting of an HA tag, a Flag tag, a His-tag, or a V5 tag.

In some aspects, the one or more vectors further comprises one or more nucleic acid sequences encoding at least one immune modulator. In some aspects, the immune modulator is an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof. In some aspects, the antibody or antigen-binding fragment thereof is a Fab fragment, a Fab′ fragment, a single chain Fv (scFv), a single domain antibody (sdAb) either as single specific or multiple specificities linked together (e.g., camelid antibody domains), or full-length single-chain antibody (e.g., full-length IgG with heavy and light chains linked by a flexible linker). In some aspects, the heavy and light chain sequences of the antibody are a contiguous sequence separated by either a self-cleaving sequence such as 2A or IRES; or the heavy and light chain sequences of the antibody are linked by a flexible linker such as consecutive glycine residues. In some aspects, the immune modulator is a cytokine. In some aspects, the cytokine is at least one of IL-2, IL-7, IL-12, IL-15, or IL-21 or variants thereof of each.

Also provided herein is a vector or set of vectors comprising the nucleotide sequence of any of the compositions described herein.

Also provided herein is an isolated cell comprising the nucleotide sequence or set of isolated nucleotide sequences of any of the compositions described herein, optionally wherein the cell is a BHK-21, CHO, HEK293 or variants thereof, 911, HeLa, A549, LP-293, PER.C6, or AE1-2a cell.

Also provided herein is a kit comprising the composition of any of the compositions described herein and instructions for use.

Also provided herein is a method for treating a subject, the method comprising administering to the subject any of the compositions described herein or any of the pharmaceutical compositions described herein.

Also provided herein is a method for inducing an immune response in a subject, the method comprising administering to the subject any of the compositions described herein or any of the pharmaceutical compositions described herein.

In some aspects, the subject expresses at least one HLA allele predicted or known to present a MHC class I or MHC class II epitope encoded by the epitope-encoding nucleic acid sequence of the at least one antigen-encoding nucleic acid sequence.

In some aspects, any of the above compositions further comprise a nanoparticulate delivery vehicle. The nanoparticulate delivery vehicle, in some aspects, may be a lipid nanoparticle (LNP). In some aspects, the LNP comprises ionizable amino lipids. In some aspects, the ionizable amino lipids comprise MC3-like (dilinoleylmethyl-4-dimethylaminobutyrate) molecules. In some aspects, the nanoparticulate delivery vehicle encapsulates the antigen expression system.

In some aspects, any of the above compositions further comprise a plurality of LNPs, wherein the LNPs comprise: the antigen expression system; a cationic lipid; a non-cationic lipid; and a conjugated lipid that inhibits aggregation of the LNPs, wherein at least about 95% of the LNPs in the plurality of LNPs either: have a non-lamellar morphology; or are electron-dense.

In some aspects, the non-cationic lipid is a mixture of (1) a phospholipid and (2) cholesterol or a cholesterol derivative.

In some aspects, the conjugated lipid that inhibits aggregation of the LNPs is a polyethyleneglycol (PEG)-lipid conjugate. In some aspects, the PEG-lipid conjugate is selected from the group consisting of: a PEG-diacylglycerol (PEG-DAG) conjugate, a PEG dialkyloxypropyl (PEG-DAA) conjugate, a PEG-phospholipid conjugate, a PEG-ceramide (PEG-Cer) conjugate, and a mixture thereof. In some aspects the PEG-DAA conjugate is a member selected from the group consisting of: a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, a PEG-distearyloxypropyl (C₁₈) conjugate, and a mixture thereof.

In some aspects, the antigen expression system is fully encapsulated in the LNPs.

In some aspects, the non-lamellar morphology of the LNPs comprises an inverse hexagonal (H_{II}) or cubic phase structure.

In some aspects, the cationic lipid comprises from about 10 mol % to about 50 mol % of the total lipid present in the LNPs. In some aspects, the cationic lipid comprises from about 20 mol % to about 50 mol % of the total lipid present in the LNPs. In some aspects, the cationic lipid comprises from about 20 mol % to about 40 mol % of the total lipid present in the LNPs.

In some aspects, the non-cationic lipid comprises from about 10 mol % to about 60 mol % of the total lipid present in the LNPs. In some aspects, the non-cationic lipid comprises from about 20 mol % to about 55 mol % of the total lipid present in the LNPs. In some aspects, the non-cationic lipid comprises from about 25 mol % to about 50 mol % of the total lipid present in the LNPs.

In some aspects, the conjugated lipid comprises from about 0.5 mol % to about 20 mol % of the total lipid present in the LNPs. In some aspects, the conjugated lipid comprises from about 2 mol % to about 20 mol % of the total lipid present in the LNPs. In some aspects, the conjugated lipid comprises from about 1.5 mol % to about 18 mol % of the total lipid present in the LNPs.

In some aspects, greater than 95% of the LNPs have a non-lamellar morphology. In some aspects, greater than 95% of the LNPs are electron dense.

In some aspects, any of the above compositions further comprise a plurality of LNPs, wherein the LNPs comprise: a cationic lipid comprising from 50 mol % to 65 mol % of the total lipid present in the LNPs; a conjugated lipid that inhibits aggregation of LNPs comprising from 0.5 mol % to 2 mol % of the total lipid present in the LNPs; and a non-cationic lipid comprising either: a mixture of a phospholipid and cholesterol or a derivative thereof, wherein the phospholipid comprises from 4 mol % to 10 mol % of the total lipid present in the LNPs and the cholesterol or derivative thereof comprises from 30 mol % to 40 mol % of the total lipid present in the LNPs; a mixture of a phospholipid and cholesterol or a derivative thereof, wherein the phospholipid comprises from 3 mol % to 15 mol % of the total lipid present in the LNPs and the cholesterol or derivative thereof comprises from 30 mol % to 40 mol % of the total lipid present in the LNPs; or up to 49.5 mol % of the total lipid present in the LNPs and comprising a mixture of a phospholipid and cholesterol or a derivative thereof, wherein the cholesterol or derivative thereof comprises from 30 mol % to 40 mol % of the total lipid present in the LNPs.

In some aspects, any of the above compositions further comprise a plurality of LNPs, wherein the LNPs comprise: a cationic lipid comprising from 50 mol % to 85 mol % of the total lipid present in the LNPs; a conjugated lipid that inhibits aggregation of LNPs comprising from 0.5 mol % to 2 mol % of the total lipid present in the LNPs; and a non-cationic lipid comprising from 13 mol % to 49.5 mol % of the total lipid present in the LNPs.

In some aspects, the phospholipid comprises dipalmitoylphosphatidylcholine (DPPC), distearoylphosphatidylcholine (DSPC), or a mixture thereof.

In some aspects, the conjugated lipid comprises a polyethyleneglycol (PEG)-lipid conjugate. In some aspects, the PEG-lipid conjugate comprises a PEG-diacylglycerol (PEG-DAG) conjugate, a PEG-dialkyloxypropyl (PEG-DAA) conjugate, or a mixture thereof. In some aspects, the PEG-DAA conjugate comprises a PEG-dimyristyloxypropyl (PEG-DMA) conjugate, a PEG-distearyloxypropyl (PEG-DSA) conjugate, or a mixture thereof. In some aspects, the PEG portion of the conjugate has an average molecular weight of about 2,000 daltons.

In some aspects, the conjugated lipid comprises from 1 mol % to 2 mol % of the total lipid present in the LNPs.

In some aspects, the LNP comprises a compound having a structure of Formula I:

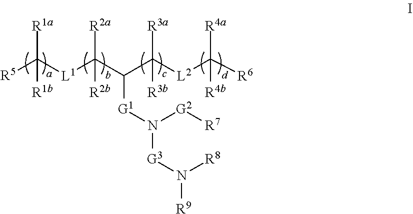

or a pharmaceutically acceptable salt, tautomer, prodrug or stereoisomer thereof, wherein: L¹ and L² are each independently -0(C=0)-, —(C=0)0-, —C(=0)-, -0-, —S(0)ₓ-, —S—S—, —C(=0)S—, —SC(=0)-, —R^{a}C(=0)-, —C(=0) R^{a}—, —R^{a}C(=0) R^{a}—, —OC(=0) R^{a}—, —R^{a}C(=0)0- or a direct bond; G¹ is Ci-C₂ alkylene, —(C=0)-, -0(C=0)-, —SC(=0)-, —R^{a}C(=0)- or a direct bond: —C(=0)-, —(C=0)0-, —C(=0)S—, —C(=0) R^{a}— or a direct bond; G is Ci-C₆ alkylene; R^{a} is H or C1-C12 alkyl; R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond; R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond; R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond; R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond; R⁵ and R⁶ are each independently H or methyl; R⁷ is C₄-C₂₀ alkyl; R⁸ and R⁹ are each independently C₁-C₁₂ alkyl; or R⁸ and R⁹, together with the nitrogen atom to which they are attached, form a 5, 6 or 7-membered heterocyclic ring; a, b, c and d are each independently an integer from 1 to 24; and x is 0, 1 or 2.

In some aspects, the LNP comprises a compound having a structure of Formula II:

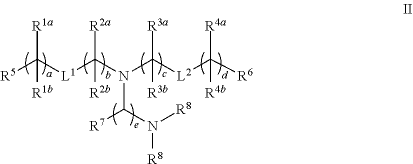

or a pharmaceutically acceptable salt, tautomer, prodrug or stereoisomer thereof, wherein:
L¹ and L² are each independently -0(C=0)-, —(C=0)0- or a carbon-carbon double bond; R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl; R⁷ is, at each occurrence, independently H or C₁-C₁₂ alkyl; R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom; a and d are each independently an integer from 0 to 24; b and c are each independently an integer from 1 to 24; and e is 1 or 2, provided that: at least one of R^{1a}, R^{2a}, R^{3a} or R^{4a} is C₁-C₁₂ alkyl, or at least one of L¹ or L² is -0(C=0)- or —(C=0)0-; and R^{1a} and R^{1b} are not isopropyl when a is 6 or n-butyl when a is 8.

In some aspects, any of the above compositions further comprise one or more excipients comprising a neutral lipid, a steroid, and a polymer conjugated lipid. In some aspects, the neutral lipid comprises at least one of 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), and 1,2-Dioleoyl-sn-glycero-3-phosphoethanolamine (DOPE). In some aspects, the neutral lipid is DSPC.

In some aspects, the molar ratio of the compound to the neutral lipid ranges from about 2:1 to about 8:1.

In some aspects, the steroid is cholesterol. In some aspects, the molar ratio of the compound to cholesterol ranges from about 2:1 to 1:1.

In some aspects, the polymer conjugated lipid is a pegylated lipid. In some aspects, the molar ratio of the compound to the pegylated lipid ranges from about 100:1 to about 25:1. In some aspects, the pegylated lipid is PEG-DAG, a PEG polyethylene (PEG-PE), a PEG-succinoyl-diacylglycerol (PEG-S-DAG), PEG-cer or a PEG dialkyoxypropylcarbamate. In some aspects, the pegylated lipid has the following structure III:

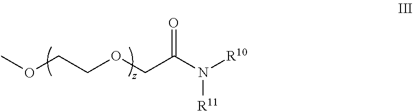

or a pharmaceutically acceptable salt, tautomer or stereoisomer thereof, wherein: R¹⁰ and R¹¹ are each independently a straight or branched, saturated or unsaturated alkyl chain containing from 10 to 30 carbon atoms, wherein the alkyl chain is optionally interrupted by one or more ester bonds; and z has a mean value ranging from 30 to 60. In some aspects, R¹⁰ and R¹¹ are each independently straight, saturated alkyl chains having 12 to 16 carbon atoms. In some aspects, the average z is about 45.

### Start Here

In some aspects, the LNP self-assembles into non-bilayer structures when mixed with polyanionic nucleic acid. In some aspects, the non-bilayer structures have a diameter between 60 nm and 120 nm. In some aspects, the non-bilayer structures have a diameter of about 70 nm, about 80 nm, about 90 nm, or about 100 nm. In some aspects, wherein the nanoparticulate delivery vehicle has a diameter of about 100 nm.

Also provided for herein is a vector or set of vectors comprising any of the nucleotide sequence described herein. Also disclosed herein is a vector comprising an isolated nucleotide sequence disclosed herein.

Also provided for herein is an isolated cell comprising any of the nucleotide sequences or set of isolated nucleotide sequences described herein, optionally wherein the cell is a BHK-21, CHO, HEK293 or variants thereof, 911, HeLa, A549, LP-293, PER.C6, or AE1-2a cell.

Also provided for herein is a kit comprising any of the compositions described herein and instructions for use. Also disclosed herein is a kit comprising a vector or a composition disclosed herein and instructions for use.

Also provided for herein is a method for treating a subject suffering from Covid-19, the method comprising administering to the subject any of the compositions or any of the pharmaceutical compositions described herein.

Also provided for herein is a method for treating a subject, the method comprising administering to the subject any of the compositions or any of the pharmaceutical compositions described herein.

Also provided for herein is a method for stimulating an immune response in a subject, the method comprising administering to the subject any of the compositions or any of the pharmaceutical compositions described herein.

Also disclosed herein is a method for treating a subject, the method comprising administering to the subject a vector disclosed herein or a pharmaceutical composition disclosed herein.

Also disclosed herein is a method of manufacturing the one or more vectors of any of the above compositions.

Also disclosed herein is a method of manufacturing any of the compositions disclosed herein.

### BRIEF DESCRIPTION OF THE SEVERAL VIEWS OF THE DRAWINGS

These and other features, aspects, and advantages of the present invention will become better understood with regard to the following description, and accompanying drawings, where:

Figure (FIG.) 1 illustrates a self-amplifying mRNA (SAM) system featuring a single alphavirus-derived subgenomic promoter (SGP).

FIG. 2 illustrates a SAM system featuring multiple expression cassettes driven by separate SGPs.

FIG. 3 presents antigen-specific cellular immune responses measured using ELISpot for mice vaccinated with a SAM system featuring multiple expression cassettes driven by separate alphavirus-derived subgenomic promoters (SGPs). Shown is IFNγ ELISpot results 2 weeks post immunization. T cell response to overlapping peptide pools spanning either Spike, Nucleocapsid, or Orf3a is shown.

FIG. 4 presents antigen-specific cellular immune responses measured using ELISpot for mice vaccinated with a ChAdV system featuring multiple expression cassettes driven by separate CMV promoters. Shown is IFNγ ELISpot results 2 weeks post immunization. T cell response to overlapping peptide pools spanning either Spike, Nucleocapsid, or Orf3a is shown.

FIG. 5 presents antigen-specific cellular immune responses measured using ELISpot for mice vaccinated with a SAM system featuring multiple expression cassettes (Spike “IDT-Spike” and T cell epitope cassette 5 “TCE5”) driven by separate SGPs. Schematics for the various cassette organizations are shown at the top. Left panel shows the sum of response to 8 overlapping peptide pools spanning Spike antigen. Right panel shows the sum of response to 3 overlapping peptide pools spanning NCap, Membrane, and Orf3a. Data are for Balb/c mice immunized with 10 ug of each SAM vaccine, n=6/group, and splenocyte isolation at 2-weeks post immunization.

FIG. 6 presents the Spike-specific IgG response for mice vaccinated with a SAM system featuring multiple expression cassettes (Spike “IDT-Spike” and T cell epitope cassette 5 “TCE5”) driven by separate SGPs. Schematics for the various cassette organizations are shown at the top. Balb/c mice were immunized with 10 ug of each SAM vaccine, n=4/group. Serum was collected and analyzed at 4-weeks post immunization. 51 IgG binding measured by MSD ELISA. Interpolated endpoint titer. Geomean, geometric SD.

FIG. 7 presents antigen-specific cellular immune responses measured using ELISpot for mice vaccinated with a SAM system featuring multiple expression cassettes (Spike “IDT-Spike” and T cell epitope cassettes 6 or 7 “TCE6/7”) driven by separate SGPs. Schematics for the various cassette organizations are shown at the top. Top panel shows the sum of response to 8 overlapping peptide pools spanning Spike antigen. Bottom panel shows the sum of response to 3 overlapping peptide pools spanning NCap, Membrane, and Orf3a. Data are for Balb/c mice immunized with 10 ug of each SAM vaccine, n=6/group, and splenocyte isolation at 2-weeks post immunization.

FIG. 8 presents the Spike-specific IgG response for mice vaccinated with a SAM system featuring multiple expression cassettes (Spike “IDT-Spike” and T cell epitope cassettes 6 or 7 “TCE6/7”) driven by separate SGPs. Schematics for the various cassette organizations are shown at the top. Balb/c mice were immunized with 10 ug of each SAM vaccine, n=4/group. Serum was collected and analyzed at 4-weeks post immunization. 51 IgG binding measured by MSD ELISA. Interpolated endpoint titer. Geomean, geometric SD.

FIG. 9 presents antigen-specific cellular immune responses measured using ELISpot for mice vaccinated with a SAM system featuring multiple expression cassettes (Spike “IDT-Spike” and T cell epitope cassettes 5 or 8 “TCE5/8”) driven by separate SGPs. Schematics for the various cassette organizations are shown at the top. Top panel shows the sum of response to 8 overlapping peptide pools spanning Spike antigen. Bottom panel shows the sum of response to 3 overlapping peptide pools spanning NCap, Membrane, and Orf3a. Data are for Balb/c mice immunized with 10 ug of each SAM vaccine, n=6/group, and splenocyte isolation at 2-weeks post immunization.

FIG. 10 presents the Spike-specific IgG response for mice vaccinated with a SAM system featuring multiple expression cassettes (Spike “IDT-Spike” and T cell epitope cassettes 5 or 8 “TCE5/8”) driven by separate SGPs. Schematics for the various cassette organizations are shown at the top. Balb/c mice were immunized with 10 ug of each SAM vaccine, n=4/group. Serum was collected and analyzed at 4-weeks post immunization. 51 IgG binding measured by MSD ELISA. Interpolated endpoint titer. Geomean, geometric SD.

FIG. 11 presents antigen-specific cellular immune responses measured using ELISpot for mice vaccinated with a SAM system featuring multiple expression cassettes (Spike “SA-Spike”; and T cell epitope cassette 9 “TCE9” or a combination of Nucleocapsid and TCE11 “Nuc-TCE11) driven by separate SGPs. Schematics for the various cassette organizations are shown at the top. Left panel shows the sum of response to 8 overlapping peptide pools spanning Spike antigen. Middle panel shows the sum of T-cell responses detected by ELISpot with protein specific peptide pools for TCE (Orf3a, Membrane & NSP 3, 4, 6 & 12 genes). Right panel shows the sum of response to peptide pools spanning Nucleocapsid. Data are for Balb/c mice immunized with 10 ug of each SAM vaccine, n=6/group, and splenocyte isolation at 2-weeks post immunization. Spleens were harvested from 6 mice in each group at d14 for T-cell analysis. Sera from the remaining mice (n=6) were analyzed at 0, 4, 8 and 12 weeks for IgG responses by anti-IgG MSD ELISA. Four week IgG data is shown.

FIG. 12 presents the Spike-specific IgG response for mice vaccinated with a SAM system featuring multiple expression cassettes (Spike “SA-Spike”; and T cell epitope cassette 9 “TCE9” or a combination of Nucleocapsid and TCE11 “Nuc-TCE11) driven by separate SGPs. Schematics for the various cassette organizations are shown at the top. Balb/c mice were immunized with 10 ug of each SAM vaccine, n=6/group. Serum was collected and analyzed at 0, 4, 8 and 12 weeks post immunization. Shown is 4-weeks post immunization. 51 IgG binding measured by MSD ELISA. Interpolated endpoint titer. Geomean, geometric SD.

### DETAILED DESCRIPTION

Described herein are multicistronic alphavirus-derived self-amplifying mRNA (SAM) vectors and compositions for delivery of payload/antigen expression systems described herein. The multicistronic SAM constructs include (A) a self-replicating alphavirus-based expression system, wherein the self-replicating alphavirus-based expression system includes one or more vectors, wherein the one or more vectors include: (a) an RNA alphavirus backbone, wherein the RNA alphavirus backbone includes: (i) at least one promoter nucleotide sequence, and (ii) at least one polyadenylation (poly(A)) sequence; and (b) at least two cassettes expressing a payload sequence, wherein a first of the at least two cassettes, oriented from 5′ to 3′, is operably linked to a promoter nucleotide sequence comprising a first subgenomic alphavirus-derived promoter (SGP1) comprising a core conserved promoter sequence comprising the polynucleotide sequence ctacggcTAAcctgaa(+1)tgga (SEQ ID NO: 27974), and wherein at least a second of the at least two cassettes is operably linked to a promoter nucleotide sequence comprising a second subgenomic alphavirus-derived promoter (SGP2) comprising the core conserved promoter sequence, and wherein the SGP1 and/or the SGP2 subgenomic promoter comprises an extended 3′ promoter region derived from an alphavirus encoded 3′ of the core conserved promoter sequence.

In general, and without wishing to be bound by theory, the multicistronic SAM vectors herein address potential technical limitations present in the field, including, but not limited to: (1) improving expression of multiple payloads that include large cassettes (e.g., greater than the size of a native cassette expressed from a native alphavirus subgenomic promoter, such as cassettes approximately 4 kb or greater in length); (2) improved control of expression of multiple payloads, e.g., controlling the relative expression of different payloads; and (3) improved vector stability, such as by reducing vector recombination events (e.g., intra-vector promoter recombination between alphavirus subgenomic promoters).

In illustrative vaccine contexts, each of the cassettes can independently comprise: (i) at least one antigen-encoding nucleic acid sequence comprising: a. an epitope-encoding nucleic acid sequence, b. optionally a 5′ linker sequence, and c. optionally a 3′ linker sequence.

A cassette can optionally include at least one second poly(A) sequence, wherein the second poly(A) sequence is a native poly(A) sequence or an exogenous poly(A) sequence to the alphavirus backbone.

Multicistronic SAM vectors herein include multiple subgenomic promoters that include core conserved promoter sequences. In general, the minimum “conserved” promoter sequence includes the polynucleotide sequence ctacggcTAAcctgaa(+1)tgga (SEQ ID NO: 27974), where “+1” indicates the putative transcriptional start site of the subgenomic promoter. Additional flanking sequences of alphavirus subgenomic promoters can influence promoter activity and be considered part of the core promoter. For example, additional flanking sequences may be needed to produce detectable expression in vitro and/or efficacy in vivo (e.g., efficacy in stimulating an immune response).

A subgenomic promoter (SGP1, SGP2, or both) can include an extended 3′ promoter region. An extended 3′ promoter region can be derived from the same alphavirus as the alphavirus used to derive the core conserved promoter sequence. An extended 3′ promoter region can be derived from a different alphavirus as the alphavirus used to derive the core conserved promoter sequence. An extended 3′ promoter region can include the polynucleotide sequence CTACGACAT (SEQ ID NO: 27997). An extended 3′ promoter region can include the polynucleotide sequence CTACGACAT (SEQ ID NO: 27997) and additional extended 3′ promoter region polynucleotides derived from an alphavirus, e.g., the same alphavirus used to derive the core conserved promoter sequence. An extended 3′ promoter region can include the polynucleotide sequence AGTCTAGTCCGCCAAG (SEQ ID NO: 27982). An extended 3′ promoter region can include the polynucleotide sequence CTACGACATAGTCTAGTCCGCCAAG (SEQ ID NO: 27975). An extended 3′ promoter region can include the polynucleotide sequence CTACGACAT (SEQ ID NO: 27997) and be encoded immediately 3′ of the core conserved promoter sequence (i.e., no interspersing nucleotides are included between the core conserved promoter and the extended 3′ promoter region) resulting in a subgenomic promoter including the polynucleotide sequence CTACGGCTAACCTGAATGGACTACGACAT (SEQ ID NO: 27983) or CTCTCTACGGCTAACCTGAATGGACTACGACAT (SEQ ID NO: 27984). An extended 3′ promoter region can include the polynucleotide sequence CTACGACATAGTCTAGTCCGCCAAG (SEQ ID NO: 27975) and be encoded immediately 3′ of the core conserved promoter sequence resulting in a subgenomic promoter including the polynucleotide sequence CTACGGCTAACCTGAATGGACTACGACATAGTCTAGTCCGCCAAG (SEQ ID NO: 27985) or CTCTCTACGGCTAACCTGAATGGACTACGACATAGTCTAGTCCGCCAAG (SEQ ID NO: 27986).

One of the subgenomic promoters (i.e., either SGP1 or SGP2, but not both) can include an extended 3′ promoter region. One of the subgenomic promoters can include an extended 3′ promoter region that is different than the other extended 3′ promoter region (e.g., SGP1 and SGP2 have different extended 3′ promoter regions). For example, and without wishing to be bound by theory, an extended 3′ promoter region can improve vector stability, such as by reducing vector recombination events (e.g., intra-vector promoter recombination between alphavirus subgenomic promoters) through including an extended 3′ promoter region in only one of the subgenomic promoters (SGP1 or SGP2, but not both).

In another example, and without wishing to be bound by theory, an extended 3′ promoter region can improve control of expression, such as controlling the relative strength of expression of a cassette from various subgenomic promoters through including an extended 3′ promoter region in only one of the subgenomic promoters (SGP1 or SGP2, but not both).

Only one of the subgenomic promoters (i.e., either SGP1 or SGP2, but not both) can include an extended 3′ promoter region including the polynucleotide sequence CTACGACAT (SEQ ID NO: 27997). Only one of the subgenomic promoters (i.e., either SGP1 or SGP2, but not both) can include an extended 3′ promoter region including the polynucleotide sequence CTACGACAT (SEQ ID NO: 27997) and additional polynucleotides derived from an alphavirus, e.g., the same alphavirus used to derive the core conserved promoter sequence. Only one of the subgenomic promoters (i.e., either SGP1 or SGP2, but not both) can include an extended 3′ promoter region sequence including the polynucleotide sequence AGTCTAGTCCGCCAAG (SEQ ID NO: 27982).

One or all of the subgenomic promoters can include an extended 3′ promoter region including the polynucleotide sequence CTACGACAT (SEQ ID NO: 27997) but only one of the subgenomic promoters (i.e., either SGP1 or SGP2, but not both) can include additional extended 3′ promoter region polynucleotides derived from an alphavirus, e.g., the same alphavirus used to derive the core conserved promoter sequence. One or all of the subgenomic promoters can include an extended 3′ promoter region including the polynucleotide sequence CTACGACAT (SEQ ID NO: 27997) but only one of the subgenomic promoters (i.e., either SGP1 or SGP2, but not both) can include an extended 3′ promoter region sequence including the polynucleotide sequence AGTCTAGTCCGCCAAG (SEQ ID NO: 27982).

Only one of the subgenomic promoters (i.e., either SGP1 or SGP2, but not both) can include an extended 3′ promoter region including the polynucleotide sequence CTACGACATAGTCTAGTCCGCCAAG (SEQ ID NO: 27975). One or all of the subgenomic promoters can include an extended 3′ promoter region including the polynucleotide sequence CTACGACAT (SEQ ID NO: 27997) but only one of the subgenomic promoters (i.e., either SGP1 or SGP2, but not both) can include an extended 3′ promoter region including the polynucleotide sequence CTACGACATAGTCTAGTCCGCCAAG (SEQ ID NO: 27975).

Only one of the subgenomic promoters (i.e., either SGP1 or SGP2, but not both) can include an extended 3′ promoter region including the polynucleotide sequence CTACGACAT (SEQ ID NO: 27997) and be encoded immediately 3′ of the core conserved promoter sequence resulting in a subgenomic promoter including the polynucleotide sequence CTACGGCTAACCTGAATGGACTACGACAT (SEQ ID NO: 27983) or CTCTCTACGGCTAACCTGAATGGACTACGACAT (SEQ ID NO: 27984).

Only one of the subgenomic promoters (i.e., either SGP1 or SGP2, but not both) can include an extended 3′ promoter region including the polynucleotide sequence CTACGACATAGTCTAGTCCGCCAAG (SEQ ID NO: 27975) and be encoded immediately 3′ of the core conserved promoter sequence resulting in a subgenomic promoter including the polynucleotide sequence CTACGGCTAACCTGAATGGACTACGACATAGTCTAGTCCGCCAAG (SEQ ID NO: 27985) or CTCTCTACGGCTAACCTGAATGGACTACGACATAGTCTAGTCCGCCAAG (SEQ ID NO: 27986).

One or all of the subgenomic promoters can include an extended 3′ promoter region including the polynucleotide sequence CTACGACAT (SEQ ID NO: 27997) encoded immediately 3′ of the core conserved promoter sequence but only one of the subgenomic promoters (i.e., either SGP1 or SGP2, but not both) can include an extended 3′ promoter region including the polynucleotide sequence CTACGACATAGTCTAGTCCGCCAAG (SEQ ID NO: 27975) and be encoded immediately 3′ of the core conserved promoter sequence.

In some instances, the paired subgenomic promoter sequences (i.e., SGP1 and SGP2) include either CTACGGCTAACCTGAATGGACTACGACATAGTCTAGTCCGCCAAG (SEQ ID NO: 27985) or CTCTCTACGGCTAACCTGAATGGACTACGACATAGTCTAGTCCGCCAAG (SEQ ID NO: 27986) paired with either CTACGGCTAACCTGAATGGACTACGAC (SEQ ID NO: 27987) that does not include an additional extended 3′ promoter region or CTCTCTACGGCTAACCTGAATGGACTACGAC (SEQ ID NO: 27988) that does not include an additional extended 3′ promoter region. In some instances, the paired subgenomic promoter sequences (i.e., SGP1 and SGP2) include either CTACGGCTAACCTGAATGGACTACGACATAGTCTAGTCCGCCAAG (SEQ ID NO: 27985) or CTCTCTACGGCTAACCTGAATGGACTACGACATAGTCTAGTCCGCCAAG (SEQ ID NO: 27986) paired with either CTACGGCTAACCTGAATGGA (SEQ ID NO: 27974) that does not include an additional extended 3′ promoter region or CTCTCTACGGCTAACCTGAATGGA (SEQ ID NO: 27989) that does not include an additional extended 3′ promoter region.

In some instances, the paired subgenomic promoter sequences (i.e., SGP1 and SGP2) include CTCTCTACGGCTAACCTGAATGGACTACGACATAGTCTAGTCCGCCAAG (SEQ ID NO: 27986) paired with either CTCTCTACGGCTAACCTGAATGGACTACGAC (SEQ ID NO: 27988) that does not include an additional extended 3′ promoter region. In some instances, the paired subgenomic promoter sequences (i.e., SGP1 and SGP2) include CTACGGCTAACCTGAATGGACTACGACATAGTCTAGTCCGCCAAG (SEQ ID NO: 27985) paired with CTCTCTACGGCTAACCTGAATGGACTACGAC (SEQ ID NO: 27988) that does not include an additional extended 3′ promoter region.

In some instances, the paired subgenomic promoter sequences (i.e., SGP1 and SGP2) include CTCTCTACGGCTAACCTGAATGGACTACGACATAGTCTAGTCCGCCAAG (SEQ ID NO: 27986) paired with either CTCTCTACGGCTAACCT (SEQ ID NO: 27990) that does not include an additional extended 3′ promoter region. In some instances, the paired subgenomic promoter sequences (i.e., SGP1 and SGP2) include CTACGGCTAACCTGAATGGACTACGACATAGTCTAGTCCGCCAAG (SEQ ID NO: 27985) paired with CTCTCTACGGCTAACCTGAATGGA (SEQ ID NO: 27989) that does not include an additional extended 3′ promoter region.

In some instances, the paired subgenomic promoter sequences (i.e., SGP1 and SGP2) include CTCTCTACGGCTAACCTGAATGGACTACGACATAGTCTAGTCCGCCAAG (SEQ ID NO: 27986) paired with CTACGGCTAACCTGAATGGACTACGAC (SEQ ID NO: 27987) that does not include an additional extended 3′ promoter region. In some instances, the paired subgenomic promoter sequences (i.e., SGP1 and SGP2) include CTACGGCTAACCTGAATGGACTACGACATAGTCTAGTCCGCCAAG (SEQ ID NO: 27985) paired with CTACGGCTAACCTGAATGGACTACGAC (SEQ ID NO: 27987) that does not include an additional extended 3′ promoter region.

In some instances, the paired subgenomic promoter sequences (i.e., SGP1 and SGP2) include CTCTCTACGGCTAACCTGAATGGACTACGACATAGTCTAGTCCGCCAAG (SEQ ID NO: 27986) paired with CTACGGCTAACCTGAATGGA (SEQ ID NO: 27974) that does not include an additional extended 3′ promoter region. In some instances, the paired subgenomic promoter sequences (i.e., SGP1 and SGP2) include CTACGGCTAACCTGAATGGACTACGACATAGTCTAGTCCGCCAAG (SEQ ID NO: 27985) paired with CTACGGCTAACCTGAATGGA (SEQ ID NO: 27974) that does not include an additional extended 3′ promoter region.

A subgenomic promoter (SGP1, SGP2, or both) can include an extended 5′ promoter region. Without wishing to be bound by theory, an extended 5′ promoter region can improve control of expression, such as controlling the relative strength of expression of a cassette from various subgenomic promoters through including an extended 5′ promoter region. An extended 5′ promoter region can be derived from the same alphavirus as the alphavirus used to derive the core conserved promoter sequence. An extended 5′ promoter region can be derived from a different alphavirus as the alphavirus used to derive the core conserved promoter sequence. An extended 5′ promoter region include the polynucleotide sequence ctct encoded immediately 5′ of the core conserved promoter sequence (i.e., no interspersing nucleotides are included between the core conserved promoter and the extended 5′ promoter region). In one embodiment, each of the one or more of the subgenomic promoters includes the minimum sequence ctctctacggcTAAcctgaa(+1)tgga (SEQ ID NO: 27989).

An extended 5′ promoter region can include a polynucleotide sequence derived from the polynucleotide sequence acttccatcatagttatggccatgactactctagctagcagtgttaaatcattcagctacctgagaggggcccctataactct (SEQ ID NO: 27977). An extended 5′ promoter region can include the polynucleotide sequence acttccatcatagttatggccatgactactctagctagcagtgttaaatcattcagctacctgagaggggcccctataactct (SEQ ID NO: 27977). An extended 5′ promoter region can include the polynucleotide sequence acttccatcatagttatggccatgactactctagctagcagtgttaaatcattcagctacctgagaggggcccctataactct (SEQ ID NO: 27977) encoded immediately 5′ of the core conserved promoter sequence. An extended 5′ promoter region can include the polynucleotide sequence the polynucleotide sequence acctgagaggggcccctataactct (SEQ ID NO: 27978). An extended 5′ promoter region can include the polynucleotide sequence acctgagaggggcccctataactct (SEQ ID NO: 27978) encoded immediately 5′ of the core conserved promoter sequence. An extended 5′ promoter region can include the polynucleotide sequence gggcccctataactct (SEQ ID NO: 27979). An extended 5′ promoter region can include the polynucleotide sequence gggcccctataactct (SEQ ID NO: 27979) encoded immediately 5′ of the core conserved promoter sequence. An extended 5′ promoter region can include the polynucleotide sequence ggggcccctataactct (SEQ ID NO: 27991). An extended 5′ promoter region can include the polynucleotide sequence ggggcccctataactct (SEQ ID NO: 27991) encoded immediately 5′ of the core conserved promoter sequence.

An extended 5′ promoter region can consist of the polynucleotide sequence gggcccctataactct (SEQ ID NO: 27979). An extended 5′ promoter region can consist of the polynucleotide sequence gggcccctataactct (SEQ ID NO: 27979) encoded immediately 5′ of the core conserved promoter sequence. An extended 5′ promoter region can consist of the polynucleotide sequence ggggcccctataactct (SEQ ID NO: 27991). An extended 5′ promoter region can consist of the polynucleotide sequence ggggcccctataactct (SEQ ID NO: 27991) encoded immediately 5′ of the core conserved promoter sequence.

An extended 5′ promoter region of an SGP2 subgenomic promoter can include a polynucleotide sequence derived from the polynucleotide sequence acttccatcatagttatggccatgactactctagctagcagtgttaaatcattcagctacctgagaggggcccctataactct (SEQ ID NO: 27977). An extended 5′ promoter region of an SGP2 subgenomic promoter can include the polynucleotide sequence acttccatcatagttatggccatgactactctagctagcagtgttaaatcattcagctacctgagaggggcccctataactct (SEQ ID NO: 27977). An extended 5′ promoter region of an SGP2 subgenomic promoter can include the polynucleotide sequence acttccatcatagttatggccatgactactctagctagcagtgttaaatcattcagctacctgagaggggcccctataactct (SEQ ID NO: 27977) encoded immediately 5′ of the core conserved promoter sequence. An extended 5′ promoter region of an SGP2 subgenomic promoter can include the polynucleotide sequence the polynucleotide sequence acctgagaggggcccctataactct (SEQ ID NO: 27978). An extended 5′ promoter region of an SGP2 subgenomic promoter can include the polynucleotide sequence acctgagaggggcccctataactct (SEQ ID NO: 27978) encoded immediately 5′ of the core conserved promoter sequence. An extended 5′ promoter region of an SGP2 subgenomic promoter can include the polynucleotide sequence gggcccctataactct (SEQ ID NO: 27979). An extended 5′ promoter region of an SGP2 subgenomic promoter can include the polynucleotide sequence gggcccctataactct (SEQ ID NO: 27979) encoded immediately 5′ of the core conserved promoter sequence. An extended 5′ promoter region of an SGP2 subgenomic promoter can consist of the polynucleotide sequence gggcccctataactct (SEQ ID NO: 27979). An extended 5′ promoter region of an SGP2 subgenomic promoter can consist of the polynucleotide sequence gggcccctataactct (SEQ ID NO: 27979) encoded immediately 5′ of the core conserved promoter sequence. An extended 5′ promoter region of an SGP2 subgenomic promoter can consist of the polynucleotide sequence ggggcccctataactct (SEQ ID NO: 27991). An extended 5′ promoter region of an SGP2 subgenomic promoter can consist of the polynucleotide sequence ggggcccctataactct (SEQ ID NO: 27991) encoded immediately 5′ of the core conserved promoter sequence.

An extended promoter region can include one or more transcriptional enhancer elements. Enhancer elements can be encoded 3′ or 5′ of the core promoter sequence(s). Either SGP1, SGP2, or both can include enhancer elements. An enhancer element can be derived from the same alphavirus as the alphavirus used to derive the core conserved promoter sequence. An enhancer element can be derived from a different alphavirus as the alphavirus used to derive the core conserved promoter sequence. In particular, a polynucleotide encoding the C-terminal portion of a nsp4 gene in a native alphavirus overlaps with the core conserved promoter sequence of alphavirus subgenomic promoter in the context of a native 26S subgenomic transcriptional promoter. Accordingly, either SGP1, SGP2, or both can include enhancer elements that include a polynucleotide sequence derived from an alphavirus nonstructural protein 4 (nsp4), which is generally encoded 5′ of the core conserved promoter sequence, e.g., encoded immediately 5′ of the core conserved promoter sequence. A polynucleotide encoding the C-terminal portion of a nsp4 can include an additional sequence GGGCCCCTATAA (SEQ ID NO: 27992) resulting in a subgenomic promoter can include the sequence GGGCCCCTATAACTCTCTACGGCTAACCTGAATGGACTACGAC (SEQ ID NO: 27981). Either SGP1, SGP2, or both can include the sequence GGGCCCCTATAACTCTCTACGGCTAACCTGAATGGACTACGAC (SEQ ID NO: 27981). Inclusion of a polynucleotide encoding the C-terminal portion of a nsp4 5′ of the core conserved promoter sequence and an extended 3′ promoter region of CTACGACATAGTCTAGTCCGCCAAG (SEQ ID NO: 27975) 3′ of the core conserved promoter sequence can result in subgenomic promoter including the sequence GGGCCCCTATAACTCTCTACGGCTAACCTGAATGGACTACGACATAGTCTAGTCCGC CAAG (SEQ ID NO: 27980) (which is generally limited to either SGP1 or SGP2, but not both). A polynucleotide encoding the C-terminal portion of a nsp4 can include an additional sequence GGGGCCCCTATAA (SEQ ID NO: 27993) resulting in a subgenomic promoter can include the sequence GGGGCCCCTATAACTCTCTACGGCTAACCTGAATGGACTACGAC (SEQ ID NO: 27994). Either SGP1, SGP2, or both can include the sequence GGGGCCCCTATAACTCTCTACGGCTAACCTGAATGGACTACGAC (SEQ ID NO: 27994). Inclusion of a polynucleotide encoding the C-terminal portion of a nsp4 5′ of the core conserved promoter sequence and an extended promoter region of CTACGACATAGTCTAGTCCGCCAAG (SEQ ID NO: 27975) 3′ of the core conserved promoter sequence can result in subgenomic promoter including the sequence GGGGCCCCTATAACTCTCTACGGCTAACCTGAATGGACTACGACATAGTCTAGTCCG CCAAG (SEQ ID NO: 27995) (which is generally limited to either SGP1 or SGP2, but not both).

Inclusion of a polynucleotide encoding the C-terminal portion of a nsp4 5′ of the core conserved promoter sequence and an extended promoter region of CTACGACATAGTCTAGTCCGCCAAG (SEQ ID NO: 27975) 3′ of the core conserved promoter sequence can result subgenomic promoters including the following pairs of sequences:

(1) SGP1 including
(SEQ ID NO: 27981)
GGGCCCCTATAACTCTCTACGGCTAACCTGAATGGACTACGAC
and
SGP2 including
(SEQ ID NO: 27980)
GGGCCCCTATAACTCTCTACGGCTAACCTGAATGGACTACGACATAGTCTAGTCCGC
CAAG;
(2) SGP2 including
(SEQ ID NO: 27981)
GGGCCCCTATAACTCTCTACGGCTAACCTGAATGGACTACGAC
and
SGP1 including
(SEQ ID NO: 27980)
GGGCCCCTATAACTCTCTACGGCTAACCTGAATGGACTACGACATAGTCTAGTCCGC
CAAG;
(3) SGP1 including
(SEQ ID NO: 27988)
CTCTCTACGGCTAACCTGAATGGACTACGAC
and
SGP2 including
(SEQ ID NO: 27980)
GGGCCCCTATAACTCTCTACGGCTAACCTGAATGGACTACGACATAGTCTAGTCCGC
CAAG;
(4) SGP2 including
(SEQ ID NO: 27988)
CTCTCTACGGCTAACCTGAATGGACTACGAC
and
SGP1 including
(SEQ ID NO: 27980)
GGGCCCCTATAACTCTCTACGGCTAACCTGAATGGACTACGACATAGTCTAGTCCGC
CAAG;
(5) SGP1 including
(SEQ ID NO: 27981)
GGGCCCCTATAACTCTCTACGGCTAACCTGAATGGACTACGAC
and
SGP2 including
(SEQ ID NO: 27986)
CTCTCTACGGCTAACCTGAATGGACTACGACATAGTCTAGTCCGCCAAG;
(6) SGP2 including
(SEQ ID NO: 27981)
GGGCCCCTATAACTCTCTACGGCTAACCTGAATGGACTACGAC
and
SGP1 including
(SEQ ID NO: 27986)
CTCTCTACGGCTAACCTGAATGGACTACGACATAGTCTAGTCCGCCAAG.

Inclusion of a polynucleotide encoding the C-terminal portion of a nsp4 5′ of the core conserved promoter sequence and an extended promoter region of atagtctagtccgccaag (SEQ ID NO: 27976) 3′ of the core conserved promoter sequence can result subgenomic promoters consisting of the following pairs of sequences:

(1) SGP1 including
(SEQ ID NO: 27981)
GGGCCCCTATAACTCTCTACGGCTAACCTGAATGGACTACGAC
and
SGP2 including
(SEQ ID NO: 27980)
GGGCCCCTATAACTCTCTACGGCTAACCTGAATGGACTACGACATAGTCTAGTCCGC
CAAG;
(2) SGP2 including
(SEQ ID NO: 27981)
GGGCCCCTATAACTCTCTACGGCTAACCTGAATGGACTACGAC
and
SGP1 including
(SEQ ID NO: 27980)
GGGCCCCTATAACTCTCTACGGCTAACCTGAATGGACTACGACATAGTCTAGTCCGC
CAAG;
(3) SGP1 including
(SEQ ID NO: 27988)
CTCTCTACGGCTAACCTGAATGGACTACGAC
and
SGP2 including
(SEQ ID NO: 27980)
GGGCCCCTATAACTCTCTACGGCTAACCTGAATGGACTACGACATAGTCTAGTCCGC
CAAG;
(4) SGP2 including
(SEQ ID NO: 27988)
CTCTCTACGGCTAACCTGAATGGACTACGAC
and
SGP1 including
(SEQ ID NO: 27980)
GGGCCCCTATAACTCTCTACGGCTAACCTGAATGGACTACGACATAGTCTAGTCCGC
CAAG;
(5) SGP1 including
(SEQ ID NO: 27981)
GGGCCCCTATAACTCTCTACGGCTAACCTGAATGGACTACGAC
and
SGP2 including
(SEQ ID NO: 27986)
CTCTCTACGGCTAACCTGAATGGACTACGACATAGTCTAGTCCGCCAAG;
(6) SGP2 including
(SEQ ID NO: 27981)
GGGCCCCTATAACTCTCTACGGCTAACCTGAATGGACTACGAC
and
SGP1 including
(SEQ ID NO: 27986)
CTCTCTACGGCTAACCTGAATGGACTACGACATAGTCTAGTCCGCCAAG.

The at least one promoter nucleotide sequence of the RNA alphavirus backbone can include the SGP1 subgenomic promoter. For example, SGP1 can refer to the “native” subgenomic promoter provided by the alphavirus backbone.

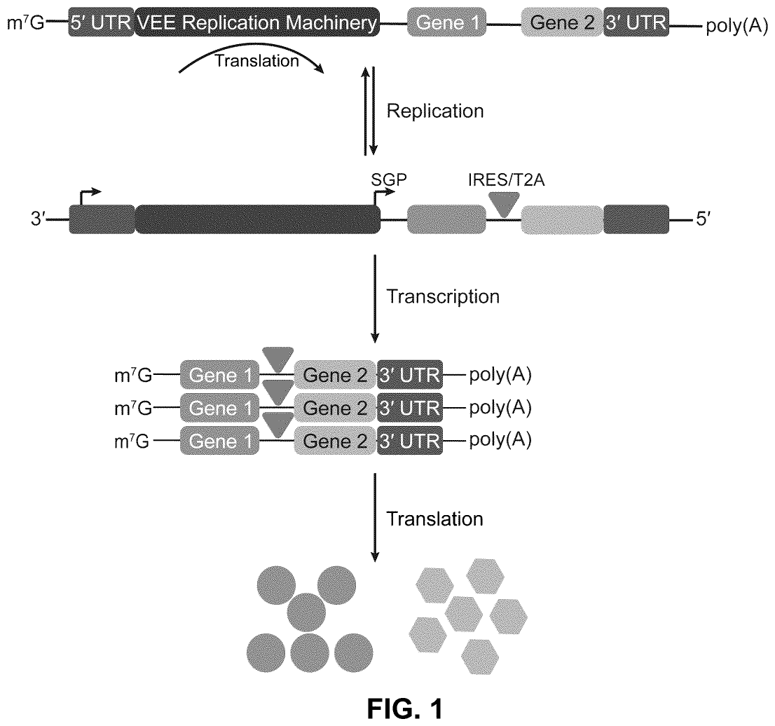

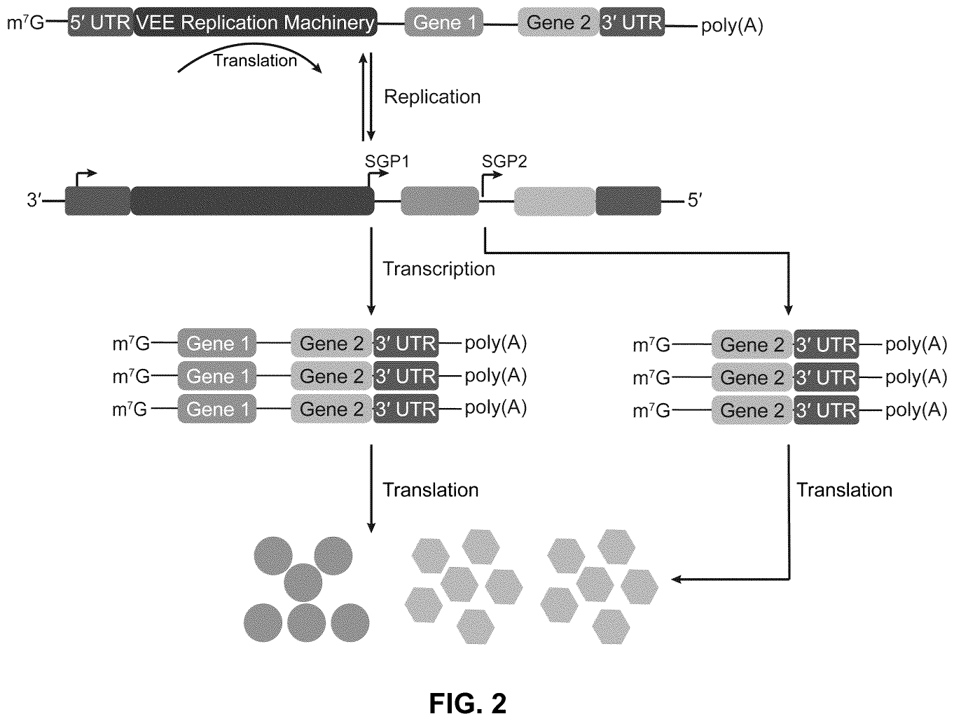

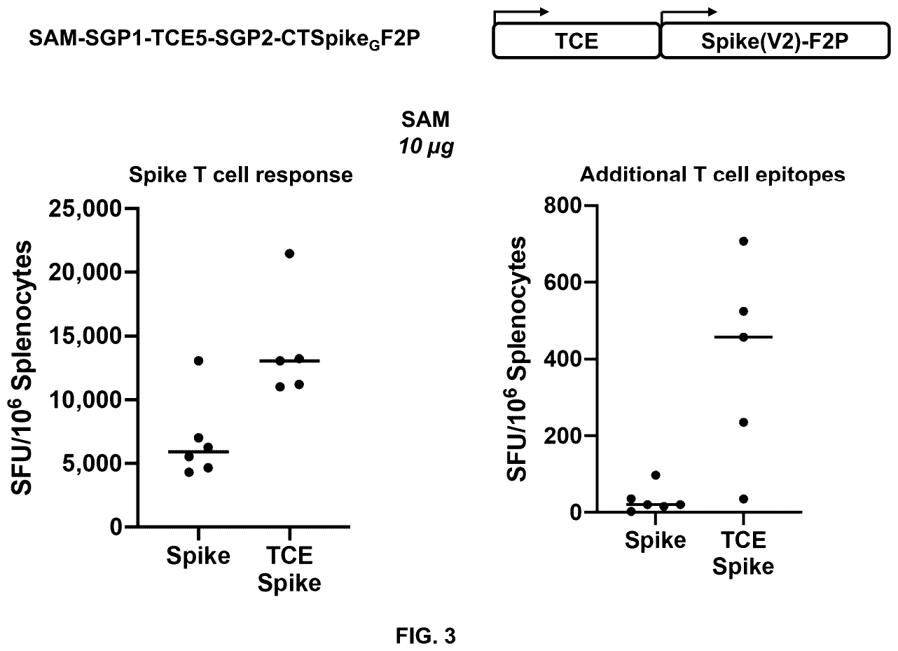

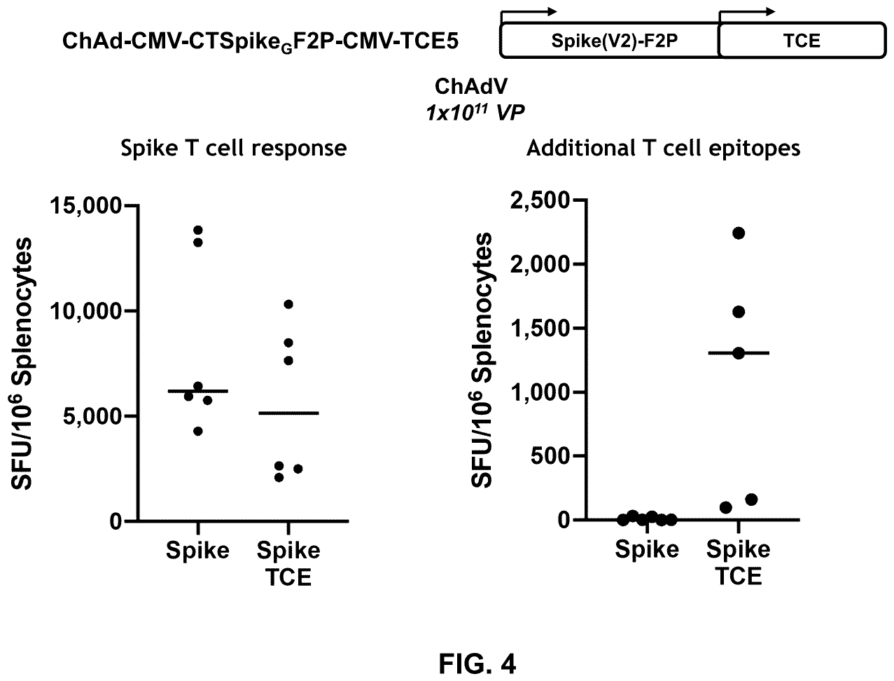

Cassettes can be ordered to provide control of expression levels. For example, without wishing to be bound by theory and as FIG. 2 illustrates, SAM system featuring multiple expression cassettes driven by separate SGPs can drive higher expression of a gene under control of the second SGP (SGP2) given both SGP1 and SGP2 will produce transcripts encoding the second gene. Accordingly, multicistronic SAM vectors can have an SGP2 subgenomic promoter that is capable of promoting expression of a cassette at least 2-fold greater relative to the same cassette operably linked to the SGP1 subgenomic promoter. In some embodiments, an extended promoter region (e.g., the 5′ nsp4 polynucleotide and/or atagtctagtccgccaag (SEQ ID NO: 27976) described above) is included in SGP2 that is capable of promoting expression of a cassette at least 2-fold greater relative to the same cassette operably linked to the SGP1 subgenomic promoter.

A cassette can be configured so the cassette is operably linked to a subgenomic promoter capable of driving expression of a payload from the cassette. For example, a subgenomic promoter can be immediately 5′ of a cassette, including immediately 5′ of a Kozak sequence of a cassette.

### I. Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

As used herein the term “antigen” is a substance that stimulates an immune response. An antigen can be a neoantigen. An antigen can be a “shared antigen” that is an antigen found among a specific population, e.g., a specific population of cancer patients.

As used herein the term “neoantigen” is an antigen that has at least one alteration that makes it distinct from the corresponding wild-type antigen, e.g., via mutation in a tumor cell or post-translational modification specific to a tumor cell. A neoantigen can include a polypeptide sequence or a nucleotide sequence. A mutation can include a frameshift or non-frameshift indel, missense or nonsense substitution, splice site alteration, genomic rearrangement or gene fusion, or any genomic or expression alteration giving rise to a neoORF. A mutations can also include a splice variant. Post-translational modifications specific to a tumor cell can include aberrant phosphorylation. Post-translational modifications specific to a tumor cell can also include a proteasome-generated spliced antigen. See Liepe et al., A large fraction of HLA class I ligands are proteasome-generated spliced peptides; Science. 2016 Oct. 21; 354(6310):354-358. The subject can be identified for administration through the use of various diagnostic methods, e.g., patient selection methods described further below.

As used herein the term “tumor antigen” is an antigen present in a subject's tumor cell or tissue but not in the subject's corresponding normal cell or tissue, or derived from a polypeptide known to or have been found to have altered expression in a tumor cell or cancerous tissue in comparison to a normal cell or tissue.

As used herein the term “antigen-based vaccine” is a vaccine composition based on one or more antigens, e.g., a plurality of antigens. The vaccines can be nucleotide-based (e.g., virally based, RNA based, or DNA based), protein-based (e.g., peptide based), or a combination thereof.

As used herein the term “candidate antigen” is a mutation or other aberration giving rise to a sequence that may represent an antigen.

As used herein the term “coding region” is the portion(s) of a gene that encode protein.

As used herein the term “coding mutation” is a mutation occurring in a coding region.

As used herein the term “ORF” means open reading frame.

As used herein the term “NEO-ORF” is a tumor-specific ORF arising from a mutation or other aberration such as splicing.

As used herein the term “missense mutation” is a mutation causing a substitution from one amino acid to another.

As used herein the term “nonsense mutation” is a mutation causing a substitution from an amino acid to a stop codon or causing removal of a canonical start codon.

As used herein the term “frameshift mutation” is a mutation causing a change in the frame of the protein.

As used herein the term “indel” is an insertion or deletion of one or more nucleic acids.

As used herein, the term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Alternatively, sequence similarity or dissimilarity can be established by the combined presence or absence of particular nucleotides, or, for translated sequences, amino acids at selected sequence positions (e.g., sequence motifs).

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

As used herein the term “non-stop or read-through” is a mutation causing the removal of the natural stop codon.

As used herein the term “epitope” is the specific portion of an antigen typically bound by an antibody or T cell receptor.

As used herein the term “immunogenic” is the ability to stimulate an immune response, e.g., via T cells, B cells, or both.

As used herein the term “HLA binding affinity” “MHC binding affinity” means affinity of binding between a specific antigen and a specific MHC allele.

As used herein the term “bait” is a nucleic acid probe used to enrich a specific sequence of DNA or RNA from a sample.

As used herein the term “variant” is a difference between a subject's nucleic acids and the reference human genome used as a control.

As used herein the term “variant call” is an algorithmic determination of the presence of a variant, typically from sequencing.

As used herein the term “polymorphism” is a germline variant, i.e., a variant found in all DNA-bearing cells of an individual.

As used herein the term “somatic variant” is a variant arising in non-germline cells of an individual.

As used herein the term “allele” is a version of a gene or a version of a genetic sequence or a version of a protein.

As used herein the term “HLA type” is the complement of HLA gene alleles.

As used herein the term “nonsense-mediated decay” or “NMD” is a degradation of an mRNA by a cell due to a premature stop codon.

As used herein the term “truncal mutation” is a mutation originating early in the development of a tumor and present in a substantial portion of the tumor's cells.

As used herein the term “subclonal mutation” is a mutation originating later in the development of a tumor and present in only a subset of the tumor's cells.

As used herein the term “exome” is a subset of the genome that codes for proteins. An exome can be the collective exons of a genome.

As used herein the term “logistic regression” is a regression model for binary data from statistics where the logit of the probability that the dependent variable is equal to one is modeled as a linear function of the dependent variables.

As used herein the term “neural network” is a machine learning model for classification or regression consisting of multiple layers of linear transformations followed by element-wise nonlinearities typically trained via stochastic gradient descent and back-propagation.

As used herein the term “proteome” is the set of all proteins expressed and/or translated by a cell, group of cells, or individual.

As used herein the term “peptidome” is the set of all peptides presented by MHC-I or MHC-II on the cell surface. The peptidome may refer to a property of a cell or a collection of cells (e.g., the tumor peptidome, meaning the union of the peptidomes of all cells that comprise the tumor, or the infectious disease peptidome, meaning the union of the peptidomes of all cells that are infected by the infectious disease).

As used herein the term “ELISPOT” means Enzyme-linked immunosorbent spot assay—which is a common method for monitoring immune responses in humans and animals.

As used herein the term “dextramers” is a dextran-based peptide-MHC multimers used for antigen-specific T-cell staining in flow cytometry.

As used herein the term “tolerance or immune tolerance” is a state of immune non-responsiveness to one or more antigens, e.g. self-antigens.

As used herein the term “central tolerance” is a tolerance affected in the thymus, either by deleting self-reactive T-cell clones or by promoting self-reactive T-cell clones to differentiate into immunosuppressive regulatory T-cells (Tregs).

As used herein the term “peripheral tolerance” is a tolerance affected in the periphery by downregulating or anergizing self-reactive T-cells that survive central tolerance or promoting these T cells to differentiate into Tregs.

The term “sample” can include a single cell or multiple cells or fragments of cells or an aliquot of body fluid, taken from a subject, by means including venipuncture, excretion, ejaculation, massage, biopsy, needle aspirate, lavage sample, scraping, surgical incision, or intervention or other means known in the art.

The term “subject” encompasses a cell, tissue, or organism, human or non-human, whether in vivo, ex vivo, or in vitro, male or female. The term subject is inclusive of mammals including humans.

The term “mammal” encompasses both humans and non-humans and includes but is not limited to humans, non-human primates, canines, felines, murines, bovines, equines, and porcines.

The term “clinical factor” refers to a measure of a condition of a subject, e.g., disease activity or severity. “Clinical factor” encompasses all markers of a subject's health status, including non-sample markers, and/or other characteristics of a subject, such as, without limitation, age and gender. A clinical factor can be a score, a value, or a set of values that can be obtained from evaluation of a sample (or population of samples) from a subject or a subject under a determined condition. A clinical factor can also be predicted by markers and/or other parameters such as gene expression surrogates. Clinical factors can include tumor type, tumor sub-type, infection type, infection sub-type, and smoking history.

The term “antigen-encoding nucleic acid sequences derived from a tumor” refers to nucleic acid sequences obtained from the tumor, e.g. via RT-PCR; or sequence data obtained by sequencing the tumor and then synthesizing the nucleic acid sequences using the sequencing data, e.g., via various synthetic or PCR-based methods known in the art. Derived sequences can include nucleic acid sequence variants, such as sequence-optimized nucleic acid sequence variants (e.g., codon-optimized and/or otherwise optimized for expression), that encode the same polypeptide sequence as the corresponding native nucleic acid sequence obtained from a tumor.

The term “antigen-encoding nucleic acid sequences derived from an infection” refers to nucleic acid sequences obtained from infected cells or an infectious disease organism, e.g. via RT-PCR; or sequence data obtained by sequencing the infected cell or infectious disease organism and then synthesizing the nucleic acid sequences using the sequencing data, e.g., via various synthetic or PCR-based methods known in the art. Derived sequences can include nucleic acid sequence variants, such as sequence-optimized nucleic acid sequence variants (e.g., codon-optimized and/or otherwise optimized for expression), that encode the same polypeptide sequence as the corresponding native infectious disease organism nucleic acid sequence. Derived sequences can include nucleic acid sequence variants that encode a modified infectious disease organism polypeptide sequence having one or more (e.g., 1, 2, 3, 4, or 5) mutations relative to a native infectious disease organism polypeptide sequence. For example, a modified polypeptide sequence can have one or more missense mutations relative to the native polypeptide sequence of an infectious disease organism protein.

The term “alphavirus” refers to members of the family Togaviridae, and are positive-sense single-stranded RNA viruses. Alphaviruses are typically classified as either Old World, such as Sindbis, Ross River, Mayaro, Chikungunya, and Semliki Forest viruses, or New World, such as eastern equine encephalitis, Aura, Fort Morgan, or Venezuelan equine encephalitis and its derivative strain TC-83. Alphaviruses are typically self-replicating RNA viruses.

The term “alphavirus backbone” refers to minimal sequence(s) of an alphavirus that allow for self-replication of the viral genome. Minimal sequences can include conserved sequences for nonstructural protein-mediated amplification, a nonstructural protein 1 (nsP1) gene, a nsP2 gene, a nsP3 gene, a nsP4 gene, and a polyA sequence, as well as sequences for expression of subgenomic viral RNA including a subgenomic (e.g., a 26S) promoter element.

The term “sequences for nonstructural protein-mediated amplification” includes alphavirus conserved sequence elements (CSE) well known to those in the art. CSEs include, but are not limited to, an alphavirus 5′ UTR, a 51-nt CSE, a 24-nt CSE, a subgenomic promoter sequence (e.g., a 26S subgenomic promoter sequence), a 19-nt CSE, and an alphavirus 3′ UTR.

The term “RNA polymerase” includes polymerases that catalyze the production of RNA polynucleotides from a DNA template. RNA polymerases include, but are not limited to, bacteriophage derived polymerases including T3, T7, and SP6.

The term “lipid” includes hydrophobic and/or amphiphilic molecules. Lipids can be cationic, anionic, or neutral. Lipids can be synthetic or naturally derived, and in some instances biodegradable. Lipids can include cholesterol, phospholipids, lipid conjugates including, but not limited to, polyethyleneglycol (PEG) conjugates (PEGylated lipids), waxes, oils, glycerides, fats, and fat-soluble vitamins Lipids can also include dilinoleylmethyl-4-dimethylaminobutyrate (MC3) and MC3-like molecules.

The term “lipid nanoparticle” or “LNP” includes vesicle like structures formed using a lipid containing membrane surrounding an aqueous interior, also referred to as liposomes. Lipid nanoparticles includes lipid-based compositions with a solid lipid core stabilized by a surfactant. The core lipids can be fatty acids, acylglycerols, waxes, and mixtures of these surfactants. Biological membrane lipids such as phospholipids, sphingomyelins, bile salts (sodium taurocholate), and sterols (cholesterol) can be utilized as stabilizers. Lipid nanoparticles can be formed using defined ratios of different lipid molecules, including, but not limited to, defined ratios of one or more cationic, anionic, or neutral lipids. Lipid nanoparticles can encapsulate molecules within an outer-membrane shell and subsequently can be contacted with target cells to deliver the encapsulated molecules to the host cell cytosol. Lipid nanoparticles can be modified or functionalized with non-lipid molecules, including on their surface. Lipid nanoparticles can be single-layered (unilamellar) or multi-layered (multilamellar). Lipid nanoparticles can be complexed with nucleic acid. Unilamellar lipid nanoparticles can be complexed with nucleic acid, wherein the nucleic acid is in the aqueous interior. Multilamellar lipid nanoparticles can be complexed with nucleic acid, wherein the nucleic acid is in the aqueous interior, or to form or sandwiched between

Abbreviations: MHC: major histocompatibility complex; HLA: human leukocyte antigen, or the human MHC gene locus; NGS: next-generation sequencing; PPV: positive predictive value; TSNA: tumor-specific neoantigen; FFPE: formalin-fixed, paraffin-embedded; NMD: nonsense-mediated decay; NSCLC: non-small-cell lung cancer; DC: dendritic cell.

It should be noted that, as used in the specification and the appended claims, the singular forms “a,” “an,” and “the” include plural referents unless the context clearly dictates otherwise.

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

All references, issued patents and patent applications cited within the body of the specification are hereby incorporated by reference in their entirety, for all purposes.

### II. Antigen Identification

Research methods for NGS analysis of tumor and normal exome and transcriptomes have been described and applied in the antigen identification space.^{6,14,15} Certain optimizations for greater sensitivity and specificity for antigen identification in the clinical setting can be considered. These optimizations can be grouped into two areas, those related to laboratory processes and those related to the NGS data analysis. The research methods described can also be applied to identification of antigens in other settings, such as identification of identifying antigens from an infectious disease organism, an infection in a subject, or an infected cell of a subject. Examples of optimizations are known to those skilled in the art, for example the methods described in more detail in U.S. Pat. No. 10,055,540, US Application Pub. No. US20200010849A1, U.S. application Ser. No. 16/606,577, and international patent application publications WO2020181240A1, WO/2018/195357 and WO/2018/208856, each herein incorporated by reference, in their entirety, for all purposes.

Methods for identifying antigens (e.g., antigens derived from a tumor or an infectious disease organism) include identifying antigens that are likely to be presented on a cell surface (e.g., presented by MHC on a tumor cell, an infected cell, or an immune cell, including professional antigen presenting cells such as dendritic cells), and/or are likely to be immunogenic. As an example, one such method may comprise the steps of: obtaining at least one of exome, transcriptome or whole genome nucleotide sequencing and/or expression data from a tumor, an infected cell, or an infectious disease organism, wherein the nucleotide sequencing data and/or expression data is used to obtain data representing peptide sequences of each of a set of antigens (e.g., antigens derived from a tumor or an infectious disease organism); inputting the peptide sequence of each antigen into one or more presentation models to generate a set of numerical likelihoods that each of the antigens is presented by one or more MHC alleles on a cell surface, such as a tumor cell or an infected cell of the subject, the set of numerical likelihoods having been identified at least based on received mass spectrometry data; and selecting a subset of the set of antigens based on the set of numerical likelihoods to generate a set of selected antigens.

II.B. Identification of Tumor Specific Mutations in Neoantigens

Also disclosed herein are methods for the identification of certain mutations (e.g., the variants or alleles that are present in cancer cells). In particular, these mutations can be present in the genome, transcriptome, proteome, or exome of cancer cells of a subject having cancer but not in normal tissue from the subject. Specific methods for identifying neoantigens, including shared neoantigens, that are specific to tumors are known to those skilled in the art, for example the methods described in more detail in U.S. Pat. No. 10,055,540, US Application Pub. No. US20200010849A1, and international patent application publications WO/2018/195357 and WO/2018/208856, each herein incorporated by reference, in their entirety, for all purposes. Examples of shared neoantigens that are specific to tumors are described in more detail in international patent application publication WO2019226941A1, herein incorporated by reference in its entirety, for all purposes. Shared neoantigens include, but are not limited to, KRAS-associated mutations (e.g., KRAS G12C, KRAS G12V, KRAS G12D, and/or KRAS Q61H mutations). For example, KRAS-associated MHC class I neoepitope can include those mutations with reference to wild-type (WT) human KRAS, such as with reference to the following exemplary amino acid sequence:

(SEQ ID NO: 27996)
MTEYKLVVVGAGGVGKSALTIQLIQNHFVDEYDPTIEDSYRKQVVIDGE
TCLLDILDTAGQEEYSAMRDQYMRTGEGFLCVFAINNTKSFEDIHHYRE
QIKRVKDSEDVPMVLVGNKCDLPSRTVDTKQAQDLARSYGIPFIETSAK
TRQRVEDAFYTLVREIRQYRLKKISKEEKTPGCVKIKKCIIM.

Genetic mutations in tumors can be considered useful for the immunological targeting of tumors if they lead to changes in the amino acid sequence of a protein exclusively in the tumor. Useful mutations include: (1) non-synonymous mutations leading to different amino acids in the protein; (2) read-through mutations in which a stop codon is modified or deleted, leading to translation of a longer protein with a novel tumor-specific sequence at the C-terminus; (3) splice site mutations that lead to the inclusion of an intron in the mature mRNA and thus a unique tumor-specific protein sequence; (4) chromosomal rearrangements that give rise to a chimeric protein with tumor-specific sequences at the junction of 2 proteins (i.e., gene fusion); (5) frameshift mutations or deletions that lead to a new open reading frame with a novel tumor-specific protein sequence. Mutations can also include one or more of non-frameshift indel, missense or nonsense substitution, splice site alteration, genomic rearrangement or gene fusion, or any genomic or expression alteration giving rise to a neoORF.

Peptides with mutations or mutated polypeptides arising from for example, splice-site, frameshift, readthrough, or gene fusion mutations in tumor cells can be identified by sequencing DNA, RNA or protein in tumor versus normal cells.

Also mutations can include previously identified tumor specific mutations. Known tumor mutations can be found at the Catalogue of Somatic Mutations in Cancer (COSMIC) database.

A variety of methods are available for detecting the presence of a particular mutation or allele in an individual's DNA or RNA. Advancements in this field have provided accurate, easy, and inexpensive large-scale SNP genotyping. For example, several techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods utilize amplification of a target genetic region, typically by PCR. Still other methods, based on the generation of small signal molecules by invasive cleavage followed by mass spectrometry or immobilized padlock probes and rolling-circle amplification. Several of the methods known in the art for detecting specific mutations are summarized below.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

Several methods have been developed to facilitate analysis of single nucleotide polymorphisms in genomic DNA or cellular RNA. For example, a single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide(s) present in the polymorphic site of the target molecule is complementary to that of the nucleotide derivative used in the reaction. This method has the advantage that it does not require the determination of large amounts of extraneous sequence data.

A solution-based method can be used for determining the identity of a nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. can be a heterogeneous phase assay, in which the primer or the target molecule is immobilized to a solid phase.

Several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA in that they utilize incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

A number of initiatives obtain sequence information directly from millions of individual molecules of DNA or RNA in parallel. Real-time single molecule sequencing-by-synthesis technologies rely on the detection of fluorescent nucleotides as they are incorporated into a nascent strand of DNA that is complementary to the template being sequenced. In one method, oligonucleotides 30-50 bases in length are covalently anchored at the 5′ end to glass cover slips. These anchored strands perform two functions. First, they act as capture sites for the target template strands if the templates are configured with capture tails complementary to the surface-bound oligonucleotides. They also act as primers for the template directed primer extension that forms the basis of the sequence reading. The capture primers function as a fixed position site for sequence determination using multiple cycles of synthesis, detection, and chemical cleavage of the dye-linker to remove the dye. Each cycle includes adding the polymerase/labeled nucleotide mixture, rinsing, imaging and cleavage of dye. In an alternative method, polymerase is modified with a fluorescent donor molecule and immobilized on a glass slide, while each nucleotide is color-coded with an acceptor fluorescent moiety attached to a gamma-phosphate. The system detects the interaction between a fluorescently-tagged polymerase and a fluorescently modified nucleotide as the nucleotide becomes incorporated into the de novo chain. Other sequencing-by-synthesis technologies also exist.

Any suitable sequencing-by-synthesis platform can be used to identify mutations. As described above, four major sequencing-by-synthesis platforms are currently available: the Genome Sequencers from Roche/454 Life Sciences, the 1G Analyzer from Illumina/Solexa, the SOLiD system from Applied BioSystems, and the Heliscope system from Helicos Biosciences. Sequencing-by-synthesis platforms have also been described by Pacific BioSciences and VisiGen Biotechnologies. In some embodiments, a plurality of nucleic acid molecules being sequenced is bound to a support (e.g., solid support). To immobilize the nucleic acid on a support, a capture sequence/universal priming site can be added at the 3′ and/or 5′ end of the template. The nucleic acids can be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer.

As an alternative to a capture sequence, a member of a coupling pair (such as, e.g., antibody/antigen, receptor/ligand, or the avidin-biotin pair as described in, e.g., US Patent Application No. 2006/0252077) can be linked to each fragment to be captured on a surface coated with a respective second member of that coupling pair.

Subsequent to the capture, the sequence can be analyzed, for example, by single molecule detection/sequencing, e.g., as described in the Examples and in U.S. Pat. No. 7,283,337, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or can be done in a step-and-repeat mode. For real-time analysis, different optical labels to each nucleotide can be incorporated and multiple lasers can be utilized for stimulation of incorporated nucleotides.

Sequencing can also include other massively parallel sequencing or next generation sequencing (NGS) techniques and platforms. Additional examples of massively parallel sequencing techniques and platforms are the Illumina HiSeq or MiSeq, Thermo PGM or Proton, the Pac Bio RS II or Sequel, Qiagen's Gene Reader, and the Oxford Nanopore MinION. Additional similar current massively parallel sequencing technologies can be used, as well as future generations of these technologies.

Any cell type or tissue can be utilized to obtain nucleic acid samples for use in methods described herein. For example, a DNA or RNA sample can be obtained from a tumor or a bodily fluid, e.g., blood, obtained by known techniques (e.g. venipuncture) or saliva. Alternatively, nucleic acid tests can be performed on dry samples (e.g. hair or skin). In addition, a sample can be obtained for sequencing from a tumor and another sample can be obtained from normal tissue for sequencing where the normal tissue is of the same tissue type as the tumor. A sample can be obtained for sequencing from a tumor and another sample can be obtained from normal tissue for sequencing where the normal tissue is of a distinct tissue type relative to the tumor.

Tumors can include one or more of lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, and T cell lymphocytic leukemia, non-small cell lung cancer, and small cell lung cancer.

Alternatively, protein mass spectrometry can be used to identify or validate the presence of mutated peptides bound to MHC proteins on tumor cells. Peptides can be acid-eluted from tumor cells or from HLA molecules that are immunoprecipitated from tumor, and then identified using mass spectrometry.

### III. Immune Modulators

Vectors described herein, such as ChAdV vectors described herein or alphavirus vectors described herein, can comprise a nucleic acid which encodes at least one antigen and the same or a separate vector can comprise a nucleic acid which encodes at least one immune modulator. An immune modulator can include a binding molecule (e.g., an antibody such as an scFv) which binds to and blocks the activity of an immune checkpoint molecule. An immune modulator can include a cytokine, such as IL-2, IL-7, IL-12 (including IL-12 p35, p40, p70, and/or p70-fusion constructs), IL-15, or IL-21. An immune modulator can include a modified cytokine (e.g., pegIL-2). Vectors can comprise an antigen cassette and one or more nucleic acid molecules encoding an immune modulator.

Illustrative immune checkpoint molecules that can be targeted for blocking or inhibition include, but are not limited to, CTLA-4, 4-1BB (CD137), 4-1BBL (CD137L), PDL1, PDL2, PD1, B7-H3, B7-H4, BTLA, HVEM, TIM3, GAL9, LAG3, TIM3, B7H3, B7H4, VISTA, KIR, 2B4 (belongs to the CD2 family of molecules and is expressed on all NK, γδ, and memory CD8+(4) T cells), CD160 (also referred to as BY55), and CGEN-15049 Immune checkpoint inhibitors include antibodies, or antigen binding fragments thereof, or other binding proteins, that bind to and block or inhibit the activity of one or more of CTLA-4, PDL1, PDL2, PD1, B7-H3, B7-H4, BTLA, HVEM, TIM3, GAL9, LAG3, TIM3, B7H3, B7H4, VISTA, KIR, 2B4, CD160, and CGEN-15049. Illustrative immune checkpoint inhibitors include Tremelimumab (CTLA-4 blocking antibody), anti-OX40, PD-L1 monoclonal Antibody (Anti-B7-H1; MEDI4736), ipilimumab, MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor). Antibody-encoding sequences can be engineered into vectors such as C68 using ordinary skill in the art. An exemplary method is described in Fang et al., Stable antibody expression at therapeutic levels using the 2A peptide. Nat Biotechnol. 2005 May; 23(5):584-90. Epub 2005 Apr. 17; herein incorporated by reference for all purposes.

### IV. Payloads and Antigens

A payload nucleic acid sequence can be any nucleic acid sequence desired to be delivered to a cell of interest. In general, the payload is a nucleic acid sequence linked to a promoter to drive expression of the nucleic acid sequence. The payload nucleic acid sequence can encode a polypeptide (i.e., a nucleic acid sequence capable of being transcribed and translated into a protein). In general, a payload nucleic acid sequence encoding a peptide can encode any protein desired to be expressed in a cell. Examples of proteins include, but are not limited to, an antigen (e.g., a MHC class I epitope, a MHC class II epitope, or an epitope capable of stimulating a B cell response), an antibody, a cytokine, a chimeric antigen receptor (CAR), a T-cell receptor, or a genome-editing system component (e.g., a nuclease used in a genome-editing system). Genome-editing systems include, but are not limited to, a CRISPR system, a zinc-finger system, a meganuclease system, or a TALEN system. The payload nucleic acid sequence can be non-coding (i.e., a nucleic acid sequence capable of being transcribed but is not translated into a protein). In general, a non-coding payload nucleic acid sequence can be any non-coding polynucleotide desired to be expressed in a cell. Examples of non-coding polynucleotides include, but are not limited to, RNA interference (RNAi) polynucleotides (e.g., antisense oligonucleotides, shRNAs, siRNAs, miRNAs etc.) or genome-editing system polynucleotide (e.g., a guide RNA [gRNA], a single-guide RNA [sgRNA], a trans-activating CRISPR [tracrRNA], and/or a CRISPR RNA [crRNA]). A payload nucleic acid sequence can encode two or more (e.g., 2, 3, 4, 5 or more) distinct polypeptides (e.g., two or more distinct epitope sequences linked together) or contain two or more distinct non-coding nucleic acid sequences (e.g., two or more distinct RNAi polynucleotides). A payload nucleic acid sequence can have a combination of polypeptide-encoding nucleic acid sequences and non-coding nucleic acid sequences.

A vector can contain between 1 and 30 payload-encoding nucleic acid sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different payload-encoding nucleic acid sequences, 6, 7, 8, 9, 10 11, 12, 13, or 14 different payload-encoding nucleic acid sequences, or 12, 13 or 14 different payload-encoding nucleic acid sequences. Payload-encoding nucleic acid sequences can refer to the payload encoding portion of a “cassette.” Features of a cassette are described in greater detail herein. A cassette can contain two or more payload-encoding nucleic acid sequences linked together in a cassette (e.g., as an illustrative non-limiting example, concatenated antigen-encoding nucleic acid sequence encoding concatenated T cell epitopes)

A vector can contain between 1 and 30 distinct payload-encoding nucleic acid sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more distinct payload-encoding nucleic acid sequences, 6, 7, 8, 9, 10 11, 12, 13, or 14 distinct payload-encoding nucleic acid sequences, or 12, 13 or 14 distinct payload-encoding nucleic acid sequences. Payload-encoding nucleic acid sequences can refer to sequences for individual payload sequences, e.g., as an illustrative non-limiting example, each of the concatenated T cell epitopes of two or more payload-encoding nucleic acid sequences linked together in a cassette.

Antigens can include nucleotides or polypeptides. For example, an antigen can be an RNA sequence that encodes for a polypeptide sequence. Antigens useful in vaccines can therefore include nucleotide sequences or polypeptide sequences. Antigens that can be used for cancer vaccines are described in international patent application publication WO/2019/226941, which is herein incorporated by reference, in its entirety, for all purposes.

Disclosed herein are isolated peptides that comprise tumor specific mutations identified by the methods disclosed herein, peptides that comprise known tumor specific mutations, and mutant polypeptides or fragments thereof identified by methods disclosed herein. Neoantigen peptides can be described in the context of their coding sequence where a neoantigen includes the nucleotide sequence (e.g., DNA or RNA) that codes for the related polypeptide sequence.

Also disclosed herein are peptides derived from any polypeptide known to or have been found to have altered expression in a tumor cell or cancerous tissue in comparison to a normal cell or tissue, for example any polypeptide known to or have been found to be aberrantly expressed in a tumor cell or cancerous tissue in comparison to a normal cell or tissue. Suitable polypeptides from which the antigenic peptides can be derived can be found for example in the COSMIC database. COSMIC curates comprehensive information on somatic mutations in human cancer. The peptide contains the tumor specific mutation. Tumor antigens (e.g., shared tumor antigens and tumor neoantigens) can include, but are not limited to, those described in U.S. application Ser. No. 17/058,128, herein incorporated by reference for all purposes. Antigen peptides can be described in the context of their coding sequence where an antigen includes the nucleotide sequence (e.g., DNA or RNA) that codes for the related polypeptide sequence.

One or more polypeptides encoded by an antigen nucleotide sequence can comprise at least one of: a binding affinity with MHC with an IC50 value of less than 1000 nM, for MHC Class I peptides a length of 8-15, 8, 9, 10, 11, 12, 13, 14, or 15 amino acids, presence of sequence motifs within or near the peptide promoting proteasome cleavage, and presence or sequence motifs promoting TAP transport. For MHC Class II peptides a length 6-30, 6, 7, 8, 9, 10, 11, 12, 13, 14, 15, 16, 17, 18, 19, 20, 21, 22, 23, 24, 25, 26, 27, 28, 29, or 30 amino acids, presence of sequence motifs within or near the peptide promoting cleavage by extracellular or lysosomal proteases (e.g., cathepsins) or HLA-DM catalyzed HLA binding.

One or more antigens can be presented on the surface of a tumor.

One or more antigens can be is immunogenic in a subject having a tumor, e.g., capable of eliciting a T cell response or a B cell response in the subject.

One or more antigens that induce an autoimmune response in a subject can be excluded from consideration in the context of vaccine generation for a subject having a tumor.

The size of at least one antigenic peptide molecule can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In specific embodiments the antigenic peptide molecules are equal to or less than 50 amino acids.

Antigenic peptides and polypeptides can be: for MHC Class I 15 residues or less in length and usually consist of between about 8 and about 11 residues, particularly 9 or 10 residues; for MHC Class II, 6-30 residues, inclusive.

Antigenic peptides and polypeptides can be presented on an HLA protein. In some aspects antigenic peptides and polypeptides are presented on an HLA protein with greater affinity than a wild-type peptide. In some aspects, an antigenic peptide or polypeptide can have an IC50 of at least less than 5000 nM, at least less than 1000 nM, at least less than 500 nM, at least less than 250 nM, at least less than 200 nM, at least less than 150 nM, at least less than 100 nM, at least less than 50 nM or less.

In some aspects, antigenic peptides and polypeptides do not induce an autoimmune response and/or invoke immunological tolerance when administered to a subject.

Also provided are compositions comprising at least two or more antigenic peptides. In some embodiments the composition contains at least two distinct peptides. At least two distinct peptides can be derived from the same polypeptide. By distinct polypeptides is meant that the peptide vary by length, amino acid sequence, or both. The peptides are derived from any polypeptide known to or have been found to contain a tumor specific mutation or peptides derived from any polypeptide known to or have been found to have altered expression in a tumor cell or cancerous tissue in comparison to a normal cell or tissue, for example any polypeptide known to or have been found to be aberrantly expressed in a tumor cell or cancerous tissue in comparison to a normal cell or tissue. Suitable polypeptides from which the antigenic peptides can be derived can be found for example in the COSMIC database or the AACR Genomics Evidence Neoplasia Information Exchange (GENIE) database. COSMIC curates comprehensive information on somatic mutations in human cancer. AACR GENIE aggregates and links clinical-grade cancer genomic data with clinical outcomes from tens of thousands of cancer patients. The peptide contains the tumor specific mutation. In some aspects the tumor specific mutation is a driver mutation for a particular cancer type.

Also disclosed herein are peptides derived from any polypeptide associated with an infectious disease organism, an infection in a subject, or an infected cell of a subject. Antigens can be derived from nucleotide sequences or polypeptide sequences of an infectious disease organism. Polypeptide sequences of an infectious disease organism include, but are not limited to, a pathogen-derived peptide, a virus-derived peptide, a bacteria-derived peptide, a fungus-derived peptide, and/or a parasite-derived peptide. Infectious disease organism include, but are not limited to, Severe acute respiratory syndrome-related coronavirus (SARS), severe acute respiratory syndrome coronavirus 2 (SARS-CoV-2), Ebola, HIV, Hepatitis B virus (HBV), influenza, Hepatitis C virus (HCV), Human papillomavirus (HPV), Cytomegalovirus (CMV), Chikungunya virus, Respiratory syncytial virus (RSV), Dengue virus, an orthymyxoviridae family virus, and tuberculosis.

Disclosed herein are isolated peptides that comprise infectious disease organism specific antigens or epitopes identified by the methods disclosed herein, peptides that comprise known infectious disease organism specific antigens or epitopes, and mutant polypeptides or fragments thereof identified by methods disclosed herein. Antigen peptides can be described in the context of their coding sequence where an antigen includes the nucleotide sequence (e.g., DNA or RNA) that codes for the related polypeptide sequence.

Vectors and associated compositions described herein can be used to deliver antigens from any organism, including their toxins or other by-products, to prevent and/or treat infection or other adverse reactions associated with the organism or its by-product.

Antigens that can be incorporated into a vaccine (e.g., encoded in a cassette) include immunogens which are useful to immunize a human or non-human animal against viruses, such as pathogenic viruses which infect human and non-human vertebrates. Antigens may be selected from a variety of viral families Example of desirable viral families against which an immune response would be desirable include, the picornavirus family, which includes the genera rhinoviruses, which are responsible for about 50% of cases of the common cold; the genera enteroviruses, which include polioviruses, coxsackieviruses, echoviruses, and human enteroviruses such as hepatitis A virus; and the genera apthoviruses, which are responsible for foot and mouth diseases, primarily in non-human animals. Within the picornavirus family of viruses, target antigens include the VP1, VP2, VP3, VP4, and VPG. Another viral family includes the calicivirus family, which encompasses the Norwalk group of viruses, which are an important causative agent of epidemic gastroenteritis. Still another viral family desirable for use in targeting antigens for stimulating immune responses in humans and non-human animals is the togavirus family, which includes the genera alphavirus, which include Sindbis viruses, RossRiver virus, and Venezuelan, Eastern & Western Equine encephalitis, and rubivirus, including Rubella virus. The Flaviviridae family includes dengue, yellow fever, Japanese encephalitis, St. Louis encephalitis and tick borne encephalitis viruses. Other target antigens may be generated from the Hepatitis C or the coronavirus family, which includes a number of non-human viruses such as infectious bronchitis virus (poultry), porcine transmissible gastroenteric virus (pig), porcine hemagglutinating encephalomyelitis virus (pig), feline infectious peritonitis virus (cats), feline enteric coronavirus (cat), canine coronavirus (dog), and human respiratory coronaviruses, which may cause the common cold and/or non-A, B or C hepatitis. Within the coronavirus family, target antigens include the E1 (also called M or matrix protein), E2 (also called S or Spike protein), E3 (also called HE or hemagglutin-elterose) glycoprotein (not present in all coronaviruses), or N (nucleocapsid). Still other antigens may be targeted against the rhabdovirus family, which includes the genera vesiculovirus (e.g., Vesicular Stomatitis Virus), and the general lyssavirus (e.g., rabies). Within the rhabdovirus family, suitable antigens may be derived from the G protein or the N protein. The family filoviridae, which includes hemorrhagic fever viruses such as Marburg and Ebola virus, may be a suitable source of antigens. The paramyxovirus family includes parainfluenza Virus Type 1, parainfluenza Virus Type 3, bovine parainfluenza Virus Type 3, rubulavirus (mumps virus), parainfluenza Virus Type 2, parainfluenza virus Type 4, Newcastle disease virus (chickens), rinderpest, morbillivirus, which includes measles and canine distemper, and pneumovirus, which includes respiratory syncytial virus (e.g., the glyco-(G) protein and the fusion (F) protein, for which sequences are available from GenBank). Influenza virus is classified within the family orthomyxovirus and can be suitable source of antigens (e.g., the HA protein, the N1 protein). The bunyavirus family includes the genera bunyavirus (California encephalitis, La Crosse), phlebovirus (Rift Valley Fever), hantavirus (puremala is a hemahagin fever virus), nairovirus (Nairobi sheep disease) and various unassigned bungaviruses. The arenavirus family provides a source of antigens against LCM and Lassa fever virus. The reovirus family includes the genera reovirus, rotavirus (which causes acute gastroenteritis in children), orbiviruses, and cultivirus (Colorado Tick fever, Lebombo (humans), equine encephalosis, blue tongue). The retrovirus family includes the sub-family oncorivirinal which encompasses such human and veterinary diseases as feline leukemia virus, HTLVI and HTLVII, lentivirinal (which includes human immunodeficiency virus (HIV), simian immunodeficiency virus (SIV), feline immunodeficiency virus (FIV), equine infectious anemia virus, and spumavirinal). Among the lentiviruses, many suitable antigens have been described and can readily be selected. Examples of suitable HIV and SIV antigens include, without limitation the gag, pol, Vif, Vpx, VPR, Env, Tat, Nef, and Rev proteins, as well as various fragments thereof. For example, suitable fragments of the Env protein may include any of its subunits such as the gp120, gp160, gp41, or smaller fragments thereof, e.g., of at least about 8 amino acids in length. Similarly, fragments of the tat protein may be selected. [See, U.S. Pat. Nos. 5,891,994 and 6,193,981.] See, also, the HIV and SIV proteins described in D. H. Barouch et al, J. Virol., 75(5):2462-2467 (March 2001), and R. R Amara, et al, Science, 292:69-74 (6 Apr. 2001). In another example, the HIV and/or SIV immunogenic proteins or peptides may be used to form fusion proteins or other immunogenic molecules. See, e.g., the HIV-1 Tat and/or Nef fusion proteins and immunization regimens described in WO 01/54719, published Aug. 2, 2001, and WO 99/16884, published Apr. 8, 1999. The invention is not limited to the HIV and/or SIV immunogenic proteins or peptides described herein. In addition, a variety of modifications to these proteins have been described or could readily be made by one of skill in the art. See, e.g., the modified gag protein that is described in U.S. Pat. No. 5,972,596. Further, any desired HIV and/or SIV immunogens may be delivered alone or in combination. Such combinations may include expression from a single vector or from multiple vectors. The papovavirus family includes the sub-family polyomaviruses (BKU and JCU viruses) and the sub-family papillomavirus (associated with cancers or malignant progression of papilloma). The adenovirus family includes viruses (EX, AD7, ARD, O.B.) which cause respiratory disease and/or enteritis. The parvovirus family feline parvovirus (feline enteritis), feline panleucopeniavirus, canine parvovirus, and porcine parvovirus. The herpesvirus family includes the sub-family alphaherpesvirinae, which encompasses the genera simplexvirus (HSVI, HSVII), varicellovirus (pseudorabies, varicella zoster) and the sub-family betaherpesvirinae, which includes the genera cytomegalovirus (Human CMV), muromegalovirus) and the sub-family gammaherpesvirinae, which includes the genera lymphocryptovirus, EBV (Burkitts lymphoma), infectious rhinotracheitis, Marek's disease virus, and rhadinovirus. The poxvirus family includes the sub-family chordopoxyirinae, which encompasses the genera orthopoxvirus (Variola (Smallpox) and Vaccinia (Cowpox)), parapoxvirus, avipoxvirus, capripoxvirus, leporipoxvirus, suipoxvirus, and the sub-family entomopoxyirinae. The hepadnavirus family includes the Hepatitis B virus. One unclassified virus which may be suitable source of antigens is the Hepatitis delta virus. Still other viral sources may include avian infectious bursal disease virus and porcine respiratory and reproductive syndrome virus. The alphavirus family includes equine arteritis virus and various Encephalitis viruses.

Antigens that can be incorporated into a vaccine (e.g., encoded in a cassette) also include immunogens which are useful to immunize a human or non-human animal against pathogens including bacteria, fungi, parasitic microorganisms or multicellular parasites which infect human and non-human vertebrates. Examples of bacterial pathogens include pathogenic gram-positive cocci include pneumococci; staphylococci; and streptococci. Pathogenic gram-negative cocci include meningococcus; gonococcus. Pathogenic enteric gram-negative bacilli include enterobacteriaceae; Pseudomonas, acinetobacteria and Eikenella; melioidosis; Salmonella; Shigella; haemophilus (Haemophilus influenzae, Haemophilus somnus); moraxella; H. ducreyi (which causes chancroid); brucella; Franisella tularensis (which causes tularemia); yersinia (pasteurella); streptobacillus moniliformis and spirillum. Gram-positive bacilli include Listeria monocytogenes; erysipelothrix rhusiopathiae; Corynebacterium diphtheria (diphtheria); cholera; B. anthracis (anthrax); donovanosis (granuloma inguinale); and bartonellosis. Diseases caused by pathogenic anaerobic bacteria include tetanus; botulism; other clostridia; tuberculosis; leprosy; and other mycobacteria. Examples of specific bacterium species are, without limitation, Streptococcus pneumoniae, Streptococcus pyogenes, Streptococcus agalactiae, Streptococcus faecalis, Moraxella catarrhalis, Helicobacter pylori, Neisseria meningitidis, Neisseria gonorrhoeae, Chlamydia trachomatis, Chlamydia pneumoniae, Chlamydia psittaci, Bordetella pertussis, Salmonella typhi, Salmonella typhimurium, Salmonella choleraesuis, Escherichia coli, Shigella, Vibrio cholerae, Corynebacterium diphtheriae, Mycobacterium tuberculosis, Mycobacterium avium, Mycobacterium intracellulare complex, Proteus mirabilis, Proteus vulgaris, Staphylococcus aureus, Clostridium tetani, Leptospira interrogans, Borrelia burgdorferi, Pasteurella haemolytica, Pasteurella multocida, Actinobacillus pleuropneumoniae and Mycoplasma gallisepticum. Pathogenic spirochetal diseases include syphilis; treponematoses: yaws, pinta and endemic syphilis; and leptospirosis. Other infections caused by higher pathogen bacteria and pathogenic fungi include actinomycosis; nocardiosis; cryptococcosis (Cryptococcus), blastomycosis (Blastomyces), histoplasmosis (Histoplasma) and coccidioidomycosis (Coccidiodes); candidiasis (Candida), aspergillosis (Aspergillis), and mucormycosis; sporotrichosis; paracoccidiodomycosis, petriellidiosis, torulopsosis, mycetoma and chromomycosis; and dermatophytosis. Rickettsial infections include Typhus fever, Rocky Mountain spotted fever, Q fever, and Rickettsialpox. Examples of mycoplasma and chlamydial infections include: Mycoplasma pneumoniae; lymphogranuloma venereum; psittacosis; and perinatal chlamydial infections. Pathogenic eukaryotes encompass pathogenic protozoans and helminths and infections produced thereby include: amebiasis; malaria; leishmaniasis (e.g., caused by Leishmania major); trypanosomiasis; toxoplasmosis (e.g., caused by Toxoplasma gondii); Pneumocystis carinii; Trichans; Toxoplasma gondii; babesiosis; giardiasis (e.g., caused by Giardia); trichinosis (e.g., caused by Trichomonas); filariasis; schistosomiasis (e.g., caused by Schistosoma); nematodes; trematodes or flukes; and cestode (tapeworm) infections. Other parasitic infections may be caused by Ascaris, Trichuris, Cryptosporidium, and Pneumocystis carinii, among others.

Also disclosed herein are peptides derived from any polypeptide associated with an infectious disease organism, an infection in a subject, or an infected cell of a subject. Antigens can be derived from nucleic acid sequences or polypeptide sequences of an infectious disease organism. Polypeptide sequences of an infectious disease organism include, but are not limited to, a pathogen-derived peptide, a virus-derived peptide, a bacteria-derived peptide, a fungus-derived peptide, and/or a parasite-derived peptide. Infectious disease organism include, but are not limited to, Severe acute respiratory syndrome-related coronavirus (SARS), severe acute respiratory syndrome coronavirus 2 (SARS-CoV-2), Ebola, HIV, Hepatitis B virus (HBV), influenza, Hepatitis C virus (HCV), Human papillomavirus (HPV), Cytomegalovirus (CMV), Chikungunya virus, Respiratory syncytial virus (RSV), Dengue virus, an orthymyxoviridae family virus, and tuberculosis.

Antigens can be selected that are predicted to be presented on the cell surface of a cell, such as a tumor cell, an infected cell, or an immune cell, including professional antigen presenting cells such as dendritic cells. Antigens can be selected that are predicted to be immunogenic.

One or more polypeptides encoded by an antigen nucleotide sequence can comprise at least one of: a binding affinity with MHC with an IC50 value of less than 1000 nM, for MHC Class I peptides a length of 8-15, 8, 9, 10, 11, 12, 13, 14, or 15 amino acids, presence of sequence motifs within or near the peptide promoting proteasome cleavage, and presence or sequence motifs promoting TAP transport. For MHC Class II peptides a length 6-30, 6, 7, 8, 9, 10, 11, 12, 13, 14, 15, 16, 17, 18, 19, 20, 21, 22, 23, 24, 25, 26, 27, 28, 29, or 30 amino acids, presence of sequence motifs within or near the peptide promoting cleavage by extracellular or lysosomal proteases (e.g., cathepsins) or HLA-DM catalyzed HLA binding.

One or more antigens can be presented on the surface of a tumor. One or more antigens can be presented on the surface of an infected cell.

One or more antigens can be immunogenic in a subject having a tumor, e.g., capable of stimulating a T cell response and/or a B cell response in the subject. One or more antigens can be immunogenic in a subject having or suspected to have an infection, e.g., capable of stimulating a T cell response and/or a B cell response in the subject. One or more antigens can be immunogenic in a subject at risk of an infection, e.g., capable of stimulating a T cell response and/or a B cell response in the subject that provides immunological protection (i.e., immunity) against the infection, e.g., such as stimulating the production of memory T cells, memory B cells, or antibodies specific to the infection.

One or more antigens can be capable of stimulating a B cell response, such as the production of antibodies that recognize the one or more antigens (e.g., antibodies that recognize a tumor or an infectious disease antigen). Antibodies can recognize linear polypeptide sequences or recognize secondary and tertiary structures. Accordingly, B cell antigens can include linear polypeptide sequences or polypeptides having secondary and tertiary structures, including, but not limited to, full-length proteins, protein subunits, protein domains, or any polypeptide sequence known or predicted to have secondary and tertiary structures. Antigens capable of stimulating a B cell response to a tumor or an infectious disease antigen can be an antigen found on the surface of tumor cell or an infectious disease organism, respectively. Antigens capable of eliciting a B cell response to a tumor or an infectious disease antigen can be an intracellular neoantigen expressed in a tumor or an infectious disease organism, respectively.

One or more antigens can include a combination of antigens capable of stimulating a T cell response (e.g., peptides including predicted T cell epitope sequences) and distinct antigens capable of stimulating a B cell response (e.g., full-length proteins, protein subunits, protein domains).

One or more antigens that stimulate an autoimmune response in a subject can be excluded from consideration in the context of vaccine generation for a subject.

The size of at least one antigenic peptide molecule (e.g., an epitope sequence) can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In specific embodiments the antigenic peptide molecules are equal to or less than 50 amino acids.

Antigenic peptides and polypeptides can be: for MHC Class I 15 residues or less in length and usually include between about 8 and about 11 residues, particularly 9 or 10 residues; for MHC Class II, 6-30 residues, inclusive.

If desirable, a longer peptide can be designed in several ways. In one case, when presentation likelihoods of peptides on HLA alleles are predicted or known, a longer peptide could include either: (1) individual presented peptides with an extensions of 2-5 amino acids toward the N- and C-terminus of each corresponding gene product; (2) a concatenation of some or all of the presented peptides with extended sequences for each. In another case, when sequencing reveals a long (>10 residues) neoepitope sequence present in the tumor (e.g. due to a frameshift, read-through or intron inclusion that leads to a novel peptide sequence), a longer peptide would include: (3) the entire stretch of novel tumor-specific or infectious disease-specific amino acids—thus bypassing the need for computational or in vitro test-based selection of the strongest HLA-presented shorter peptide. In both cases, use of a longer peptide allows endogenous processing by patient cells and may lead to more effective antigen presentation and stimulation of T cell responses. Longer peptides can also include a full-length protein, a protein subunit, a protein domain, and combinations thereof of a peptide, such as those expressed in a tumor or an infectious disease organism, respectively. Longer peptides (e.g., full-length protein, protein subunit, or protein domain) and combinations thereof can be included to stimulate a B cell response.

Antigenic peptides and polypeptides can be presented on an HLA protein. In some aspects antigenic peptides and polypeptides are presented on an HLA protein with greater affinity than a wild-type peptide. In some aspects, an antigenic peptide or polypeptide can have an IC50 of at least less than 5000 nM, at least less than 1000 nM, at least less than 500 nM, at least less than 250 nM, at least less than 200 nM, at least less than 150 nM, at least less than 100 nM, at least less than 50 nM or less.

In some aspects, antigenic peptides and polypeptides do not induce an autoimmune response and/or invoke immunological tolerance when administered to a subject.

Also provided are compositions comprising at least two or more antigenic peptides. In some embodiments the composition contains at least two distinct peptides. At least two distinct peptides can be derived from the same polypeptide. By distinct polypeptides is meant that the peptide vary by length, amino acid sequence, or both. A peptide can include a tumor-specific mutation. Tumor-specific peptides can be derived from any polypeptide known to or have been found to contain a tumor specific mutation or peptides derived from any polypeptide known to or have been found to have altered expression in a tumor cell or cancerous tissue in comparison to a normal cell or tissue, for example any polypeptide known to or have been found to be aberrantly expressed in a tumor cell or cancerous tissue in comparison to a normal cell or tissue. The peptides can be derived from any polypeptide known to or suspected to be associated with an infectious disease organism, or peptides derived from any polypeptide known to or have been found to have altered expression in an infected cell in comparison to a normal cell or tissue (e.g., an infectious disease polynucleotide or polypeptide, including infectious disease polynucleotides or polypeptides with expression restricted to a host cell). Suitable polypeptides from which the antigenic peptides can be derived can be found for example in the COSMIC database or the AACR Genomics Evidence Neoplasia Information Exchange (GENIE) database. COSMIC curates comprehensive information on somatic mutations in human cancer. AACR GENIE aggregates and links clinical-grade cancer genomic data with clinical outcomes from tens of thousands of cancer patients. In some aspects the tumor specific mutation is a driver mutation for a particular cancer type.

Antigenic peptides and polypeptides having a desired activity or property can be modified to provide certain desired attributes, e.g., improved pharmacological characteristics, while increasing or at least retaining substantially all of the biological activity of the unmodified peptide to bind the desired MHC molecule and activate the appropriate T cell. For instance, antigenic peptide and polypeptides can be subject to various changes, such as substitutions, either conservative or non-conservative, where such changes might provide for certain advantages in their use, such as improved MHC binding, stability or presentation. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gln; Ser, Thr; Lys, Arg; and Phe, Tyr. The effect of single amino acid substitutions may also be probed using D-amino acids. Such modifications can be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, Ill., Pierce), 2d Ed. (1984).

Modifications of peptides and polypeptides with various amino acid mimetics or unnatural amino acids can be particularly useful in increasing the stability of the peptide and polypeptide in vivo. Stability can be assayed in a number of ways. For instance, peptidases and various biological media, such as human plasma and serum, have been used to test stability. See, e.g., Verhoef et al., Eur. J. Drug Metab Pharmacokin. 11:291-302 (1986). Half-life of the peptides can be conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloracetic acid or ethanol. The cloudy reaction sample is cooled (4 degrees C.) for 15 minutes and then spun to pellet the precipitated serum proteins. The presence of the peptides is then determined by reversed-phase HPLC using stability-specific chromatography conditions.

The peptides and polypeptides can be modified to provide desired attributes other than improved serum half-life. For instance, the ability of the peptides to stimulate CTL activity can be enhanced by linkage to a sequence which contains at least one epitope that is capable of stimulating a T helper cell response Immunogenic peptides/T helper conjugates can be linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus can be a hetero- or homo-oligomer. When present, the spacer will usually be at least one or two residues, more usually three to six residues. Alternatively, the peptide can be linked to the T helper peptide without a spacer.

An antigenic peptide can be linked to the T helper peptide either directly or via a spacer either at the amino or carboxy terminus of the peptide. The amino terminus of either the antigenic peptide or the T helper peptide can be acylated. Exemplary T helper peptides include tetanus toxoid 830-843, influenza 307-319, malaria circumsporozoite 382-398 and 378-389.

Proteins or peptides can be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and can be found at computerized databases known to those of ordinary skill in the art. One such database is the National Center for Biotechnology Information's Genbank and GenPept databases located at the National Institutes of Health website. The coding regions for known genes can be amplified and/or expressed using the techniques disclosed herein or as would be known to those of ordinary skill in the art. Alternatively, various commercial preparations of proteins, polypeptides and peptides are known to those of skill in the art.

In a further aspect an antigen includes a nucleic acid (e.g. polynucleotide) that encodes an antigenic peptide or portion thereof. The polynucleotide can be, e.g., DNA, cDNA, PNA, CNA, RNA (e.g., mRNA), either single- and/or double-stranded, or native or stabilized forms of polynucleotides, such as, e.g., polynucleotides with a phosphorothioate backbone, or combinations thereof and it may or may not contain introns. A polynucleotide sequence encoding an antigen can be sequence-optimized to improve expression, such as through improving transcription, translation, post-transcriptional processing, and/or RNA stability. For example, polynucleotide sequence encoding an antigen can be codon-optimized. “Codon-optimization” herein refers to replacing infrequently used codons, with respect to codon bias of a given organism, with frequently used synonymous codons. Polynucleotide sequences can be optimized to improve post-transcriptional processing, for example optimized to reduce unintended splicing, such as through removal of splicing motifs (e.g., canonical and/or cryptic/non-canonical splice donor, branch, and/or acceptor sequences) and/or introduction of exogenous splicing motifs (e.g., splice donor, branch, and/or acceptor sequences) to bias favored splicing events. Exogenous intron sequences include, but are not limited to, those derived from SV40 (e.g., an SV40 mini-intron) and derived from immunoglobulins (e.g., human (3-globin gene). Exogenous intron sequences can be incorporated between a promoter/enhancer sequence and the antigen(s) sequence. Exogenous intron sequences for use in expression vectors are described in more detail in Callendret et al. (Virology. 2007 Jul. 5; 363(2): 288-302), herein incorporated by reference for all purposes. Polynucleotide sequences can be optimized to improve transcript stability, for example through removal of RNA instability motifs (e.g., AU-rich elements and 3′ UTR motifs) and/or repetitive nucleotide sequences. Polynucleotide sequences can be optimized to improve accurate transcription, for example through removal of cryptic transcriptional initiators and/or terminators. Polynucleotide sequences can be optimized to improve translation and translational accuracy, for example through removal of cryptic AUG start codons, premature polyA sequences, and/or secondary structure motifs. Polynucleotide sequences can be optimized to improve nuclear export of transcripts, such as through addition of a Constitutive Transport Element (CTE), RNA Transport Element (RTE), or Woodchuck Posttranscriptional Regulatory Element (WPRE). Nuclear export signals for use in expression vectors are described in more detail in Callendret et al. (Virology. 2007 Jul. 5; 363(2): 288-302), herein incorporated by reference for all purposes. Polynucleotide sequences can be optimized with respect to GC content, for example to reflect the average GC content of a given organism. Sequence optimization can balance one or more sequence properties, such as transcription, translation, post-transcriptional processing, and/or RNA stability. Sequence optimization can generate an optimal sequence balancing each of transcription, translation, post-transcriptional processing, and RNA stability. Sequence optimization algorithms are known to those of skill in the art, such as GeneArt (Thermo Fisher), Codon Optimization Tool (IDT), Cool Tool (University of Singapore), SGI-DNA (La Jolla California). One or more regions of an antigen-encoding protein can be sequence-optimized separately.

A still further aspect provides an expression vector capable of expressing a polypeptide or portion thereof. Expression vectors for different cell types are well known in the art and can be selected without undue experimentation. Generally, DNA is inserted into an expression vector, such as a plasmid, in proper orientation and correct reading frame for expression. If necessary, DNA can be linked to the appropriate transcriptional and translational regulatory control nucleotide sequences recognized by the desired host, although such controls are generally available in the expression vector. The vector is then introduced into the host through standard techniques. Guidance can be found e.g. in Sambrook et al. (1989) Molecular Cloning, A Laboratory Manual, Cold Spring Harbor Laboratory, Cold Spring Harbor, N.Y.

IV.A. Cassette

The methods employed for the selection of one or more payloads, the cloning and construction of a “cassette” and its insertion into a viral vector are within the skill in the art given the teachings provided herein. By “payload cassette” or “cassette” or “antigen cassette” is meant the combination of a selected payload or plurality of payloads (e.g., payload-encoding nucleic acid sequences, such as antigen-encoding nucleic acid sequences) and the other regulatory elements necessary to transcribe the payload(s) and express the transcribed product. The selected payload or plurality of payloads can refer to distinct payload sequences, e.g., a payload-encoding nucleic acid sequence in the cassette can encode a payload-encoding nucleic acid sequence (or plurality of payload-encoding nucleic acid sequences) such that the payloads are transcribed and expressed. A payload or plurality of payloads can be operatively linked to regulatory components in a manner which permits transcription. Such components include conventional regulatory elements that can drive expression of the payload(s) in a cell transfected with the viral vector. Thus the payload cassette can also contain a selected promoter which is linked to the payload(s) and located, with other, optional regulatory elements, within the selected viral sequences of the recombinant vector. A cassette can include one or more payloads, such as one or more sequences encoding any of the payloads described herein. A cassette can have one or more payload-encoding nucleic acid sequences, such as a cassette containing multiple payload-encoding nucleic acid sequences each independently operably linked to separate promoters and/or linked together using other multicistonic systems, such as 2A ribosome skipping sequence elements (e.g., E2A, P2A, F2A, or T2A sequences) or Internal Ribosome Entry Site (IRES) sequence elements. A linker can also have a cleavage site, such as a TEV or furin cleavage site. Linkers with cleavage sites can be used in combination with other elements, such as those in a multicistronic system. In a non-limiting illustrative example, a furin protease cleavage site can be used in conjunction with a 2A ribosome skipping sequence element such that the furin protease cleavage site is configured to facilitate removal of the 2A sequence following translation.

In a cassette containing more than one payload-encoding nucleic acid sequences, each payload-encoding nucleic acid sequence can be concatenated (e.g., in an illustrative non-limiting example, concatenated payload-encoding nucleic acid sequences encoding concatenated T cell epitopes). In illustrative examples of multicistronic formats, cassettes encoding payloads are configured as follows: (1) endogenous 26S promoter-payload 1-T2A-payload 2 protein, or (2) endogenous 26S promoter-payload 1-26S promoter-payload 2. In further illustrative examples of multicistronic formats, cassettes encoding SARS-CoV-2 payloads are configured as follows: (1) endogenous 26S promoter-Spike protein-T2A-Membrane protein, or (2) endogenous 26S promoter-Spike protein-26S promoter-concatenated T cell epitopes.

In addition to the subgenomic alphavirus-derived promoter described herein, additional promoter or promoter elements can be employed. Useful promoters can be constitutive promoters or regulated (inducible) promoters, which will enable control of the amount of payload(s) to be expressed. For example, a desirable promoter is that of the cytomegalovirus immediate early promoter/enhancer [see, e.g., Boshart et al, Cell, 41:521-530 (1985)]. Another desirable promoter includes the Rous sarcoma virus LTR promoter/enhancer. Still another promoter/enhancer sequence is the chicken cytoplasmic beta-actin promoter [T. A. Kost et al, Nucl. Acids Res., 11(23):8287 (1983)]. Other suitable or desirable promoters can be selected by one of skill in the art.

A cassette can also include nucleic acid sequences heterologous to the viral vector sequences including sequences providing signals for efficient polyadenylation of the transcript (poly(A), poly-A or pA) and introns with functional splice donor and acceptor sites. A common poly-A sequence which is employed in the exemplary vectors of this invention is that derived from the papovavirus SV-40. A poly-A sequence (e.g., a non-native poly-A) generally can be inserted in the cassette following the payload-based sequences and before the viral vector sequences. A common intron sequence can also be derived from SV-40, and is referred to as the SV-40 T intron sequence. An cassette can also contain such an intron, located between the promoter/enhancer sequence and the payload(s). Selection of these and other common vector elements are conventional [see, e.g., Sambrook et al, “Molecular Cloning. A Laboratory Manual.”, 2d edit, Cold Spring Harbor Laboratory, New York (1989) and references cited therein] and many such sequences are available from commercial and industrial sources as well as from Genbank.

A cassette can have one or more payloads (e.g., one or more payload-encoding nucleic acid sequences). For example, a given cassette can include 1-10, 1-20, 1-30, 10-20, 15-25, 15-20, 1, 2, 3, 4, 5, 6, 7, 8, 9, 10, 11, 12, 13, 14, 15, 16, 17, 18, 19, 20, or more payloads. Payloads can be linked directly to one another. Payloads can also be linked to one another with linkers. Payloads can be in any orientation relative to one another including N to C or C to N.

As described elsewhere herein, a cassette can be located in the site of any selected deletion in a viral vector, such as the deleted structural proteins of a VEE backbone or the site of the E1 gene region deletion or E3 gene region deletion of a ChAd-based vector, among others which may be selected.

The multicistronic SAM vectors can be described using the following formula to describe the ordered sequence of each element, from 5′ to 3′:
P1-(L⁵b-Nc-L³d)X-P2-(L⁵b-Nc-L³d)X-Pa-(L⁵b-Nc-L³d)X-(G5e-UDY-G3g wherein P1 comprises the SGP1 subgenomic promoter, P2 comprises the SGP2 subgenomic promoter where for Pa a=0 or 1 for additional cassettes, N comprises a payload-encoding nucleic acid sequences, where c=1, L⁵ comprises the 5′ linker sequence, where b=0 or 1, L³ comprises the 3′ linker sequence, where d=0 or 1, G5 comprises one of the at least one nucleic acid sequences encoding a GPGPG amino acid linker (SEQ ID NO: 56), where e=0 or 1, G3 comprises one of the at least one nucleic acid sequences encoding a GPGPG amino acid linker (SEQ ID NO: 56), where g=0 or 1, U comprises one of the at least one MHC class II epitope-encoding nucleic acid sequence, where f=1, X=1 to 400, where for each X the corresponding Nc is a corresponding payload-encoding nucleic acid sequence, and Y=0, 1, or 2, where for each Y the corresponding Uf is a universal MHC class II epitope-encoding nucleic acid sequence, optionally wherein the at least one universal sequence comprises at least one of Tetanus toxoid and PADRE.

A payload encoding sequence (e.g., cassette or one or more of the nucleic acid sequences encoding a payload in the cassette) can be described using the following formula to describe the ordered sequence of each element, from 5′ to 3′:

Pₐ-(L5_{b}-N_{c}-L3_{d})_{X}-(G5^{e}-U^{f})^{Y}-G3_{g}

wherein P comprises the second promoter nucleotide sequence, where a=0 or 1, where c=1, N comprises one of the payload-derived nucleic acid sequences described herein (e.g., any of the antigen-encoding nucleic acid sequences described herein), L5 comprises the 5′ linker sequence, where b=0 or 1, L3 comprises the 3′ linker sequence, where d=0 or 1, G5 comprises one of the at least one nucleic acid sequences encoding a GPGPG amino acid linker (SEQ ID NO: 56), where e=0 or 1, G3 comprises one of the at least one nucleic acid sequences encoding a GPGPG amino acid linker (SEQ ID NO: 56), where g=0 or 1, U comprises one of the at least one MHC class II epitope-encoding nucleic acid sequence, where f=1, X=1 to 400, where for each X the corresponding N_{c} is a payload-encoding nucleic acid sequence, and Y=0, 1, or 2, where for each Y the corresponding U_{f} is a (1) universal MHC class II epitope-encoding nucleic acid sequence, optionally wherein the at least one universal sequence comprises at least one of Tetanus toxoid and PADRE, or (2) a MHC class II epitope-encoding nucleic acid sequence. In some aspects, for each X the corresponding N_{c} is a distinct payload-encoding nucleic nucleic acid sequence. In some aspects, for each Y the corresponding U_{f} is a distinct universal MHC class II epitope-encoding nucleic acid sequence or a distinct MHC class II antigen-encoding nucleic nucleic acid sequence. The above payload encoding sequence formula in some instances only describes the portion of an cassette encoding concatenated payload sequences, such as concatenated T cell epitopes. For example, as an illustrative non-limiting example, in cassettes encoding concatenated T cell epitopes and one or more full-length SARS-CoV-2 proteins, the above payload encoding sequence formula describes the concatenated T cell epitopes and separately the cassette encodes one or more full-length SARS-CoV-2 proteins that are linked optionally using a multicistonic system, such as 2A ribosome skipping sequence elements (e.g., E2A, P2A, F2A, or T2A sequences) and/or a Internal Ribosome Entry Site (IRES) sequence elements.

In one example, elements present include where b=1, d=1, e=1, g=1, h=1, X=18, Y=2, and the vector backbone comprises a ChAdV68 vector, a=1, P is a CMV promoter, the at least one second poly(A) sequence is present, wherein the second poly(A) sequence is an exogenous poly(A) sequence to the vector backbone, and optionally wherein the exogenous poly(A) sequence comprises an SV40 poly(A) signal sequence or a BGH poly(A) signal sequence, and each N encodes a MHC class I epitope 7-15 amino acids in length, a MHC class II epitope, an epitope capable of stimulating a B cell response, or combinations thereof, L5 is a native 5′ linker sequence that encodes a native N-terminal amino acid sequence of the epitope, and wherein the 5′ linker sequence encodes a peptide that is at least 3 amino acids in length, L3 is a native 3′ linker sequence that encodes a native C-terminal amino acid sequence of the epitope, and wherein the 3′ linker sequence encodes a peptide that is at least 3 amino acids in length, and U is each of a PADRE class II sequence and a Tetanus toxoid MHC class II sequence. The above payload encoding sequence formula in some instances only describes the portion of a payload cassette encoding concatenated epitope sequences, such as concatenated T cell epitopes.

In one example, elements present include where b=1, d=1, e=1, g=1, h=1, X=18, Y=2, and the vector backbone comprises a Venezuelan equine encephalitis virus vector, a=0, and the payload cassette is operably linked to an endogenous 26S promoter, and the at least one polyadenylation poly(A) sequence is a poly(A) sequence of at least 80 consecutive A nucleotides (SEQ ID NO: 27940) provided by the backbone, and each N encodes a MHC class I epitope 7-15 amino acids in length, a MHC class II epitope, an epitope capable of stimulating a B cell response, or combinations thereof, L5 is a native 5′ linker sequence that encodes a native N-terminal amino acid sequence of the epitope, and wherein the 5′ linker sequence encodes a peptide that is at least 3 amino acids in length, L3 is a native 3′ linker sequence that encodes a native C-terminal amino acid sequence of the epitope, and wherein the 3′ linker sequence encodes a peptide that is at least 3 amino acids in length, and U is each of a PADRE class II sequence and a Tetanus toxoid MHC class II sequence.

The payload cassette can be described using the following formula to describe the ordered sequence of each element, from 5′ to 3′:

(Pₐ-(L5_{b}-N_{c}-L3_{d})^{X})^{Z}-(P2^{h}-(G5^{e}-U_{f})_{Y})_{W}-G3_{g}

wherein P and P2 comprise promoter nucleotide sequences, N comprises an MHC class I epitope-encoding nucleic acid sequence, L5 comprises a 5′ linker sequence, L3 comprises a 3′ linker sequence, G5 comprises a nucleic acid sequences encoding an amino acid linker, G3 comprises one of the at least one nucleic acid sequences encoding an amino acid linker, U comprises an MHC class II antigen-encoding nucleic acid sequence, where for each X the corresponding Nc is an epitope encoding nucleic acid sequence, where for each Y the corresponding Uf is a MHC class II epitope-encoding nucleic acid sequence (e.g., universal MHC class II epitope-encoding nucleic acid sequence). A universal sequence can comprise at least one of Tetanus toxoid and PADRE. A universal sequence can comprise a Tetanus toxoid peptide. A universal sequence can comprise a PADRE peptide. A universal sequence can comprise a Tetanus toxoid and PADRE peptides. The composition and ordered sequence can be further defined by selecting the number of elements present, for example where a=0 or 1, where b=0 or 1, where c=1, where d=0 or 1, where e=0 or 1, where f=1, where g=0 or 1, where h=0 or 1, X=1 to 400, Y=0, 1, 2, 3, 4 or 5, Z=1 to 400, and W=0, 1, 2, 3, 4 or 5

In one example, elements present include where a=0, b=1, d=1, e=1, g=1, h=0, X=10, Y=2, Z=1, and W=1, describing where no additional promoter is present (e.g., only the promoter nucleotide sequence provided by a vector backbone, such as an RNA alphavirus or ChAdV backbone is present), 10 MHC class I epitopes are present, a 5′ linker is present for each N, a 3′ linker is present for each N, 2 MHC class II epitopes are present, a linker is present linking the two MHC class II epitopes, a linker is present linking the 5′ end of the two MHC class II epitopes to the 3′ linker of the final MHC class I epitope, and a linker is present linking the 3′ end of the two MHC class II epitopes to a vector backbone (e.g., a ChAdV or RNA alphavirus backbone).

Examples of linking the 3′ end of the cassette to a vector backbone (e.g., an RNA alphavirus backbone) include linking directly to the 3′ UTR elements provided by the vector backbone, such as a 3′ 19-nt CSE. Examples of linking the 5′ end of the cassette to a vector backbone (e.g., an RNA alphavirus backbone) include linking directly to a promoter or 5′ UTR element of the vector backbone, such as a subgenomic promoter sequence (e.g., a 26S subgenomic promoter sequence), an alphavirus 5′ UTR, a 51-nt CSE, or a 24-nt CSE.

Other examples include: where a=1 describing where a promoter other than the promoter nucleotide sequence provided by a vector backbone (e.g., a ChAdV or RNA alphavirus backbone) is present; where a=1 and Z is greater than 1 where multiple promoters other than the promoter nucleotide sequence provided by the vector backbone are present each driving expression of 1 or more distinct MHC class I epitope encoding nucleic acid sequences; where h=1 describing where a separate promoter is present to drive expression of the MHC class II epitope-encoding nucleic acid sequences; and where g=0 describing the MHC class II epitope-encoding nucleic acid sequence, if present, is directly linked to a vector backbone (e.g., a ChAdV or RNA alphavirus backbone). For example, a ChAdV vector backbone can have the cassette placed under the control of a CMV promoter/enhancer.

Other examples include where each MHC class I epitope that is present can have a 5′ linker, a 3′ linker, neither, or both. In examples where more than one MHC class I epitope is present in the same antigen cassette, some MHC class I epitopes may have both a 5′ linker and a 3′ linker, while other MHC class I epitopes may have either a 5′ linker, a 3′ linker, or neither. In other examples where more than one MHC class I epitope is present in the same antigen cassette, some MHC class I epitopes may have either a 5′ linker or a 3′ linker, while other MHC class I epitopes may have either a 5′ linker, a 3′ linker, or neither.

In examples where more than one MHC class II epitope is present in the same antigen cassette, some MHC class II epitopes may have both a 5′ linker and a 3′ linker, while other MHC class II epitopes may have either a 5′ linker, a 3′ linker, or neither. In other examples where more than one MHC class II epitope is present in the same antigen cassette, some MHC class II epitopes may have either a 5′ linker or a 3′ linker, while other MHC class II epitopes may have either a 5′ linker, a 3′ linker, or neither.

Other examples include where each payload that is present can have a 5′ linker, a 3′ linker, neither, or both. In examples where more than one payload is present in the same payload cassette, some payloads may have both a 5′ linker and a 3′ linker, while other payloads may have either a 5′ linker, a 3′ linker, or neither. In other examples where more than one payload is present in the same payload cassette, some payloads may have either a 5′ linker or a 3′ linker, while other payloads may have either a 5′ linker, a 3′ linker, or neither.

The promoter nucleotide sequences P and/or P2 can be the same as a promoter nucleotide sequence provided by a vector backbone, such as an RNA alphavirus backbone. For example, the promoter sequence provided by the RNA alphavirus backbone, Pn and P2, can each comprise a subgenomic promoter sequence (e.g., a 26S subgenomic promoter sequence) or a CMV promoter. The promoter nucleotide sequences P and/or P2 can be different from the promoter nucleotide sequence provided by a vector backbone (e.g., a ChAdV or RNA alphavirus backbone), as well as can be different from each other.

The 5′ linker L5 can be a native sequence or a non-natural sequence. Non-natural sequence include, but are not limited to, AAY, RR, and DPP. The 3′ linker L3 can also be a native sequence or a non-natural sequence. Additionally, L5 and L3 can both be native sequences, both be non-natural sequences, or one can be native and the other non-natural. For each X, the amino acid linkers can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. For each X, the amino acid linkers can also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length. For each X, the amino acid linkers can also be between 2-10, 2-15, 2-20, 2-25, 2-30, 2-40, 2-50, 3-10, 3-15, 3-20, 3-25, 3-30, 3-40, 3-50, 4-10, 4-15, 4-20, 4-25, 4-30, 4-40, 4-50, 5-10, 5-15, 5-20, 5-25, 5-30, 5-40, 5-50, 6-10, 6-15, 6-20, 6-25, 6-30, 6-40, 6-50, 7-10, 7-15, 7-20, 7-25, 7-30, 7-40, 7-50, 8-10, 8-15, 8-20, 8-25, 8-30, 8-40, or 8-50 amino acids in length.

The amino acid linker G5, for each Y, can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. For each Y, the amino acid linkers can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length. G5 can also be between 2-10, 2-15, 2-20, 2-25, 2-30, 2-40, 2-50, 3-10, 3-15, 3-20, 3-25, 3-30, 3-40, 3-50, 4-10, 4-15, 4-20, 4-25, 4-30, 4-40, 4-50, 5-10, 5-15, 5-20, 5-25, 5-30, 5-40, 5-50, 6-10, 6-15, 6-20, 6-25, 6-30, 6-40, 6-50, 7-10, 7-15, 7-20, 7-25, 7-30, 7-40, 7-50, 8-10, 8-15, 8-20, 8-25, 8-30, 8-40, or 8-50 amino acids in length.

The amino acid linker G3 can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. G3 can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length. G3 can also be between 2-10, 2-15, 2-20, 2-25, 2-30, 2-40, 2-50, 3-10, 3-15, 3-20, 3-25, 3-30, 3-40, 3-50, 4-10, 4-15, 4-20, 4-25, 4-30, 4-40, 4-50, 5-10, 5-15, 5-20, 5-25, 5-30, 5-40, 5-50, 6-10, 6-15, 6-20, 6-25, 6-30, 6-40, 6-50, 7-10, 7-15, 7-20, 7-25, 7-30, 7-40, 7-50, 8-10, 8-15, 8-20, 8-25, 8-30, 8-40, or 8-50 amino acids in length.

For each X, each N can encode a MHC class I epitope, a MHC class II epitope, an epitope/antigen capable of stimulating a B cell response, or a combination thereof. For each X, each N can encode a combination of a MHC class I epitope, a MHC class II epitope, and an epitope/antigen capable of stimulating a B cell response. For each X, each N can encode a combination of a MHC class I epitope and a MHC class II epitope. For each X, each N can encode a combination of a MHC class I epitope and an epitope/antigen capable of stimulating a B cell response. For each X, each N can encode a combination of a MHC class II epitope and an epitope/antigen capable of stimulating a B cell response. For each X, each N can encode a MHC class II epitope. For each X, each N can encode an epitope/antigen capable of stimulating a B cell response. For each X, each N can encode a MHC class I epitope 7-15 amino acids in length. For each X, each N can also encodes a MHC class I epitope 5, 6, 7, 8, 9, 10, 11, 12, 13, 14, 15, 16, 17, 18, 19, 20, 21, 22, 23, 24, 25, 26, 27, 28, 29, or 30 amino acids in length. For each X, each N can also encodes a MHC class I epitope at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length. For each X, each N can encode a MHC class II epitope. For each X, each N can encode an epitope capable of stimulating a B cell response.

A cassette, including each cassette respectively in a multicistronic system, can be at least 100, 200, 300, 400, 500, 600, 700, 800, or 900 nucleotides in length. A cassette can be at least 1000, 2000, 3000, 4000, 5000, 6000, 7000, 8000, 9000, or 10000 nucleotides in length. A cassette can be at least 1000 nucleotides in length. A cassette can be at least 2000 nucleotides in length. A cassette can be at least 3000 nucleotides in length. A cassette can be at least 4000 nucleotides in length. A cassette can be at least 5000 nucleotides in length. A cassette can be at least 6000 nucleotides in length. A cassette can be at least 7000 nucleotides in length. A cassette can be at least 8000 nucleotides in length. A cassette can be at least 9000 nucleotides in length. A cassette can be between 100-1000, 100-2000, 100-3000, 100-4000, 100-5000, 100-6000, 100-7000, 100-8000, 100-9000, or 100-10000 nucleotides in length. A cassette can be between 500-1000, 500-2000, 500-3000, 500-4000, 500-5000, 500-6000, 500-7000, 500-8000, 500-9000, or 500-10000 nucleotides in length. A cassette can be between 1000-2000, 1000-3000, 1000-4000, 1000-5000, 1000-6000, 1000-7000, 1000-8000, 1000-9000, or 1000-10000 nucleotides in length. A cassette can be about the length deleted from an alphavirus (e.g., the length of deleted structural proteins in a VEE backbone). A cassette can be less than the length deleted from an alphavirus. A cassette can be more than the length deleted from an alphavirus.

For vectors including multiple cassettes, the total length of all cassettes combined can be at least 100, 200, 300, 400, 500, 600, 700, 800, or 900 nucleotides in length. For vectors including multiple cassettes, the total length of all cassettes combined can be at least 1000, 2000, 3000, 4000, 5000, 6000, 7000, 8000, 9000, or 10000 nucleotides in length For vectors including multiple cassettes, the total length of all cassettes combined can be between 100-1000, 100-2000, 100-3000, 100-4000, 100-5000, 100-6000, 100-7000, 100-8000, 100-9000, or 100-10000 nucleotides in length. For vectors including multiple cassettes, the total length of all cassettes combined can be between 500-1000, 500-2000, 500-3000, 500-4000, 500-5000, 500-6000, 500-7000, 500-8000, 500-9000, or 500-10000 nucleotides in length. For vectors including multiple cassettes, the total length of all cassettes combined can be between 1000-2000, 1000-3000, 1000-4000, 1000-5000, 1000-6000, 1000-7000, 1000-8000, 1000-9000, or 1000-10000 nucleotides in length.

A cassette can be 700 nucleotides or less. A cassette can be 700 nucleotides or less and encode 2 distinct epitope-encoding nucleic acid sequences (e.g., encode 2 distinct infectious disease or tumor derived nucleic acid sequences encoding an immunogenic polypeptide). A cassette can be 700 nucleotides or less and encode at least 2 distinct epitope-encoding nucleic acid sequences. A cassette can be 700 nucleotides or less and encode 3 distinct epitope-encoding nucleic acid sequences. A cassette can be 700 nucleotides or less and encode at least 3 distinct epitope-encoding nucleic acid sequences. A cassette can be 700 nucleotides or less and include 1-10, 1-5, 1, 2, 3, 4, 5, 6, 7, 8, 9, 10, or more payloads.

A cassette can be between 375-700 nucleotides in length. A cassette can be between 375-700 nucleotides in length and encode 2 distinct epitope-encoding nucleic acid sequences (e.g., encode 2 distinct infectious disease or tumor derived nucleic acid sequences encoding an immunogenic polypeptide). A cassette can be between 375-700 nucleotides in length and encode at least 2 distinct epitope-encoding nucleic acid sequences. A cassette can be between 375-700 nucleotides in length and encode 3 distinct epitope-encoding nucleic acid sequences. A cassette be between 375-700 nucleotides in length and encode at least 3 distinct epitope-encoding nucleic acid sequences. A cassette can be between 375-700 nucleotides in length and include 1-10, 1-5, 1, 2, 3, 4, 5, 6, 7, 8, 9, 10, or more payloads.

A cassette can be 600, 500, 400, 300, 200, or 100 nucleotides in length or less. A cassette can be 600, 500, 400, 300, 200, or 100 nucleotides in length or less and encode 2 distinct epitope-encoding nucleic acid sequences. A cassette can be 600, 500, 400, 300, 200, or 100 nucleotides in length or less and encode at least 2 distinct epitope-encoding nucleic acid sequences. A cassette can be 600, 500, 400, 300, 200, or 100 nucleotides in length or less and encode 3 distinct epitope-encoding nucleic acid sequences. A cassette can be 600, 500, 400, 300, 200, or 100 nucleotides in length or less and encode at least 3 distinct epitope-encoding nucleic acid sequences. A cassette can be 600, 500, 400, 300, 200, or 100 nucleotides in length or less and include 1-10, 1-5, 1, 2, 3, 4, 5, 6, 7, 8, 9, 10, or more payloads.

A cassette can be between 375-600, between 375-500, or between 375-400 nucleotides in length. A cassette can be between 375-600, between 375-500, or between 375-400 nucleotides in length and encode 2 distinct epitope-encoding nucleic acid sequences. A cassette can be between 375-600, between 375-500, or between 375-400 nucleotides in length and encode at least 2 distinct epitope-encoding nucleic acid sequences. A cassette can be between 375-600, between 375-500, or between 375-400 nucleotides in length and encode 3 distinct epitope-encoding nucleic acid sequences. A cassette can be between 375-600, between 375-500, or between 375-400 nucleotides in length and encode at least 3 distinct epitope-encoding nucleic acid sequences. A cassette can be between 375-600, between 375-500, or between 375-400 nucleotides in length and include 1-10, 1-5, 1, 2, 3, 4, 5, 6, 7, 8, 9, 10, or more payloads.

### V. Vaccine Compositions

Also disclosed herein is an immunogenic composition, e.g., a vaccine composition, capable of raising a specific immune response, e.g., a tumor-specific immune response or an infectious disease organism-specific immune response. Vaccine compositions typically comprise one or a plurality of antigens, e.g., selected using a method described herein, or selected from a pathogen-derived peptide, a virus-derived peptide, a bacteria-derived peptide, a fungus-derived peptide, and/or a parasite-derived peptide. Vaccine compositions can also be referred to as vaccines.

A vaccine can contain between 1 and 30 peptides, 2, 3, 4, 5, 6, 7, 8, 9, 10, 11, 12, 13, 14, 15, 16, 17, 18, 19, 20, 21, 22, 23, 24, 25, 26, 27, 28, 29, or 30 different peptides, 6, 7, 8, 9, 10 11, 12, 13, or 14 different peptides, or 12, 13 or 14 different peptides. Peptides can include post-translational modifications. A vaccine can contain between 1 and 100 or more nucleotide sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different nucleotide sequences, 6, 7, 8, 9, 10 11, 12, 13, or 14 different nucleotide sequences, or 12, 13 or 14 different nucleotide sequences. A vaccine can contain between 1 and 30 antigen sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different antigen sequences, 6, 7, 8, 9, 10 11, 12, 13, or 14 different antigen sequences, or 12, 13 or 14 different antigen sequences.

A vaccine can contain between 1 and 30 antigen-encoding nucleic acid sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different antigen-encoding nucleic acid sequences, 6, 7, 8, 9, 10 11, 12, 13, or 14 different antigen-encoding nucleic acid sequences, or 12, 13 or 14 different antigen-encoding nucleic acid sequences. Antigen-encoding nucleic acid sequences can refer to the antigen encoding portion of an antigen “cassette.” Features of an antigen cassette are described in greater detail herein. A cassette can contain two or more antigen-encoding nucleic acid sequences linked together in a cassette (e.g., concatenated antigen-encoding nucleic acid sequence encoding concatenated T cell epitopes).

A vaccine can contain between 1 and 30 distinct epitope-encoding nucleic acid sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more distinct epitope-encoding nucleic acid sequences, 6, 7, 8, 9, 10 11, 12, 13, or 14 distinct epitope-encoding nucleic acid sequences, or 12, 13 or 14 distinct epitope-encoding nucleic acid sequences. Epitope-encoding nucleic acid sequences can refer to sequences for individual epitope sequences, such as each of the concatenated T cell epitopes of two or more antigen-encoding nucleic acid sequences linked together in a cassette.

A vaccine can contain at least two repeats of an epitope-encoding nucleic acid sequence. A used herein, an “iteration” (or interchangeably a “repeat”) refers to two or more iterations of an identical nucleic acid epitope-encoding nucleic acid sequences (inclusive of the optional 5′ linker sequence and/or the optional 3′ linker sequences described herein) within an antigen-encoding nucleic acid sequence. In one example, the antigen-encoding nucleic acid sequence portion of a cassette encodes at least two iterations of an epitope-encoding nucleic acid sequence. In further non-limiting examples, the antigen-encoding nucleic acid sequence portion of a cassette encodes more than one distinct epitope, and at least one of the distinct epitopes is encoded by at least two iterations of the nucleic acid sequence encoding the distinct epitope (i.e., at least two distinct epitope-encoding nucleic acid sequences). In illustrative non-limiting examples, an antigen-encoding nucleic acid sequence encodes epitopes A, B, and C encoded by epitope-encoding nucleic acid sequences epitope-encoding sequence A (E_{A}), epitope-encoding sequence B (E_{B}), and epitope-encoding sequence C (E_{C}), and exemplary antigen-encoding nucleic acid sequences having iterations of at least one of the distinct epitopes are illustrated by, but is not limited to, the formulas below:
Iteration of one distinct epitope (iteration of epitope A):

E_{A}-E_{B}-E_{C}-E_{A}; or

E_{A}-E_{A}-E_{B}-E_{C}
Iteration of multiple distinct epitopes (iterations of epitopes A, B, and C):

E_{A}-E_{B}-E_{C}-E_{A}-E_{B}-E_{C}; or

E_{A}-E_{A}-E_{B}-E_{B}-E_{C}-E_{C}
Multiple iterations of multiple distinct epitopes (iterations of epitopes A, B, and C):

E_{A}-E_{B}-E_{C}-E_{A}-E_{B}-E_{C}-E_{A}-E_{B}-E_{C}; or

E_{A}-E_{A}-E_{A}-E_{B}-E_{B}-E_{B}-E_{C}-E_{C}-E_{C}

The above examples are not limiting and the antigen-encoding nucleic acid sequences having iterations of at least one of the distinct epitopes can encode each of the distinct epitopes in any order or frequency. For example, the order and frequency can be a random arrangement of the distinct epitopes, e.g., in an example with epitopes A, B, and C, by the formula E_{A}-E_{B}-E_{C}-E_{C}-E_{A}-E_{B}-E_{A}-E_{C}-E_{A}-E_{C}-E_{C}-E_{B}.

Also provided for herein is an antigen-encoding cassette, the antigen-encoding cassette having at least one antigen-encoding nucleic acid sequence described, from 5′ to 3′, by the formula:

(Eₓ−(Eₙ^{N})_{y})_{z}
where E represents a nucleotide sequence including a distinct epitope-encoding nucleic acid sequences,
n represents the number of separate distinct epitope-encoding nucleic acid sequences and is any integer including 0,
E^{N} represents a nucleotide sequence comprising the separate distinct epitope-encoding nucleic acid sequence for each corresponding n,
for each iteration of z: x=0 or 1, y=0 or 1 for each n, and at least one of x or y=1, and
z=2 or greater, wherein the antigen-encoding nucleic acid sequence comprises at least two iterations of E, a given E^{N}, or a combination thereof.

Each E or E^{N} can independently comprise any epitope-encoding nucleic acid sequence described herein (e.g., a peptide encoding an infectious disease T cell epitope and/or a neoantigen epitope). For example, Each E or E^{N} can independently comprises a nucleotide sequence described, from 5′ to 3′, by the formula (L5_{b}-N_{c}-L3_{d}), where N comprises the distinct epitope-encoding nucleic acid sequence associated with each E or E^{N}, where c=1, L5 comprises a 5′ linker sequence, where b=0 or 1, and L3 comprises a 3′ linker sequence, where d=0 or 1. Epitopes and linkers that can be used are further described herein, e.g., see IV.A. Cassette.

Iterations of an epitope-encoding nucleic acid sequences (inclusive of optional 5′ linker sequence and/or the optional 3′ linker sequences) can be linearly linked directly to one another (e.g., E_{A}-E_{A}- . . . as illustrated above). Iterations of an epitope-encoding nucleic acid sequences can be separated by one or more additional nucleotides sequences. In general, iterations of an epitope-encoding nucleic acid sequences can be separated by any size nucleotide sequence applicable for the compositions described herein. In one example, iterations of an epitope-encoding nucleic acid sequences can be separated by a separate distinct epitope-encoding nucleic acid sequence (e.g., E_{A}-E_{B}-E_{C}-E_{A} . . . , as illustrated above). In examples where iterations are separated by a single separate distinct epitope-encoding nucleic acid sequence, and each epitope-encoding nucleic acid sequences (inclusive of optional 5′ linker sequence and/or the optional 3′ linker sequences) encodes a peptide 25 amino acids in length, the iterations can be separated by 75 nucleotides, such as in antigen-encoding nucleic acid represented by E_{A}-E_{B}-E_{A} . . . , E_{A} is separated by 75 nucleotides. In an illustrative example, an antigen-encoding nucleic acid having the sequence VTNTEMFVTAPDNLGYMYEVQWPGQTQPQIANCSVYDFFVWLHYYSVRDTVTNTEMF VTAPDNLGYMYEVQWPGQTQPQIANCSVYDFFVWLHYYSVRDT (SEQ ID NO: 115) encoding iterations of 25mer antigens Trp1 (VTNTEMFVTAPDNLGYMYEVQWPGQ (SEQ ID NO: 116)) and Trp2 (TQPQIANCSVYDFFVWLHYYSVRDT (SEQ ID NO: 117)), the iterations of Trp1 are separated by the 25mer Trp2 and thus the iterations of the Trp1 epitope-encoding nucleic acid sequences are separated the 75 nucleotide Trp2 epitope-encoding nucleic acid sequence. In examples where iterations are separated by 2, 3, 4, 5, 6, 7, 8, or 9 separate distinct epitope-encoding nucleic acid sequence, and each epitope-encoding nucleic acid sequences (inclusive of optional 5′ linker sequence and/or the optional 3′ linker sequences) encodes a peptide 25 amino acids in length, the iterations can be separated by 150, 225, 300, 375, 450, 525, 600, or 675 nucleotides, respectively.

In one embodiment, different peptides and/or polypeptides or nucleotide sequences encoding them are selected so that the peptides and/or polypeptides capable of associating with different MHC molecules, such as different MHC class I molecules and/or different MHC class II molecules. In some aspects, one vaccine composition comprises coding sequence for peptides and/or polypeptides capable of associating with the most frequently occurring MHC class I molecules and/or different MHC class II molecules. Hence, vaccine compositions can comprise different fragments capable of associating with at least 2 preferred, at least 3 preferred, or at least 4 preferred MHC class I molecules and/or different MHC class II molecules.

The vaccine composition can be capable of stimulating a specific cytotoxic T-cell response and/or a specific helper T-cell response. The vaccine composition can be capable of stimulating a specific cytotoxic T-cell response and a specific helper T-cell response.

The vaccine composition can be capable of stimulating a specific B-cell response (e.g., an antibody response).

The vaccine composition can be capable of stimulating a specific cytotoxic T-cell response, a specific helper T-cell response, and/or a specific B-cell response. The vaccine composition can be capable of stimulating a specific cytotoxic T-cell response and a specific B-cell response. The vaccine composition can be capable of stimulating a specific helper T-cell response and a specific B-cell response. The vaccine composition can be capable of stimulating a specific cytotoxic T-cell response, a specific helper T-cell response, and a specific B-cell response.

A vaccine composition can further comprise an adjuvant and/or a carrier. Examples of useful adjuvants and carriers are given herein below. A composition can be associated with a carrier such as e.g. a protein or an antigen-presenting cell such as, e.g., a dendritic cell (DC) capable of presenting the peptide to a T-cell.

Adjuvants are any substance whose admixture into a vaccine composition increases or otherwise modifies the immune response to an antigen. Carriers can be scaffold structures, for example a polypeptide or a polysaccharide, to which an antigen, is capable of being associated. Optionally, adjuvants are conjugated covalently or non-covalently.

The ability of an adjuvant to increase an immune response to an antigen is typically manifested by a significant or substantial increase in an immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th response into a primarily cellular, or Th response.

Suitable adjuvants include, but are not limited to 1018 ISS, alum, aluminium salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants such as incomplete Freund's or GM-CSF are useful. Several immunological adjuvants (e.g., MF59) specific for dendritic cells and their preparation have been described previously (Dupuis M, et al., Cell Immunol. 1998; 186(1):18-27; Allison A C; Dev Biol Stand. 1998; 92:3-11). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418).

CpG immunostimulatory oligonucleotides have also been reported to enhance the effects of adjuvants in a vaccine setting. Other TLR binding molecules such as RNA binding TLR7, TLR8 and/or TLR9 may also be used.

Other examples of useful adjuvants include, but are not limited to, chemically modified CpGs (e.g. CpR, Idera), Poly(I:C)(e.g. polyi:CI2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives can readily be determined by the skilled artisan without undue experimentation. Additional adjuvants include colony-stimulating factors, such as Granulocyte Macrophage Colony Stimulating Factor (GM-CSF, sargramostim).

A vaccine composition can comprise more than one different adjuvant. Furthermore, a therapeutic composition can comprise any adjuvant substance including any of the above or combinations thereof. It is also contemplated that a vaccine and an adjuvant can be administered together or separately in any appropriate sequence.

A carrier (or excipient) can be present independently of an adjuvant. The function of a carrier can for example be to increase the molecular weight of in particular mutant to increase activity or immunogenicity, to confer stability, to increase the biological activity, or to increase serum half-life. Furthermore, a carrier can aid presenting peptides to T-cells. A carrier can be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier is generally a physiologically acceptable carrier acceptable to humans and safe. However, tetanus toxoid and/or diphtheria toxoid are suitable carriers. Alternatively, the carrier can be dextrans for example sepharose.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is possible if a trimeric complex of peptide antigen, MHC molecule, and APC is present. Correspondingly, it may enhance the immune response if not only the peptide is used for activation of CTLs, but if additionally APCs with the respective MHC molecule are added. Therefore, in some embodiments a vaccine composition additionally contains at least one antigen presenting cell.

Antigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al, Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol. (1998) 72 (12): 9873-9880). Dependent on the packaging capacity of the above mentioned viral vector-based vaccine platforms, this approach can deliver one or more nucleotide sequences that encode one or more antigen peptides. The sequences may be flanked by non-mutated sequences, may be separated by linkers or may be preceded with one or more sequences targeting a subcellular compartment (See, e.g., Gros et al., Prospective identification of neoantigen-specific lymphocytes in the peripheral blood of melanoma patients, Nat Med. (2016) 22 (4):433-8, Stronen et al., Targeting of cancer neoantigens with donor-derived T cell receptor repertoires, Science. (2016) 352 (6291):1337-41, Lu et al., Efficient identification of mutated cancer antigens recognized by T cells associated with durable tumor regressions, Clin Cancer Res. (2014) 20(13):3401-10). Upon introduction into a host, infected cells express the antigens, and thereby stimulate a host immune (e.g., CTL) response against the peptide(s). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of antigens, e.g., Salmonella typhi vectors, and the like will be apparent to those skilled in the art from the description herein.

V.A. Additional Considerations for Vaccine Design and Manufacture

V.A.1. Determination of a Set of Peptides that Cover all Tumor Subclones

Truncal peptides, meaning those presented by all or most tumor subclones, can be prioritized for inclusion into a vaccine. Optionally, if there are no truncal peptides predicted to be presented and immunogenic with high probability, or if the number of truncal peptides predicted to be presented and immunogenic with high probability is small enough that additional non-truncal peptides can be included in the vaccine, then further peptides can be prioritized by estimating the number and identity of tumor subclones and choosing peptides so as to maximize the number of tumor subclones covered by a vaccine.

V.A.2. Antigen Prioritization

After all of the above antigen filters are applied, more candidate antigens may still be available for vaccine inclusion than the vaccine technology can support. Additionally, uncertainty about various aspects of the antigen analysis may remain and tradeoffs may exist between different properties of candidate vaccine antigens. Thus, in place of predetermined filters at each step of the selection process, an integrated multi-dimensional model can be considered that places candidate antigens in a space with at least the following axes and optimizes selection using an integrative approach.
1. Risk of auto-immunity or tolerance (risk of germline) (lower risk of auto-immunity is typically preferred)
2. Probability of sequencing artifact (lower probability of artifact is typically preferred)
3. Probability of immunogenicity (higher probability of immunogenicity is typically preferred)
4. Probability of presentation (higher probability of presentation is typically preferred)
5. Gene expression (higher expression is typically preferred)
6. Coverage of HLA genes (larger number of HLA molecules involved in the presentation of a set of antigens may lower the probability that a tumor, an infectious disease, and/or an infected cell will escape immune attack via downregulation or mutation of HLA molecules)
7. Coverage of HLA classes (covering both HLA-I and HLA-II may increase the probability of therapeutic response and decrease the probability of tumor or infectious disease escape)

Additionally, optionally, antigens can be deprioritized (e.g., excluded) from the vaccination if they are predicted to be presented by HLA alleles lost or inactivated in either all or part of the patient's tumor or infected cell. HLA allele loss can occur by either somatic mutation, loss of heterozygosity, or homozygous deletion of the locus. Methods for detection of HLA allele somatic mutation are well known in the art, e.g. (Shukla et al., 2015). Methods for detection of somatic LOH and homozygous deletion (including for HLA locus) are likewise well described. (Carter et al., 2012; McGranahan et al., 2017; Van Loo et al., 2010). Antigens can also be deprioritized if mass-spectrometry data indicates a predicted antigen is not presented by a predicted HLA allele.

V.C. Self-Amplifying RNA Vectors

In general, all self-amplifying RNA (SAM) vectors contain a self-amplifying backbone derived from a self-replicating virus. The term “self-amplifying backbone” refers to minimal sequence(s) of a self-replicating virus that allows for self-replication of the viral genome. For example, minimal sequences that allow for self-replication of an alphavirus can include conserved sequences for nonstructural protein-mediated amplification (e.g., a nonstructural protein 1 (nsP1) gene, a nsP2 gene, a nsP3 gene, a nsP4 gene, and/or a polyA sequence). A self-amplifying backbone can also include sequences for expression of subgenomic viral RNA (e.g., a 26S promoter element for an alphavirus). SAM vectors can be positive-sense RNA polynucleotides or negative-sense RNA polynucleotides, such as vectors with backbones derived from positive-sense or negative-sense self-replicating viruses. Self-replicating viruses include, but are not limited to, alphaviruses, flaviviruses (e.g., Kunjin virus), measles viruses, and rhabdoviruses (e.g., rabies virus and vesicular stomatitis virus). Examples of SAM vector systems derived from self-replicating viruses are described in greater detail in Lundstrom (Molecules. 2018 Dec. 13; 23 (12). pii: E3310. doi: 10.3390/molecules23123310), herein incorporated by reference for all purposes.

V.C.1. Alphavirus Biology

Alphaviruses are members of the family Togaviridae, and are positive-sense single stranded RNA viruses. Members are typically classified as either Old World, such as Sindbis, Ross River, Mayaro, Chikungunya, and Semliki Forest viruses, or New World, such as eastern equine encephalitis, Aura, Fort Morgan, or Venezuelan equine encephalitis virus and its derivative strain TC-83 (Strauss Microbial Review 1994). A natural alphavirus genome is typically around 12 kb in length, the first two-thirds of which contain genes encoding non-structural proteins (nsPs) that form RNA replication complexes for self-replication of the viral genome, and the last third of which contains a subgenomic expression cassette encoding structural proteins for virion production (Frolov RNA 2001).

A model lifecycle of an alphavirus involves several distinct steps (Strauss Microbrial Review 1994, Jose Future Microbiol 2009). Following virus attachment to a host cell, the virion fuses with membranes within endocytic compartments resulting in the eventual release of genomic RNA into the cytosol. The genomic RNA, which is in a plus-strand orientation and comprises a 5′ methylguanylate cap and 3′ polyA tail, is translated to produce non-structural proteins nsP1-4 that form the replication complex. Early in infection, the plus-strand is then replicated by the complex into a minus-stand template. In the current model, the replication complex is further processed as infection progresses, with the resulting processed complex switching to transcription of the minus-strand into both full-length positive-strand genomic RNA, as well as the 26S subgenomic positive-strand RNA containing the structural genes. Several conserved sequence elements (CSEs) of alphavirus have been identified to potentially play a role in the various RNA replication steps including; a complement of the 5′ UTR in the replication of plus-strand RNAs from a minus-strand template, a 51-nt CSE in the replication of minus-strand synthesis from the genomic template, a 24-nt CSE in the junction region between the nsPs and the 26S RNA in the transcription of the subgenomic RNA from the minus-strand, and a 3′ 19-nt CSE in minus-strand synthesis from the plus-strand template.

Following the replication of the various RNA species, virus particles are then typically assembled in the natural lifecycle of the virus. The 26S RNA is translated and the resulting proteins further processed to produce the structural proteins including capsid protein, glycoproteins E1 and E2, and two small polypeptides E3 and 6K (Strauss 1994). Encapsidation of viral RNA occurs, with capsid proteins normally specific for only genomic RNA being packaged, followed by virion assembly and budding at the membrane surface.

V.C.2. Alphavirus as a Delivery Vector

Alphaviruses (including alphavirus sequences, features, and other elements) can be used to generate alphavirus-based delivery vectors (also be referred to as alphavirus vectors, alphavirus viral vectors, alphavirus vaccine vectors, self-replicating RNA (srRNA) vectors, or self-amplifying mRNA (SAM) vectors). Alphaviruses have previously been engineered for use as expression vector systems (Pushko 1997, Rheme 2004). Alphaviruses offer several advantages, particularly in a vaccine setting where heterologous antigen expression can be desired. Due to its ability to self-replicate in the host cytosol, alphavirus vectors are generally able to produce high copy numbers of the expression cassette within a cell resulting in a high level of heterologous antigen production. Additionally, the vectors are generally transient, resulting in improved biosafety as well as reduced induction of immunological tolerance to the vector. The public, in general, also lacks pre-existing immunity to alphavirus vectors as compared to other standard viral vectors, such as human adenovirus. Alphavirus based vectors also generally result in cytotoxic responses to infected cells. Cytotoxicity, to a certain degree, can be important in a vaccine setting to properly stimulate an immune response to the heterologous antigen expressed. However, the degree of desired cytotoxicity can be a balancing act, and thus several attenuated alphaviruses have been developed, including the TC-83 strain of VEE. Thus, an example of an antigen expression vector described herein can utilize an alphavirus backbone that allows for a high level of antigen expression, stimulates a robust immune response to antigen, does not stimulate an immune response to the vector itself, and can be used in a safe manner. Furthermore, the antigen expression cassette can be designed to stimulate different levels of an immune response through optimization of which alphavirus sequences the vector uses, including, but not limited to, sequences derived from VEE or its attenuated derivative TC-83.

Several expression vector design strategies have been engineered using alphavirus sequences (Pushko 1997). In one strategy, a alphavirus vector design includes inserting a second copy of the 26S promoter sequence elements downstream of the structural protein genes, followed by a heterologous gene (Frolov 1993). Thus, in addition to the natural non-structural and structural proteins, an additional subgenomic RNA is produced that expresses the heterologous protein. In this system, all the elements for production of infectious virions are present and, therefore, repeated rounds of infection of the expression vector in non-infected cells can occur.

Another expression vector design makes use of helper virus systems (Pushko 1997). In this strategy, the structural proteins are replaced by a heterologous gene. Thus, following self-replication of viral RNA mediated by still intact non-structural genes, the 26S subgenomic RNA provides for expression of the heterologous protein. Traditionally, additional vectors that expresses the structural proteins are then supplied in trans, such as by co-transfection of a cell line, to produce infectious virus. A system is described in detail in U.S. Pat. No. 8,093,021, which is herein incorporated by reference in its entirety, for all purposes. The helper vector system provides the benefit of limiting the possibility of forming infectious particles and, therefore, improves biosafety. In addition, the helper vector system reduces the total vector length, potentially improving the replication and expression efficiency. Thus, an example of an antigen expression vector described herein can utilize an alphavirus backbone wherein the structural proteins are replaced by an antigen cassette, the resulting vector both reducing biosafety concerns, while at the same time promoting efficient expression due to the reduction in overall expression vector size.

V.C.3. Self-Amplifying Virus Production In Vitro

A convenient technique well-known in the art for RNA production is in vitro transcription (IVT). In this technique, a DNA template of the desired vector is first produced by techniques well-known to those in the art, including standard molecular biology techniques such as cloning, restriction digestion, ligation, gene synthesis (e.g., chemical and/or enzymatic synthesis), and polymerase chain reaction (PCR).

The DNA template contains a RNA polymerase promoter at the 5′ end of the sequence desired to be transcribed into RNA (e.g., SAM). Promoters include, but are not limited to, bacteriophage polymerase promoters such as T3, T7, K11, or SP6. Depending on the specific RNA polymerase promoter sequence chosen, additional 5′ nucleotides can transcribed in addition to the desired sequence. For example, the canonical T7 promoter can be referred to by the sequence TAATACGACTCACTATAGG (SEQ ID NO: 118), in which an IVT reaction using the DNA template TAATACGACTCACTATAGGN (SEQ ID NO: 119) for the production of desired sequence N will result in the mRNA sequence GG-N. In general, and without wishing to be bound by theory, T7 polymerase more efficiently transcribes RNA transcripts beginning with guanosine. In instances where additional 5′ nucleotides are not desired (e.g., no additional GG), the RNA polymerase promoter contained in the DNA template can be a sequence the results in transcripts containing only the 5′ nucleotides of the desired sequence, e.g., a SAM having the native 5′ sequence of the self-replicating virus from which the SAM vector is derived. For example, a minimal T7 promoter can be referred to by the sequence TAATACGACTCACTATA (SEQ ID NO: 120), in which an IVT reaction using the DNA template TAATACGACTCACTATAN (SEQ ID NO: 121) for the production of desired sequence N will result in the mRNA sequence N. Likewise, a minimal SP6 promoter referred to by the sequence ATTTAGGTGACACTATA (SEQ ID NO: 122) can be used to generate transcripts without additional 5′ nucleotides. In a typical IVT reaction, the DNA template is incubated with the appropriate RNA polymerase enzyme, buffer agents, and nucleotides (NTPs).

The resulting RNA polynucleotide can optionally be further modified including, but limited to, addition of a 5′ cap structure such as 7-methylguanosine or a related structure, and optionally modifying the 3′ end to include a polyadenylate (polyA) tail. In a modified IVT reaction, RNA is capped with a 5′ cap structure co-transcriptionally through the addition of cap analogues during IVT. Cap analogues can include dinucleotide (m⁷G-ppp-N) cap analogues or trinucleotide (m⁷G-ppp-N-N) cap analogues, where N represents a nucleotide or modified nucleotide (e.g., ribonucleosides including, but not limited to, adenosine, guanosine, cytidine, and uradine). Exemplary cap analogues and their use in IVT reactions are also described in greater detail in U.S. Pat. No. 10,519,189, herein incorporated by reference for all purposes. As discussed, T7 polymerase more efficiently transcribes RNA transcripts beginning with guanosine. To improve transcription efficiency in templates that do not begin with guanosine, a trinucleotide cap analogue (m⁷G-ppp-N-N) can be used. The trinucleotide cap analogue can increase transcription efficiency 2, 3, 4, 5, 6, 7, 8, 9, 10, 11, 12, 13, 14, 15, 16, 17, 18, 19, 20-fold or more relative to an IVT reaction using a dinucleotide cap analogue (m⁷G-ppp-N).

A 5′ cap structure can also be added following transcription, such as using a vaccinia capping system (e.g., NEB Cat. No. M2080) containing mRNA 2′-O-methyltransferase and S-Adenosyl methionine.

The resulting RNA polynucleotide can optionally be further modified separately from or in addition to the capping techniques described including, but limited to, modifying the 3′ end to include a polyadenylate (polyA) tail.

The RNA can then be purified using techniques well-known in the field, such as phenol-chloroform extraction or column purification (e.g., chromatography-based purification).

V.C.4. Delivery Via Lipid Nanoparticle

An important aspect to consider in vaccine vector design is immunity against the vector itself (Riley 2017). This may be in the form of preexisting immunity to the vector itself, such as with certain human adenovirus systems, or in the form of developing immunity to the vector following administration of the vaccine. The latter is an important consideration if multiple administrations of the same vaccine are performed, such as separate priming and boosting doses, or if the same vaccine vector system is to be used to deliver different antigen cassettes.

In the case of alphavirus vectors, the standard delivery method is the previously discussed helper virus system that provides capsid, E1, and E2 proteins in trans to produce infectious viral particles. However, it is important to note that the E1 and E2 proteins are often major targets of neutralizing antibodies (Strauss 1994). Thus, the efficacy of using alphavirus vectors to deliver antigens of interest to target cells may be reduced if infectious particles are targeted by neutralizing antibodies.

An alternative to viral particle mediated gene delivery is the use of nanomaterials to deliver expression vectors (Riley 2017). Nanomaterial vehicles, importantly, can be made of non-immunogenic materials and generally avoid stimulating immunity to the delivery vector itself. These materials can include, but are not limited to, lipids, inorganic nanomaterials, and other polymeric materials. Lipids can be cationic, anionic, or neutral. The materials can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluble vitamins.

Lipid nanoparticles (LNPs) are an attractive delivery system due to the amphiphilic nature of lipids enabling formation of membranes and vesicle like structures (Riley 2017). In general, these vesicles deliver the expression vector by absorbing into the membrane of target cells and releasing nucleic acid into the cytosol. In addition, LNPs can be further modified or functionalized to facilitate targeting of specific cell types. Another consideration in LNP design is the balance between targeting efficiency and cytotoxicity. Lipid compositions generally include defined mixtures of cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids.

Nucleic-acid vectors, such as expression vectors, exposed directly to serum can have several undesirable consequences, including degradation of the nucleic acid by serum nucleases or off-target stimulation of the immune system by the free nucleic acids. Therefore, encapsulation of the alphavirus vector can be used to avoid degradation, while also avoiding potential off-target effects. In certain examples, an alphavirus vector is fully encapsulated within the delivery vehicle, such as within the aqueous interior of an LNP. Encapsulation of the alphavirus vector within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with the alphavirus delivery vector and other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the expression vectors can be further treated or modified to prepare them for administration.

V.D. Chimpanzee Adenovirus (ChAd)

V.D.1. Viral Delivery with Chimpanzee Adenovirus

Vaccine compositions for delivery of one or more antigens (e.g., via an antigen cassette) can be created by providing adenovirus nucleotide sequences of chimpanzee origin, a variety of novel vectors, and cell lines expressing chimpanzee adenovirus genes. A nucleotide sequence of a chimpanzee C68 adenovirus (also referred to herein as ChAdV68) can be used in a vaccine composition for antigen delivery (See SEQ ID NO: 1). Use of C68 adenovirus derived vectors is described in further detail in U.S. Pat. No. 6,083,716, which is herein incorporated by reference in its entirety, for all purposes. ChAdV68-based vectors and delivery systems are described in detail in US App. Pub. No. US20200197500A1 and international patent application publication WO2020243719A1, each of which is herein incorporated by reference for all purposes.

In a further aspect, provided herein is a recombinant adenovirus comprising the DNA sequence of a chimpanzee adenovirus such as C68 and an antigen cassette operatively linked to regulatory sequences directing its expression. The recombinant virus is capable of infecting a mammalian, preferably a human, cell and capable of expressing the antigen cassette product in the cell. In this vector, the native chimpanzee E1 gene, and/or E3 gene, and/or E4 gene can be deleted. An antigen cassette can be inserted into any of these sites of gene deletion. The antigen cassette can include an antigen against which a primed immune response is desired.

In another aspect, provided herein is a mammalian cell infected with a chimpanzee adenovirus such as C68.

In still a further aspect, a novel mammalian cell line is provided which expresses a chimpanzee adenovirus gene (e.g., from C68) or functional fragment thereof.

In still a further aspect, provided herein is a method for delivering an antigen cassette into a mammalian cell comprising the step of introducing into the cell an effective amount of a chimpanzee adenovirus, such as C68, that has been engineered to express the antigen cassette.

Still another aspect provides a method for stimulating an immune response in a mammalian host to treat cancer. The method can comprise the step of administering to the host an effective amount of a recombinant chimpanzee adenovirus, such as C68, comprising an antigen cassette that encodes one or more antigens from the tumor against which the immune response is targeted.

Still another aspect provides a method for stimulating an immune response in a mammalian host to treat or prevent a disease in a subject, such as an infectious disease. The method can comprise the step of administering to the host an effective amount of a recombinant chimpanzee adenovirus, such as C68, comprising an antigen cassette that encodes one or more antigens, such as from the infectious disease against which the immune response is targeted.

Also disclosed is a non-simian mammalian cell that expresses a chimpanzee adenovirus gene obtained from the sequence of SEQ ID NO: 1. The gene can be selected from the group consisting of the adenovirus E1A, E1B, E2A, E2B, E3, E4, L1, L2, L3, L4 and L5 of SEQ ID NO: 1.

Also disclosed is a nucleic acid molecule comprising a chimpanzee adenovirus DNA sequence comprising a gene obtained from the sequence of SEQ ID NO: 1. The gene can be selected from the group consisting of said chimpanzee adenovirus E1A, E1B, E2A, E2B, E3, E4, L1, L2, L3, L4 and L5 genes of SEQ ID NO: 1. In some aspects the nucleic acid molecule comprises SEQ ID NO: 1. In some aspects the nucleic acid molecule comprises the sequence of SEQ ID NO: 1, lacking at least one gene selected from the group consisting of E1A, E1B, E2A, E2B, E3, E4, L1, L2, L3, L4 and L5 genes of SEQ ID NO: 1.

Also disclosed is a vector comprising a chimpanzee adenovirus DNA sequence obtained from SEQ ID NO: 1 and an antigen cassette operatively linked to one or more regulatory sequences which direct expression of the cassette in a heterologous host cell, optionally wherein the chimpanzee adenovirus DNA sequence comprises at least the cis-elements necessary for replication and virion encapsidation, the cis-elements flanking the antigen cassette and regulatory sequences. In some aspects, the chimpanzee adenovirus DNA sequence comprises a gene selected from the group consisting of E1A, E1B, E2A, E2B, E3, E4, L1, L2, L3, L4 and L5 gene sequences of SEQ ID NO: 1. In some aspects the vector can lack the E1A and/or E1B gene.

Also disclosed herein is a adenovirus vector comprising: a partially deleted E4 gene comprising a deleted or partially-deleted E4orf2 region and a deleted or partially-deleted E4orf3 region, and optionally a deleted or partially-deleted E4orf4 region. The partially deleted E4 can comprise an E4 deletion of at least nucleotides 34,916 to 35,642 of the sequence shown in SEQ ID NO:1, and wherein the vector comprises at least nucleotides 2 to 36,518 of the sequence set forth in SEQ ID NO:1. The partially deleted E4 can comprise an E4 deletion of at least a partial deletion of nucleotides 34,916 to 34,942 of the sequence shown in SEQ ID NO:1, at least a partial deletion of nucleotides 34,952 to 35,305 of the sequence shown in SEQ ID NO:1, and at least a partial deletion of nucleotides 35,302 to 35,642 of the sequence shown in SEQ ID NO:1, and wherein the vector comprises at least nucleotides 2 to 36,518 of the sequence set forth in SEQ ID NO:1 The partially deleted E4 can comprise an E4 deletion of at least nucleotides 34,980 to 36,516 of the sequence shown in SEQ ID NO:1, and wherein the vector comprises at least nucleotides 2 to 36,518 of the sequence set forth in SEQ ID NO:1. The partially deleted E4 can comprise an E4 deletion of at least nucleotides 34,979 to 35,642 of the sequence shown in SEQ ID NO:1, and wherein the vector comprises at least nucleotides 2 to 36,518 of the sequence set forth in SEQ ID NO:1. The partially deleted E4 can comprise an E4 deletion of at least a partial deletion of E4Orf2, a fully deleted E4Orf3, and at least a partial deletion of E4Orf4. The partially deleted E4 can comprise an E4 deletion of at least a partial deletion of E4Orf2, at least a partial deletion of E4Orf3, and at least a partial deletion of E4Orf4. The partially deleted E4 can comprise an E4 deletion of at least a partial deletion of E4Orf1, a fully deleted E4Orf2, and at least a partial deletion of E4Orf3. The partially deleted E4 can comprise an E4 deletion of at least a partial deletion of E4Orf2 and at least a partial deletion of E4Orf3. The partially deleted E4 can comprise an E4 deletion between the start site of E4Orf1 to the start site of E4Orf5. The partially deleted E4 can be an E4 deletion adjacent to the start site of E4Orf1. The partially deleted E4 can be an E4 deletion adjacent to the start site of E4Orf2. The partially deleted E4 can be an E4 deletion adjacent to the start site of E4Orf3. The partially deleted E4 can be an E4 deletion adjacent to the start site of E4Orf4. The E4 deletion can be at least 50, at least 100, at least 200, at least 300, at least 400, at least 500, at least 600, at least 700, at least 800, at least 900, at least 1000, at least 1100, at least 1200, at least 1300, at least 1400, at least 1500, at least 1600, at least 1700, at least 1800, at least 1900, or at least 2000 nucleotides. The E4 deletion can be at least 700 nucleotides. The E4 deletion can be at least 1500 nucleotides. The E4 deletion can be 50 or less, 100 or less, 200 or less, 300 or less, 400 or less, 500 or less, 600 or less, 700 or less, 800 or less, 900 or less, 1000 or less, 1100 or less, 1200 or less, 1300 or less, 1400 or less, 1500 or less, 1600 or less, 1700 or less, 1800 or less, 1900 or less, or 2000 or less nucleotides. The E4 deletion can be 750 nucleotides or less. The E4 deletion can be at least 1550 nucleotides or less.

The partially deleted E4 gene can be the E4 gene sequence shown in SEQ ID NO:1 that lacks at least nucleotides 34,916 to 35,642 of the sequence shown in SEQ ID NO:1. The partially deleted E4 gene can be the E4 gene sequence shown in SEQ ID NO:1 that lacks the E4 gene sequence shown in SEQ ID NO:1 and that lacks at least nucleotides 34,916 to 34,942, nucleotides 34,952 to 35,305 of the sequence shown in SEQ ID NO:1, and nucleotides 35,302 to 35,642 of the sequence shown in SEQ ID NO:1. The partially deleted E4 gene can be the E4 gene sequence shown in SEQ ID NO:1 and that lacks at least nucleotides 34,980 to 36,516 of the sequence shown in SEQ ID NO:1. The partially deleted E4 gene can be the E4 gene sequence shown in SEQ ID NO:1 and that lacks at least nucleotides 34,979 to 35,642 of the sequence shown in SEQ ID NO:1. The adenovirus vector having the partially deleted E4 gene can have a cassette, wherein the cassette comprises at least one payload nucleic acid sequence, and wherein the cassette comprises at least one promoter sequence operably linked to the at least one payload nucleic acid sequence. The adenovirus vector having the partially deleted E4 gene can have one or more genes or regulatory sequences of the ChAdV68 sequence shown in SEQ ID NO: 1, optionally wherein the one or more genes or regulatory sequences comprise at least one of the chimpanzee adenovirus inverted terminal repeat (ITR), E1A, E1B, E2A, E2B, E3, E4, L1, L2, L3, L4, and L5 genes of the sequence shown in SEQ ID NO: 1. The adenovirus vector having the partially deleted E4 gene can have nucleotides 2 to 34,916 of the sequence shown in SEQ ID NO:1, wherein the partially deleted E4 gene is 3′ of the nucleotides 2 to 34,916, and optionally the nucleotides 2 to 34,916 additionally lack nucleotides 577 to 3403 of the sequence shown in SEQ ID NO:1 corresponding to an E1 deletion and/or lack nucleotides 27,125 to 31,825 of the sequence shown in SEQ ID NO:1 corresponding to an E3 deletion. The adenovirus vector having the partially deleted E4 gene can have nucleotides 35,643 to 36,518 of the sequence shown in SEQ ID NO:1, and wherein the partially deleted E4 gene is 5′ of the nucleotides 35,643 to 36,518. The adenovirus vector having the partially deleted E4 gene can have nucleotides 2 to 34,916 of the sequence shown in SEQ ID NO:1, wherein the partially deleted E4 gene is 3′ of the nucleotides 2 to 34,916, the nucleotides 2 to 34,916 additionally lack nucleotides 577 to 3403 of the sequence shown in SEQ ID NO:1 corresponding to an E1 deletion and lack nucleotides 27,125 to 31,825 of the sequence shown in SEQ ID NO:1 corresponding to an E3 deletion. The adenovirus vector having the partially deleted E4 gene can have nucleotides 2 to 34,916 of the sequence shown in SEQ ID NO:1, wherein the partially deleted E4 gene is 3′ of the nucleotides 2 to 34,916, the nucleotides 2 to 34,916 additionally lack nucleotides 577 to 3403 of the sequence shown in SEQ ID NO:1 corresponding to an E1 deletion and lack nucleotides 27,125 to 31,825 of the sequence shown in SEQ ID NO:1 corresponding to an E3 deletion, and have nucleotides 35,643 to 36,518 of the sequence shown in SEQ ID NO:1, and wherein the partially deleted E4 gene is 5′ of the nucleotides 35,643 to 36,518.

The partially deleted E4 gene can be the E4 gene sequence shown in SEQ ID NO:1 that lacks at least nucleotides 34,916 to 35,642 of the sequence shown in SEQ ID NO:1, nucleotides 2 to 34,916 of the sequence shown in SEQ ID NO:1, wherein the partially deleted E4 gene is 3′ of the nucleotides 2 to 34,916, the nucleotides 2 to 34,916 additionally lack nucleotides 577 to 3403 of the sequence shown in SEQ ID NO:1 corresponding to an E1 deletion and lack nucleotides 27,125 to 31,825 of the sequence shown in SEQ ID NO:1 corresponding to an E3 deletion, and have nucleotides 35,643 to 36,518 of the sequence shown in SEQ ID NO:1, and wherein the partially deleted E4 gene is 5′ of the nucleotides 35,643 to 36,518.

Also disclosed herein is a host cell transfected with a vector disclosed herein such as a C68 vector engineered to expression an antigen cassette. Also disclosed herein is a human cell that expresses a selected gene introduced therein through introduction of a vector disclosed herein into the cell.

Also disclosed herein is a method for delivering an antigen cassette to a mammalian cell comprising introducing into said cell an effective amount of a vector disclosed herein such as a C68 vector engineered to expression the antigen cassette.

Also disclosed herein is a method for producing an antigen comprising introducing a vector disclosed herein into a mammalian cell, culturing the cell under suitable conditions and producing the antigen.

V.D.2. E1-Expressing Complementation Cell Lines

To generate recombinant chimpanzee adenoviruses (Ad) deleted in any of the genes described herein, the function of the deleted gene region, if essential to the replication and infectivity of the virus, can be supplied to the recombinant virus by a helper virus or cell line, i.e., a complementation or packaging cell line. For example, to generate a replication-defective chimpanzee adenovirus vector, a cell line can be used which expresses the E1 gene products of the human or chimpanzee adenovirus; such a cell line can include HEK293 or variants thereof. The protocol for the generation of the cell lines expressing the chimpanzee E1 gene products (Examples 3 and 4 of U.S. Pat. No. 6,083,716) can be followed to generate a cell line which expresses any selected chimpanzee adenovirus gene.

An AAV augmentation assay can be used to identify a chimpanzee adenovirus E1-expressing cell line. This assay is useful to identify E1 function in cell lines made by using the E1 genes of other uncharacterized adenoviruses, e.g., from other species. That assay is described in Example 4B of U.S. Pat. No. 6,083,716.

A selected chimpanzee adenovirus gene, e.g., E1, can be under the transcriptional control of a promoter for expression in a selected parent cell line. Inducible or constitutive promoters can be employed for this purpose. Among inducible promoters are included the sheep metallothionine promoter, inducible by zinc, or the mouse mammary tumor virus (MMTV) promoter, inducible by a glucocorticoid, particularly, dexamethasone. Other inducible promoters, such as those identified in International patent application WO95/13392, incorporated by reference herein can also be used in the production of packaging cell lines. Constitutive promoters in control of the expression of the chimpanzee adenovirus gene can be employed also.

A parent cell can be selected for the generation of a novel cell line expressing any desired C68 gene. Without limitation, such a parent cell line can be HeLa [ATCC Accession No. CCL 2], A549 [ATCC Accession No. CCL 185], KB [CCL 17], Detroit [e.g., Detroit 510, CCL 72] and WI-38 [CCL 75] cells. Other suitable parent cell lines can be obtained from other sources. Parent cell lines can include CHO, HEK293 or variants thereof, 911, HeLa, A549, LP-293, PER.C6, or AE1-2a.

An E1-expressing cell line can be useful in the generation of recombinant chimpanzee adenovirus E1 deleted vectors. Cell lines constructed using essentially the same procedures that express one or more other chimpanzee adenoviral gene products are useful in the generation of recombinant chimpanzee adenovirus vectors deleted in the genes that encode those products. Further, cell lines which express other human Ad E1 gene products are also useful in generating chimpanzee recombinant Ads.

V.D.3. Recombinant Viral Particles as Vectors

The compositions disclosed herein can comprise viral vectors, that deliver at least one antigen to cells. Such vectors comprise a chimpanzee adenovirus DNA sequence such as C68 and an antigen cassette operatively linked to regulatory sequences which direct expression of the cassette. The C68 vector is capable of expressing the cassette in an infected mammalian cell. The C68 vector can be functionally deleted in one or more viral genes. An antigen cassette comprises at least one antigen under the control of one or more regulatory sequences such as a promoter. Optional helper viruses and/or packaging cell lines can supply to the chimpanzee viral vector any necessary products of deleted adenoviral genes.

The term “functionally deleted” means that a sufficient amount of the gene region is removed or otherwise altered, e.g., by mutation or modification, so that the gene region is no longer capable of producing one or more functional products of gene expression. Mutations or modifications that can result in functional deletions include, but are not limited to, nonsense mutations such as introduction of premature stop codons and removal of canonical and non-canonical start codons, mutations that alter mRNA splicing or other transcriptional processing, or combinations thereof. If desired, the entire gene region can be removed.

Modifications of the nucleic acid sequences forming the vectors disclosed herein, including sequence deletions, insertions, and other mutations may be generated using standard molecular biological techniques and are within the scope of this invention.

V.D.4. Construction of the Viral Plasmid Vector

The chimpanzee adenovirus C68 vectors useful in this invention include recombinant, defective adenoviruses, that is, chimpanzee adenovirus sequences functionally deleted in the E1a or E1b genes, and optionally bearing other mutations, e.g., temperature-sensitive mutations or deletions in other genes. It is anticipated that these chimpanzee sequences are also useful in forming hybrid vectors from other adenovirus and/or adeno-associated virus sequences. Homologous adenovirus vectors prepared from human adenoviruses are described in the published literature [see, for example, Kozarsky I and II, cited above, and references cited therein, U.S. Pat. No. 5,240,846].

In the construction of useful chimpanzee adenovirus C68 vectors for delivery of an antigen cassette to a human (or other mammalian) cell, a range of adenovirus nucleic acid sequences can be employed in the vectors. A vector comprising minimal chimpanzee C68 adenovirus sequences can be used in conjunction with a helper virus to produce an infectious recombinant virus particle. The helper virus provides essential gene products required for viral infectivity and propagation of the minimal chimpanzee adenoviral vector. When only one or more selected deletions of chimpanzee adenovirus genes are made in an otherwise functional viral vector, the deleted gene products can be supplied in the viral vector production process by propagating the virus in a selected packaging cell line that provides the deleted gene functions in trans.

V.D.5. Recombinant Minimal Adenovirus

A minimal chimpanzee Ad C68 virus is a viral particle containing just the adenovirus cis-elements necessary for replication and virion encapsidation. That is, the vector contains the cis-acting 5′ and 3′ inverted terminal repeat (ITR) sequences of the adenoviruses (which function as origins of replication) and the native 5′ packaging/enhancer domains (that contain sequences necessary for packaging linear Ad genomes and enhancer elements for the E1 promoter). See, for example, the techniques described for preparation of a “minimal” human Ad vector in International Patent Application WO96/13597 and incorporated herein by reference.

V.D.6. Other Defective Adenoviruses

Recombinant, replication-deficient adenoviruses can also contain more than the minimal chimpanzee adenovirus sequences. These other Ad vectors can be characterized by deletions of various portions of gene regions of the virus, and infectious virus particles formed by the optional use of helper viruses and/or packaging cell lines.

As one example, suitable vectors may be formed by deleting all or a sufficient portion of the C68 adenoviral immediate early gene E1a and delayed early gene E1b, so as to eliminate their normal biological functions. Replication-defective E1-deleted viruses are capable of replicating and producing infectious virus when grown on a chimpanzee adenovirus-transformed, complementation cell line containing functional adenovirus E1a and E1b genes which provide the corresponding gene products in trans. Based on the homologies to known adenovirus sequences, it is anticipated that, as is true for the human recombinant E1-deleted adenoviruses of the art, the resulting recombinant chimpanzee adenovirus is capable of infecting many cell types and can express antigen(s), but cannot replicate in most cells that do not carry the chimpanzee E1 region DNA unless the cell is infected at a very high multiplicity of infection.

As another example, all or a portion of the C68 adenovirus delayed early gene E3 can be eliminated from the chimpanzee adenovirus sequence which forms a part of the recombinant virus.

Chimpanzee adenovirus C68 vectors can also be constructed having a deletion of the E4 gene. Still another vector can contain a deletion in the delayed early gene E2a.

Deletions can also be made in any of the late genes L1 through L5 of the chimpanzee C68 adenovirus genome. Similarly, deletions in the intermediate genes IX and IVa2 can be useful for some purposes. Other deletions may be made in the other structural or non-structural adenovirus genes.

The above discussed deletions can be used individually, i.e., an adenovirus sequence can contain deletions of E1 only. Alternatively, deletions of entire genes or portions thereof effective to destroy or reduce their biological activity can be used in any combination. For example, in one exemplary vector, the adenovirus C68 sequence can have deletions of the E1 genes and the E4 gene, or of the E1, E2a and E3 genes, or of the E1 and E3 genes, or of E1, E2a and E4 genes, with or without deletion of E3, and so on. As discussed above, such deletions can be used in combination with other mutations, such as temperature-sensitive mutations, to achieve a desired result.

The cassette comprising antigen(s) be inserted optionally into any deleted region of the chimpanzee C68 Ad virus. Alternatively, the cassette can be inserted into an existing gene region to disrupt the function of that region, if desired.

V.D.7. Helper Viruses

Depending upon the chimpanzee adenovirus gene content of the viral vectors employed to carry the antigen cassette, a helper adenovirus or non-replicating virus fragment can be used to provide sufficient chimpanzee adenovirus gene sequences to produce an infective recombinant viral particle containing the cassette.

Useful helper viruses contain selected adenovirus gene sequences not present in the adenovirus vector construct and/or not expressed by the packaging cell line in which the vector is transfected. A helper virus can be replication-defective and contain a variety of adenovirus genes in addition to the sequences described above. The helper virus can be used in combination with the E1-expressing cell lines described herein.

For C68, the “helper” virus can be a fragment formed by clipping the C terminal end of the C68 genome with SspI, which removes about 1300 bp from the left end of the virus. This clipped virus is then co-transfected into an E1-expressing cell line with the plasmid DNA, thereby forming the recombinant virus by homologous recombination with the C68 sequences in the plasmid.

Helper viruses can also be formed into poly-cation conjugates as described in Wu et al, J. Biol. Chem., 264:16985-16987 (1989); K. J. Fisher and J. M. Wilson, Biochem. J., 299:49 (Apr. 1, 1994). Helper virus can optionally contain a reporter gene. A number of such reporter genes are known to the art. The presence of a reporter gene on the helper virus which is different from the antigen cassette on the adenovirus vector allows both the Ad vector and the helper virus to be independently monitored. This second reporter is used to enable separation between the resulting recombinant virus and the helper virus upon purification.

V.D.8. Assembly of Viral Particle and Infection of a Cell Line

Assembly of the selected DNA sequences of the adenovirus, the antigen cassette, and other vector elements into various intermediate plasmids and shuttle vectors, and the use of the plasmids and vectors to produce a recombinant viral particle can all be achieved using conventional techniques. Such techniques include conventional cloning techniques of cDNA, in vitro recombination techniques (e.g., Gibson assembly), use of overlapping oligonucleotide sequences of the adenovirus genomes, polymerase chain reaction, and any suitable method which provides the desired nucleotide sequence. Standard transfection and co-transfection techniques are employed, e.g., CaPO4 precipitation techniques or liposome-mediated transfection methods such as lipofectamine Other conventional methods employed include homologous recombination of the viral genomes, plaquing of viruses in agar overlay, methods of measuring signal generation, and the like.

For example, following the construction and assembly of the desired antigen cassette-containing viral vector, the vector can be transfected in vitro in the presence of a helper virus into the packaging cell line. Homologous recombination occurs between the helper and the vector sequences, which permits the adenovirus-antigen sequences in the vector to be replicated and packaged into virion capsids, resulting in the recombinant viral vector particles.

The resulting recombinant chimpanzee C68 adenoviruses are useful in transferring an antigen cassette to a selected cell. In in vivo experiments with the recombinant virus grown in the packaging cell lines, the E1-deleted recombinant chimpanzee adenovirus demonstrates utility in transferring a cassette to a non-chimpanzee, preferably a human, cell.

V.D.9. Use of the Recombinant Virus Vectors

The resulting recombinant chimpanzee C68 adenovirus containing the antigen cassette (produced by cooperation of the adenovirus vector and helper virus or adenoviral vector and packaging cell line, as described above) thus provides an efficient gene transfer vehicle which can deliver antigen(s) to a subject in vivo or ex vivo.

The above-described recombinant vectors are administered to humans according to published methods for gene therapy. A chimpanzee viral vector bearing an antigen cassette can be administered to a patient, preferably suspended in a biologically compatible solution or pharmaceutically acceptable delivery vehicle. A suitable vehicle includes sterile saline. Other aqueous and non-aqueous isotonic sterile injection solutions and aqueous and non-aqueous sterile suspensions known to be pharmaceutically acceptable carriers and well known to those of skill in the art may be employed for this purpose.

The chimpanzee adenoviral vectors are administered in sufficient amounts to transduce the human cells and to provide sufficient levels of antigen transfer and expression to provide a therapeutic benefit without undue adverse or with medically acceptable physiological effects, which can be determined by those skilled in the medical arts. Conventional and pharmaceutically acceptable routes of administration include, but are not limited to, direct delivery to the liver, intranasal, intravenous, intramuscular, subcutaneous, intradermal, oral and other parental routes of administration. Routes of administration may be combined, if desired.

Dosages of the viral vector will depend primarily on factors such as the condition being treated, the age, weight and health of the patient, and may thus vary among patients. The dosage will be adjusted to balance the therapeutic benefit against any side effects and such dosages may vary depending upon the therapeutic application for which the recombinant vector is employed. The levels of expression of antigen(s) can be monitored to determine the frequency of dosage administration.

Recombinant, replication defective adenoviruses can be administered in a “pharmaceutically effective amount”, that is, an amount of recombinant adenovirus that is effective in a route of administration to transfect the desired cells and provide sufficient levels of expression of the selected gene to provide a vaccinal benefit, i.e., some measurable level of protective immunity. C68 vectors comprising an antigen cassette can be co-administered with adjuvant. Adjuvant can be separate from the vector (e.g., alum) or encoded within the vector, in particular if the adjuvant is a protein. Adjuvants are well known in the art.

Conventional and pharmaceutically acceptable routes of administration include, but are not limited to, intranasal, intramuscular, intratracheal, subcutaneous, intradermal, rectal, oral and other parental routes of administration. Routes of administration may be combined, if desired, or adjusted depending upon the immunogen or the disease. For example, in prophylaxis of rabies, the subcutaneous, intratracheal and intranasal routes are preferred. The route of administration primarily will depend on the nature of the disease being treated.

The levels of immunity to antigen(s) can be monitored to determine the need, if any, for boosters. Following an assessment of antibody titers in the serum, for example, optional booster immunizations may be desired

### VI. Therapeutic and Manufacturing Methods

Also provided is a method of stimulating a tumor specific immune response in a subject, vaccinating against a tumor, treating and/or alleviating a symptom of cancer in a subject by administering to the subject one or more antigens such as a plurality of antigens identified using methods disclosed herein.

Also provided is a method of stimulating an infectious disease organism-specific immune response in a subject, vaccinating against an infectious disease organism, treating and/or alleviating a symptom of an infection associated with an infectious disease organism in a subject by administering to the subject one or more antigens such as a plurality of antigens identified using methods disclosed herein.

In some aspects, a subject has been diagnosed with cancer or is at risk of developing cancer. A subject can be a human, dog, cat, horse or any animal in which a tumor specific immune response is desired. A tumor can be any solid tumor such as breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas.

In some aspects, a subject has been diagnosed with an infection or is at risk of an infection (e.g., age, geographical/travel, and/or work-related increased risk of or predisposition to an infection, or at risk to a seasonal and/or novel disease infection).

An antigen can be administered in an amount sufficient to stimulate a CTL response. An antigen can be administered in an amount sufficient to stimulate a T cell response. An antigen can be administered in an amount sufficient to stimulate a B cell response. An antigen can be administered in an amount sufficient to stimulate both a T cell response and a B cell response.

An antigen can be administered alone or in combination with other therapeutic agents. Therapeutic agents can include those that target an infectious disease organism, such as an anti-viral or antibiotic agent.

In addition, a subject can be further administered an anti-immunosuppressive/immunostimulatory agent such as a checkpoint inhibitor. For example, the subject can be further administered an anti-CTLA antibody or anti-PD-1 or anti-PD-L1. Blockade of CTLA-4 or PD-L1 by antibodies can enhance the immune response to cancerous cells in the patient. In particular, CTLA-4 blockade has been shown effective when following a vaccination protocol.

The optimum amount of each antigen to be included in a vaccine composition and the optimum dosing regimen can be determined. For example, an antigen or its variant can be prepared for intravenous (i.v.) injection, sub-cutaneous (s.c.) injection, intradermal (i.d.) injection, intraperitoneal (i.p.) injection, intramuscular (i.m.) injection. Methods of injection include s.c., i.d., i.p., i.m., and i.v. Methods of DNA or RNA injection include i.d., i.m., s.c., i.p. and i.v. Other methods of administration of the vaccine composition are known to those skilled in the art.

A vaccine can be compiled so that the selection, number and/or amount of antigens present in the composition is/are tissue, cancer, infectious disease, and/or patient-specific. For instance, the exact selection of peptides can be guided by expression patterns of the parent proteins in a given tissue or guided by mutation or disease status of a patient. The selection can be dependent on the specific type of cancer, the specific infectious disease (e.g. a specific infectious disease isolate/strain the subject is infected with or at risk for infection by), the status of the disease, the goal of the vaccination (e.g., preventative or targeting an ongoing disease), earlier treatment regimens, the immune status of the patient, and, of course, the HLA-haplotype of the patient. Furthermore, a vaccine can contain individualized components, according to personal needs of the particular patient. Examples include varying the selection of antigens according to the expression of the antigen in the particular patient or adjustments for secondary treatments following a first round or scheme of treatment.

A patient can be identified for administration of an antigen vaccine through the use of various diagnostic methods, e.g., patient selection methods described further below. Patient selection can involve identifying mutations in, or expression patterns of, one or more genes. Patient selection can involve identifying the infectious disease of an ongoing infection. Patient selection can involve identifying risk of an infection by an infectious disease. In some cases, patient selection involves identifying the haplotype of the patient. The various patient selection methods can be performed in parallel, e.g., a sequencing diagnostic can identify both the mutations and the haplotype of a patient. The various patient selection methods can be performed sequentially, e.g., one diagnostic test identifies the mutations and separate diagnostic test identifies the haplotype of a patient, and where each test can be the same (e.g., both high-throughput sequencing) or different (e.g., one high-throughput sequencing and the other Sanger sequencing) diagnostic methods.

For a composition to be used as a vaccine for cancer or an infectious disease, antigens with similar normal self-peptides that are expressed in high amounts in normal tissues can be avoided or be present in low amounts in a composition described herein. On the other hand, if it is known that the tumor or infected cell of a patient expresses high amounts of a certain antigen, the respective pharmaceutical composition for treatment of this cancer or infection can be present in high amounts and/or more than one antigen specific for this particularly antigen or pathway of this antigen can be included.

Compositions comprising an antigen can be administered to an individual already suffering from cancer or an infection. In therapeutic applications, compositions are administered to a subject in an amount sufficient to stimulate an effective CTL response to the tumor antigen or infectious disease organism antigen and to cure or at least partially arrest symptoms and/or complications. An amount adequate to accomplish this is defined as “therapeutically effective dose.” Amounts effective for this use will depend on, e.g., the composition, the manner of administration, the stage and severity of the disease being treated, the weight and general state of health of the patient, and the judgment of the prescribing physician. It should be kept in mind that compositions can generally be employed in serious disease states, that is, life-threatening or potentially life threatening situations, especially when a cancer has metastasized or an infectious disease organism has induced organ damage and/or other immune pathology. In such cases, in view of the minimization of extraneous substances and the relative nontoxic nature of an antigen, it is possible and can be felt desirable by the treating physician to administer substantial excesses of these compositions.

For therapeutic use, administration can begin at the detection or surgical removal of tumors, or begin at the detection or treatment of an infection. This can be followed by boosting doses until at least symptoms are substantially abated and for a period thereafter, or immunity is considered to be provided (e.g., a memory B cell or T cell population, or antigen specific B cells or antibodies are produced).

The pharmaceutical compositions (e.g., vaccine compositions) for therapeutic treatment are intended for parenteral, topical, nasal, oral or local administration. A pharmaceutical compositions can be administered parenterally, e.g., intravenously, subcutaneously, intradermally, or intramuscularly. The compositions can be administered at the site of surgical excision to stimulate a local immune response to a tumor. The compositions can be administered to target specific infected tissues and/or cells of a subject. Disclosed herein are compositions for parenteral administration which comprise a solution of the antigen and vaccine compositions are dissolved or suspended in an acceptable carrier, e.g., an aqueous carrier. A variety of aqueous carriers can be used, e.g., water, buffered water, 0.9% saline, 0.3% glycine, hyaluronic acid and the like. These compositions can be sterilized by conventional, well known sterilization techniques, or can be sterile filtered. The resulting aqueous solutions can be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

Antigens can also be administered via liposomes, which target them to a particular cells tissue, such as lymphoid tissue. Liposomes are also useful in increasing half-life. Liposomes include emulsions, foams, micelles, insoluble monolayers, liquid crystals, phospholipid dispersions, lamellar layers and the like. In these preparations the antigen to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired antigen can be directed to the site of lymphoid cells, where the liposomes then deliver the selected therapeutic/immunogenic compositions. Liposomes can be formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369.

For targeting to the immune cells, a ligand to be incorporated into the liposome can include, e.g., antibodies or fragments thereof specific for cell surface determinants of the desired immune system cells. A liposome suspension can be administered intravenously, locally, topically, etc. in a dose which varies according to, inter alia, the manner of administration, the peptide being delivered, and the stage of the disease being treated.

For therapeutic or immunization purposes, nucleic acids encoding a peptide and optionally one or more of the peptides described herein can also be administered to the patient. A number of methods are conveniently used to deliver the nucleic acids to the patient. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles. Approaches for delivering nucleic acid sequences can include viral vectors, mRNA vectors, and DNA vectors with or without electroporation.

The nucleic acids can also be delivered complexed to cationic compounds, such as cationic lipids. Lipid-mediated gene delivery methods are described, for instance, in 9618372WOAWO 96/18372; 9324640WOAWO 93/24640; Mannino & Gould-Fogerite, BioTechniques 6(7): 682-691 (1988); U.S. Pat. No. 5,279,833 Rose U.S. Pat. Nos. 5,279,833; 9,106,309WOAWO 91/06309; and Feigner et al., Proc. Natl. Acad. Sci. USA 84: 7413-7414 (1987).

Antigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al, Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol. (1998) 72 (12): 9873-9880). Dependent on the packaging capacity of the above mentioned viral vector-based vaccine platforms, this approach can deliver one or more nucleotide sequences that encode one or more antigen peptides. The sequences may be flanked by non-mutated sequences, may be separated by linkers or may be preceded with one or more sequences targeting a subcellular compartment (See, e.g., Gros et al., Prospective identification of neoantigen-specific lymphocytes in the peripheral blood of melanoma patients, Nat Med. (2016) 22 (4):433-8, Stronen et al., Targeting of cancer neoantigens with donor-derived T cell receptor repertoires, Science. (2016) 352 (6291):1337-41, Lu et al., Efficient identification of mutated cancer antigens recognized by T cells associated with durable tumor regressions, Clin Cancer Res. (2014) 20(13):3401-10). Upon introduction into a host, infected cells express the antigens, and thereby stimulate a host immune (e.g., CTL) response against the peptide(s). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of antigens, e.g., Salmonella typhi vectors, and the like will be apparent to those skilled in the art from the description herein.

A means of administering nucleic acids uses minigene constructs encoding one or multiple epitopes. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include: helper T lymphocyte, epitopes, a leader (signal) sequence, and an endoplasmic reticulum retention signal. In addition, MHC presentation of CTL epitopes can be improved by including synthetic (e.g. poly-alanine) or naturally-occurring flanking sequences adjacent to the CTL epitopes. The minigene sequence is converted to DNA by assembling oligonucleotides that encode the plus and minus strands of the minigene. Overlapping oligonucleotides (30-100 bases long) are synthesized, phosphorylated, purified and annealed under appropriate conditions using well known techniques. The ends of the oligonucleotides are joined using T4 DNA ligase. This synthetic minigene, encoding the CTL epitope polypeptide, can then cloned into a desired expression vector.

Purified plasmid DNA can be prepared for injection using a variety of formulations. The simplest of these is reconstitution of lyophilized DNA in sterile phosphate-buffer saline (PBS). A variety of methods have been described, and new techniques can become available. As noted above, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

Also disclosed is a method of manufacturing a vaccine, comprising performing the steps of a method disclosed herein; and producing a vaccine comprising a plurality of antigens or a subset of the plurality of antigens.

Antigens disclosed herein can be manufactured using methods known in the art. For example, a method of producing an antigen or a vector (e.g., a vector including at least one sequence encoding one or more antigens) disclosed herein can include culturing a host cell under conditions suitable for expressing the antigen or vector wherein the host cell comprises at least one polynucleotide encoding the antigen or vector, and purifying the antigen or vector. Standard purification methods include chromatographic techniques, electrophoretic, immunological, precipitation, dialysis, filtration, concentration, and chromatofocusing techniques.

Host cells can include a Chinese Hamster Ovary (CHO) cell, NS0 cell, yeast, or a HEK293 cell. Host cells can be transformed with one or more polynucleotides comprising at least one nucleic acid sequence that encodes an antigen or vector disclosed herein, optionally wherein the isolated polynucleotide further comprises a promoter sequence operably linked to the at least one nucleic acid sequence that encodes the antigen or vector. In certain embodiments the isolated polynucleotide can be cDNA.

### VII. Antigen Use and Administration

Vaccination methods, protocols, and schedules that can also be used include, but are not limited to, those described in international application publication WO2021092095, herein incorporated by reference for all purposes.

Each vector in a prime/boost strategy typically includes a cassette that includes antigens. Cassettes can include about 1-50 antigens, separated by linkers such as the natural sequence that normally surrounds each antigen or other non-natural linker sequences such as AAY. Cassettes can also include MHCII antigens such a tetanus toxoid antigen and PADRE antigen, which can be considered universal class II antigens. Cassettes can also include a targeting sequence such as a ubiquitin targeting sequence. In addition, each vaccine dose can be administered to the subject in conjunction with (e.g., concurrently, before, or after) an immune modulator. Each vaccine dose can be administered to the subject in conjunction with (e.g., concurrently, before, or after) a checkpoint inhibitor (CPI). CPI's can include those that inhibit CTLA4, PD1, and/or PDL1 such as antibodies or antigen-binding portions thereof. Such antibodies can include tremelimumab or durvalumab. Each vaccine dose can be administered to the subject in conjunction with (e.g., concurrently, before, or after) a cytokine, such as IL-2, IL-7, IL-12 (including IL-12 p35, p40, p70, and/or p70-fusion constructs), IL-15, or IL-21. Each vaccine dose can be administered to the subject in conjunction with (e.g., concurrently, before, or after) a modified cytokine (e.g., pegIL-2).

A vaccination protocol can be used to dose a subject with one or more antigens. A priming vaccine and a boosting vaccine can be used to dose the subject. The priming vaccine can be with any of the antigen encoding vectors described herein, such as vectors based on ChAdV68 (e.g., the sequences shown in SEQ ID NO:1 or 2). The boosting dose can be with any of the antigen encoding vectors described herein, such as vectors based on ChAdV68 (e.g., the sequences shown in SEQ ID NO:1 or 2) or SAM-based vectors (e.g., the sequences shown in SEQ ID NO:3 or 4). One or more boosting doses can be administered and can be serial administration of the same boosting vaccine (e.g., serial administration of the same ChAdV68-based vectors or serial administration of the same SAM-based vectors) or can be serial administration of different boosting vaccines (e.g., administration of a SAM-based vector followed by administration of a ChAdV68-based vector). Serial administration of different vaccines can include any combination of different vaccines. For example, a vaccine strategy can use a ChAdV68-based prime, followed by one or more SAM-based boosts, and the SAM-based boosts followed by a ChAdV68-based boost. Illustrative non-limiting vaccine strategies include, but are not limited to: ChAdV prime-SAM boost-SAM boost-ChAdV boost; or ChAdV prime-SAM boost-SAM boost-SAM boost-SAM boost-ChAdV boost.

ChAdV68-based vaccines can be administered at a dose ranging from 1×10¹¹ viral particles to 1×10¹² viral particles. ChAdV68-based vaccines can be administered at a dose of 1×10¹¹ viral particles. ChAdV68-based vaccines can be administered at a dose of 5×10¹¹ viral particles. ChAdV68-based vaccines can be administered at a dose of 1×10¹² viral particles. The selected dosage for ChAdV68-based vaccines will depend on, e.g., the composition, the manner of administration, the stage and severity of the disease being treated, the weight and general state of health of the patient, and the judgment of the prescribing physician.

SAM-based vaccines can be administered at a dose ranging 10-300 μg RNA. SAM-based vaccines can be administered at a dose ranging 100-300 μg RNA. SAM-based vaccines can be administered at a dose of 100 μg RNA. SAM-based vaccines can be administered at a dose of 300 μg RNA. The selected dosage for SAM-based vaccines will depend on, e.g., the composition, the manner of administration, the stage and severity of the disease being treated, the weight and general state of health of the patient, and the judgment of the prescribing physician.

A priming vaccine can be injected (e.g., intramuscularly) in a subject. Bilateral injections per dose can be used. For example, one or more injections of ChAdV68 (C68) can be used (e.g., total dose 1×10¹² viral particles); one or more injections of SAM vectors at low vaccine dose selected from the range 0.001 to 1 ug RNA, in particular 0.1 or 1 ug can be used; or one or more injections of SAM vectors at high vaccine dose selected from the range 1 to 100 ug RNA, in particular 10, 100, or 300 ug can be used.

A vaccine boost (boosting vaccine) can be injected (e.g., intramuscularly) after prime vaccination. Bilateral injections per dose can be used. For example, one or more injections of ChAdV68 (C68) can be used (e.g., total dose 1×10¹² viral particles); one or more injections of SAM vectors at low vaccine dose selected from the range 0.001 to 1 ug RNA, in particular 0.1 or 1 ug can be used; or one or more injections of SAM vectors at high vaccine dose selected from the range 1 to 100 ug RNA, in particular 10, 100 or 300 ug can be used.

A boosting vaccine can be administered about every 1, 2, 3, 4, 5, 6, 7, 8, 9, or 10 weeks, e.g., every 4 weeks and/or 8 weeks after the prime. A boosting vaccine can be administered every 4 weeks after the prime. A boosting vaccine can be administered every 6 weeks after the prime. A boosting vaccine can be administered every 12 weeks after the prime. Boosting doses can be administered at different intervals during the course of a vaccination protocol. For example, illustrative non-limiting examples include prime-4 w-boost-12 w-boost -12 w-boost; or prime-4 w-boost-6 w-boost-6 w-boost-6 w-boost-6 w-boost, where “w” represents weeks.

One or more of the vaccine administrations can include co-administration of one or more checkpoint inhibitors. Illustrative immune checkpoint inhibitors include Tremelimumab (CTLA-4 blocking antibody), anti-OX40, PD-L1 monoclonal Antibody (Anti-B7-H1; MEDI4736), ipilimumab, MK-3475 (PD-1 blocker), Nivolumamb (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor). In illustrative non-limiting examples, Nivolumamb, Yervoy/ipilimumab, or a combination thereof.

Anti-CTLA-4 (e.g., tremelimumab) can also be administered to the subject. For example, anti-CTLA4 can be administered subcutaneously near the site of the intramuscular vaccine injection (ChAdV68 prime or SAM low doses) to ensure drainage into the same lymph node. Tremelimumab is a selective human IgG2 mAb inhibitor of CTLA-4. Target Anti-CTLA-4 (tremelimumab) subcutaneous dose is typically 70-75 mg (in particular 75 mg) with a dose range of, e.g., 1-100 mg or 5-420 mg.

In certain instances an anti-PD-L1 antibody can be used such as durvalumab (MEDI 4736). Durvalumab is a selective, high affinity human IgG1 mAb that blocks PD-L1 binding to PD-1 and CD80. Durvalumab is generally administered at 20 mg/kg i.v. every 4 weeks.

Immune monitoring can be performed before, during, and/or after vaccine administration. Such monitoring can inform safety and efficacy, among other parameters.

To perform immune monitoring, PBMCs are commonly used. PBMCs can be isolated before prime vaccination, and after prime vaccination (e.g. 4 weeks and 8 weeks). PBMCs can be harvested just prior to boost vaccinations and after each boost vaccination (e.g. 4 weeks and 8 weeks).

Immune responses, such as T cell responses and B cells responses, can be assessed as part of an immune monitoring protocol. For example, the ability of a vaccine composition described herein to stimulate an immune response can be monitored and/or assessed. As used herein, “stimulate an immune response” refers to any increase in a immune response, such as initiating an immune response (e.g., a priming vaccine stimulating the initiation of an immune response in a naïve subject) or enhancement of an immune response (e.g., a boosting vaccine stimulating the enhancement of an immune response in a subject having a pre-existing immune response to an antigen, such as a pre-existing immune response initiated by a priming vaccine). T cell responses can be measured using one or more methods known in the art such as ELISpot, intracellular cytokine staining, cytokine secretion and cell surface capture, T cell proliferation, MHC multimer staining, or by cytotoxicity assay. T cell responses to epitopes encoded in vaccines can be monitored from PBMCs by measuring induction of cytokines, such as IFN-gamma, using an ELISpot assay. Specific CD4 or CD8 T cell responses to epitopes encoded in vaccines can be monitored from PBMCs by measuring induction of cytokines captured intracellularly or extracellularly, such as IFN-gamma, using flow cytometry. Specific CD4 or CD8 T cell responses to epitopes encoded in the vaccines can be monitored from PBMCs by measuring T cell populations expressing T cell receptors specific for epitope/MHC class I complexes using MHC multimer staining. Specific CD4 or CD8 T cell responses to epitopes encoded in the vaccines can be monitored from PBMCs by measuring the ex vivo expansion of T cell populations following 3H-thymidine, bromodeoxyuridine and carboxyfluoresceine-diacetate-succinimidylester (CFSE) incorporation. The antigen recognition capacity and lytic activity of PBMC-derived T cells that are specific for epitopes encoded in vaccines can be assessed functionally by chromium release assay or alternative colorimetric cytotoxicity assays.

B cell responses can be measured using one or more methods known in the art such as assays used to determine B cell differentiation (e.g., differentiation into plasma cells), B cell or plasma cell proliferation, B cell or plasma cell activation (e.g., upregulation of costimulatory markers such as CD80 or CD86), antibody class switching, and/or antibody production (e.g., an ELISA).

Disease status of a subject can be monitored following administration of any of the vaccine compositions described herein. For example, disease status may be monitored using isolated cell-free DNA (cfDNA) from a subject. In addition, the efficacy of a vaccine therapy may be monitored using isolated cfDNA from a subject. cfDNA monitoring can include the steps of: a. isolating or having isolated cfDNA from a subject; b. sequencing or having sequenced the isolated cfDNA; c. determining or having determined a frequency of one or more mutations in the cfDNA relative to a wild-type germline nucleic acid sequence of the subject, and d. assessing or having assessed from step (c) the status of a disease in the subject. The method can also include, following step (c) above, d. performing more than one iteration of steps (a)-(c) for the given subject and comparing the frequency of the one or more mutations determined in the more than one iterations; and f. assessing or having assessed from step (d) the status of a disease in the subject. The more than one iterations can be performed at different time points, such as a first iteration of steps (a)-(c) performed prior to administration of the vaccine composition and a second iteration of steps (a)-(c) is performed subsequent to administration of the vaccine composition. Step (c) can include comparing: the frequency of the one or more mutations determined in the more than one iterations, or the frequency of the one or more mutations determined in the first iteration to the frequency of the one or more mutations determined in the second iteration. An increase in the frequency of the one or more mutations determined in subsequent iterations or the second iteration can be assessed as disease progression. A decrease in the frequency of the one or more mutations determined in subsequent iterations or the second iteration can be assessed as a response. In some aspects, the response is a Complete Response (CR) or a Partial Response (PR). A therapy can be administered to a subject following an assessment step, such as where assessment of the frequency of the one or more mutations in the cfDNA indicates the subject has the disease. The cfDNA isolation step can use centrifugation to separate cfDNA from cells or cellular debris. cfDNA can be isolated from whole blood, such as by separating the plasma layer, buffy coat, and red bloods. cfDNA sequencing can use next generation sequencing (NGS), Sanger sequencing, duplex sequencing, whole-exome sequencing, whole-genome sequencing, de novo sequencing, phased sequencing, targeted amplicon sequencing, shotgun sequencing, or combinations thereof, and may include enriching the cfDNA for one or more polynucleotide regions of interest prior to sequencing (e.g., polynucleotides known or suspected to encode the one or more mutations, coding regions, and/or tumor exome polynucleotides). Enriching the cfDNA may include hybridizing one or more polynucleotide probes, which may be modified (e.g., biotinylated), to the one or more polynucleotide regions of interest. In general, any number of mutations may be monitored simultaneously or in parallel.

Homologous vaccination regimens can include an interval between homologous doses to improve efficacy of the second dose. For example, a ChAdV68-based vaccine can be administered as an initial dose and include an interval prior to re-administration of the ChAdV68-based vaccine as a boosting dose to improve efficacy, such as reducing the impact of ChAdV-specific neutralizing antibody titers on the efficacy of the boosting dose. For example, an initial dose may induce production of neutralizing antibodies which then subsequently wane over time. In illustrative non-limiting examples for ChAdV68-based vaccines described herein, the interval is at least 27 weeks. The interval can be 27 weeks. The interval can be 28 weeks. The interval can be 29 weeks. The interval can be 30 weeks. The interval can be 31 weeks. The interval can be 32 weeks. The interval can be 33 weeks. The interval can be at least 27 weeks. The interval can be at least 28 weeks. The interval can be at least 29 weeks. The interval can be at least 30 weeks. The interval can be at least 31 weeks. The interval can be at least 32 weeks. The interval can be at least 33 weeks.

The interval between ChAdV68-based vaccine administrations in a homologous prime-boost strategy can be as few as 8 weeks. The interval can be 8 weeks. The interval can be 9 weeks. The interval can be 10 weeks. The interval can be 11 weeks. The interval can be 12 weeks. The interval can be 13 weeks. The interval can be 14 weeks. The interval can be 15 weeks. The interval can be 16 weeks. The interval can be 17 weeks. The interval can be 18 weeks. The interval can be 19 weeks. The interval can be 20 weeks. The interval can be 21 weeks. The interval can be 23 weeks. The interval can be 24 weeks. The interval can be 25 weeks. The interval can be 26 weeks.

The interval between ChAdV68-based vaccine administrations in a homologous prime-boost strategy can be at least 8 weeks. The interval can be at least 9 weeks. The interval can be at least 10 weeks. The interval can be at least 11 weeks. The interval can be at least 12 weeks. The interval can be at least 13 weeks. The interval can be at least 14 weeks. The interval can be at least 15 weeks. The interval can be at least 16 weeks. The interval can be at least 17 weeks. The interval can be at least 18 weeks. The interval can be at least 19 weeks. The interval can be at least 20 weeks. The interval can be at least 21 weeks. The interval can be at least 23 weeks. The interval can be at least 24 weeks. The interval can be at least 25 weeks. The interval can be at least 26 weeks.

The interval between ChAdV68-based vaccine administrations in a homologous prime-boost strategy can be 2 months. The interval can be 2.5 months. The interval can be 3 months. The interval can be 3.5 months. The interval can be 4 months. The interval can be 4.5 months. The interval can be 5 months. The interval can be 5.5 months. The interval can be 6 months. The interval can be 6.5 months. The interval can be 7 months. The interval can be 7.5 months. The interval can be 8 months. The interval can be 8.5 months. The interval can be at least 2 months. The interval can be at least 2.5 months. The interval can be at least 3 months. The interval can be at least 3.5 months. The interval can be at least 4 months. The interval can be at least 4.5 months. The interval can be at least 5 months. The interval can be at least 5.5 months. The interval can be at least 6 months. The interval can be at least 6.5 months. The interval can be at least 7 months. The interval can be at least 7.5 months. The interval can be at least 8 months. The interval can be at least 8.5 months.

### VIII. Isolation and Detection of HLA Peptides

Isolation of HLA-peptide molecules was performed using classic immunoprecipitation (IP) methods after lysis and solubilization of the tissue sample (55-58). A clarified lysate was used for HLA specific IP. Examples and methods are described in more detail in international patent application publication WO/2018/208856, herein incorporated by reference, in its entirety, for all purposes.

### IX. Presentation Model

Presentation models can be used to identify likelihoods of peptide presentation in patients. Various presentation models are known to those skilled in the art, for example the presentation models described in more detail in U.S. Pat. No. 10,055,540, US Application Pub. No. US20200010849A1 and US20110293637, and international patent application publications WO/2018/195357, WO/2018/208856, and WO2016187508, each herein incorporated by reference, in their entirety, for all purposes.

### X. Training Module

Training modules can be used to construct one or more presentation models based on training data sets that generate likelihoods of whether peptide sequences will be presented by MHC alleles associated with the peptide sequences. Various training modules are known to those skilled in the art, for example the presentation models described in more detail in U.S. Pat. No. 10,055,540, US Application Pub. No. US20200010849A1, and international patent application publications WO/2018/195357 and WO/2018/208856, each herein incorporated by reference, in their entirety, for all purposes. A training module can construct a presentation model to predict presentation likelihoods of peptides on a per-allele basis. A training module can also construct a presentation model to predict presentation likelihoods of peptides in a multiple-allele setting where two or more MHC alleles are present.

### XI. Prediction Module

A prediction module can be used to receive sequence data and select candidate antigens in the sequence data using a presentation model. Specifically, the sequence data may be DNA sequences, RNA sequences, and/or protein sequences extracted from tumor tissue cells of patients, infected cells patients, or infectious disease organisms themselves. A prediction module may identify candidate neoantigens that are mutated peptide sequences by comparing sequence data extracted from normal tissue cells of a patient with the sequence data extracted from tumor tissue cells of the patient to identify portions containing one or more mutations. A prediction module may identify candidate antigens that are pathogen-derived peptides, virally-derived peptides, bacterially-derived peptides, fungally-derived peptides, and parasitically-derived peptides, such as by comparing sequence data extracted from normal tissue cells of a patient with the sequence data extracted from infected cells of the patient to identify portions containing one or more infectious disease organism associated antigens. A prediction module may identify candidate antigens that have altered expression in a tumor cell or cancerous tissue in comparison to a normal cell or tissue by comparing sequence data extracted from normal tissue cells of a patient with the sequence data extracted from tumor tissue cells of the patient to identify improperly expressed candidate antigens. A prediction module may identify candidate antigens that are expressed in an infected cell or infected tissue in comparison to a normal cell or tissue by comparing sequence data extracted from normal tissue cells of a patient with the sequence data extracted from infected tissue cells of the patient to identify expressed candidate antigens (e.g., identifying expressed polynucleotides and/or polypeptides specific to an infectious disease).

A presentation module can apply one or more presentation model to processed peptide sequences to estimate presentation likelihoods of the peptide sequences. Specifically, the prediction module may select one or more candidate antigen peptide sequences that are likely to be presented on tumor HLA molecules or infected cell HLA molecules by applying presentation models to the candidate antigens. In one implementation, the presentation module selects candidate antigen sequences that have estimated presentation likelihoods above a predetermined threshold. In another implementation, the presentation model selects the N candidate antigen sequences that have the highest estimated presentation likelihoods (where N is generally the maximum number of epitopes that can be delivered in a vaccine). A vaccine including the selected candidate antigens for a given patient can be injected into a subject to stimulate immune responses.

XI.B.Cassette Design Module

XI.B.1 Overview

A cassette design module can be used to generate a vaccine cassette sequence based on selected candidate peptides for injection into a patient. For example, a cassette design module can be used to generate a sequence encoding concatenated epitope sequences, such as concatenated T cell epitopes. Various cassette design modules are known to those skilled in the art, for example the cassette design modules described in more detail in U.S. Pat. No. 10,055,540, US Application Pub. No. US20200010849A1, and international patent application publications WO/2018/195357 and WO/2018/208856, each herein incorporated by reference, in their entirety, for all purposes.

A set of therapeutic epitopes may be generated based on the selected peptides determined by a prediction module associated with presentation likelihoods above a predetermined threshold, where the presentation likelihoods are determined by the presentation models. However it is appreciated that in other embodiments, the set of therapeutic epitopes may be generated based on any one or more of a number of methods (alone or in combination), for example, based on binding affinity or predicted binding affinity to HLA class I or class II alleles of the patient, binding stability or predicted binding stability to HLA class I or class II alleles of the patient, random sampling, and the like.

Therapeutic epitopes may correspond to selected peptides themselves. Therapeutic epitopes may also include C- and/or N-terminal flanking sequences in addition to the selected peptides. N- and C-terminal flanking sequences can be the native N- and C-terminal flanking sequences of the therapeutic vaccine epitope in the context of its source protein. Therapeutic epitopes can represent a fixed-length epitope Therapeutic epitopes can represent a variable-length epitope, in which the length of the epitope can be varied depending on, for example, the length of the C- or N-flanking sequence. For example, the C-terminal flanking sequence and the N-terminal flanking sequence can each have varying lengths of 2-5 residues, resulting in 16 possible choices for the epitope.

A cassette design module can also generate cassette sequences by taking into account presentation of junction epitopes that span the junction between a pair of therapeutic epitopes in the cassette. Junction epitopes are novel non-self but irrelevant epitope sequences that arise in the cassette due to the process of concatenating therapeutic epitopes and linker sequences in the cassette. The novel sequences of junction epitopes are different from the therapeutic epitopes of the cassette themselves.

A cassette design module can generate a cassette sequence that reduces the likelihood that junction epitopes are presented in the patient. Specifically, when the cassette is injected into the patient, junction epitopes have the potential to be presented by HLA class I or HLA class II alleles of the patient, and stimulate a CD8 or CD4 T-cell response, respectively. Such reactions are often times undesirable because T-cells reactive to the junction epitopes have no therapeutic benefit, and may diminish the immune response to the selected therapeutic epitopes in the cassette by antigenic competition.⁷⁶

A cassette design module can iterate through one or more candidate cassettes, and determine a cassette sequence for which a presentation score of junction epitopes associated with that cassette sequence is below a numerical threshold. The junction epitope presentation score is a quantity associated with presentation likelihoods of the junction epitopes in the cassette, and a higher value of the junction epitope presentation score indicates a higher likelihood that junction epitopes of the cassette will be presented by HLA class I or HLA class II or both.

In one embodiment, a cassette design module may determine a cassette sequence associated with the lowest junction epitope presentation score among the candidate cassette sequences.

A cassette design module may iterate through one or more candidate cassette sequences, determine the junction epitope presentation score for the candidate cassettes, and identify an optimal cassette sequence associated with a junction epitope presentation score below the threshold.

A cassette design module may further check the one or more candidate cassette sequences to identify if any of the junction epitopes in the candidate cassette sequences are self-epitopes for a given patient for whom the vaccine is being designed. To accomplish this, the cassette design module checks the junction epitopes against a known database such as BLAST. In one embodiment, the cassette design module may be configured to design cassettes that avoid junction self-epitopes.

A cassette design module can perform a brute force approach and iterate through all or most possible candidate cassette sequences to select the sequence with the smallest junction epitope presentation score. However, the number of such candidate cassettes can be prohibitively large as the capacity of the vaccine increases. For example, for a vaccine capacity of 20 epitopes, the cassette design module has to iterate through ˜10¹⁸ possible candidate cassettes to determine the cassette with the lowest junction epitope presentation score. This determination may be computationally burdensome (in terms of computational processing resources required), and sometimes intractable, for the cassette design module to complete within a reasonable amount of time to generate the vaccine for the patient. Moreover, accounting for the possible junction epitopes for each candidate cassette can be even more burdensome. Thus, a cassette design module may select a cassette sequence based on ways of iterating through a number of candidate cassette sequences that are significantly smaller than the number of candidate cassette sequences for the brute force approach.

A cassette design module can generate a subset of randomly or at least pseudo-randomly generated candidate cassettes, and selects the candidate cassette associated with a junction epitope presentation score below a predetermined threshold as the cassette sequence. Additionally, the cassette design module may select the candidate cassette from the subset with the lowest junction epitope presentation score as the cassette sequence. For example, the cassette design module may generate a subset of −1 million candidate cassettes for a set of 20 selected epitopes, and select the candidate cassette with the smallest junction epitope presentation score. Although generating a subset of random cassette sequences and selecting a cassette sequence with a low junction epitope presentation score out of the subset may be sub-optimal relative to the brute force approach, it requires significantly less computational resources thereby making its implementation technically feasible. Further, performing the brute force method as opposed to this more efficient technique may only result in a minor or even negligible improvement in junction epitope presentation score, thus making it not worthwhile from a resource allocation perspective. A cassette design module can determine an improved cassette configuration by formulating the epitope sequence for the cassette as an asymmetric traveling salesman problem (TSP). Given a list of nodes and distances between each pair of nodes, the TSP determines a sequence of nodes associated with the shortest total distance to visit each node exactly once and return to the original node. For example, given cities A, B, and C with known distances between each other, the solution of the TSP generates a closed sequence of cities, for which the total distance traveled to visit each city exactly once is the smallest among possible routes. The asymmetric version of the TSP determines the optimal sequence of nodes when the distance between a pair of nodes are asymmetric. For example, the “distance” for traveling from node A to node B may be different from the “distance” for traveling from node B to node A. By solving for an improved optimal cassette using an asymmetric TSP, the cassette design module can find a cassette sequence that results in a reduced presentation score across the junctions between epitopes of the cassette. The solution of the asymmetric TSP indicates a sequence of therapeutic epitopes that correspond to the order in which the epitopes should be concatenated in a cassette to minimize the junction epitope presentation score across the junctions of the cassette. A cassette sequence determined through this approach can result in a sequence with significantly less presentation of junction epitopes while potentially requiring significantly less computational resources than the random sampling approach, especially when the number of generated candidate cassette sequences is large. Illustrative examples of different computational approaches and comparisons for optimizing cassette design are described in more detail in U.S. Pat. No. 10,055,540, US Application Pub. No. US20200010849A1, and international patent application publications WO/2018/195357 and WO/2018/208856, each herein incorporated by reference, in their entirety, for all purposes.

Shared (neo)antigen sequences for inclusion in a shared antigen vaccine and appropriate patients for treatment with such vaccine can be chosen by one of skill in the art, e.g., as described in U.S. application Ser. No. 17/058,128, herein incorporated by reference for all purposes. Mass spectrometry (MS) validation of candidate shared (neo)antigens can performed as part of the selection process.

A cassette design module can also generate cassette sequences by taking into account additional protein sequences encoded in the vaccine. For example, a cassette design module used to generate a sequence encoding concatenated T cell epitopes can take into account T cell epitopes already encoded by additional protein sequences present in the vaccine (e.g., full-length protein sequences), such as by removing T cell epitopes already encoded by the additional protein sequences from the list of candidate sequences.

A cassette design module can also generate cassette sequences by taking into account the size of the sequences. Without wishing to be bound by theory, in general, increased cassette size can negatively impact vaccine aspects, such as vaccine production and/or vaccine efficacy. In one example, the cassette design module can take into account overlapping sequences, such as overlapping T cell epitope sequences. In general, a single sequence containing overlapping T cell epitope sequences (also referred to as a “frame”) is more efficient than separately linking individual T cell epitope sequences as it reduces the sequence size needed to encode the multiple peptides. Accordingly, in an illustrative example, a cassette design module used to generate a sequence encoding concatenated T cell epitopes can take into account the cost/benefit of extending a candidate T cell epitope to encode one or more additional T cell epitopes, such as determining the benefit gained in additional population coverage for an MHC presenting the additional T cell epitope versus the cost of increasing the size of the sequence.

A cassette design module can also generate cassette sequences by taking into account the magnitude of stimulation of an immune response generated by validated epitopes.

A cassette design module can also generate cassette sequences by taking into account presentation of encoded epitopes across a population, for example that at least one immunogenic epitope is presented by at least one HLA across a proportion of a population, for example by at least 85%, 90%, or 95% of a population (e.g., HLA-A, HLA-B and HLA-C genes over four major ethnic groups, namely European (EUR), African American (AFA), Asian and Pacific Islander (APA) and Hispanic (HIS)). As an illustrative non-limiting example, a cassette design module can also generate cassette sequences such that at least one HLA is present at least across 85%, 90%, or 95% of a population that presents at least one validated epitope or presents at least 4, 5, 6, or 7 predicted epitopes.

A cassette design module can also generate cassette sequences by taking into account other aspects that improve potential safety, such as limiting encoding or the potential to encode a functional protein, functional protein domain, functional protein subunit, or functional protein fragment potentially presenting a safety risk. In some cases, a cassette design module can limit sequence size of encoded peptides such that they are less than 50%, less than 49%, less than 48%, less than 47%, less than 46%, less than 45%, less than 45%, less than 43%, less than 42%, less than 41%, less than 40%, less than 39%, less than 38%, less than 37%, less than 36%, less than 35%, less than 34%, or less than 33% of the translated, corresponding full-length protein. In some cases, a cassette design module can limit sequence size of encoded peptides such that a single contiguous sequence is less than 50% of the translated, corresponding full-length protein, but more than one sequence may be derived from the same translated, corresponding full-length protein and together encode more than 50%. In an illustrative example, if a single sequence containing overlapping T cell epitope sequences (“frame”) is larger than 50% of the translated, corresponding full-length protein, the frame can be split into multiple frames (e.g., f1, f2 etc.) such that each frame is less than 50% of the translated, corresponding full-length protein. A cassette design module can also limit sequence size of encoded peptides such that a single contiguous sequence is less than 49%, less than 48%, less than 47%, less than 46%, less than 45%, less than 45%, less than 43%, less than 42%, less than 41%, less than 40%, less than 39%, less than 38%, less than 37%, less than 36%, less than 35%, less than 34%, or less than 33% of the translated, corresponding full-length protein. Where multiple frames from the same gene are encoded, the multiple frames can have overlapping sequences with each other, in other words each separately encode the same sequence. Where multiple frames from the same gene are encoded, the two or more nucleic acid sequences derived from the same gene can be ordered such that a first nucleic acid sequence cannot be immediately followed by or linked to a second nucleic acid sequence if the second nucleic acid sequence follows, immediately or not, the first nucleic acid sequence in the corresponding gene. For example, if there are 3 frames within the same gene (f1,f2,f3 in increasing order of amino acid position):
The following cassette orderings are not allowed:
   f1 immediately followed by f2
   f2 immediately followed by f3
   f1 immediately followed by f3
The following cassette orderings are allowed:
   f3 immediately followed by f2
   f2 immediately followed by f1

### XIII. Example Computer

A computer can be used for any of the computational methods described herein. One skilled in the art will recognize a computer can have different architectures. Examples of computers are known to those skilled in the art, for example the computers described in more detail in U.S. Pat. No. 10,055,540, US Application Pub. No. US20200010849A1, and international patent application publications WO/2018/195357 and WO/2018/208856, each herein incorporated by reference, in their entirety, for all purposes.

### XIV. Examples

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

xiv.A. Spike Protein Sequence Optimization

Various sequence-optimized nucleotide sequences encoding the Spike protein were evaluated in ChAdV68 vaccine vectors.

### Sequence-Optimization of Spike Sequence

The Spike nucleotide sequence from Wuhan Hu/1 (SEQ ID NO:78) was sequence-optimized by substituting synonymous codons such that the amino acid sequence was unaffected. An IDT algorithm was used for enhanced expression in humans and for reduced complexity to aid synthesis (see, e.g., SEQ ID NOs:66-74). The Spike sequence was additionally sequence-optimized using two additional algorithms; (1) a single sequence (SEQ ID NO:87) generated using SGI DNA (La Jolla, CA); (2) 6 sequences designated CT1, CT20, CT56, CT83, CT131, and CT 199 (SEQ ID NOs:79-84) generated using COOL from the University of Singapore (COOL algorithm generates multiple sequences and 6 were selected). The sequences of each are presented in Table 1.

Splicing events were identified in cDNA from 293A cells infected with ChAdV68 viruses or transfected with ChAdV68 genomic DNA. Specifically, total RNA from 10e5-10e6 cells was purified using Qiagen's RNeasy columns. Residual DNA was removed by DNAse treatment, and cDNA was produced using SuperScriptIV reverse transcriptase (Thermo). Subsequently, primers specific for the 5′ UTR and 3′ UTR of the Gritstone ChAdV68 cassette were used to generate PCR products, analyzed by agarose gel electrophoresis, gel-purified, and Sanger-sequenced to identify regions deleted by splicing.

Splice donor sites were removed by site-directed mutagenesis disrupting the nucleotide sequence motif while not disturbing the amino acid sequence. Mutagenesis was accomplished by incorporating above mutations into PCR primers, amplifying several fragments in parallel, and running a Gibson assembly on the fragments (overlapping by 30-60 nt). Optimized clone CT1-2C (SEQ ID NO:85) had Sanger sequence-identified splice donor motifs at NT385 and NT539 mutated, and clone IDT-4C (SEQ ID NO:86) had Sanger sequence-identified splice donor motifs at NT385, NT539, and predicted donor motifs at NT2003, and NT2473 mutated. Additionally, a possible polyadenylation site AATAAA at nt 445 was mutated to AAcAAA in IDT-4C clone.

The sequences described are presented in Table 1.

TABLE 1
Sequence-optimized Spike Sequences

| Spike | | |
|---|---|---|
| Sequence | Nucleic Acid Sequence | SEQ ID NO: |
| Spike Native; | atgtttgtttttcttgttttattgccactagtctctagtcagtgtgttaatcttacaaccagaactcaattaccccctgcatacact | 78 |
| NC_045512.2 | aattctttcacacgtggtgtttattaccctgacaaagttttcagatcctcagttttacattcaactcaggacttgttcttacctttc | |
| Severe acute | ttttccaatgttacttggttccatgctatacatgtctctgggaccaatggtactaagaggtttgataaccctgtcctaccattta | |
| respiratory | atgatggtgtttattttgcttccactgagaagtctaacataataagaggctggatttttggtactactttagattcgaagaccc | |
| syndrome | agtccctacttattgttaataacgctactaatgttgttattaaagtctgtgaatttcaattttgtaatgatccatttttgggtgtttat | |
| coronavirus | taccacaaaaacaacaaaagttggatggaaagtgagttcagagtttattctagtgcgaataattgcacttttgaatatgtctc | |
| 2 isolate | tcagccttttcttatggaccttgaaggaaaacagggtaatttcaaaaatcttagggaatttgtgtttaagaatattgatggttat | |
| Wuhan-Hu-1 | tttaaaatatattctaagcacacgcctattaatttagtgcgtgatctccctcagggtttttcggctttagaaccattggtagattt | |
| | gccaataggtattaacatcactaggtttcaaactttacttgctttacatagaagttatttgactcctggtgattcttcttcaggtt | |
| | ggacagctggtgctgcagcttattatgtgggttatcttcaacctaggacttttctattaaaatataatgaaaatggaaccatta | |
| | cagatgctgtagactgtgcacttgaccctctctcagaaacaaagtgtacgttgaaatccttcactgtagaaaaaggaatct | |
| | atcaaacttctaactttagagtccaaccaacagaatctattgttagatttcctaatattacaaacttgtgcccttttggtgaagt | |
| | ttttaacgccaccagatttgcatctgtttatgcttggaacaggaagagaatcagcaactgtgttgctgattattctgtcctata | |
| | taattccgcatcattttccacttttaagtgttatggagtgtctcctactaaattaaatgatctctgctttactaatgtctatgcaga | |
| | ttcatttgtaattagaggtgatgaagtcagacaaatcgctccagggcaaactggaaagattgctgattataattataaatta | |
| | ccagatgattttacaggctgcgttatagcttggaattctaacaatcttgattctaaggttggtggtaattataattacctgtata | |
| | gattgtttaggaagtctaatctcaaaccttttgagagagatatttcaactgaaatctatcaggccggtagcacaccttgtaat | |
| | ggtgttgaaggttttaattgttactttcctttacaatcatatggtttccaacccactaatggtgttggttaccaaccatacagag | |
| | tagtagtactttcttttgaacttctacatgcaccagcaactgtttgtggacctaaaaagtctactaatttggttaaaaacaaat | |
| | gtgtcaatttcaacttcaatggtttaacaggcacaggtgttcttactgagtctaacaaaaagtttctgcctttccaacaatttg | |
| | gcagagacattgctgacactactgatgctgtccgtgatccacagacacttgagattcttgacattacaccatgttcttttggt | |
| | ggtgtcagtgttataacaccaggaacaaatacttctaaccaggttgctgttctttatcaggatgttaactgcacagaagtcc | |
| | ctgttgctattcatgcagatcaacttactcctacttggcgtgtttattctacaggttctaatgtttttcaaacacgtgcaggctgt | |
| | ttaataggggctgaacatgtcaacaactcatatgagtgtgacatacccattggtgcaggtatatgcgctagttatcagact | |
| | cagactaattctcctcgggggcacgtagtgtagctagtcaatccatcattgcctacactatgtcacttggtgcagaaaatt | |
| | cagttgcttactctaataactctattgccatacccacaaattttactattagtgttaccacagaaattctaccagtgtctatgac | |
| | caagacatcagtagattgtacaatgtacatttgtggtgattcaactgaatgcagcaatcttttgttgcaatatggcagtttttgt | |
| | acacaattaaaccgtgctttaactggaatagctgttgaacaagacaaaaacacccaagaagtttttgcacaagtcaaaca | |
| | aatttacaaaacaccaccaattaaagattttggtggttttaatttttcacaaatattaccagatccatcaaaaccaagcaaga | |
| | ggtcatttattgaagatctacttttcaacaaagtgacacttgcagatgctggcttcatcaaacaatatggtgattgccttggt | |
| | gatattgctgctagagacctcatttgtgcacaaaagtttaacggccttactgttttgccacctttgctcacagatgaaatgatt | |
| | gctcaatacacttctgcactgttagcgggtacaatcacttctggttggacctttggtgcaggtgctgcattacaaataccatt | |
| | tgctatgcaaatggcttataggtttaatggtattggagttacacagaatgttctctatgagaaccaaaaattgattgccaacc | |
| | aatttaatagtgctattggcaaaattcaagactcactttcttccacagcaagtgcacttggaaaacttcaagatgtggtcaa | |
| | ccaaaatgcacaagctttaaacacgcttgttaaacaacttagctccaattttggtgcaatttcaagtgttttaaatgatatcctt | |
| | tcacgtcttgacaaagttgaggctgaagtgcaaattgataggttgatcacaggcagacttcaaagtttgcagacatatgtg | |
| | actcaacaattaattagagctgcagaaatcagagcttctgctaatcttgctgctactaaaatgtcagagtgtgtacttggac | |
| | aatcaaaaagagttgatttttgtggaaagggctatcatcttatgtccttccctcagtcagcacctcatggtgtagtcttcttgc | |
| | atgtgacttatgtccctgcacaagaaaagaacttcacaactgctcctgccatttgtcatgatggaaaagcacactttcctcg | |
| | tgaaggtgtctttgtttcaaatggcacacactggtttgtaacacaaaggaatttttatgaaccacaaatcattactacagaca | |
| | acacatttgtgtctggtaactgtgatgttgtaataggaattgtcaacaacacagtttatgatcctttgcaacctgaattagact | |
| | cattcaaggaggagttagataaatattttaagaatcatacatcaccagatgttgatttaggtgacatctctggcattaatgctt | |
| | cagttgtaaacattcaaaaagaaattgaccgcctcaatgaggttgccaagaatttaaatgaatctctcatcgatctccaag | |
| | aacttggaaagtatgagcagtatataaaatggccatggtacatttggctaggttttatagctggcttgattgccatagtaatg | |
| | gtgacaattatgctttgctgtatgaccagttgctgtagttgtctcaagggctgttgttcttgtggatcctgctgcaaatttgatg | |
| | aagacgactctgagccagtgctcaaaggagtcaaattacattacacataa | |
| Spike CT1 | ATGTTTGTCTTCCTGGTCTTGCTGCCGCTGGTGAGCAGCCAGTGCGTGA | 79 |
| Optimized | ATCTCACCACCCGCACCCAGCTTCCACCTGCCTACACTAACAGCTTCAC | |
| | CCGAGGGGTGTATTACCCTGACAAGGTATTCCGGTCCTCCGTCCTCCAT | |
| | AGCACGCAGGACCTTTTTCTGCCCTTCTTCTCAAATGTGACATGGTTCCA | |
| | TGCCATTCACGTGAGCGGCACGAATGGAACGAAGCGCTTTGATAACCC | |
| | CGTGCTGCCTTTCAATGACGGCGTCTACTTCGCCTCCACTGAAAAGTCA | |
| | AACATCATCCGGGGCTGGATCTTTGGCACCACTCTTGATTCAAAGACCC | |
| | AGTCACTGCTGATTGTGAACAATGCTACAAACGTGGTTATCAAGGTGTG | |
| | TGAGTTTCAGTTCTGTAACGATCCATTTTTGGGAGTGTACTACCACAAG | |
| | AACAACAAGTCCTGGATGGAGTCTGAGTTCAGAGTGTATAGCTCTGCTA | |
| | ACAACTGCACCTTCGAGTACGTGTCCCAGCCTTTCCTTATGGACCTGGA | |
| | AGGCAAACAGGGCAATTTCAAAAACCTGAGAGAGTTCGTGTTTAAGAA | |
| | CATTGACGGATACTTCAAAATTTATTCTAAGCACACACCAATTAACTTA | |
| | GTGCGGGACCTACCCCAAGGCTTTAGCGCCCTAGAGCCCCTGGTTGACC | |
| | TGCCCATTGGGATCAATATAACAAGGTTCCAAACTCTACTGGCTCTGCA | |
| | TAGAAGTTATCTGACCCCAGGAGACAGCTCTAGTGGTTGGACCGCCGGC | |
| | GCAGCAGCCTACTATGTCGGGTACTTACAGCCACGCACGTTCCTTCTGA | |
| | AGTACAATGAGAACGGGACAATCACTGACGCAGTAGACTGTGCACTGG | |
| | ACCCGCTAAGCGAGACTAAGTGCACACTTAAATCCTTCACGGTGGAGA | |
| | AAGGCATTTATCAGACCTCTAACTTCAGGGTGCAGCCAACAGAAAGCA | |
| | TTGTGCGATTCCCAAATATTACTAATCTTTGCCCTTTCGGGGAGGTCTTT | |
| | AATGCAACTAGATTCGCATCAGTCTATGCGTGGAACCGCAAACGCATTT | |
| | CCAATTGTGTCGCAGACTACTCAGTGCTGTACAACTCTGCCTCTTTCAGT | |
| | ACGTTCAAGTGTTACGGAGTGTCACCCACTAAACTGAACGACCTGTGCT | |
| | TTACAAATGTCTACGCTGACTCCTTCGTGATTAGGGGAGACGAGGTGAG | |
| | ACAAATTGCCCCCGGACAGACTGGGAAGATTGCCGACTACAATTATAA | |
| | GCTTCCTGATGATTTCACTGGCTGTGTTATTGCCTGGAATAGTAACAATC | |
| | TGGATAGCAAGGTGGGAGGCAACTATAACTACTTATATCGACTGTTTAG | |
| | GAAGAGTAATCTGAAACCATTTGAGCGGGATATTTCCACAGAAATTTAC | |
| | CAGGCCGGGAGCACACCATGTAATGGGGTGGAGGGATTTAATTGTTAC | |
| | TTCCCACTCCAGAGCTATGGTTTCCAACCCACCAATGGAGTGGGTTACC | |
| | AGCCCTATAGAGTCGTGGTGCTTAGTTTTGAGCTGCTTCACGCCCCAGC | |
| | AACCGTCTGCGGTCCCAAAAAGTCGACCAATCTCGTGAAAAACAAATG | |
| | CGTAAACTTCAACTTTAACGGCTTAACAGGAACCGGCGTGCTCACCGAA | |
| | AGCAACAAGAAATTCCTTCCATTTCAGCAATTCGGAAGGGACATCGCCG | |
| | ACACAACAGACGCGGTGAGGGACCCACAGACTCTGGAGATACTGGACA | |
| | TCACTCCTTGTTCGTTTGGGGGCGTCTCGGTCATCACACCCGGGACTAA | |
| | TACTAGTAATCAGGTAGCAGTTTTATATCAAGGCGTCAACTGTACCGAA | |
| | GTACCTGTGGCCATACACGCTGATCAGCTAACGCCAACATGGCGAGTCT | |
| | ATTCCACCGGCTCTAACGTTTTTCAGACCAGGGCTGGGTGCCTGATAGG | |
| | GGCAGAGCACGTCAATAATTCCTATGAGTGTGATATCCCCATAGGTGCG | |
| | GGGATCTGTGCCAGCTATCAAACCCAAACCAATTCACCAAGGCGAGCA | |
| | CGGTCTGTGGCTTCTCAGAGCATAATTGCATATACAATGTCACTGGGCG | |
| | CTGAGAATAGCGTTGCATACTCTAATAACAGCATAGCCATTCCCACGAA | |
| | CTTTACTATCAGTGTGACAACCGAAATATTGCCAGTTTCGATGACCAAA | |
| | ACTAGCGTGGATTGCACGATGTACATCTGTGGAGACTCTACCGAATGCA | |
| | GCAATCTGCTATTACAATATGGCAGCTTCTGTACACAGTTAAATCGAGC | |
| | CTTGACAGGCATCGCAGTGGAACAGGACAAAAATACTCAAGAGGTGTT | |
| | TGCACAGGTGAAGCAAATCTACAAAACGCCCCCCATTAAAGATTTTGGC | |
| | GGGTTCAATTTTTCACAAATTCTCCCCGACCCGTCTAAGCCGAGTAAGC | |
| | GGTCCTTCATCGAAGATCTGCTCTTTAACAAAGTAACCCTCGCCGATGC | |
| | CGGCTTTATTAAGCAGTATGGCGACTGCCTGGGGGATATAGCCGCTCGT | |
| | GACCTGATTTGCGCCCAGAAGTTCAATGGTCTGACCGTGTTGCCTCCTTT | |
| | ATTGACCGATGAAATGATTGCCCAGTACACTAGTGCCCTGCTGGCCGGC | |
| | ACTATCACGTCTGGGTGGACCTTCGGAGCTGGTGCCGCCTTGCAGATAC | |
| | CTTTTGCAATGCAGATGGCCTATAGGTTTAATGGTATCGGAGTGACTCA | |
| | GAACGTACTGTACGAGAACCAGAAGCTCATCGCTAATCAATTTAACTCC | |
| | GCTATCGGAAAAATCCAGGACAGCCTCTCTTCTACAGCTAGCGCTCTGG | |
| | GCAAACTGCAGGATGTCGTTAATCAGAATGCCCAGGCCCTGAACACCTT | |
| | GGTTAAACAACTATCTTCCAACTTCGGGGCCATATCCAGTGTGTTGAAT | |
| | GATATTCTCTCCCGCTTGGATAAGGTGGAAGCTGAGGTGCAGATCGATC | |
| | GCTTGATCACCGGCAGACTGCAGTCCCTCCAGACATATGTAACTCAGCA | |
| | GCTGATTAGAGCCGCCGAGATAAGGGCAAGTGCGAATCTGGCTGCCAC | |
| | CAAGATGAGCGAATGTGTATTGGGCCAGAGCAAACGAGTTGATTTTTGC | |
| | GGTAAGGGGTATCATTTAATGTCTTTCCCTCAATCCGCACCTCATGGCG | |
| | TAGTTTTCCTGCATGTGACTTATGTCCCGGCTCAGGAGAAGAATTTTAC | |
| | CACAGCCCCCGCGATCTGCCATGACGGAAAGGCCCACTTCCCCCGGGA | |
| | AGGCGTGTTTGTATCCAATGGGACTCACTGGTTTGTCACTCAGCGAAAT | |
| | TTTTATGAACCACAGATCATCACCACTGACAACACATTTGTTAGTGGAA | |
| | ACTGCGATGTGGTCATCGGCATCGTGAATAACACTGTCTATGATCCACT | |
| | GCAACCTGAACTGGATTCTTTTAAAGAGGAACTCGACAAGTATTTTAAA | |
| | AACCACACTAGCCCTGACGTGGATCTCGGTGACATTTCTGGCATCAACG | |
| | CTAGCGTAGTGAACATTCAGAAAGAGATAGATAGACTTAATGAGGTGG | |
| | CCAAGAACCTCAACGAAAGTCTGATCGACCTCCAGGAACTGGGGAAAT | |
| | ACGAGCAGTACATTAAATGGCCTTGGTACATATGGCTGGGGTTCATTGC | |
| | TGGGCTGATCGCAATAGTGATGGTGACCATAATGCTCTGTTGCATGACT | |
| | AGCTGCTGCAGCTGCCTGAAGGGCTGCTGTAGTTGTGGGTCATGTTGTA | |
| | AGTTTGACGAAGATGATAGCGAGCCTGTCCTTAAAGGAGTGAAGCTCC | |
| | ACTACACCTAG | |
| Spike CT20 | Not shown | 80 |
| Optimized | | |
| Spike CT56 | Not shown | 81 |
| Optimized | | |
| Spike CT83 | Not shown | 82 |
| Optimized | | |
| Spike | Not shown | 83 |
| CT131 | | |
| Optimized | | |
| Spike | No shown | 84 |
| CT199 | | |
| Optimized | | |
| Spike CT1- | ATGTTTGTCTTCCTGGTCTTGCTGCCGCTcGTGtctAGCCAGTGCGTGAATC | 85 |
| 2C | TCACCACCCGCACCCAGCTTCCACCTGCCTACACTAACAGCTTCACCCG | |
| | AGGGGTGTATTACCCTGACAAGGTATTCCGGTCCTCCGTCCTCCATAGC | |
| | ACGCAGGACCTTTTTCTGCCCTTCTTCTCAAATGTGACATGGTTCCATGC | |
| | CATTCACGTGAGCGGCACGAATGGAACGAAGCGCTTTGATAACCCCGT | |
| | GCTGCCTTTCAATGACGGCGTCTACTTCGCCTCCACTGAAAAGTCAAAC | |
| | ATCATCCGGGGCTGGATCTTTGGCACCACTCTTGATTCAAAGACCCAGT | |
| | CACTGCTGATTGTGAACAATGCTACAAACGTGGTTATCAAaGTcTGcGAG | |
| | TTTCAGTTCTGTAACGATCCATTTTTGGGAGTGTACTACCACAAGAACA | |
| | ACAAGTCCTGGATGGAGTCTGAGTTCAGAGTGTATAGCTCTGCTAACAA | |
| | CTGCACCTTCGAGTACGTGTCCCAGCCTTTCCTTATGGACCTGGAAGGC | |
| | AAACAGGGCAATTTCAAAAACCTGAGAGAGTTCGTGTTTAAGAACATT | |
| | GACGGATACTTCAAAATTTATTCTAAGCACACACCAATTAACTTAGTGC | |
| | GGGACCTACCCCAAGGCTTTAGCGCCCTAGAGCCCCTGGTTGACCTGCC | |
| | CATTGGGATCAATATAACAAGGTTCCAAACTCTACTGGCTCTGCATAGA | |
| | AGTTATCTGACCCCAGGAGACAGCTCTAGTGGTTGGACCGCCGGCGCA | |
| | GCAGCCTACTATGTCGGGTACTTACAGCCACGCACGTTCCTTCTGAAGT | |
| | ACAATGAGAACGGGACAATCACTGACGCAGTAGACTGTGCACTGGACC | |
| | CGCTAAGCGAGACTAAGTGCACACTTAAATCCTTCACGGTGGAGAAAG | |
| | GCATTTATCAGACCTCTAACTTCAGGGTGCAGCCAACAGAAAGCATTGT | |
| | GCGATTCCCAAATATTACTAATCTTTGCCCTTTCGGGGAGGTCTTTAATG | |
| | CAACTAGATTCGCATCAGTCTATGCGTGGAACCGCAAACGCATTTCCAA | |
| | TTGTGTCGCAGACTACTCAGTGCTGTACAACTCTGCCTCTTTCAGTACGT | |
| | TCAAGTGTTACGGAGTGTCACCCACTAAACTGAACGACCTGTGCTTTAC | |
| | AAATGTCTACGCTGACTCCTTCGTGATTAGGGGAGACGAGGTGAGACA | |
| | AATTGCCCCCGGACAGACTGGGAAGATTGCCGACTACAATTATAAGCTT | |
| | CCTGATGATTTCACTGGCTGTGTTATTGCCTGGAATAGTAACAATCTGG | |
| | ATAGCAAGGTGGGAGGCAACTATAACTACTTATATCGACTGTTTAGGAA | |
| | GAGTAATCTGAAACCATTTGAGCGGGATATTTCCACAGAAATTTACCAG | |
| | GCCGGGAGCACACCATGTAATGGGGTGGAGGGATTTAATTGTTACTTCC | |
| | CACTCCAGAGCTATGGTTTCCAACCCACCAATGGAGTGGGTTACCAGCC | |
| | CTATAGAGTCGTGGTGCTTAGTTTTGAGCTGCTTCACGCCCCAGCAACC | |
| | GTCTGCGGTCCCAAAAAGTCGACCAATCTCGTGAAAAACAAATGCGTA | |
| | AACTTCAACTTTAACGGCTTAACAGGAACCGGCGTGCTCACCGAAAGC | |
| | AACAAGAAATTCCTTCCATTTCAGCAATTCGGAAGGGACATCGCCGACA | |
| | CAACAGACGCcGTcAGGGACCCACAGACTCTGGAGATACTGGACATCAC | |
| | TCCTTGTTCGTTTGGGGGCGTCTCGGTCATCACACCCGGGACTAATACT | |
| | AGTAATCAGGTAGCAGTTTTATATCAAGGCGTCAACTGTACCGAAGTAC | |
| | CTGTGGCCATACACGCTGATCAGCTAACGCCAACATGGCGAGTCTATTC | |
| | CACCGGCTCTAACGTTTTTCAGACCAGGGCTGGGTGCCTGATAGGGGCA | |
| | GAGCACGTCAATAATTCCTATGAGTGTGATATCCCCATAGGTGCGGGGA | |
| | TCTGTGCCAGCTATCAAACCCAAACCAATTCACCAAGGCGAGCACGGTC | |
| | TGTGGCTTCTCAGAGCATAATTGCATATACAATGTCACTGGGCGCTGAG | |
| | AATAGCGTTGCATACTCTAATAACAGCATAGCCATTCCCACGAACTTTA | |
| | CTATCAGTGTGACAACCGAAATATTGCCAGTTTCGATGACCAAAACTAG | |
| | CGTGGATTGCACGATGTACATCTGTGGAGACTCTACCGAATGCAGCAAT | |
| | CTGCTATTACAATATGGCAGCTTCTGTACACAGTTAAATCGAGCCTTGA | |
| | CAGGCATCGCAGTGGAACAGGACAAAAATACTCAAGAGGTGTTTGCAC | |
| | AGGTGAAGCAAATCTACAAAACGCCCCCCATTAAAGATTTTGGCGGGTT | |
| | CAATTTTTCACAAATTCTCCCCGACCCGTCTAAGCCGAGTAAGCGGTCC | |
| | TTCATCGAAGATCTGCTCTTTAACAAAGTAACCCTCGCCGATGCCGGCT | |
| | TTATTAAGCAGTATGGCGACTGCCTGGGGGATATAGCCGCTCGTGACCT | |
| | GATTTGCGCCCAGAAGTTCAATGGTCTGACCGTGTTGCCTCCTTTATTGA | |
| | CCGATGAAATGATTGCCCAGTACACTAGTGCCCTGCTGGCCGGCACTAT | |
| | CACGTCTGGGTGGACCTTCGGAGCTGGTGCCGCCTTGCAGATACCTTTT | |
| | GCAATGCAGATGGCCTATAGGTTTAATGGTATCGGAGTGACTCAGAAC | |
| | GTACTGTACGAGAACCAGAAGCTCATCGCTAATCAATTTAACTCCGCTA | |
| | TCGGAAAAATCCAGGACAGCCTCTCTTCTACAGCTAGCGCTCTGGGCAA | |
| | ACTGCAGGATGTCGTTAATCAGAATGCCCAGGCCCTGAACACCTTGGTT | |
| | AAACAACTATCTTCCAACTTCGGGGCCATATCCAGTGTGTTGAATGATA | |
| | TTCTCTCCCGCTTGGATAAGGTGGAAGCTGAGGTGCAGATCGATCGCTT | |
| | GATCACCGGCAGACTGCAGTCCCTCCAGACATATGTAACTCAGCAGCTG | |
| | ATTAGAGCCGCCGAGATAAGGGCAAGTGCGAATCTGGCTGCCACCAAG | |
| | ATGAGCGAATGTGTATTGGGCCAGAGCAAACGAGTTGATTTTTGCGGTA | |
| | AGGGGTATCATTTAATGTCTTTCCCTCAATCCGCACCTCATGGCGTAGTT | |
| | TTCCTGCATGTGACTTATGTCCCGGCTCAGGAGAAGAATTTTACCACAG | |
| | CCCCCGCGATCTGCCATGACGGAAAGGCCCACTTCCCCCGGGAAGGCG | |
| | TGTTTGTATCCAATGGGACTCACTGGTTTGTCACTCAGCGAAATTTTTAT | |
| | GAACCACAGATCATCACCACTGACAACACATTTGTTAGTGGAAACTGCG | |
| | ATGTGGTCATCGGCATCGTGAATAACACTGTCTATGATCCACTGCAACC | |
| | TGAACTGGATTCTTTTAAAGAGGAACTCGACAAGTATTTTAAAAACCAC | |
| | ACTAGCCCTGACGTGGATCTCGGTGACATTTCTGGCATCAACGCTAGCG | |
| | TAGTGAACATTCAGAAAGAGATAGATAGACTTAATGAGGTGGCCAAGA | |
| | ACCTCAACGAAAGTCTGATCGACCTCCAGGAACTGGGGAAATACGAGC | |
| | AGTACATTAAATGGCCTTGGTACATATGGCTGGGGTTCATTGCTGGGCT | |
| | GATCGCAATAGTGATGGTGACCATAATGCTCTGTTGCATGACTAGCTGC | |
| | TGCAGCTGCCTGAAGGGCTGCTGTAGTTGTGGGTCATGTTGTAAGTTTG | |
| | ACGAAGATGATAGCGAGCCTGTCCTTAAAGGAGTGAAGCTCCACTACA | |
| | CCTAG | |
| Spike IDT- | ATGTTTGTCTTTCTGGTCCTGCTTCCCCTCGTTAGTTCTCAGTGTGTGAA | 86 |
| 4C | CCTCACCACACGGACGCAACTCCCTCCAGCTTACACAAATTCTTTTACG | |
| | CGAGGCGTGTATTATCCGGATAAAGTTTTCAGGTCCTCCGTCCTGCACT | |
| | CCACGCAGGACCTTTTTTTGCCGTTCTTTTCTAACGTAACATGGTTTCAT | |
| | GCCATTCATGTTTCCGGGACAAACGGTACGAAACGCTTTGATAACCCTG | |
| | TGCTGCCGTTCAATGATGGCGTTTACTTTGCCTCTACGGAAAAGAGTAA | |
| | CATAATCCGAGGCTGGATCTTCGGGACCACCCTGGATAGCAAGACTCA | |
| | GAGTCTTCTCATCGTTAATAACGCTACAAACGTTGTTATCAAaGTcTGcG | |
| | AATTCCAGTTTTGCAACGATCCCTTTTTGGGGGTATACTATCACAAAAA | |
| | CAAcAAAAGTTGGATGGAATCAGAGTTTCGCGTGTATTCTTCTGCGAAC | |
| | AACTGCACGTTTGAATACGTTAGTCAGCCTTTTCTCATGGACTTGGAgGG | |
| | cAAaCAGGGGAACTTTAAAAACTTGCGGGAGTTCGTTTTTAAGAACATA | |
| | GATGGTTACTTCAAAATTTATAGCAAACATACACCGATCAACCTCGTGA | |
| | GAGATCTCCCACAGGGTTTTTCCGCACTCGAACCGCTCGTGGATCTGCC | |
| | GATTGGAATTAACATTACCCGCTTCCAGACCCTGTTGGCTCTGCACAGA | |
| | AGCTATCTGACGCCAGGGGATTCCAGCAGTGGATGGACGGCGGGTGCG | |
| | GCCGCGTATTATGTAGGCTATCTCCAACCCCGAACGTTCTTGCTGAAGT | |
| | ACAATGAGAATGGGACCATTACGGATGCTGTGGATTGTGCATTGGATCC | |
| | TCTCTCTGAGACAAAATGCACCCTTAAAAGCTTCACTGTAGAAAAGGGT | |
| | ATTTATCAGACTAGCAACTTCCGCGTACAACCAACGGAGTCTATCGTTA | |
| | GGTTCCCCAACATTACTAACTTGTGCCCATTTGGCGAAGTGTTTAACGC | |
| | AACTAGGTTTGCTAGTGTCTATGCTTGGAATCGAAAGAGAATAAGCAAT | |
| | TGTGTCGCAGATTACTCCGTATTGTATAATTCTGCAAGCTTTTCAACATT | |
| | CAAGTGCTACGGAGTGTCTCCCACCAAATTGAACGACCTGTGTTTTACT | |
| | AACGTGTATGCCGACTCTTTCGTTATCCGAGGCGATGAGGTCAGGCAAA | |
| | TTGCCCCCGGACAAACTGGGAAAATTGCGGATTATAATTACAAGCTTCC | |
| | AGACGACTTTACGGGCTGTGTTATCGCATGGAACTCCAACAATCTCGAC | |
| | AGCAAAGTGGGTGGAAATTATAATTATTTGTATAGATTGTTTCGCAAGT | |
| | CCAACCTGAAGCCATTCGAGAGAGACATCTCCACCGAGATTTATCAAGC | |
| | CGGCTCAACTCCTTGCAACGGAGTCGAAGGCTTCAACTGTTATTTTCCG | |
| | CTTCAGTCCTATGGTTTTCAACCTACGAACGGCGTGGGATACCAGCCGT | |
| | ATCGAGTCGTGGTACTGTCCTTTGAACTTTTGCACGCCCCAGCAACTGTT | |
| | TGCGGACCAAAAAAGTCCACAAATCTCGTCAAAAACAAGTGCGTTAAT | |
| | TTTAATTTTAACGGGCTTACGGGTACTGGTGTACTGACGGAGTCCAATA | |
| | AGAAATTCCTGCCATTCCAACAGTTTGGACGGGATATTGCTGATACGAC | |
| | CGACGCTGTGCGAGATCCCCAGACACTTGAGATCCTCGACATAACCCCC | |
| | TGTAGTTTTGGTGGAGTGTCTGTAATTACCCCCGGAACGAACACCAGCA | |
| | ACCAAGTTGCGGTGCTTTATCAGGGTGTTAATTGCACTGAGGTTCCTGT | |
| | CGCGATACATGCCGACCAACTGACGCCTACATGGCGAGTATATTCAACG | |
| | GGCTCCAACGTCTTCCAGACGCGAGCCGGTTGTCTCATTGGAGCTGAAC | |
| | ATGTGAACAACTCTTATGAATGCGATATACCAATTGGGGCtGGaATCTGC | |
| | GCCTCTTATCAGACGCAGACTAACTCACCCAGACGAGCACGGAGCGTG | |
| | GCAAGCCAATCCATTATTGCCTACACCATGTCCTTGGGAGCTGAGAATT | |
| | CAGTCGCCTATTCTAATAATAGCATTGCTATACCGACGAACTTTACAAT | |
| | TTCCGTAACTACCGAAATATTGCCCGTCAGTATGACTAAGACTAGCGTT | |
| | GACTGCACAATGTACATTTGCGGCGATAGTACCGAATGCTCTAATCTTC | |
| | TTCTGCAGTATGGTAGTTTTTGTACACAACTGAATCGAGCTCTGACTGG | |
| | GATCGCAGTCGAACAGGACAAAAATACACAGGAAGTTTTCGCGCAAGT | |
| | GAAGCAAATCTACAAAACGCCTCCCATAAAAGATTTCGGAGGATTCAA | |
| | TTTCAGTCAGATACTCCCTGATCCCTCTAAACCATCTAAACGATCCTTTA | |
| | TCGAAGATTTGCTGTTCAACAAaGTcACCCTTGCTGACGCTGGATTCATA | |
| | AAGCAGTACGGGGATTGTCTTGGCGATATCGCAGCCCGAGACCTTATTT | |
| | GTGCCCAAAAATTTAACGGACTTACGGTACTCCCTCCCCTTCTGACTGA | |
| | CGAAATGATAGCCCAGTACACCAGTGCTCTGCTGGCTGGCACCATAACG | |
| | AGCGGATGGACTTTTGGTGCGGGTGCAGCACTGCAGATCCCCTTCGCGA | |
| | TGCAAATGGCATACAGGTTTAATGGGATTGGGGTCACCCAGAATGTATT | |
| | GTACGAGAACCAGAAGCTTATAGCGAATCAATTTAACAGTGCAATTGG | |
| | TAAGATTCAGGACAGCCTTTCAAGTACCGCGAGTGCTCTCGGGAAGTTG | |
| | CAGGATGTAGTAAATCAAAATGCGCAGGCGCTGAATACGTTGGTTAAA | |
| | CAGCTCAGCAGTAATTTTGGAGCAATTTCTAGCGTGCTGAATGACATCC | |
| | TCAGCAGACTCGATAAGGTGGAGGCTGAGGTACAGATAGATAGACTCA | |
| | TCACGGGCAGATTGCAGAGTTTGCAGACATACGTCACGCAACAACTCAT | |
| | TCGAGCAGCAGAAATTAGAGCATCCGCAAATCTGGCGGCCACGAAAAT | |
| | GTCTGAGTGCGTTCTGGGGCAGTCCAAGAGAGTTGACTTTTGTGGGAAA | |
| | GGATATCATCTGATGAGTTTTCCGCAGTCAGCGCCACATGGTGTGGTCT | |
| | TTCTGCACGTTACTTATGTCCCCGCACAGGAGAAGAATTTTACGACCGC | |
| | GCCAGCTATTTGCCATGACGGTAAGGCTCACTTCCCGAGGGAAGGGGT | |
| | ATTCGTTTCTAACGGTACGCATTGGTTTGTTACGCAACGGAACTTTTATG | |
| | AACCACAGATTATTACCACCGACAACACATTCGTAAGTGGAAACTGTG | |
| | ATGTCGTTATCGGAATAGTAAATAATACCGTTTATGACCCCCTTCAGCC | |
| | TGAACTTGATTCCTTCAAGGAAGAGCTCGATAAATACTTTAAGAACCAC | |
| | ACCAGCCCCGATGTTGACCTGGGTGATATATCTGGGATCAACGCTTCTG | |
| | TCGTCAACATTCAAAAAGAGATCGATCGCCTGAATGAGGTGGCCAAAA | |
| | ACCTCAATGAAAGTCTCATTGACCTTCAAGAACTCGGTAAGTACGAGCA | |
| | ATACATCAAGTGGCCATGGTACATATGGCTGGGCTTCATTGCTGGGTTG | |
| | ATAGCTATAGTGATGGTTACGATAATGTTGTGTTGTATGACATCCTGCT | |
| | GTAGCTGCCTTAAAGGTTGTTGTTCTTGCGGTTCTTGTTGTAAGTTTGAC | |
| | GAAGATGATTCAGAGCCTGTTCTGAAAGGGGTGAAGCTCCATTATACTT | |
| | GA | |
| Spike-SGI | Not shown | 87 |

### Cloning of Sequence-Optimized Spike Sequences

Each sequence-optimized Spike sequence was ordered as a set of 3 gBlocks from IDT with each gblock between 1300-1500 bp and overlapping with each other by approximately 100 nucleotides. The gBlocks comprising the 5′ and 3′ ends of the Spike sequence overlapped with the plasmid backbone by 100 nucleotides. The gblocks were assembled by a combination of PCR and Gibson assembly into a linearized pA68-E4d AsisI/PmeI backbone to generate pA68-E4-sequence-optimized Spike clones. Clones were screened by PCR and clones of the correct size were then grown for plasmid production and sequencing by either NGS or Sanger sequencing. Once a correct clone was sequence confirmed, large scale plasmid production and purification was performed for transfection.

### Vector Production

pA68-E4-Spike plasmid DNA was digested with Pad and 2 ug DNA was transfected into 293F cells using TransIt Lenti transfection reagent. Five days post transfection, cells and media were harvested and a lysate generated by freeze-thawing at −80 C and at 37 C. A fraction of the lysate was used to re-infect 30 mL of 293F cells and incubated for 48-72 h before harvesting. Lysate was generated by freeze-thawing at −80 C and at 37 C and a fraction of the lysate was used to infect 400 mL of 293F cells seeded at 1e6 cells/mL. Next, 48-72 h later cells were harvested, lysed in 10 mM tris pH 8.0/0.1% Triton X-100 and freeze thawed 1× at 37 and −80 C. The lysate was then clarified by centrifugation at 4300×g for 10 mM prior to loading on a 1.2/1.4 CsCl gradient. The gradient was run for a minimum of 2 h before the bands were harvested diluted 2-4× in Tris and then rerun on a 1.35 CsCl gradient for at least 2 h. The viral bands were harvested and then dialyzed 3× in 1×ARM buffer. The virus infectious titer was determined by an immunostaining titer assay and the viral particle measured by Absorbance at A260 nm.

### Western Analysis

Samples for Spike expression analysis were either harvested at designated times post transfection or in the case of purified virus by setting up a controlled infection experiment with a known virus MOI and harvested at a specific time post infection, typically 24 to 48 h. 1e6 cells were typically harvested in 0.5 mL of SDS-PAGE loading buffer with 10% Beta-mercaptoethanol. Samples were boiled and run on 4-20% polyacrylamide gels under denaturing and reducing conditions. The gels were then blotted onto a PVDF membrane using a BioRad Rapid transfer device. The membrane was blocked for 2 h at room temperature in 5% Skim milk in TBST. The membrane was then probed with an anti-Spike 51 polyclonal (Sino Biologicals) or anti-Spike monoclonal antibody 1A9 (GeneTex; Cat. No. GTX632604) and incubated for 2 h. The membrane was then washed in PBST (5×) and the probed with a HRP-linked anti-mouse antibody (Bethyl labs) for 1 h. The membrane was washed as described above and then incubated with a chemiluminescent substrate ECL plus (ThermoFisher). The image was then captured using a Chemidoc (BioRad device).

### Results

Expression of Spike S2 protein was assessed during viral production in 293F cells with various Spike-encoding vectors. Using vectors encoding IDT sequence-optimized Spike cassettes, Spike S2 protein was detected by Western blot using an anti-Spike S2 antibody (GeneTex) when expressed in a SAM vector, but not when expressed in a ChAdV68 vector (“CMV-Spike (IDT)”; SEQ ID NO:69) at two different MOIs and timepoints (data not shown). Two clones engineered to express Spike variant D614G (“CMV-Spike (IDT)-D614G” SEQ ID NO:70) also did not express detectable levels of Spike protein by Western using the S2 antibody (data not shown). Clones engineered to co-express the SARS-CoV-2 Membrane protein together with Spike (“CMV-Spike (IDT)-D614G-Mem” SEQ ID NO:66) or including a R682V mutations to disrupt the Furin cleavage site did not rescue the expression phenotype (data not shown). In contrast, Spike 51 protein was detected for all IDT constructs, albeit at low levels, with the exception of the Furin R682V mutation in which no Spike 51 protein was detected.

To deconvolute if the expressions issues with the IDT sequence-optimized clones were specific to the 51 or S2 domains, vectors expressing only the 51 or S2 domains were also evaluated. A ChAdV68 vector encoding the IDT sequence-optimized Spike 51 protein alone demonstrated strong protein expression, in contrast to the lower expression observed with the full-length Spike vector (data not shown). As expected, no signal was observed for 51 with the vector encoding the S2 domain alone. In contrast, a ChAdV68 vector encoding the IDT sequence-optimized Spike S2 protein alone did not demonstrate observable protein expression, comparable to the absence of signal observed with the full-length Spike vector (data not shown). Thus, the data indicate the IDT sequence-optimized Spike S2 exhibited poor expression, including impacting expression of the full-length Spike sequence.

To address protein expression, the SARS-CoV-2 Spike-encoding nucleotide sequence was sequence-optimized using additional sequence-optimization algorithms; (1) a single sequence (SEQ ID NO:87) generated using SGI DNA (La Jolla, CA); (2) 6 sequences designated CT1, CT20, CT56, CT83, CT131, and CT 199 (SEQ ID NOs:79-84) generated using COOL from the University of Singapore (COOL algorithm generates multiple sequences and 6 were selected). Sequence-optimization with the COOL algorithm generated a sequence—CT1 (SEQ ID NO:79)—that demonstrated detectable expression using a ChAdV68 vector as assessed by Western using both an anti-S2 and anti-S1 antibody (data not shown). The additional sequences generated using the COOL algorithm and the SGI algorithm were also assessed by Western. The SGI clone and COOL sequence CT131 also demonstrated detectable levels of Spike protein by Western using an anti-S2 antibody, while other COOL generated sequences did not generate detectable signals other than the control CT1 derived sequence (lane 2). Thus, the data indicate that specific sequence-optimizations improved expression of full-length SARS-CoV-2 Spike protein in ChAdV68 vectors.

SARS-CoV-2 is a cytoplasm-replicating positive-sense RNA virus encoding its own replication machinery, and as such SARS-CoV-2 mRNA are not naturally processed by splicing and nuclear-export machineries. To assess the role of splicing in SARS-CoV-2 Spike-encoding mRNA expressed from a ChAdV68 vector, primers were designed to amplify the Spike coding region. In the presence of mRNA splicing, amplicon sizes would be smaller than the expected full-length coding region. While PCR of the plasmid encoding the SARS-CoV-2 Spike cassette demonstrated the expected amplicon size (“Spike Plasmid” left panel, right column), PCR amplification of cDNA from infected 293 cells demonstrated two smaller amplicons indicating splicing of the mRNA transcript (“ChAd-Spike (IDT) cDNA” left panel, left column). In addition, the Spike coding sequence was split into S1 and S2 encoding sequences. PCR amplification of S1 cDNA from infected 293 cells demonstrated the expected amplicon size (“SpikeS1” right panel, left column) indicating S1 was likely not undergoing undesired splicing while sequences in the S2 region may be influencing splicing.

The smaller amplicon sequences were analyzed and two splice donor sites were identified by Sanger sequencing. Three additional potential donor sites were predicted by further sequence analysis. The position and identity of the splice motif sequences are presented below (the nt triplets correspond to codons, numbering starts with reference to Spike ATG):

NT 385-:
AAG GTG TGT → AAa GTc TGc (identified by sequencing)
NT 539-:
AA GGT AAG C → Ag GGc AAa C (identified by sequencing)
NT 2003-:
CA GGT ATC T → Ct GGa ATC T (predicted)
NT 2473-:
AAG GTG ACC → AAa GTc ACC (predicted)
NT 3417-:
C CCC CTT CAG CCT GAA CTT GAT TCC (SEQ ID NO: 123) → T CCa CTg
CAa CCT GAA CTT GAT agt (SEQ ID NO: 124)

Selected splice donor sites were removed by site-directed mutagenesis disrupting the nucleotide sequence motif while not disturbing the amino acid sequence. COOL sequence-optimized clone CT1 was used as the reference sequence for clone CT1-2C (SEQ ID NO:85) having the sequence-identified splice donor motifs at NT385 and NT539 mutated. IDT sequence-optimized clone was used as the reference sequence for clone IDT-4C (SEQ ID NO:86) and had both sequence-identified and predicted splice donor motifs at NT385, NT539, NT2003, and NT2473 mutated, as well as a possible polyadenylation site AATAAA at NT445 mutated to AAcAAA. Spike protein expression was detected by Western in the clone including the sequence-identified splice donor motifs. Splicing was further assessed in the constructs by PCR analysis. Mutating the splice donor motifs and/or a potential polyA site alone did not prevent splicing indicating splicing potentially occurred from sub-dominant splice sites.

Given the identification of splicing events in the full-length Spike mRNA expressed from ChAdV68 vectors, additional constructs are generated and assessed for improved protein expression. Additional optimizations include constructs featuring exogenous nuclear export signals (e.g., Constitutive Transport Element (CTE), RNA Transport Element (RTE), or Woodchuck Posttranscriptional Regulatory Element (WPRE)) or the addition of an artificial intron through introduction of exogenous splice donor/branch/acceptor motif sequences to bias splicing, such as introducing a SV40 mini-intron (SEQ ID NO:88) between the CMV promoter and the Kozak sequence immediately upstream of the Spike gene. The identified and predicted splice donor motifs are also further evaluated in combination with additional sequence optimizations.

XIV.B. Multicistronic Self-Amplifying mRNA Vector Evaluation

Evaluation results are presented for self-amplifying mRNA (SAM) SARS-CoV-2 vaccine designs featuring multiple expression cassettes driven by separate subgenomic alphavirus-derived promoters. FIG. 1 illustrates a self-amplifying mRNA (SAM) system featuring a single alphavirus-derived subgenomic promoter (SGP). In such a system, the single SGP is solely responsible for the transcription of the payload cassette, including multicistronic transcripts expressing multiple proteins using 2A ribosome skipping sequence elements (e.g., E2A, P2A, F2A, or T2A sequences) or Internal Ribosome Entry Site (IRES) sequence elements. FIG. 2 illustrates a SAM system featuring multiple expression cassettes driven by separate SGPs. Without wishing to be bound by theory, the multiple SGPs can drive higher expression of the gene under control of the second SGP (SGP2) given both SGP1 and SGP2 will produce transcripts encoding the second gene.

Various SARS-CoV-2 vaccine designs, constructs, and dosing regimens were evaluated. The vaccines encoded various optimized versions of the Spike protein, selected predicted T cell epitopes (TCE), or a combination of Spike and TCE cassettes.

Specifically, various subgenomic alphavirus-derived promoters (SGP) including the core 24-nt conserved promoter sequence ctctctacggcTAAcctgaa(+1)tgga (SEQ ID NO: 27989) that functions as promoter for the transcription of the 26S transcripts were assessed.

In vectors with two cassettes either encoding a SARS-CoV-2 peptide (various Spike variant sequences below in Table 2) or various concatenated T cell epitope cassettes (see representative “TCE5”), the first cassette was driven by a first subgenomic alphavirus-derived promoter SGP1 (GGGCCCCTATAACTCTCTACGGCTAACCTGAATGGACTACGAC (SEQ ID NO: 27981)) and the second cassette was driven by a second subgenomic alphavirus-derived promoter SGP2 (GGGCCCCTATAACTCTCTACGGCTAACCTGAATGGACTACGACATAGTCTAGTCCG CCAAG (SEQ ID NO: 27980)). The SGP2 was flanked by nucleotides encoding the C-terminal 8 amino acids of the nonstructural protein 4 (nsP4) on the 5′ region and flanked 3′ by nucleotides encoding a 18-nt nontranslated region (e.g., atagtctagtccgccaag (SEQ ID NO: 27976)) from the VEEV alphavirus (see Am. J. Trop. Med. Hyg., 59(6), 1998, pp. 952-964, herein incorporated by reference for all purposes). Flanking sequences were included in SGP2 for various purposes, including preventing recombination with SGP1 and incorporating any potential additional transcriptional enhancer elements. In addition, SGP2 is encoded immediately 5′ of the Kozak sequence for the second cassette. A representative sequence including both SGP1 and SGP2 is shown in SEQ ID NO:93.

Specific methods are described in further detail below.

### Mouse Immunizations

All mouse studies were conducted at Murigenics under IACUC approved protocols. Balb/c mice (Envigo), 6-8 weeks old were used for all studies. Vaccines were stored at −80° C., thawed at room temperature on the day of immunization, and then diluted to 0.1 μg/mL with PBS and filtered through a 0.2 micron filter. Filtered formulations were stored at 4° C. and injected within 4 hours of preparation. All immunizations were bilateral intramuscular to the tibialis anterior, 2 injections of 50 μL each, 100 μL total.

### Splenocyte Isolation

For the evaluation of T-cell response, mouse spleens were extracted at various timepoints following immunization. Note that in some studies immunizations were staggered to enable spleens to be collected at the same time and compared. Spleens were collected and analyzed by IFNγ ELISpot and ICS. Spleens were suspended in RPMI complete (RPMI+10% FBS) and dissociated using the gentleMACS Dissociator (Milltenyi Biotec). Dissociated cells were filtered using a 40 μm strainer and red blood cells were lysed with ACK lysing buffer (150 mM NH₄Cl, 10 mM KHCO₃, 0.1 mM EDTA). Following lysis, cells were filtered with a 30 μm strainer and resuspended in RMPI complete.

### Serum Collection

At various timepoints post immunization 200 μL of blood was drawn. Blood was centrifuged at 1000 g for 10 minutes at room temperature. Serum was collected and frozen at −80° C.

### S1 IgG MSD/ELISA

96-well QuickPlex plates (Meso Scale Discovery, Rockville, MD) were coated with 50 μL of 1 μg/mL SARS-CoV-2 S1 (ACROBiosystems, Newark, DE), diluted in DPBS (Corning, Corning, NY), and incubated at 4° C. overnight. Wells were washed three times with agitation using 250 μL of PBS+0.05% Tween-20 (Teknova, Hollister, CA) and plates blocked with 150 μL Superblock PBS (Thermo Fisher Scientific, Waltham, MA) for 1 hour at room temperature on an orbital shaker. Test sera was diluted at appropriate series in 10% species-matched serum (Innovative Research, Novi, MI) and tested in single wells on each plate. Starting dilution 1:100, 3-fold dilutions, 11 dilutions per sample. Wells were washed and 50 uL of the diluted samples were added to wells and incubated for 1 hour at room temperature on an orbital shaker Wells were washed and incubated with 25 μL of 1 μg/mL SULFO-TAG labeled anti-mouse antibody (MSD), diluted in DPBS+1% BSA (Sigma-Aldrich, St. Louis, MO), for 1 hour at room temperature on an orbital shaker. Wells were washed and 150 μL tripropylamine containing read buffer (MSD) added. Plates were run immediately using the QPlex SQ 120 (MSD) ECL plate reader. Endpoint titer is defined as the reciprocal dilution for each sample at which the signal is twice the background value, and is interpolated by fitting a line between the final two values that are greater than twice the background value. The background values is the average value (calculated for each plate) of the control wells containing 10% species-matched serum only.

### IFNγ ELISpot Analysis

IFNγ ELISpot assays were performed using pre-coated 96-well plates (MAbtech, Mouse IFNγ ELISpot PLUS, ALP) following manufacturer's protocol. Samples were stimulated overnight with various overlapping peptide pools (15 amino acids in length, 11 amino acid overlap), at a final concentration of 1 μg/mL per peptide. For Spike—eight different overlapping peptide pools spanning the SARS-CoV-2 Spike antigen (Genscript, 36-40 peptides per pool). Splenocytes were plated in duplicate at 1×10⁵ cells per well for each Spike pool, and 2.5×10⁴ cells per well (mixed with 7.5×10⁴ naïve cells) for Spike pools 2, 4, and 7. To measure response to the TCE cassette—one pool spanning Nucleocapsid protein (JPT, NCap-1, 102 peptides), one spanning Membrane protein (JPT, VME-1, 53 peptides), and one spanning the Orf3a regions encoded in the cassette (Genscript, 38 peptides). For TCE peptide pools, splenocytes were plated in duplicate at 2×10⁵ cells per well for each pool. A DMSO only control was plated for each sample and cell number. Following overnight incubation at 37° C., plates were washed with PBS and incubated with anti-monkey IFNγ mAb biotin (MAbtech) for two hours, followed by an additional wash and incubation with Streptavidin-ALP (MAbtech) for one hour. After final wash, plates were incubated for ten minutes with BCIP/NBT (MAbtech) to develop the immunospots. Spots were imaged and enumerated using an AID reader (Autoimmun Diagnostika). For data processing and analysis, samples with replicate well variability (Variability=Variance/(median+1)) greater than 10 and median greater than 10 were excluded. Spot values were adjusted based on the well saturation according to the formula:

AdjustedSpots=RawSpots+2*(RawSpots*Saturation/(100-Saturation)

Each sample was background corrected by subtracting the average value of the negative control peptide wells. Data is presented as spot forming colonies (SFC) per 1×10⁶ splenocytes. Wells with well saturation values greater than 35% were labeled as “too numerous to count” (TNTC) and excluded. For samples and peptides that were TNTC, the value measured with 2.5×10⁴ cells/well was used.

The various sequences evaluated are as follows:
“IDTSpike_{g}”: SARS-CoV-2 Spike protein encoded by IDT optimized sequence (see SEQ ID NO:69) and including a D614G mutation with reference to SEQ ID NO:59 (see corresponding nucleotide mutation in SEQ ID NO:70); also referred to as “Spike V1”
“CTSpike_{g}”: SARS-CoV-2 Spike protein encoded by Cool Tool optimized sequence version 1 (SEQ ID NO:79) including a D614G mutation with reference to SEQ ID NO:59 (see corresponding nucleotide mutation in SEQ ID NO:70); also referred to as “Spike V2.” In versions referred to as “CTSpike_{D}” D614 is not altered.
“CTSpikeF2P_{g}”: SARS-CoV-2 Spike protein encoded by Cool Tool optimized sequence version 1 (SEQ ID NO:79) including a R682V to disrupt the Furin cleavage site (682-685 RRAR (SEQ ID NO: 125) to GSAS (SEQ ID NO: 126)); and K986P and V987P to interfere with the secondary structure of Spike with reference to the reference Spike protein (SEQ ID NO:59). The nucleotide sequence is shown in SEQ ID NO:89 and protein sequence shown in SEQ ID NO:90
“TCE5”: Selected CD8+ epitopes predicted by the EDGE platform to be presented on MHC molecules for SARS-CoV-2 proteins other than Spike. The nucleotide sequence is shown in SEQ ID NO:91 and protein sequence shown in SEQ ID NO:92
“TCE6”: Selected CD8+ epitopes predicted by the EDGE platform to be presented on MHC molecules for SARS-CoV-2 proteins other than Spike.
“TCE7”: Selected CD8+ epitopes predicted by the EDGE platform to be presented on MHC molecules for SARS-CoV-2 proteins other than Spike.
“TCE7”: Selected CD8+ epitopes predicted by the EDGE platform to be presented on MHC molecules for SARS-CoV-2 proteins other than Spike.
“TCE9”: Selected CD8+ epitopes predicted by the EDGE platform to be presented on MHC molecules for SARS-CoV-2 proteins other than Spike, including validated epitopes conserved between SARS and SARS-2 (e.g., as a pan-coronavirus vaccine), with certain frames extended (21 additional amino acids across all frames) to include additional predicted epitopes for alleles (i.e., not validated epitopes), for a total size of 556 amino acids
“TCE11”: Selected CD8+ epitopes predicted by the EDGE platform to be presented on MHC molecules for SARS-CoV-2 proteins other than Spike and Nucleocapsid; validated epitopes for a total size of 616 amino acids (197aa+full N) in addition to Spike
A representative SAM vector SAM-SGP1-TCE5-SGP2-CTSpike_{G}F2P is shown in SEQ ID NO:93

TABLE 2
Encoded Spike Variants

CTSpikeF2Pg nucleotide (SEQ ID NO: 89); Bold Italic Furin Mutation 682-685 RRAR (SEQ ID NO: 125)
to GSAS (SEQ ID NO: 126), Bold Lower Case K986P and V987P
ATGTTTGTCTTCCTGGTCTTGCTGCCGCTGGTGAGCAGCCAGTGCGTGAATCTCACCACCCGCACCC
AGCTTCCACCTGCCTACACTAACAGCTTCACCCGAGGGGTGTATTACCCTGACAAGGTATTCCGGTC
CTCCGTCCTCCATAGCACGCAGGACCTTTTTCTGCCCTTCTTCTCAAATGTGACATGGTTCCATGCCA
TTCACGTGAGCGGCACGAATGGAACGAAGCGCTTTGATAACCCCGTGCTGCCTTTCAATGACGGCG
TCTACTTCGCCTCCACTGAAAAGTCAAACATCATCCGGGGCTGGATCTTTGGCACCACTCTTGATTC
AAAGACCCAGTCACTGCTGATTGTGAACAATGCTACAAACGTGGTTATCAAGGTGTGTGAGTTTCA
GTTCTGTAACGATCCATTTTTGGGAGTGTACTACCACAAGAACAACAAGTCCTGGATGGAGTCTGA
GTTCAGAGTGTATAGCTCTGCTAACAACTGCACCTTCGAGTACGTGTCCCAGCCTTTCCTTATGGAC
CTGGAAGGCAAACAGGGCAATTTCAAAAACCTGAGAGAGTTCGTGTTTAAGAACATTGACGGATA
CTTCAAAATTTATTCTAAGCACACACCAATTAACTTAGTGCGGGACCTACCCCAAGGCTTTAGCGCC
CTAGAGCCCCTGGTTGACCTGCCCATTGGGATCAATATAACAAGGTTCCAAACTCTACTGGCTCTGC
ATAGAAGTTATCTGACCCCAGGAGACAGCTCTAGTGGTTGGACCGCCGGCGCAGCAGCCTACTATG
TCGGGTACTTACAGCCACGCACGTTCCTTCTGAAGTACAATGAGAACGGGACAATCACTGACGCAG
TAGACTGTGCACTGGACCCGCTAAGCGAGACTAAGTGCACACTTAAATCCTTCACGGTGGAGAAAG
GCATTTATCAGACCTCTAACTTCAGGGTGCAGCCAACAGAAAGCATTGTGCGATTCCCAAATATTA
CTAATCTTTGCCCTTTCGGGGAGGTCTTTAATGCAACTAGATTCGCATCAGTCTATGCGTGGAACCG
CAAACGCATTTCCAATTGTGTCGCAGACTACTCAGTGCTGTACAACTCTGCCTCTTTCAGTACGTTC
AAGTGTTACGGAGTGTCACCCACTAAACTGAACGACCTGTGCTTTACAAATGTCTACGCTGACTCCT
TCGTGATTAGGGGAGACGAGGTGAGACAAATTGCCCCCGGACAGACTGGGAAGATTGCCGACTAC
AATTATAAGCTTCCTGATGATTTCACTGGCTGTGTTATTGCCTGGAATAGTAACAATCTGGATAGCA
AGGTGGGAGGCAACTATAACTACTTATATCGACTGTTTAGGAAGAGTAATCTGAAACCATTTGAGC
GGGATATTTCCACAGAAATTTACCAGGCCGGGAGCACACCATGTAATGGGGTGGAGGGATTTAATT
GTTACTTCCCACTCCAGAGCTATGGTTTCCAACCCACCAATGGAGTGGGTTACCAGCCCTATAGAGT
CGTGGTGCTTAGTTTTGAGCTGCTTCACGCCCCAGCAACCGTCTGCGGTCCCAAAAAGTCGACCAAT
CTCGTGAAAAACAAATGCGTAAACTTCAACTTTAACGGCTTAACAGGAACCGGCGTGCTCACCGAA
AGCAACAAGAAATTCCTTCCATTTCAGCAATTCGGAAGGGACATCGCCGACACAACAGACGCGGTG
AGGGACCCACAGACTCTGGAGATACTGGACATCACTCCTTGTTCGTTTGGGGGCGTCTCGGTCATC
ACACCCGGGACTAATACTAGTAATCAGGTAGCAGTTTTATATCAAGGCGTCAACTGTACCGAAGTA
CCTGTGGCCATACACGCTGATCAGCTAACGCCAACATGGCGAGTCTATTCCACCGGCTCTAACGTTT
TTCAGACCAGGGCTGGGTGCCTGATAGGGGCAGAGCACGTCAATAATTCCTATGAGTGTGATATCC
CCATAGGTGCGGGGATCTGTGCCAGCTATCAAACCCAAACCAATTCACCAgGGaGcGCAaGcTCTGT
GGCTTCTCAGAGCATAATTGCATATACAATGTCACTGGGCGCTGAGAATAGCGTTGCATACTCTAA
TAACAGCATAGCCATTCCCACGAACTTTACTATCAGTGTGACAACCGAAATATTGCCAGTTTCGATG
ACCAAAACTAGCGTGGATTGCACGATGTACATCTGTGGAGACTCTACCGAATGCAGCAATCTGCTA
TTACAATATGGCAGCTTCTGTACACAGTTAAATCGAGCCTTGACAGGCATCGCAGTGGAACAGGAC
AAAAATACTCAAGAGGTGTTTGCACAGGTGAAGCAAATCTACAAAACGCCCCCCATTAAAGATTTT
GGCGGGTTCAATTTTTCACAAATTCTCCCCGACCCGTCTAAGCCGAGTAAGCGGTCCTTCATCGAAG
ATCTGCTCTTTAACAAAGTAACCCTCGCCGATGCCGGCTTTATTAAGCAGTATGGCGACTGCCTGGG
GGATATAGCCGCTCGTGACCTGATTTGCGCCCAGAAGTTCAATGGTCTGACCGTGTTGCCTCCTTTA
TTGACCGATGAAATGATTGCCCAGTACACTAGTGCCCTGCTGGCCGGCACTATCACGTCTGGGTGG
ACCTTCGGAGCTGGTGCCGCCTTGCAGATACCTTTTGCAATGCAGATGGCCTATAGGTTTAATGGTA
TCGGAGTGACTCAGAACGTACTGTACGAGAACCAGAAGCTCATCGCTAATCAATTTAACTCCGCTA
TCGGAAAAATCCAGGACAGCCTCTCTTCTACAGCTAGCGCTCTGGGCAAACTGCAGGATGTCGTTA
ATCAGAATGCCCAGGCCCTGAACACCTTGGTTAAACAACTATCTTCCAACTTCGGGGCCATATCCA
GTGTGTTGAATGATATTCTCTCCCGCTTGGATccacctGAAGCTGAGGTGCAGATCGATCGCTTGATCA
CCGGCAGACTGCAGTCCCTCCAGACATATGTAACTCAGCAGCTGATTAGAGCCGCCGAGATAAGGG
CAAGTGCGAATCTGGCTGCCACCAAGATGAGCGAATGTGTATTGGGCCAGAGCAAACGAGTTGATT
TTTGCGGTAAGGGGTATCATTTAATGTCTTTCCCTCAATCCGCACCTCATGGCGTAGTTTTCCTGCAT
GTGACTTATGTCCCGGCTCAGGAGAAGAATTTTACCACAGCCCCCGCGATCTGCCATGACGGAAAG
GCCCACTTCCCCCGGGAAGGCGTGTTTGTATCCAATGGGACTCACTGGTTTGTCACTCAGCGAAATT
TTTATGAACCACAGATCATCACCACTGACAACACATTTGTTAGTGGAAACTGCGATGTGGTCATCG
GCATCGTGAATAACACTGTCTATGATCCACTGCAACCTGAACTGGATTCTTTTAAAGAGGAACTCG
ACAAGTATTTTAAAAACCACACTAGCCCTGACGTGGATCTCGGTGACATTTCTGGCATCAACGCTA
GCGTAGTGAACATTCAGAAAGAGATAGATAGACTTAATGAGGTGGCCAAGAACCTCAACGAAAGT
CTGATCGACCTCCAGGAACTGGGGAAATACGAGCAGTACATTAAATGGCCTTGGTACATATGGCTG
GGGTTCATTGCTGGGCTGATCGCAATAGTGATGGTGACCATAATGCTCTGTTGCATGACTAGCTGCT
GCAGCTGCCTGAAGGGCTGCTGTAGTTGTGGGTCATGTTGTAAGTTTGACGAAGATGATAGCGAGC
CTGTCCTTAAAGGAGTGAAGCTCCACTACACCTAG
CTSpikeF2Pg amino acid (SEQ ID NO: 90); Bold Italic Furin Mutation 682-685 RRAR (SEQ ID NO: 125)
to GSAS (SEQ ID NO: 126), Bold Lower Case K986P and V987P
MFVFLVLLPLVSSQCVNLTTRTQLPPAYTNSFTRGVYYPDKVFRSSVLHSTQDLFLPFFSNVTWFHAIHV
SGTNGTKRFDNPVLPFNDGVYFASTEKSNIIRGWIFGTTLDSKTQSLLIVNNATNVVIKVCEFQFCNDPFL
GVYYHKNNKSWMESEFRVYSSANNCTFEYVSQPFLMDLEGKQGNFKNLREFVFKNIDGYFKIYSKHTPI
NLVRDLPQGFSALEPLVDLPIGINITRFQTLLALHRSYLTPGDSSSGWTAGAAAYYVGYLQPRTFLLKYN
ENGTITDAVDCALDPLSETKCTLKSFTVEKGIYQTSNFRVQPTESIVRFPNITNLCPFGEVFNATRFASVY
AWNRKRISNCVADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGSTPCNGVEGENCY
FPLQSYGFQPTNGVGYQPYRVVVLSFELLHAPATVCGPKKSTNLVKNKCVNFNFNGLTGTGVLTESNK
KFLPFQQFGRDIADTTDAVRDPQTLEILDITPCSFGGVSVITPGTNTSNQVAVLYQGVNCTEVPVAIHAD
QLTPTWRVYSTGSNVFQTRAGCLIGAEHVNNSYECDIPIGAGICASYQTQTNSPGSASSVASQSIIAYTMS
LGAENSVAYSNNSIAIPTNFTISVTTEILPVSMTKTSVDCTMYICGDSTECSNLLLQYGSFCTQLNRALTGI
AVEQDKNTQEVFAQVKQIYKTPPIKDFGGFNFSQILPDPSKPSKRSFIEDLLFNKVTLADAGFIKQYGDCL
GDIAARDLICAQKFNGLTVLPPLLTDEMIAQYTSALLAGTITSGWTFGAGAALQIPFAMQMAYRFNGIG
VTQNVLYENQKLIANQFNSAIGKIQDSLSSTASALGKLQDVVNQNAQALNTLVKQLSSNFGAISSVLNDI
LSRLDppEAEVQIDRLITGRLQSLQTYVTQQLIRAAEIRASANLAATKMSECVLGQSKRVDFCGKGYHL
MSFPQSAPHGVVFLHVTYVPAQEKNFTTAPAICHDGKAHFPREGVFVSNGTHWFVTQRNFYEPQIITTD
NTFVSGNCDVVIGIVNNTVYDPLQPELDSFKEELDKYFKNHTSPDVDLGDISGINASVVNIQKEIDRLNE
VAKNLNESLIDLQELGKYEQYIKWPWYIWLGFIAGLIAIVMVTIMLCCMTSCCSCLKGCCSCGSCCKFD
EDDSEPVLKGVKLHYT*
B.1.351Spike-FurinMt Amino Acid sequence (South African Spike Variant) (SEQ ID NO:112)
MFVFLVLLPLVSSQCVNFTTRTQLPPAYTNSFTRGVYYPDKVFRSSVLHSTQDLFLPFFSNVTWFHAIHV
SGTNGTKRFANPVLPFNDGVYFASTEKSNIIRGWIFGTTLDSKTQSLLIVNNATNVVIKVCEFQFCNDPFL
GVYYHKNNKSWMESEFRVYSSANNCTFEYVSQPFLMDLEGKQGNFKNLREFVFKNIDGYFKIYSKHTPI
NLVRGLPQGFSALEPLVDLPIGINITRFQTLHISYLTPGDSSSGWTAGAAAYYVGYLQPRTFLLKYNENG
TITDAVDCALDPLSETKCTLKSFTVEKGIYQTSNFRVQPTESIVRFPNITNLCPFGEVENATRFASVYAWN
RKRISNCVADYSVLYNSASFSTFKCYGVSPTKLNDLCFTNVYADSFVIRGDEVRQIAPGQTGNIADYNY
KLPDDFTGCVIAWNSNNLDSKVGGNYNYLYRLFRKSNLKPFERDISTEIYQAGSTPCNGVKGFNCYFPL
QSYGFQPTYGVGYQPYRVVVLSFELLHAPATVCGPKKSTNLVKNKCVNFNFNGLTGTGVLTESNKKFL
PFQQFGRDIADTTDAVRDPQTLEILDITPCSFGGVSVITPGTNTSNQVAVLYQGVNCTEVPVAIHADQLTP
TWRVYSTGSNVFQTRAGCLIGAEHVNNSYECDIPIGAGICASYQTQTNSPGSASSVASQSIIAYTMSLGV
ENSVAYSNNSIAIPTNFTISVTTEILPVSMTKTSVDCTMYICGDSTECSNLLLQYGSFCTQLNRALTGIAVE
QDKNTQEVFAQVKQIYKTPPIKDFGGFNFSQILPDPSKPSKRSFIEDLLFNKVTLADAGFIKQYGDCLGDI
AARDLICAQKFNGLTVLPPLLTDEMIAQYTSALLAGTITSGWTFGAGAALQIPFAMQMAYRENGIGVTQ
NVLYENQKLIANQFNSAIGKIQDSLSSTASALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGKGYHLMSF
PQSAPHGVVFLHVTYVPAQEKNFTTAPAICHDGKAHFPREGVFVSNGTHWFVTQRNFYEPQIITTDNTF
VSGNCDVVIGIVNNTVYDPLQPELDSFKEELDKYFKNHTSPDVDLGDISGINASVVNIQKEIDRLNEVAK
NLNESLIDLQELGKYEQYIKWPWYIWLGFIAGLIAIVMVTIMLCCMTSCCSCLKGCCSCGSCCKFDEDDS
EPVLKGVKLHYT

TABLE 8
TCE9 Cassette (Order of Frames as Shown)

| | Frame Start | Frame End | | SEQ |
|---|---|---|---|---|
| Gene | in Gene | in Gene | Frame sequence | ID NO: |
| nsp12 | 4719 | 4745 | GPLVRKIFVDGVPFVVSTGYHFRELGV | 27961 |
| nsp4 | 3096 | 3134 | PVYSFLPGVYSVIYLYLTFYLTNDVSFLAHIQWMVM | 27962 |
| | | | FTP | |
| M | 89 | 109 | GLMWLSYFIASFRLFARTRSM | 27951 |
| nsp12 | 4888 | 4905 | NNLDKSAGFPFNKWGKAR | 27963 |
| nsp3 | 2745 | 2761 | ATTRQVVNVVTTKIALK | 27957 |
| M | 61 | 79 | TLACFVLAAVYRINWITGG | 27964 |
| nsp3 | 1919 | 1935 | PYPNASFDNFKFVCDNI | 27953 |
| nsp3 | 2676 | 2692 | TYNKVENMTPRDLGACI | 27965 |
| ORF3a | 29 | 52 | VRATATIPIQASLPFGWLIVGVAL | 27949 |
| nsp12 | 4806 | 4826 | NFNKDFYDFAVSKGFFKEGSS | 27954 |
| N | 96 | 117 | GGDGKMKDLSPRWYFYYLGTGP | 27960 |
| nsp12 | 5213 | 5234 | KQGDDYVYLPYPDPSRILGAGC | 27966 |
| N | 356 | 378 | HIDAYKTFPPTEPKKDKKKKADE | 27944 |
| ORF3a | 133 | 149 | CRSKNPLLYDANYFLCW | 27945 |
| nsp6 | 3643 | 3668 | SLATVAYFNMVYMPASWVMRIMTWLD | 27967 |
| nsp12 | 4529 | 4545 | GNCDTLKEILVTYNCCD | 27968 |
| nsp3 | 1630 | 1660 | EAFEYYHTTDPSFLGRYMSALNHTKKWKYPQ | 27969 |
| nsp3 | 1360 | 1377 | ISNEKQEILGTVSWNLRE | 27947 |
| M | 160 | 203 | DIKDLPKEITVATSRTLSYYKLGASQRVAGDSGFAA | 27948 |
| | | | YSRYRIGN | |
| N | 301 | 337 | WPQIAQFAPSASAFFGMSRIGMEVTPSGTWLTYTGAI | 27970 |
| nsp12 | 4553 | 4571 | DWYDFVENPDILRVYANLG | 27952 |
| nsp4 | 2850 | 2909 | ACPLIAAVITREVGFVVPGLPGTILRTTNGDFLHFLPR | 27971 |
| | | | VFSAVGNICYTPSKLIEYTDFA | |

TABLE 9
TCE11 Cassette (Order of Frames as Shown)

| | Frame Start | Frame End | | SEQ |
|---|---|---|---|---|
| Gene | in Gene | in Gene | Frame sequence | ID NO: |
| nsp12 | 4896 | 4826 | NVAFQTVKPGNFNKDFYDFAVSKGFFKEGSS | 27972 |
| M | 182 | 203 | GASQRVAGDSGFAAYSRYRIGN | 27973 |
| nsp12 | 4728 | 4745 | DGVPFVVSTGYHFRELGV | 27955 |
| nsp4 | 3111 | 3134 | YLTFYLTNDVSFLAHIQWMVMFTP | 27950 |
| M | 89 | 109 | GLMWLSYFIASFRLFARTRSM | 27951 |
| nsp3 | 1632 | 1660 | FEYYHTTDPSFLGRYMSALNHTKKWKYPQ | 27946 |
| nsp3 | 1360 | 1377 | ISNEKQEILGTVSWNLRE | 27947 |
| nsp3 | 2745 | 2761 | ATTRQVVNVVTTKIALK | 27957 |
| M | 61 | 77 | TLACFVLAAVYRINWIT | 27959 |

TCE5 Amino Acid Sequence (SEQ ID NO: 92):
MAGEAPFLYLYALVYFLQSINFVRIIMRLWLCWKCRSKNPLLYDANYFLCWHTNLAVFQSASKI
ITLKKRWQLALSKGVHFVCNLLLVTLKQGEIKDATPSDFVRATATIPIQASLPFGWLIVGVALLA
VRRPQGLPNNTASWFTALTQHGKEDLKFPRGQGVPINTNSSPDDQIGYYRRATRRIMACLVGLM
WLSYFIASFRLKKDKKKKADETQALPQRQKKQQTVTLLPAADLDDFSKQLQGNFGDQELIRQG
TDYKHWPQIAQFAPSASAFFGMSRIGMEVTPSGTWLTYTGAIKITSGDGTTSPISEHDYQIGGYTE
KWESGVKKMSGKGQQQQGQTVTKKSAAEASKKPRQKRTATKAYNVTQAFGRRGPEQTLLWP
VTLACFVLAAVYRINWFKDQVILLNKHIDAYKTFPPTEPKKDKKKKTSPARMAGNGGDAALAL
LLLDRLNQLESKMSGKGQKMKDLSPRWYFYYLGTGPEDCVVLHSYFTSDYYQLYSTQLSTDTG
VEHVTFFIYNKIVDEPEEHVQIHTIDGSSGGIIWVATEGALNTPKDHIGTRNPANNAAIVLQLPQG
TTLPKGFYAEGPGPGAKFVAAWTLKAAAGPGPGQYIKANSKFIGITELGPGPG-
TCE11 Amino Acid Sequence: Concatenated EDGE predicted SARS-COV-2 MHC
Class I Epitope Cassette “TCE11” with N-term leader (bold) and C-term Universal MHC
Class II with GPGPG linkers (SEQ ID NO: 56) (bold italic):
MAGNVAFQTVKPGNFNKDFYDFAVSKGFFKEGSSGASQRVAGDSGFAAYSRYRIGNDGVPFVV
STGYHFRELGVYLTFYLTNDVSFLAHIQWMVMFTPGLMWLSYFIASFRLFARTRSMFEYYHTTD
PSFLGRYMSALNHTKKWKYPQISNEKQEILGTVSWNLREATTRQVVNVVTTKIALKTLACFVLA
AVYRINWITGPGPGAKFVAAWTLKAAAGPGPGQYIKANSKFIGITELGPGPG-

SAM-SGP1-TCE5-SGP2-CTSpike_{G}F2P Nucleotide Sequence (SEQ ID NO:93): (NT 1-17: T7 promoter; NT 62-7543: VEEV non-structural protein coding region; NT 7518-7560: SGP1; NT 7582-7587 and 9541-9546: Kozak sequence; NT 7588-9474: TCE5 cassette; NT 9480-9540: SGP2; NT 9547-13368: CTSpike Furin-2P)

CMV-CTSpikeGF2P-CMV-TCE5 Cassette Nucleotide Sequence (SEQ ID NO:114)

### Results

Various SAM vector organizations all featuring dual cassettes expressed by SGP1 and SGP2 were evaluated.

Examined were multicistronic SAM vectors that address potential technical limitations present in the field, including, but not limited to: (1) improving expression of multiple payloads that include large cassettes (e.g., greater than the size of a native cassette expressed from a native alphavirus subgenomic promoter, such as cassettes approximately 4 kb or greater in length); (2) improved control of expression of multiple payloads, e.g., controlling the relative expression of different payloads; and (3) improved vector stability, such as by reducing vector recombination events (e.g., intra-vector promoter recombination between alphavirus subgenomic promoters).

Expression of the cassettes was effectively driven by both promoters, as assessed by monitoring T cell responses to the encoded epitopes. Notably, the second cassette under the control of the SGP2 promoter results in better expression in a vaccine setting, as assessed by monitoring T cell responses, generally greater than two fold.

SAM vaccine platforms encoding various orders of a modified SARS-CoV-2 Spike protein and a T cell epitope (TCE) cassette encoding EDGE predicted epitopes (EPE) were assessed.

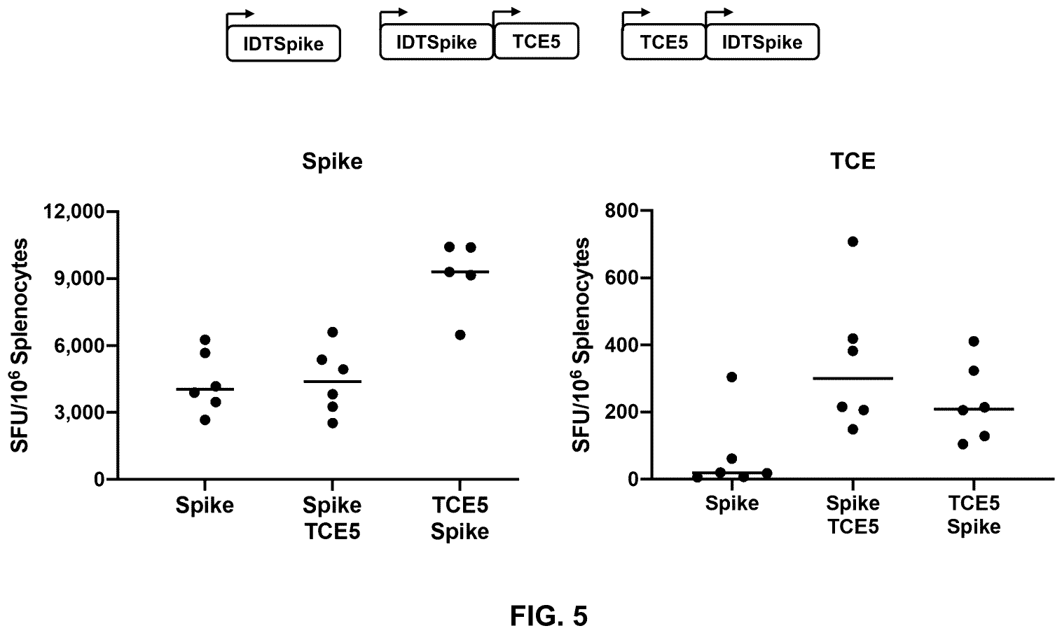

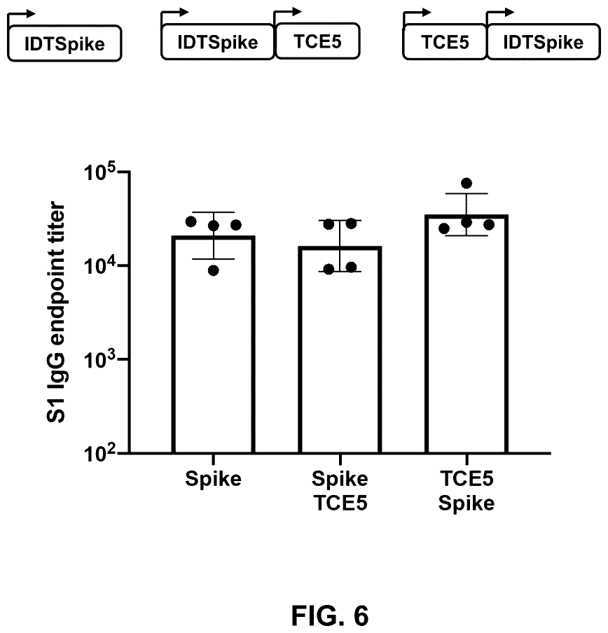

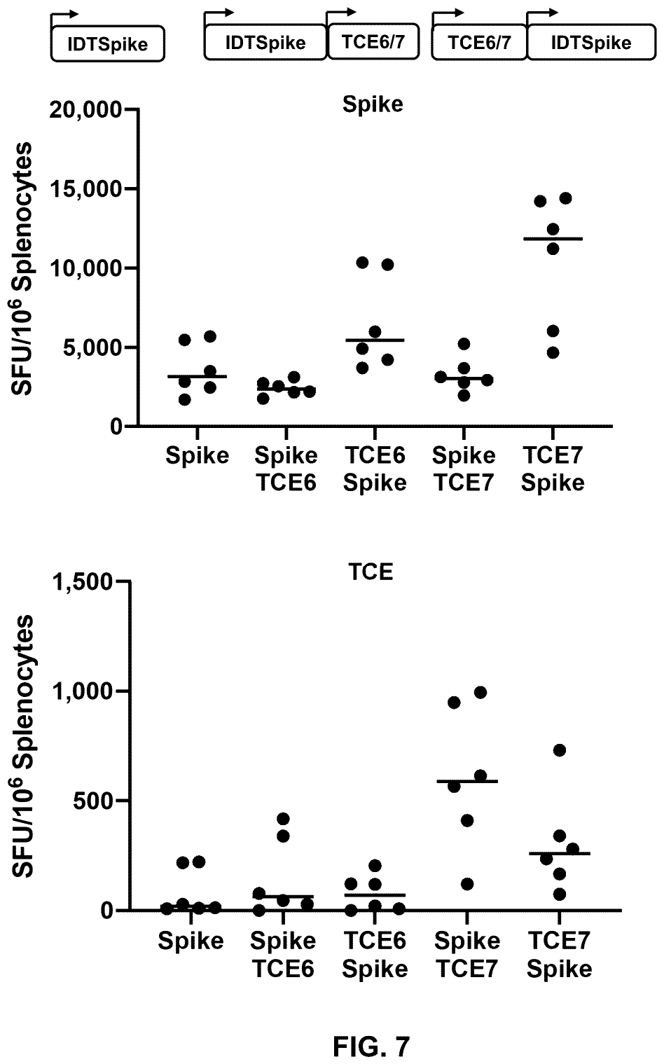

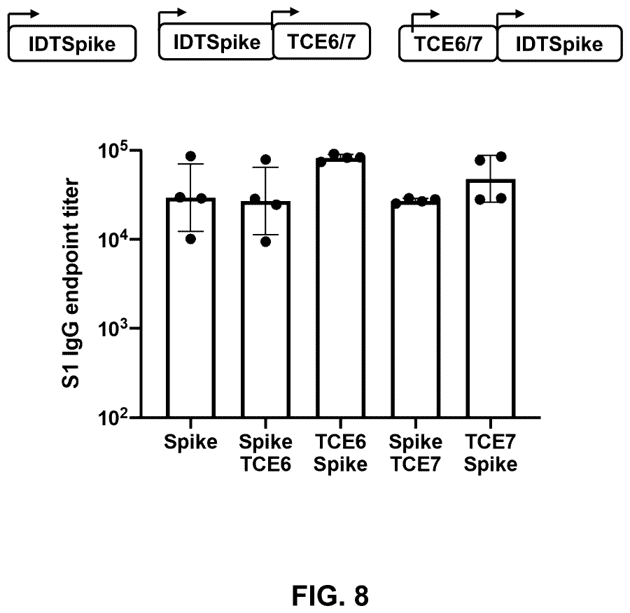

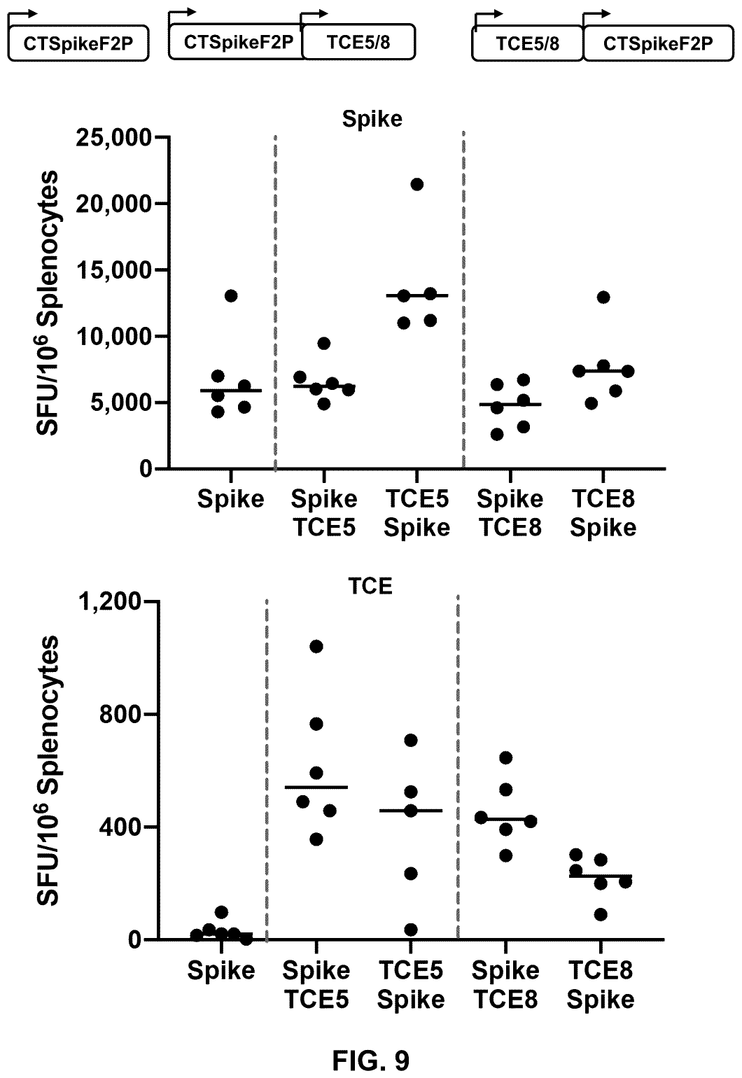

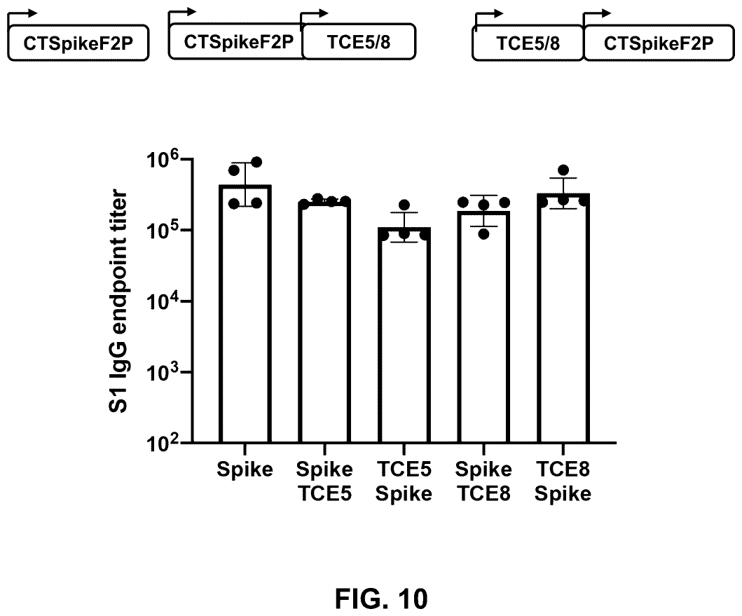

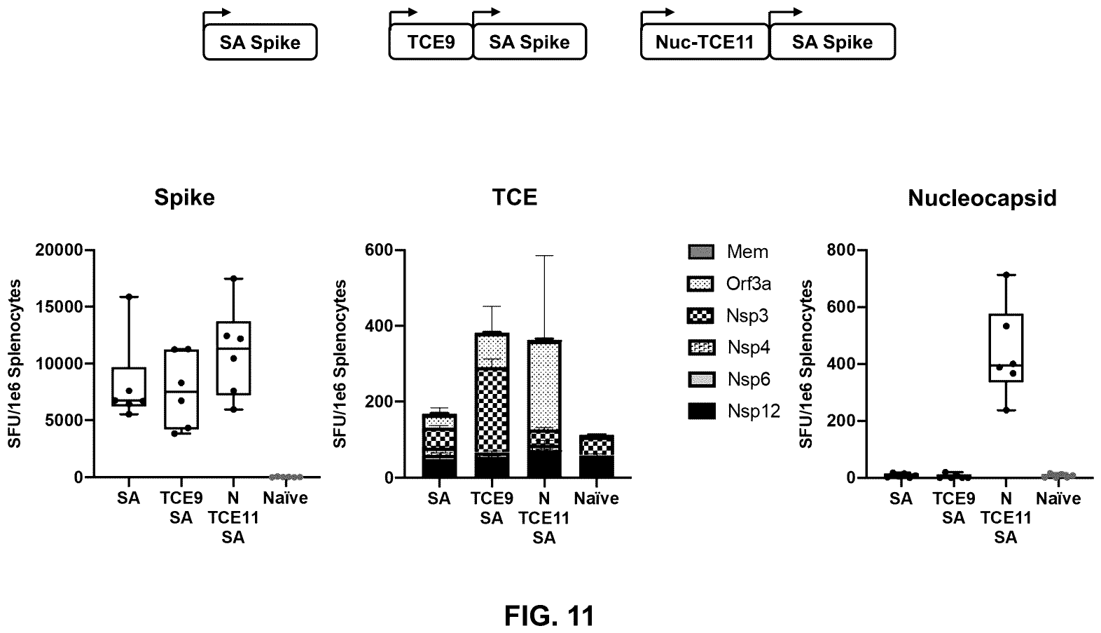

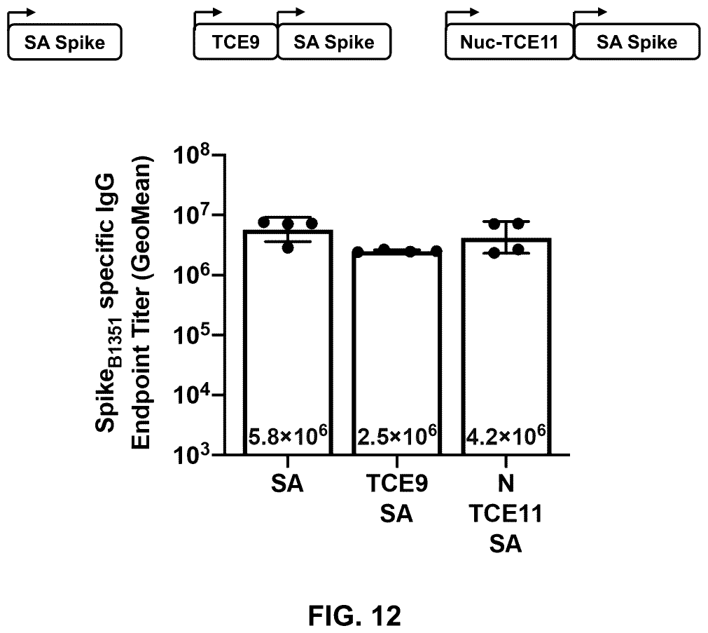

T cell responses to Spike (top panel), T cell responses to the encoded T cell epitopes (middle panel), and Spike-specific IgG antibodies (bottom panel) were produced when vaccinated with various constructs. In FIG. 5 and FIG. 6 (and quantified in Tables 4A-C), SAM constructs included “IDTSpike_{g}” (SEQ ID NO:69) alone (left columns), IDTSpike_{g} expressed from a first subgenomic promoter followed by TCE5 expressed from a second subgenomic promoter (middle columns), or TCE5 expressed from a first subgenomic promoter followed by IDTSpike_{g} expressed from a second subgenomic promoter (right columns), with immune responses assessed, as described above. In FIG. 7 and FIG. 8 (and quantified in Tables 5A-C), SAM constructs included “IDTSpike_{g}” (SEQ ID NO:69) alone (first column), IDTSpike_{g} expressed from a first subgenomic promoter followed by TCE6 or TCE7 expressed from a second subgenomic promoter (columns 2 and 4, respectively), or TCE6 or TCE7 expressed from a first subgenomic promoter followed by IDTSpike_{g} expressed from a second subgenomic promoter (columns 3 and 5, respectively), with immune responses assessed, as described above In FIG. 9 and FIG. 10 (and quantified in Tables 6A-C), SAM constructs included “CTSpike_{g}” (SEQ ID NO:79) alone (first column), CTSpike_{g} expressed from a first subgenomic promoter followed by TCE5 or TCE8 expressed from a second subgenomic promoter (columns 2 and 4, respectively), or TCE5 or TCE8 expressed from a first subgenomic promoter followed by CTSpike_{g} expressed from a second subgenomic promoter (columns 3 and 5, respectively), with immune responses assessed, as described above. In FIG. 11 and FIG. 12 (and quantified in Tables 7A-D), SAM constructs included a Spike protein (501Y.V2) from the B.1.351 (“South African” beta-lineage) SARS-CoV-2 isolate (SEQ ID NO:112; “SA Spike”) alone (first column), TCE5 (see Table 8) expressed from a first subgenomic promoter followed by SA-Spike expressed from a second subgenomic promoter (column 2), a cassette encoding both a SARS-CoV-2 Nucleocapsid protein (SEQ ID NO:62; “Nuc”; original Wuhan isolate) and TCE11 (see Table 9) expressed from a first subgenomic promoter (Nucleocapsid-T2A linker-TCE11, and including GPGPG linkers (SEQ ID NO: 56) and both PADRE and Tetanus Toxoid universal MHC class II epitopes) followed by SA-Spike expressed from a second subgenomic promoter (column 3), or naïve mice (column 4) with immune responses assessed, as described above.

Generally, and in particular for Spike proteins, T cell responses were increased when the respective epitopes were expressed from the second subgenomic promoter (SGP2), including increased Spike-directed T cell responses relative to Spike alone. A similar trend was also observed generally with increased Spike-specific IgG titers when the Spike antigen was expressed from the second subgenomic promoter except, for potentially the CTSpike g constructs.

Accordingly, the results demonstrate the use of multiple distinct SGP promoters results in effective expression of multiple cassettes within an alphavirus-derived SAM vector, particularly in a vaccine setting. Additionally, the data demonstrate sequence order of antigen cassettes in the SAM vaccine platform influenced immune responses.

TABLE 4A
Spike T cell response (SFU/1e6 splenocytes)

| | Spike | Spike-TCE5 | TCE5-Spike |
|---|---|---|---|
| | 3466 | 6604 | 10395 |
| | 3884 | 2522 | 10420 |
| | 6269 | 5372 | 9292 |
| | 5678 | 4934 | 6488 |
| | 2666 | 3257 | 9156 |
| | 4164 | 3814 | 2815 |
| Median | 4024 | 4374 | 9224 |

TABLE 4B
TCE T cell response (SFU/1e6 splenocytes)

| | Spike | Spike-TCE5 | TCE5-Spike |
|---|---|---|---|
| | 18 | 215 | 324 |
| | 5 | 708 | 104 |
| | 304 | 383 | 128 |
| | 20 | 419 | 205 |
| | 61 | 148 | 214 |
| | 7 | 206 | 411 |
| Median | 19 | 299 | 209 |

TABLE 4C
S1 IgG endpoint titer

| | Spike | Spike-TCE5 | TCE5-Spike |
|---|---|---|---|
| | 8904 | 27680 | 75450 |
| | 29451 | 28074 | 27263 |
| | 27217 | 9645 | 28979 |
| | 26670 | 9149 | 24879 |
| Geomean | 20887 | 16182 | 34897 |
| Geometric | 1.768 | 1.875 | 1.678 |
| SD factor | | | |

TABLE 5A
Spike T cell response (SFU/1e6 splenocytes)

| | | TCE6 | Spike | Spike | TCE7 |
|---|---|---|---|---|---|
| | Spike | Spike | TCE6 | TCE7 | Spike |
| | 5697 | 2558 | 4219 | 1977 | 6040 |
| | 2842 | 1781 | 4928 | 3144 | 4677 |
| | 2476 | 2183 | 10362 | 3685 | 14404 |
| | 1704 | 2748 | 3706 | 5231 | 14220 |
| | 5478 | 3126 | 5996 | 2941 | 12466 |
| | 3500 | 2212 | 10228 | 2790 | 11233 |
| Median | 3171 | 2385 | 5462 | 3042 | 11850 |

TABLE 5B
TCE T cell response (SFU/1e6 splenocytes)

| | | TCE6 | Spike | Spike | TCE7 |
|---|---|---|---|---|---|
| | Spike | Spike | TCE6 | TCE7 | Spike |
| | 28 | 78 | 204 | 614 | 167 |
| | 13 | 0 | 20 | 411 | 74 |
| | 10 | 29 | 8 | 948 | 341 |
| | 221 | 339 | 119 | 565 | 235 |
| | 8 | 419 | 122 | 121 | 281 |
| | 218 | 46 | 0 | 994 | 731 |
| Median | 20 | 62 | 70 | 590 | 258 |

TABLE 5C
S1 IgG endpoint titer

| | | Spike | TCE6 | Spike | TCE7 |
|---|---|---|---|---|---|
| | Spike | TCE6 | Spike | TCE7 | Spike |
| | 10144 | 24609 | 74421 | 26791 | 85199 |
| | 28845 | 9464 | 91647 | 28185 | 29016 |
| | 86496 | 79136 | 84008 | 25318 | 28165 |
| | 29796 | 28607 | 83058 | 29150 | 77604 |
| Geomean | 29468 | 26947 | 83057 | 27322 | 48213 |
| Geometric | 2.399 | 2.385 | 1.089 | 1.063 | 1.831 |
| SD factor | | | | | |

TABLE 6A
Spike T cell response (SFU/1e6 splenocytes)

| | | TCE5 | Spike | Spike | TCE8 |
|---|---|---|---|---|---|
| | Spike | Spike | TCE5 | TCE8 | Spike |
| | 13043 | 5977 | 11014 | 5177 | 5880 |
| | 5533 | 9466 | 11203 | 6359 | 12949 |
| | 4305 | 4897 | 21464 | 2611 | 7382 |
| | 7015 | 6451 | 13209 | 3184 | 7357 |
| | 4665 | 6026 | 13058 | 6709 | 4947 |
| | 6260 | 6936 | * | 4614 | 7772 |
| Median | 5896 | 6238 | 13058 | 4895 | 7369 |

* Sample not analyzed due to poor quality of isolated splenocytes

TABLE 6B
TCE T cell response (SFU/1e6 splenocytes)

| | | TCE5 | Spike | Spike | TCE8 |
|---|---|---|---|---|---|
| | Spike | Spike | TCE5 | TCE8 | Spike |
| | 20 | 765 | 35 | 420 | 205 |
| | 15 | 458 | 457 | 433 | 284 |
| | 20 | 357 | 524 | 391 | 199 |
| | 97 | 1041 | 707 | 646 | 301 |
| | 3 | 489 | 235 | 299 | 89 |
| | 36 | 591 | * | 533 | 246 |
| Median | 20 | 540 | 457 | 427 | 226 |

* Sample not analyzed due to poor quality of isolated splenocytes

TABLE 6C
S1 IgG endpoint titer

| | | Spike | TCE5 | Spike | TCE8 |
|---|---|---|---|---|---|
| | Spike | TCE5 | Spike | TCE8 | Spike |
| | 917961 | 256697 | 227456 | 246815 | 268408 |
| | 241746 | 277841 | 85046 | 88935 | 247788 |
| | 236694 | 253652 | 85780 | 228072 | 704747 |
| | 698916 | 232433 | 90132 | 248220 | 259580 |
| Geomean | 437723 | 254647 | 110587 | 187754 | 332120 |
| Geometric | 2.027 | 1.076 | 1.618 | 1.648 | 1.653 |
| SD factor | | | | | |

TABLE 7A
Spike T cell response (SFU/1e6 splenocytes)

| SA | TCE9-SA | N-TCE11-SA | Naive |
|---|---|---|---|
| 15900 | 8323 | 12215 | 20 |
| 6686 | 4344 | 7611 | 15 |
| 6748 | 6724 | 17488 | 20 |
| 6435 | 11265 | 10469 | 10 |
| 5547 | 3840 | 5961 | 80 |
| 7615 | 11284 | 12460 | 45 |

TABLE 7B
TCE T cell response (SFU/1e6 splenocytes) - Mean +/− SEM

| Protein | SA | TCE9-SA | N-TCE11-SA | Naive |
|---|---|---|---|---|
| Mem | 3.38 +/− 1.25 | 1.25 +/− 1.25 | 3.75 +/− 2.39 | 1.66 +/− 0.83 |
| ORF3A | 33.06 +/− 18.49 | 89.82 +/− 70.08 | 233.79 +/− 225.79 | 3.78 +/− 0.86 |
| NSP3 | 52.96 +/− 4.77 | 224.87 +/− 21.91 | 40.51 +/− 5.38 | 48.3 +/− 3.67 |
| NSP4 | 19.16 +/− 4.17 | 12.01 +/− 3.32 | 13.33 +/− 2.47 | 9.17 +/− 2.86 |
| NSP6 | 12.08 +/− 4.15 | 5.51 +/− 2.66 | 2.91 +/− 1.87 | 5.48 +/− 2.94 |
| NSP12 | 48.03 +/− 8.23 | 49.35 +/− 8.30 | 70.01 +/− 27.57 | 44.81 +/− 10.86 |

TABLE 7C
Nucleocapsid T cell response (SFU/1e6 splenocytes)

| SA | TCE9-SA | N-TCE11-SA | Naive |
|---|---|---|---|
| 2.5 | 10 | 713 | 0 |
| 15 | 0 | 400 | 0 |
| 17.5 | 0 | 238 | 7.90 |
| 7.5 | 0 | 388 | 12.5 |
| 5 | 0 | 367 | 15 |
| 0 | 19.9 | 533 | 0 |

TABLE 7D
S1 IgG endpoint titer

| | SA | TCE9-SA | N-TCE11-SA |
|---|---|---|---|
| | 7106065 | 2451309 | 2326720 |
| | 7223710 | 2663906 | 7150126 |
| | 7599055 | 2489358 | 2677973 |
| | 2857405 | 2412035 | 7082536 |
| Geomean | 5778036 | 2502344 | 4214666 |
| Geometric | 1.6 | 1.045 | 1.836 |
| SD factor | | | |

Certain additional sequences for vectors, cassettes, and antibodies referred to herein are described below and referred to by SEQ ID NO.

Tremelimumab VL (SEQ ID NO: 16)
Tremelimumab VH (SEQ ID NO: 17)
Tremelimumab VH CDR1 (SEQ ID NO: 18)
Tremelimumab VH CDR2 (SEQ ID NO: 19)
Tremelimumab VH CDR3 (SEQ ID NO: 20)
Tremelimumab VL CDR1 (SEQ ID NO: 21)
Tremelimumab VL CDR2 (SEQ ID NO: 22)
Tremelimumab VL CDR3 (SEQ ID NO: 23)
Durvalumab (MEDI4736) VL (SEQ ID NO: 24)
MEDI4736 VH (SEQ ID NO: 25)
MEDI4736 VH CDR1 (SEQ ID NO: 26)
MEDI4736 VH CDR2 (SEQ ID NO: 27)
MEDI4736 VH CDR3 (SEQ ID NO: 28)
MEDI4736 VL CDR1 (SEQ ID NO: 29)
MEDI4736 VL CDR2 (SEQ ID NO: 30)
MEDI4736 VL CDR3 (SEQ ID NO: 31)
UbA76-25merPDTT nucleotide (SEQ ID NO: 32)
UbA76-25merPDTT polypeptide (SEQ ID NO: 33)
MAG-25merPDTT nucleotide (SEQ ID NO: 34)
MAG-25merPDTT polypeptide (SEQ ID NO: 35)
Ub7625merPDTT_NoSFL nucleotide (SEQ ID NO: 36)
Ub7625merPDTT_NoSFL polypeptide (SEQ ID NO: 37)
ChAdV68.5WTnt.MAG25mer (SEQ ID NO: 2); AC_000011.1 with E1 (nt 577 to 3403) and E3 (nt 27,125-
31,825) sequences deleted; corresponding ATCC VR-594 nucleotides substituted at five positions; model
neoantigen cassette under the control of the CMV promoter/enhancer inserted in place of deleted E1; SV40
polyA 3′ of cassette
Venezuelan equine encephalitis virus [VEE] (SEQ ID NO: 3) GenBank: L01442.2
VEE-MAG25mer (SEQ ID NO: 4); contains MAG-25merPDTT nucleotide (bases 30-1755)
Venezuelan equine encephalitis virus strain TC-83 [TC-83](SEQ ID NO: 5) GenBank: L01443.1
VEE Delivery Vector (SEQ ID NO: 6); VEE genome with nucleotides 7544-11176 deleted [alphavirus structural
proteins removed]
TC-83 Delivery Vector(SEQ ID NO: 7); TC-83 genome with nucleotides 7544-11176 deleted [alphavirus
structural proteins removed]
VEE Production Vector (SEQ ID NO: 8); VEE genome with nucleotides 7544-11176 deleted, plus 5′ T7-
promoter, plus 3′ restriction sites
TC-83 Production Vector(SEQ ID NO: 9); TC-83 genome with nucleotides 7544-11176 deleted, plus 5′ T7-
promoter, plus 3′ restriction sites
VEE-UbAAY (SEQ ID NO: 14); VEE delivery vector with MHC class I mouse tumor epitopes SIINFEKL (SEQ
ID NO: 129) and AH1-A5 inserted
VEE-Luciferase (SEQ ID NO: 15); VEE delivery vector with luciferase gene inserted at 7545
ubiquitin (SEQ ID NO: 38) > UbG76 0-228
Ubiquitin A76 (SEQ ID NO: 39) > UbA76 0-228
HLA-A2 (MHC class I) signal peptide (SEQ ID NO: 40) > MHC SignalPep 0-78
HLA-A2 (MHC class I) Trans Membrane domain (SEQ ID NO: 41) > HLA A2 TM Domain 0-201
IgK Leader Seq (SEQ ID NO: 42) > IgK Leader Seq 0-60
Human DC-Lamp (SEQ ID NO: 43) > HumanDCLAMP 0-3178
Mouse LAMP1 (SEQ ID NO: 44) > MouseLamp1 0-1858
Human Lamp1 cDNA (SEQ ID NO: 45) > Human Lamp1 0-2339
Tetanus toxoid nulceic acid sequence (SEQ ID NO: 46)
Tetanus toxoid amino acid sequence (SEQ ID NO: 47)
PADRE nulceotide sequence (SEQ ID NO: 48)
PADRE amino acid sequence (SEQ ID NO: 49)
WPRE (SEQ ID NO: 50) > WPRE 0-593
IRES (SEQ ID NO: 51) > eGFP_IRES_SEAP_Insert 1746-2335
GFP (SEQ ID NO: 52)
SEAP (SEQ ID NO: 53)
Firefly Luciferase (SEQ ID NO: 54)
FMDV 2A (SEQ ID NO: 55)
GPGPG linker (SEQ ID NO: 56)
chAd68-Empty-E4deleted (SEQ ID NO: 75): AC_000011.1 with E1 (nt 577 to 3403), E3 (nt 27,125- 31,825), and
E4 region (nt 34,916 to 35,642) sequences deleted and the corresponding ATCC VR-594 (Independently
sequenced Full-Length VR-594 C68 SEQ ID NO: 10) nucleotides substituted at five positions
NC_045512.2 Severe acute respiratory syndrome coronavirus 2 isolate Wuhan-Hu-1, complete genome (SEQ ID
NO: 76)

Table A

[symbol]

Refer to Sequence Listing, SEQ ID NOS. 130-8195. Presented is each candidate MHC Class I epitope encoded by SARS-CoV-2 that was predicted to associate with a given HLA allele with an EDGE score >0.001. Each entry includes the candidate epitope sequence and cognate HLA alleles with a predicted EDGE score greater than 0.001, with each cognate pairing ranked as H (EDGE score >0.1), M (EDGE score between 0.01 and 0.1), and L (EDGE score <0.01). For example, the candidate epitope MESLVPGF (SEQ ID NO: 127) is predicted to pair with HLA-B*18:01, HLA-B*37:01, and HLA-B*07:05 with EDGE scores 0.019, 0.032, and 0.008, respectively. Accordingly, the entry for SEQ ID NO: 130 is “MESLVPGF: B18:01M; B37:01M; B07:05L.”

Table B

[symbol]

Refer to Sequence Listing, SEQ ID NOS. 8196-26740. Presented is each candidate MHC Class II epitope encoded by SARS-CoV-2 that was predicted to associate with a given HLA allele with an EDGE score >0.001. Each entry includes the candidate epitope sequence and cognate HLA alleles with a predicted EDGE score greater than 0.001, with each cognate pairing ranked as H (EDGE score >0.1), M (EDGE score between 0.01 and 0.1), and L (EDGE score <0.01). For example, the candidate epitope VELVAELEGI (SEQ ID NO: 128) is predicted to pair with HLA-DQA1*03:02-B1*03:03, HLA-DRB1*11:02, HLA-DQA1*05:05-B1*03:19, and HLA-DPA1*01:03-B1*104:01 with EDGE scores 0.003145, 0.00328, 0.041097, and 0.011613, respectively. Accordingly, the entry for SEQ ID NO: 8219 is “VELVAELEGI: DQA1*03:02-B1*03:03L; DRB1*11:02L; DQA1*05:05-B1*03:19M; DPA1*01:03-B1*104:01M.” Only HLA-DQ and HLA-DP are referred to by their alpha and beta chains. HLA-DR is referred to only by its beta chain as the alpha chain is generally invariable in the human population, with HLA-DR peptide contact regions particularly invariant.

Table C

[symbol]

Refer to Sequence Listing, SEQ ID NOS. 26741-27179. Presented are additional MHC Class I epitopes, other than those from the Spike protein, encoded within the optimized cassette that were predicted to associate with a given HLA allele with an EDGE score >0.001. The additional epitopes were determined by calculating population coverage criteria P with all initial epitopes provided by the SARS-CoV-2 Spike protein (SEQ ID NO:59) split into 51 and S2 and applying the optimization algorithms described herein.

Table D

[symbol]

Refer to Sequence Listing, SEQ ID NOS. 27180-27495, for SARS-CoV-2 Spike overlapping peptide pools. Each entry includes the stimulatory peptide, SARS-CoV-2 protein source, peptide subpool information, and Table. For example, the stimulatory peptide MFVFLVLLPLVSSQC (SEQ ID NO: 27180) is derived from SARS-CoV-2 Spike protein (Wuhan D614G variant), included in subpool S_Wu_1_2, and found in Table D. Accordingly, the entry for SEQ ID NO. 27180 is “MFVFLVLLPLVSSQC: Spike Wuhan D614G; S_Wu_1_2; Table D”.

Table E

[symbol]

Refer to Sequence Listing, SEQ ID NOS. 27496-27603, for TCE5-encoded overlapping peptide pools. Each entry includes the stimulatory peptide, SARS-CoV-2 protein source, peptide subpool information, and Table. For example, the stimulatory peptide LLWPVTLACFVLAAV (SEQ ID NO: 27496) is derived from SARS-CoV-2 Membrane protein, included in subpool OLP_Mem, and found in Table E. Accordingly, the entry for SEQ ID NO. 27496 is “LLWPVTLACFVLAAV: Membrane; OLP_Mem; Table E”.

Table F

[symbol]

Refer to Sequence Listing, SEQ ID NOS. 27604-27939, for TCE5-encoded minimal epitope peptide pools. Each entry includes the stimulatory peptide, SARS-CoV-2 protein source, peptide subpool information, and Table. For example, the stimulatory peptide ALSKGVHFV (SEQ ID NO: 27604) is derived from SARS-CoV-2 ORF3a protein (frame 52-85), included in subpool Min_validated, and found in Table F. Accordingly, the entry for SEQ ID NO. 27604 is “ALSKGVHFV: ORF3a 52-85; Min_validated; Table F”.

### REFERENCES

1. Desrichard, A., Snyder, A. & Chan, T. A. Cancer Neoantigens and Applications for Immunotherapy. Clin. Cancer Res. Off. J. Am. Assoc. Cancer Res. (2015). doi:10.1158/1078-0432.CCR-14-3175
2. Schumacher, T. N. & Schreiber, R. D. Neoantigens in cancer immunotherapy. Science 348, 69-74 (2015).
3. Gubin, M. M., Artyomov, M. N., Mardis, E. R. & Schreiber, R. D. Tumor neoantigens: building a framework for personalized cancer immunotherapy. J. Clin. Invest. 125, 3413-3421 (2015).
4. Rizvi, N. A. et al. Cancer immunology. Mutational landscape determines sensitivity to PD-1 blockade in non-small cell lung cancer. Science 348, 124-128 (2015).
5. Snyder, A. et al. Genetic basis for clinical response to CTLA-4 blockade in melanoma. N. Engl. J. Med. 371, 2189-2199 (2014).
6. Carreno, B. M. et al. Cancer immunotherapy. A dendritic cell vaccine increases the breadth and diversity of melanoma neoantigen-specific T cells. Science 348, 803-808 (2015).
7. Tran, E. et al. Cancer immunotherapy based on mutation-specific CD4+ T cells in a patient with epithelial cancer. Science 344, 641-645 (2014).
8. Hacohen, N. & Wu, C. J.-Y. United States Patent Application: 20110293637-COMPOSITIONS AND METHODS OF IDENTIFYING TUMOR SPECIFIC NEOANTIGENS. (A1). at <http://appftl.uspto.gov/netacgi/nph-Pars er?Sect1=PTO1 &S ect2=HITOFF&d=PG0l&p=l&u=inetahtml/PTO/srchnum.html&r=l&f=G&1=50&s1=20110293637.PGNR.
9. Lundegaard, C., Hoof, I., Lund, O. & Nielsen, M. State of the art and challenges in sequence based T-cell epitope prediction. Immunome Res. 6 Suppl 2, S3 (2010).
10. Yadav, M. et al. Predicting immunogenic tumour mutations by combining mass spectrometry and exome sequencing. Nature 515, 572-576 (2014).
11. Bassani-Sternberg, M., Pletscher-Frankild, S., Jensen, L. J. & Mann, M. Mass spectrometry of human leukocyte antigen class I peptidomes reveals strong effects of protein abundance and turnover on antigen presentation. Mol. Cell. Proteomics MCP 14, 658-673 (2015).
12. Van Allen, E. M. et al. Genomic correlates of response to CTLA-4 blockade in metastatic melanoma. Science 350, 207-211 (2015).
13. Yoshida, K. & Ogawa, S. Splicing factor mutations and cancer. Wiley Interdiscip. Rev. RNA 5, 445-459 (2014).
14. Cancer Genome Atlas Research Network. Comprehensive molecular profiling of lung adenocarcinoma. Nature 511, 543-550 (2014).
15. Rajasagi, M. et al. Systematic identification of personal tumor-specific neoantigens in chronic lymphocytic leukemia. Blood 124, 453-462 (2014).
16. Downing, S. R. et al. U.S. patent application Ser. No. 01/202,08706-OPTIMIZATION OF MULTIGENE ANALYSIS OF TUMOR SAMPLES. (A1). at <http://appftl.uspto.gov/netacgi/nph-Parser?Sect1=PTO1&Sect2=HITOFF&d=PG0l&p=l&u=inetahtml/PTO/srchnum.html&r=l&f=G&1=50&s1=20120208706.PGNR.
17. Target Capture for NextGen Sequencing—IDT. at <http://www.idtdna.com/pages/products/nextgen/target-capture>
18. Shukla, S. A. et al. Comprehensive analysis of cancer-associated somatic mutations in class I HLA genes. Nat. Biotechnol. 33, 1152-1158 (2015).
19. Cieslik, M. et al. The use of exome capture RNA-seq for highly degraded RNA with application to clinical cancer sequencing. Genome Res. 25, 1372-1381 (2015).
20. Bodini, M. et al. The hidden genomic landscape of acute myeloid leukemia: subclonal structure revealed by undetected mutations. Blood 125, 600-605 (2015).
21. Saunders, C. T. et al. Strelka: accurate somatic small-variant calling from sequenced tumor-normal sample pairs. Bioinforma. Oxf. Engl. 28, 1811-1817 (2012).
22. Cibulskis, K. et al. Sensitive detection of somatic point mutations in impure and heterogeneous cancer samples. Nat. Biotechnol. 31, 213-219 (2013).
23. Wilkerson, M. D. et al. Integrated RNA and DNA sequencing improves mutation detection in low purity tumors. Nucleic Acids Res. 42, e107 (2014).
24. Mose, L. E., Wilkerson, M. D., Hayes, D. N., Perou, C. M. & Parker, J. S. ABRA: improved coding indel detection via assembly-based realignment. Bioinforma. Oxf. Engl. 30, 2813-2815 (2014).
25. Ye, K., Schulz, M. H., Long, Q., Apweiler, R. & Ning, Z. Pindel: a pattern growth approach to detect break points of large deletions and medium sized insertions from paired-end short reads. Bioinforma. Oxf. Engl. 25, 2865-2871 (2009).
26. Lam, H. Y. K. et al. Nucleotide-resolution analysis of structural variants using BreakSeq and a breakpoint library. Nat. Biotechnol. 28, 47-55 (2010).
27. Frampton, G. M. et al. Development and validation of a clinical cancer genomic profiling test based on massively parallel DNA sequencing. Nat. Biotechnol. 31, 1023-1031 (2013).
28. Boegel, S. et al. HLA typing from RNA-Seq sequence reads. Genome Med. 4, 102 (2012).
29. Liu, C. et al. ATHLATES: accurate typing of human leukocyte antigen through exome sequencing. Nucleic Acids Res. 41, e142 (2013).
30. Mayor, N. P. et al. HLA Typing for the Next Generation. PloS One 10, e0127153 (2015).
31. Roy, C. K., Olson, S., Graveley, B. R., Zamore, P. D. & Moore, M. J. Assessing long-distance RNA sequence connectivity via RNA-templated DNA-DNA ligation. eLife 4, (2015).
32. Song, L. & Florea, L. CLASS: constrained transcript assembly of RNA-seq reads. BMC Bioinformatics 14 Suppl 5, S14 (2013).
33. Maretty, L., Sibbesen, J. A. & Krogh, A. Bayesian transcriptome assembly. Genome Biol. 15, 501 (2014).
34. Pertea, M. et al. StringTie enables improved reconstruction of a transcriptome from RNA-seq reads. Nat. Biotechnol. 33, 290-295 (2015).
35. Roberts, A., Pimentel, H., Trapnell, C. & Pachter, L. Identification of novel transcripts in annotated genomes using RNA-Seq. Bioinforma. Oxf. Engl. (2011). doi:10.1093/bioinformatics/btr355
36. Vitting-Seerup, K., Porse, B. T., Sandelin, A. & Waage, J. spliceR: an R package for classification of alternative splicing and prediction of coding potential from RNA-seq data. BMC Bioinformatics 15, 81 (2014).
37. Rivas, M. A. et al. Human genomics. Effect of predicted protein-truncating genetic variants on the human transcriptome. Science 348, 666-669 (2015).
38. Skelly, D. A., Johansson, M., Madeoy, J., Wakefield, J. & Akey, J. M. A powerful and flexible statistical framework for testing hypotheses of allele-specific gene expression from RNA-seq data. Genome Res. 21, 1728-1737 (2011).
39. Anders, S., Pyl, P. T. & Huber, W. HTSeq—a Python framework to work with high-throughput sequencing data. Bioinforma. Oxf. Engl. 31, 166-169 (2015).
40. Furney, S. J. et al. SF3B1 mutations are associated with alternative splicing in uveal melanoma. Cancer Discov. (2013). doi:10.1158/2159-8290.CD-13-0330
41. Zhou, Q. et al. A chemical genetics approach for the functional assessment of novel cancer genes. Cancer Res. (2015). doi:10.1158/0008-5472.CAN-14-2930
42. Maguire, S. L. et al. SF3B1 mutations constitute a novel therapeutic target in breast cancer. J. Pathol. 235, 571-580 (2015).
43. Carithers, L. J. et al. A Novel Approach to High-Quality Postmortem Tissue Procurement: The GTEx Project. Biopreservation Biobanking 13, 311-319 (2015).
44. Xu, G. et al. RNA CoMPASS: a dual approach for pathogen and host transcriptome analysis of RNA-seq datasets. PloS One 9, e89445 (2014).
45. Andreatta, M. & Nielsen, M. Gapped sequence alignment using artificial neural networks: application to the MHC class I system. Bioinforma. Oxf. Engl. (2015). doi:10.1093/bioinformatics/btv639
46. Jørgensen, K. W., Rasmussen, M., Buus, S. & Nielsen, M. NetMHCstab—predicting stability of peptide-MHC-I complexes; impacts for cytotoxic T lymphocyte epitope discovery. Immunology 141, 18-26 (2014).
47. Larsen, M. V. et al. An integrative approach to CTL epitope prediction: a combined algorithm integrating MHC class I binding, TAP transport efficiency, and proteasomal cleavage predictions. Eur. J. Immunol. 35, 2295-2303 (2005).
48. Nielsen, M., Lundegaard, C., Lund, O. & Kemir, C. The role of the proteasome in generating cytotoxic T-cell epitopes: insights obtained from improved predictions of proteasomal cleavage. Immunogenetics 57, 33-41 (2005).
49. Boisvert, F.-M. et al. A Quantitative Spatial Proteomics Analysis of Proteome Turnover in Human Cells. Mol. Cell. Proteomics 11, M111.011429-M111.011429 (2012).
50. Duan, F. et al. Genomic and bioinformatic profiling of mutational neoepitopes reveals new rules to predict anticancer immunogenicity. J. Exp. Med. 211, 2231-2248 (2014).
51. Janeway's Immunobiology: 9780815345312: Medicine & Health Science Books @ Amazon.com. at <http://www.amazon.comaaneways-Immunobiology-Kenneth-Murphy/dp/0815345313>
52. Calis, J. J. A. et al. Properties of MHC Class I Presented Peptides That Enhance Immunogenicity. PLoS Comput. Biol. 9, e1003266 (2013).
53. Zhang, J. et al. Intratumor heterogeneity in localized lung adenocarcinomas delineated by multiregion sequencing. Science 346, 256-259 (2014)
54. Walter, M. J. et al. Clonal architecture of secondary acute myeloid leukemia. N. Engl. J. Med. 366, 1090-1098 (2012).
55. Hunt D F, Henderson R A, Shabanowitz J, Sakaguchi K, Michel H, Sevilir N, Cox A L, Appella E, Engelhard V H. Characterization of peptides bound to the class I MHC molecule HLA-A2.1 by mass spectrometry. Science 1992. 255: 1261-1263.
56. Zarling A L, Polefrone J M, Evans A M, Mikesh L M, Shabanowitz J, Lewis S T, Engelhard V H, Hunt D F. Identification of class I MHC-associated phosphopeptides as targets for cancer immunotherapy. Proc Natl Acad Sci USA. 2006 Oct. 3; 103(40):14889-94.
57. Bassani-Sternberg M, Pletscher-Frankild S, Jensen L J, Mann M. Mass spectrometry of human leukocyte antigen class I peptidomes reveals strong effects of protein abundance and turnover on antigen presentation. Mol Cell Proteomics. 2015 March; 14(3):658-73. doi: 10.1074/mcp.M114.042812.
58. Abelin J G, Trantham P D, Penny S A, Patterson A M, Ward S T, Hildebrand W H, Cobbold M, Bai D L, Shabanowitz J, Hunt D F. Complementary IMAC enrichment methods for HLA-associated phosphopeptide identification by mass spectrometry. Nat Protoc. 2015 September; 10(9):1308-18. doi: 10.1038/nprot.2015.086. Epub 2015 Aug. 6
59. Barnstable C J, Bodmer W F, Brown G, Galfre G, Milstein C, Williams A F, Ziegler A. Production of monoclonal antibodies to group A erythrocytes, HLA and other human cell surface antigens-new tools for genetic analysis. Cell. 1978 May; 14(1):9-20.
60. Goldman J M, Hibbin J, Kearney L, Orchard K, Th'ng K H. HLA-DR monoclonal antibodies inhibit the proliferation of normal and chronic granulocytic leukaemia myeloid progenitor cells. Br J Haematol. 1982 November; 52(3):411-20.
61. Eng J K, Jahan T A, Hoopmann M R. Comet: an open-source MS/MS sequence database search tool. Proteomics. 2013 January; 13(1):22-4. doi: 10.1002/pmic.201200439. Epub 2012 Dec. 4.
62. Eng J K, Hoopmann M R, Jahan T A, Egertson J D, Noble W S, MacCoss MJ. A deeper look into Comet—implementation and features. J Am Soc Mass Spectrom. 2015 November; 26(11):1865-74. doi: 10.1007/s13361-015-1179-x. Epub 2015 Jun. 27.
63. Lukas Käll, Jesse Canterbury, Jason Weston, William Stafford Noble and Michael J. MacCoss. Semi-supervised learning for peptide identification from shotgun proteomics datasets. Nature Methods 4:923-925, November 2007
64. Lukas Käll, John D. Storey, Michael J. MacCoss and William Stafford Noble. Assigning confidence measures to peptides identified by tandem mass spectrometry. Journal of Proteome Research, 7(1):29-34, January 2008
65. Lukas Käll, John D. Storey and William Stafford Noble. Nonparametric estimation of posterior error probabilities associated with peptides identified by tandem mass spectrometry. Bioinformatics, 24(16):i42-i48, August 2008
66. Kinney R M, BJ Johnson, VL Brown, D W Trent. Nucleotide Sequence of the 26 S mRNA of the Virulent Trinidad Donkey Strain of Venezuelan Equine Encephalitis Virus and Deduced Sequence of the Encoded Structural Proteins. Virology 152 (2), 400-413. 1986 Jul. 30.
67. Jill E Slansky, Frédérique M Rattis, Lisa F Boyd, Tarek Fahmy, Elizabeth M Jaffee, Jonathan P Schneck, David H Margulies, Drew M Pardoll. Enhanced Antigen-Specific Antitumor Immunity with Altered Peptide Ligands that Stabilize the MHC-Peptide-TCR Complex. Immunity, Volume 13, Issue 4, 1 Oct. 2000, Pages 529-538.
68. A Y Huang, P H Gulden, A S Woods, M C Thomas, C D Tong, W Wang, V H Engelhard, G Pasternack, R Cotter, D Hunt, D M Pardoll, and E M Jaffee. The immunodominant major histocompatibility complex class I-restricted antigen of a murine colon tumor derives from an endogenous retroviral gene product. Proc Natl Acad Sci USA.; 93(18): 9730-9735, 1996 Sep. 3.
69. JOHNSON, BARBARA J. B., RICHARD M. KINNEY, CRYSTLE L. KOST AND DENNIS W. TRENT. Molecular Determinants of Alphavirus Neurovirulence: Nucleotide and Deduced Protein Sequence Changes during Attenuation of Venezuelan Equine Encephalitis Virus. J Gen Virol 67:1951-1960, 1986.
70. Aarnoudse, C. A., Krüse, M., Konopitzky, R., Brouwenstijn, N., and Schrier, P. I. (2002). TCR reconstitution in Jurkat reporter cells facilitates the identification of novel tumor antigens by cDNA expression cloning. Int J Cancer 99, 7-13.
71. Alexander, J., Sidney, J., Southwood, S., Ruppert, J., Oseroff, C., Maewal, A., Snoke, K., Serra, H. M., Kubo, R. T., and Sette, A. (1994). Development of high potency universal DR-restricted helper epitopes by modification of high affinity DR-blocking peptides. Immunity 1, 751-761.
72. Banu, N., Chia, A., Ho, Z. Z., Garcia, A. T., Paravasivam, K., Grotenbreg, G. M., Bertoletti, A., and Gehring, A. J. (2014). Building and optimizing a virus-specific T cell receptor library for targeted immunotherapy in viral infections. Scientific Reports 4, 4166.
73. Cornet, S., Miconnet, I., Menez, J., Lemonnier, F., and Kosmatopoulos, K. (2006). Optimal organization of a polypeptide-based candidate cancer vaccine composed of cryptic tumor peptides with enhanced immunogenicity. Vaccine 24, 2102-2109.
74. Depla, E., van der Aa, A., Livingston, B. D., Crimi, C., Allosery, K., de Brabandere, V., Krakover, J., Murthy, S., Huang, M., Power, S., et al. (2008). Rational design of a multiepitope vaccine encoding T-lymphocyte epitopes for treatment of chronic hepatitis B virus infections. Journal of Virology 82, 435-450.
75. Ishioka, G. Y., Fikes, J., Hermanson, G., Livingston, B., Crimi, C., Qin, M., del Guercio, M. F., Oseroff, C., Dahlberg, C., Alexander, J., et al. (1999). Utilization of MHC class I transgenic mice for development of minigene DNA vaccines encoding multiple HLA-restricted CTL epitopes. J Immunol 162, 3915-3925.
76. Janetzki, S., Price, L., Schroeder, H., Britten, C. M., Welters, M. J. P., and Hoos, A. (2015). Guidelines for the automated evaluation of Elispot assays. Nat Protoc 10, 1098-1115.
77. Lyons, G. E., Moore, T., Brasic, N., Li, M., Roszkowski, J. J., and Nishimura, M. I. (2006). Influence of human CD8 on antigen recognition by T-cell receptor-transduced cells. Cancer Res 66, 11455-11461.
78. Nagai, K., Ochi, T., Fujiwara, H., An, J., Shirakata, T., Mineno, J., Kuzushima, K., Shiku, H., Melenhorst, J. J., Gostick, E., et al. (2012). Aurora kinase A-specific T-cell receptor gene transfer redirects T lymphocytes to display effective antileukemia reactivity. Blood 119, 368-376.
79. Panina-Bordignon, P., Tan, A., Termijtelen, A., Demotz, S., Corradin, G., and Lanzavecchia, A. (1989). Universally immunogenic T cell epitopes: promiscuous binding to human MHC class II and promiscuous recognition by T cells. Eur J Immunol 19, 2237-2242.
80. Vitiello, A., Marchesini, D., Furze, J., Sherman, L. A., and Chesnut, R. W. (1991). Analysis of the HLA-restricted influenza-specific cytotoxic T lymphocyte response in transgenic mice carrying a chimeric human-mouse class I major histocompatibility complex. J Exp Med 173, 1007-1015.
81. Yachi, P. P., Ampudia, J., Zal, T., and Gascoigne, N. R. J. (2006). Altered peptide ligands induce delayed CD8-T cell receptor interaction—a role for CD8 in distinguishing antigen quality. Immunity 25, 203-211.
82. Pushko P, Parker M, Ludwig G V, Davis N L, Johnston R E, Smith J F. Replicon-helper systems from attenuated Venezuelan equine encephalitis virus: expression of heterologous genes in vitro and immunization against heterologous pathogens in vivo. Virology. 1997 Dec. 22; 239(2):389-401.
83. Strauss, JH and E G Strauss. The alphaviruses: gene expression, replication, and evolution. Microbiol Rev. 1994 September; 58(3): 491-562.
84. Rheme C, Ehrengruber M U, Grandgirard D. Alphaviral cytotoxicity and its implication in vector development. Exp Physiol. 2005 January; 90(1):45-52. Epub 2004 Nov. 12.
85. Riley, Michael K. II, and Wilfred Vermerris. Recent Advances in Nanomaterials for Gene Delivery-A Review. Nanomaterials 2017, 7(5), 94.
86. Frolov I, Hardy R, Rice C M. Cis-acting RNA elements at the 5′ end of Sindbis virus genome RNA regulate minus- and plus-strand RNA synthesis. RNA. 2001 November; 7(11):1638-51.
87. Jose J, Snyder J E, Kuhn R J. A structural and functional perspective of alphavirus replication and assembly. Future Microbiol. 2009 September; 4(7):837-56.
88. Bo Li and C. olin N. Dewey. RSEM: accurate transcript quantification from RNA-Seq data with or without a referenfe genome. BMC Bioinformatics, 12:323, August 2011
89. Hillary Pearson, Tariq Daouda, Diana Paola Granados, Chantal Durette, Eric Bonneil, Mathieu Courcelles, Anja Rodenbrock, Jean-Philippe Laverdure, Caroline Cote, Sylvie Mader, Sebastien Lemieux, Pierre Thibault, and Claude Perreault. MHC class I-associated peptides derive from selective regions of the human genome. The Journal of Clinical Investigation, 2016,
90. Juliane Liepe, Fabio Marino, John Sidney, Anita Jeko, Daniel E. Bunting, Alessandro Sette, Peter M. Kloetzel, Michael P. H. Stumpf, Albert J. R. Heck, Michele Mishto. A large fraction of HLA class I ligands are proteasome-generated spliced peptides. Science, 21, October 2016.
91. Mommen G P., Marino, F., Meiring H D., Poelen, MC., van Gaans-van den Brink, JA., Mohammed S., Heck A J., and van Els C A. Sampling From the Proteome to the Human Leukocyte Antigen-DR (HLA-DR) Ligandome Proceeds Via High Specificity. Mol Cell Proteomics 15(4): 1412-1423, April 2016.
92. Sebastian Kreiter, Mathias Vormehr, Niels van de Roemer, Mustafa Diken, Martin Löwer, Jan Diekmann, Sebastian Boegel, Barbara Schrörs, Fulvia Vascotto, John C. Castle, Arbel D. Tadmor, Stephen P. Schoenberger, Christoph Huber, Özlem Tiireci, and Ugur Sahin. Mutant MHC class II epitopes drive therapeutic immune responses to caner. Nature 520, 692-696, April 2015.
93. Tran E., Turcotte S., Gros A., Robbins P. F., Lu Y. C., Dudley M. E., Wunderlich J. R., Somerville R. P., Hogan K., Hinrichs C. S., Parkhurst M. R., Yang J. C., Rosenberg S. A. Cancer immunotherapy based on mutation-specific CD4+ T cells in a patient with epithelial cancer. Science 344(6184) 641-645, May 2014.
94. Andreatta M., Karosiene E., Rasmussen M., Stryhn A., Buus S., Nielsen M. Accurate pan-specific prediction of peptide-MHC class II binding affinity with improved binding core identification. Immunogenetics 67(11-12) 641-650, November 2015.
95. Nielsen, M., Lund, 0. NN-align. An artificial neural network-based alignment algorithm for MHC class II peptide binding prediction. BMC Bioinformatics 10:296, September 2009.
96. Nielsen, M., Lundegaard, C., Lund, O. Prediction of MHC class II binding affinity using SMM-align, a novel stabilization matrix alignment method. BMC Bioinformatics 8:238, July 2007.
97. Zhang, J., et al. PEAKS DB: de novo sequencing assisted database search for sensitive and accurate peptide identification. Molecular & Cellular Proteomics. 11(4):1-8. 1/2/2012.
98. Jensen, Kamilla Kjaergaard, et al. “Improved Methods for Prediting Peptide Binding Affinity to MHC Class II Molecules.” Immunology, 2018, doi:10.1111/imm.12889.
99. Carter, S. L., Cibulskis, K., Heiman, E., McKenna, A., Shen, H., Zack, T., Laird, P. W., Onofrio, R. C., Winckler, W., Weir, B. A., et al. (2012). Absolute quantification of somatic DNA alterations in human cancer. Nat. Biotechnol. 30, 413-421
100. McGranahan, N., Rosenthal, R., Hiley, C. T., Rowan, A. J., Watkins, T. B. K., Wilson, G. A., Birkbak, N.J., Veeriah, S., Van Loo, P., Herrero, J., et al. (2017). Allele-Specific HLA Loss and Immune Escape in Lung Cancer Evolution. Cell 171, 1259-1271.ell.
101. Shukla, S. A., Rooney, M. S., Rajasagi, M., Tiao, G., Dixon, P. M., Lawrence, M. S., Stevens, J., Lane, W. J., Dellagatta, J. L., Steelman, S., et al. (2015). Comprehensive analysis of cancer-associated somatic mutations in class I HLA genes. Nat. Biotechnol. 33, 1152-1158.
102. Van Loo, P., Nordgard, S. H., Lingjerde, O. C., Russnes, H. G., Rye, I. H., Sun, W., Weigman, V. J., Marynen, P., Zetterberg, A., Naume, B., et al. (2010). Allele-specific copy number analysis of tumors. Proc. Natl. Acad. Sci. U.S.A 107, 16910-16915.
103. Van Loo, P., Nordgard, S. H., Lingjerde, O. C., Russnes, H. G., Rye, I. H., Sun, W., Weigman, V. J., Marynen, P., Zetterberg, A., Naume, B., et al. (2010). Allele-specific copy number analysis of tumors. Proc. Natl. Acad. Sci. U.S.A 107, 16910-16915.

1. A composition for delivery of an antigen expression system comprising a self-replicating alphavirus-based expression system, wherein the composition for delivery of the self-replicating alphavirus-based expression system comprises: the self-replicating alphavirus-based expression system, wherein the self-replicating alphavirus-based expression system comprises one or more vectors, wherein the one or more vectors comprise:
(a) an RNA alphavirus backbone, wherein the RNA alphavirus backbone comprises:
(i) at least one promoter nucleotide sequence, and
(ii) at least one polyadenylation (poly(A)) sequence; and
(b) at least two cassettes, wherein each of the cassettes independently comprise:
(i) at least one antigen-encoding nucleic acid sequence comprising:
a. an epitope-encoding nucleic acid sequence,
b. optionally a 5′ linker sequence, and
c. optionally a 3′ linker sequence; and
(ii) optionally, at least one second poly(A) sequence, wherein the second poly(A) sequence is a native poly(A) sequence or an exogenous poly(A) sequence to the alphavirus,
wherein a first of the at least two cassettes, oriented from 5′ to 3′, is operably linked to a promoter nucleotide sequence comprising a first subgenomic alphavirus-derived promoter (SGP1) comprising a core conserved promoter sequence comprising the polynucleotide sequence ctacggcTAAcctgaa(+1)tgga (SEQ ID NO: 27974), and wherein at least a second of the at least two cassettes is operably linked to a promoter nucleotide sequence comprising a second subgenomic alphavirus-derived promoter (SGP2) comprising the core conserved promoter sequence, and
wherein the SGP1 and/or the SGP2 subgenomic promoter comprises an extended 3′ promoter region derived from an alphavirus encoded 3′ of the core conserved promoter sequence.

2. The composition of claim 1, wherein the extended 3′ promoter region of SGP1 is different than the extended 3′ promoter region of SGP2.

3. The composition of claim 1, wherein either the SGP1 or the SGP2 subgenomic promoter, but not both, comprises an extended 3′ promoter region derived from an alphavirus encoded 3′ of the core conserved promoter sequence.

4. The composition of claim 1, wherein the extended 3′ promoter region:
(a) comprises the polynucleotide sequence CTACGACAT (SEQ ID NO: 27997);
(b) comprises the polynucleotide sequence CTACGACATAGTCTAGTCCGCCAAG (SEQ ID NO: 27975);
(c) consists of the polynucleotide sequence CTACGACAT (SEQ ID NO: 27997); or
(d) consists of the polynucleotide sequence CTACGACATAGTCTAGTCCGCCAAG (SEQ ID NO: 27975).

5.-7. (canceled)

8. The composition of claim 1, wherein the extended 3′ promoter region comprises the polynucleotide sequence ATAGTCTAGTCCGCCAAG (SEQ ID NO: 27976).

9. The composition of claim 1, wherein (a) each of the subgenomic promoters comprise an extended 3′ promoter region comprising the polynucleotide sequence CTACGACAT (SEQ ID NO: 27997); and (b) only one of the SGP1 or the SGP2 subgenomic promoters, but not both, comprise an extended 3′ promoter region further comprising the polynucleotide sequence ATAGTCTAGTCCGCCAAG (SEQ ID NO: 27976), wherein the polynucleotide sequence ATAGTCTAGTCCGCCAAG (SEQ ID NO: 27976) is encoded 3′ of the polynucleotide sequence CTACGACAT (SEQ ID NO: 27997).

10. The composition of claim 1, wherein the SGP1 subgenomic promoter, the SGP2 subgenomic promoter, or both comprise an extended 5′ promoter region derived from an alphavirus encoded 5′ of the core conserved promoter sequence, optionally wherein the extended 5′ promoter region is encoded immediately 5′ of the core conserved promoter sequence.

11. The composition of claim 10, wherein the extended 5′ promoter region:
(a) comprises a polynucleotide sequence derived from an alphavirus nonstructural protein 4 (nsp4) and is encoded 5′ of the core conserved promoter sequence;
(b) comprises the polynucleotide sequence ctct encoded immediately 5′ of the core conserved promoter sequence;
(c) comprises the polynucleotide sequence acttccatcatagttatggccatgactactctagctagcagtgttaaatcattcagctacctgagaggggcccctataactct (SEQ ID NO: 27977) encoded immediately 5′ of the core conserved promoter sequence;
(d) comprises the polynucleotide sequence acctgagaggggcccctataactct (SEQ ID NO: 27978) encoded immediately 5′ of the core conserved promoter sequence;
(e) comprises the polynucleotide sequence gggcccctataactct (SEQ ID NO: 27979) encoded immediately 5′ of the core conserved promoter sequence; or
(f) consists of the polynucleotide sequence gggcccctataactct (SEQ ID NO: 27979) encoded immediately 5′ of the core conserved promoter sequence.

12.-17. (canceled)

18. The composition of claim 10, wherein the extended 5′ promoter region of the SGP2 subgenomic promoter:
(a) comprises the polynucleotide sequence acttccatcatagttatggccatgactactctagctagcagtgttaaatcattcagctacctgagaggggcccctataactct (SEQ ID NO: 27977) encoded immediately 5′ of the core conserved promoter sequence;
(b) comprises the polynucleotide sequence acctgagaggggcccctataactct (SEQ ID NO: 27978) encoded immediately 5′ of the core conserved promoter sequence;
(c) comprises the polynucleotide sequence gggcccctataactct (SEQ ID NO: 27979) encoded immediately 5′ of the core conserved promoter sequence; or
(d) consists of the polynucleotide sequence gggcccctataactct (SEQ ID NO: 27979) encoded immediately 5′ of the core conserved promoter sequence.

19.-21. (canceled)

22. The composition of claim 1, wherein the at least one promoter nucleotide sequence of the RNA alphavirus backbone comprises the SGP1 subgenomic promoter.

23. The composition of claim 1, wherein the extended 3′ promoter region and/or the extended 5′ promoter region:
(a) is derived from the same alphavirus as the alphavirus used to derive the core conserved promoter sequence;
(b) is capable of reducing or eliminating recombination between the SGP1 and the SGP2 subgenomic promoter; and/or
(c) comprises one or more transcriptional enhancer elements.

24.-25. (canceled)

26. The composition of claim 1, wherein the SGP2 subgenomic promoter is capable of:
(a) promoting expression of a cassette at least 2-fold greater relative to the same cassette operably linked to the SGP1 subgenomic promoter; and/or
(b) stimulating a stronger immune response to epitopes encoded by the cassette operably linked to the SGP2 subgenomic promoter following administration to a subject relative to the same cassette operably linked to the SGP1 subgenomic promoter.

27. The composition of claim 1, wherein the extended 3′ promoter region and/or the extended 5′ promoter region of SGP2 is capable of:
(a) promoting expression of a cassette at least 2-fold greater relative to the same cassette operably linked to the SGP1 subgenomic promoter; and/or
(b) stimulating a stronger immune response to epitopes encoded by the cassette operably linked to the SGP2 subgenomic promoter following administration to a subject relative to the same cassette operably linked to the SGP1 subgenomic promoter.

28.-29. (canceled)

30. The composition of claim 1, wherein the SGP2 subgenomic promoter is encoded immediately 5′ of the second cassette, optionally immediately 5′ of a Kozak sequence of the second cassette.

31. The composition of claim 1, wherein either the SGP1 or the SGP2 subgenomic promoter comprises the polynucleotide sequence GGGCCCCTATAACTCTCTACGGCTAACCTGAATGGACTACGACATAGTCTAGTC CGCCAAG (SEQ ID NO: 27980);
(b) the SGP2 subgenomic promoter comprises the polynucleotide sequence GGGCCCCTATAACTCTCTACGGCTAACCTGAATGGACTACGACATAGTCTAGTC CGCCAAG (SEQ ID NO: 27980);
(c) the SGP1 subgenomic promoter comprises the polynucleotide sequence GGGCCCCTATAACTCTCTACGGCTAACCTGAATGGACTACGAC (SEQ ID NO: 27981);
(d) either the SGP1 or the SGP2 subgenomic promoter comprises the polynucleotide sequence GGGCCCCTATAACTCTCTACGGCTAACCTGAATGGACTACGACATAGTCTAGTC CGCCAAG (SEQ ID NO: 27980), and the other subgenomic promoter comprises the polynucleotide sequence GGGCCCCTATAACTCTCTACGGCTAACCTGAATGGACTACGAC (SEQ ID NO: 27981) but does not comprise the polynucleotide sequence ATAGTCTAGTCCGCCAAG (SEQ ID NO: 27976); or
(e) the SGP2 subgenomic promoter comprises the polynucleotide sequence GGGCCCCTATAACTCTCTACGGCTAACCTGAATGGACTACGAC (SEQ ID NO: 27981) and the SGP1 subgenomic promoter comprises the polynucleotide sequence GGGCCCCTATAACTCTCTACGGCTAACCTGAATGGACTACGAC (SEQ ID NO: 27981), wherein the SGP1 subgenomic promoter does not comprise the polynucleotide sequence ATAGTCTAGTCCGCCAAG (SEQ ID NO: 27976).

32.-39. (canceled)

40. The composition of claim 1, wherein the epitope-encoding nucleic acid sequence comprises:
at least one alteration that makes the encoded epitope sequence distinct from the corresponding peptide sequence encoded by a wild-type nucleic acid sequence;
a nucleic acid sequence encoding an infectious disease organism peptide selected from the group consisting of: a pathogen-derived peptide, a virus-derived peptide, a bacteria-derived peptide, a fungus-derived peptide, and a parasite-derived peptide, and optionally wherein the epitope-encoding nucleic acid sequence encodes a MHC class I or MHC class II epitope; or
combinations thereof.

41. The composition of claim 1, the composition further comprising a nanoparticulate delivery vehicle.

42.-48. (canceled)

49. The composition of claim 1, wherein the backbone comprises at least sequences for nonstructural protein-mediated amplification and a poly(A) sequence encoded by the nucleotide sequence of a Venezuelan equine encephalitis virus, wherein sequences for nonstructural protein-mediated amplification are selected from the group consisting of: an alphavirus 5′ UTR, a 51-nt CSE, a 24-nt CSE, a 19-nt CSE, an alphavirus 3′ UTR, a 26S subgenomic promoter sequence, optionally wherein the 26S subgenomic promoter sequence comprises the SGP1 and/or the SGP2 subgenomic promoter sequence, or combinations thereof, and/or wherein the backbone does not encode structural virion proteins capsid, E2 and E1.

50.-53. (canceled)

54. The composition of claim 1, wherein the alphavirus comprises the sequence of SEQ ID NO:3 or SEQ ID NO:5 further comprising a deletion between base pair 7544 and 11176, wherein the cassettes are inserted at position 7544 to replace the deletion between base pairs 7544 and 11176 as set forth in the sequence of SEQ ID NO:3 or SEQ ID NO:5.

55.-56. (canceled)

57. The composition of claim 1, wherein one or more of the cassettes are at least 100, 200, 300, 400, 500, 600, 700, 800, 900, 1000, 2000, 3000, 4000, 5000, 6000, 7000, 8000, 9000, or 10000 nucleotides in length.

58. (canceled)

59. The composition of claim 1, wherein the one or more vectors are capable of driving expression of a cassette that is at least 3500 nucleotides in length or 6000 nucleotides in length.

60. (canceled)

61. The composition of claim 1, wherein at least one of the at least one antigen-encoding nucleic acid sequences comprises an epitope-encoding nucleic acid sequence that encodes:
(a) an epitope that is presented by MHC class I;
(b) an epitope that is presented by MHC class II; and/or
(c) a polypeptide sequence or portion thereof capable of stimulating a B cell response, optionally wherein the polypeptide sequence or portion thereof capable of stimulating a B cell response comprises a full-length protein, a protein domain, a protein subunit, or an antigenic fragment predicted or known to be capable of being bound by an antibody.

62.-63. (canceled)

64. The composition of claim 1, wherein the at least one antigen-encoding nucleic acid sequence comprises two or more antigen-encoding nucleic acid sequences.

65. (canceled)

66. The composition of claim 64, wherein each antigen-encoding nucleic acid sequence is linked to a distinct antigen-encoding nucleic acid sequence with a nucleic acid sequence encoding a linker, optionally wherein the linker comprises one or more native sequences flanking the antigen derived from the cognate protein of origin and that is at least 2, 3, 4, 5, 6, 7, 8, 9, 10, 11, 12, 13, 14, 15, 16, 17, 18, 19, 20, or 2-20 amino acid residues in length.

67. (canceled)

68. The composition of claim 1, wherein the at least one antigen-encoding nucleic acid sequence comprises at least 2-10, 2, 3, 4, 5, 6, 7, 8, 9, 10, 11-20, 15-20, 11-100, 11-200, 11-300, 11-400, 11, 12, 13, 14, 15, 16, 17, 18, 19, 20 or up to 400 nucleic acid sequences.

69.-74. (canceled)

75. The composition of claim 1, wherein each MHC class I epitope-encoding nucleic acid sequence encodes a polypeptide sequence between 8 and 35 amino acids in length, optionally 9-17, 9-25, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids in length.

76.-101. (canceled)

102. A method for treating a subject, the method comprising administering to the subject a composition for delivery of an antigen expression system comprising a self-replicating alphavirus-based expression system, wherein the composition for delivery of the self-replicating alphavirus-based expression system comprises:
the self-replicating alphavirus-based expression system, wherein the self-replicating alphavirus-based expression system comprises one or more vectors, wherein the one or more vectors comprise:
(a) an RNA alphavirus backbone, wherein the RNA alphavirus backbone comprises:
(i) at least one promoter nucleotide sequence, and
(ii) at least one polyadenylation (poly(A)) sequence; and
(b) at least two cassettes, wherein each of the cassettes independently comprise:
(i) at least one antigen-encoding nucleic acid sequence comprising:
a. an epitope-encoding nucleic acid sequence,
b. optionally a 5′ linker sequence, and
c. optionally a 3′ linker sequence; and
(ii) optionally, at least one second poly(A) sequence, wherein the second poly(A) sequence is a native poly(A) sequence or an exogenous poly(A) sequence to the alphavirus,
wherein a first of the at least two cassettes, oriented from 5′ to 3′, is operably linked to a promoter nucleotide sequence comprising a first subgenomic alphavirus-derived promoter (SGP1) comprising a core conserved promoter sequence comprising the polynucleotide sequence ctacggcTAAcctgaa(+1)tgga, and wherein at least a second of the at least two cassettes is operably linked to a promoter nucleotide sequence comprising a second subgenomic alphavirus-derived promoter (SGP2) comprising the core conserved promoter sequence, and
wherein the SGP1 and/or the SGP2 subgenomic promoter comprises an extended 3′ promoter region derived from an alphavirus encoded 3′ of the core conserved promoter sequence.

103. (canceled)

104. The method of claim 102, wherein the subject expresses at least one HLA allele predicted or known to present a MHC class I or MHC class II epitope encoded by the epitope-encoding nucleic acid sequence of the at least one antigen-encoding nucleic acid sequence.

105. A composition for delivery of a payload comprising a self-replicating alphavirus-based expression system, wherein the composition for delivery of the self-replicating alphavirus-based expression system comprises:
the self-replicating alphavirus-based expression system, wherein the self-replicating alphavirus-based expression system comprises one or more vectors, wherein the one or more vectors comprise:
(a) an RNA alphavirus backbone, wherein the RNA alphavirus backbone comprises:
(i) at least one promoter nucleotide sequence, and
(ii) at least one polyadenylation (poly(A)) sequence; and
(b) at least two cassettes, wherein each of the cassettes independently comprise:
(i) at least one payload-encoding nucleic acid sequence; and
(ii) optionally, at least one second poly(A) sequence, wherein the second poly(A) sequence is a native poly(A) sequence or an exogenous poly(A) sequence to the alphavirus,
wherein a first of the at least two cassettes, oriented from 5′ to 3′, is operably linked to a promoter nucleotide sequence comprising a first subgenomic alphavirus-derived promoter (SGP1) comprising a core conserved promoter sequence comprising the polynucleotide sequence ctacggcTAAcctgaa(+1)tgga (SEQ ID NO: 27974), and wherein at least a second of the at least two cassettes is operably linked to a promoter nucleotide sequence comprising a second subgenomic alphavirus-derived promoter (SGP2) comprising the core conserved promoter sequence, and
wherein the SGP1 and/or the SGP2 subgenomic promoter comprises an extended 3′ promoter region derived from an alphavirus encoded 3′ of the core conserved promoter sequence.
